(19) United States
(12) Patent Application Publication
(10) Pub. No.: US 2025/0221953 A1
(43) Pub. Date: Jul. 10, 2025

(54) TRANS-VACCENIC ACID (TVA) AND DERIVATIVES THEREOF IN T CELL-BASED CANCER THERAPIES

(71) Applicant: The University of Chicago, Chicago, IL (US)

(72) Inventor: Jing CHEN, Chicago, IL (US)

(21) Appl. No.: 18/852,916

(22) PCT Filed: Mar. 30, 2023
(86) PCT No.: PCT/US2023/065156

§ 371 Date: Sep. 30, 2024

Related U.S. Application Data
(60) Provisional application No. 63/325,456, filed on Mar. 30, 2022.

(51) Int. Cl. A61K 31/201; A61K 31/198; A61K 31/202; A61K 31/397; A61K 39/395; A61K 40/11; A61K 40/31; A61K 40/33; C12N 15/113

(52) U.S. Cl. CPC A61K 31/201; A61K 31/198; A61K 31/202; A61K 31/397; A61K 39/3955; A61K 40/11; A61K 40/31; A61K 40/33; C12N 15/1138; C12N 2310/11; C12N 2310/14; C12N 2310/531

(57) ABSTRACT

Provided herein are compositions and methods for stimulating T-cell-based anti-tumor immunity by administration of trans-vaccenic acid (TVA), an active derivative thereof, or an inhibitor of GPR43 expression or activity. In particular embodiments, TVA, a TVA derivative, or an inhibitor of GPR43 expression or activity is administered to boost an endogenous T-cell response and/or is co-administered with a T-cell-based therapy, such as immune checkpoint blockade therapies, CAR-T therapies, monoclonal antibody therapies, bispecific T-cell engagers therapies, etc.

### CROSS-REFERENCE TO RELATED APPLICATIONS

This application claims the benefit of U.S. Provisional Patent Application No. 63/325,456, filed on Mar. 30, 2022, which is incorporated by reference herein.

### INCORPORATION-BY-REFERENCE OF MATERIAL SUBMITTED ELECTRONICALLY

Incorporated by reference in its entirety herein is a computer-readable nucleotide sequence listing submitted concurrently herewith and identified as follows: 39,914 Byte ASCII (XML) file named “39942_601_SequenceListing,” created on Mar. 30, 2023.

### FIELD

Provided herein are compositions and methods for stimulating T-cell-based anti-tumor immunity by administration of trans-vaccenic acid (TVA), an active derivative thereof, or an inhibitor of GPR43 expression or activity. In particular embodiments, TVA, a TVA derivative, or an inhibitor of GPR43 expression or activity is administered to boost an endogenous T-cell response and/or is co-administered with a T-cell-based therapy, such as immune checkpoint blockade therapies, CAR-T therapies, monoclonal antibody therapies, bispecific T-cell engagers therapies, etc.

### BACKGROUND

Diet-derived blood chemicals have been inextricably linked to human physiology during evolution, which not only provide energy and precursors for biosynthesis for growth but also function as signaling molecules. Despite extensive studies on links between diet and cancer, little is known about how the circulating diet-derived substances affect specific human cellular functions. What is needed are metabolites and methods of use thereof that promote the prevention of the cancer progression and/or are useful in facilitating the treatment of established cancers.

### SUMMARY

Provided herein are compositions and methods for stimulating T-cell-based anti-tumor immunity by administration of trans-vaccenic acid (TVA), an active derivative thereof, or an inhibitor of GPR43 expression or activity. In particular embodiments, TVA, a TVA derivative, or an inhibitor of GPR43 expression or activity is administered to boost an endogenous T-cell response and/or is co-administered with a T-cell-based therapy, such as immune checkpoint blockade therapies, CAR-T therapies, monoclonal antibody therapies, bispecific T-cell engagers therapies, etc.

In some embodiments, provided herein are methods of treating cancer in a subject in need thereof comprising administering an effective amount of trans-vaccenic acid (TVA) or an active TVA derivative to the subject. In some embodiments, the active TVA derivative is capable of enhancing CD8+ T cell activity and/or enhancing antitumor immunity. In some embodiments, the TVA or active TVA derivative is a compound of formula (I):

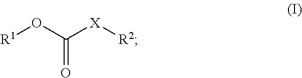

wherein: R¹ is selected from hydrogen and C₁-C₄ alkyl; X is selected from C₁-C₂₄ alkylene, C₂-C₂₄ alkenylene, and C₂-C₂₄ alkynylene; and R² is selected from hydrogen, optionally substituted aryl, optionally substituted heteroaryl, C₁-C₄ alkoxy, hydroxy, and halo. In some embodiments, R¹ is selected from hydrogen and methyl. In some embodiments, R¹ is hydrogen. In some embodiments, X is selected from C₈-C₂₄ alkylene, C₈-C₂₄ alkenylene, and C₈-C₂₄ alkynylene. In some embodiments, X is selected from C₁₂-C₂₄ alkylene, C₁₂-C₂₄ alkenylene, and C₁₂-C₂₄ alkynylene. In some embodiments, X is C₁₂-C₂₄ alkylene. In some embodiments, X is C₁₂-C₂₄ alkenylene. In some embodiments, X is C₁₂-C₂₄ alkynylene. In some embodiments, R² is selected from hydrogen and optionally substituted aryl. In some embodiments, R² is selected from hydrogen and phenyl, wherein the phenyl is unsubstituted or substituted with one substituent selected from C₁-C₄ alkyl (e.g., methyl). In some embodiments, R² is hydrogen. In some embodiments, the compound is selected from:

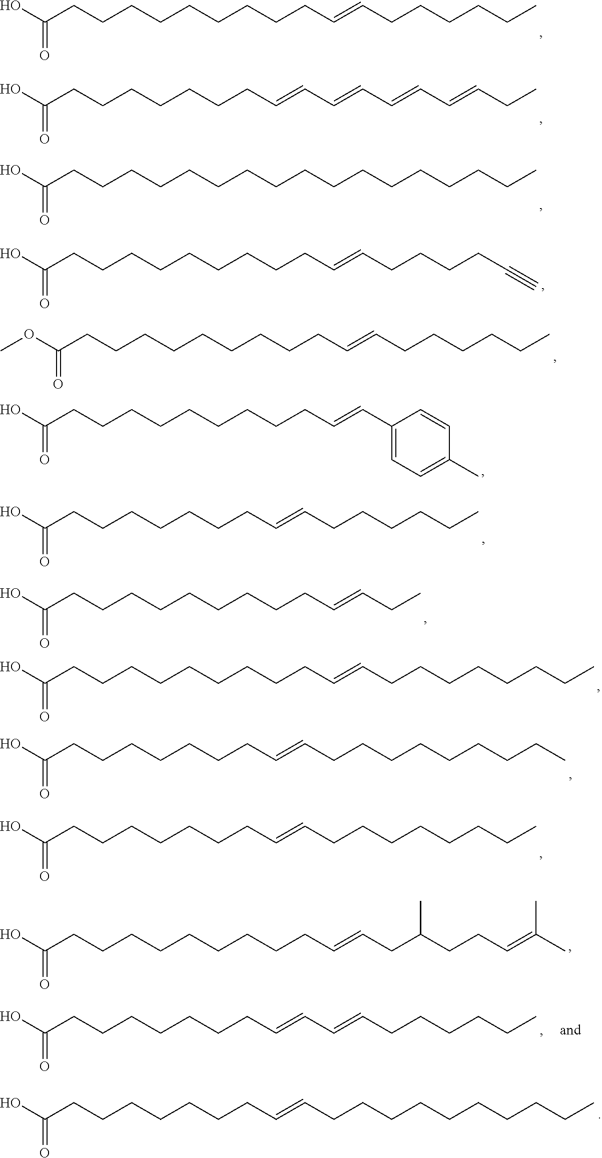

In some embodiments, TVA is administered. In some embodiments, TVA, an active TVA derivative, or an inhibitor of GPR43 expression or activity is administered orally, intravenously, transdermally, or subcutaneously. In some embodiments, TVA, an active TVA derivative, or an inhibitor of GPR43 expression or activity is administered once or more weekly. In some embodiments, TVA, an active TVA derivative, or an inhibitor of GPR43 expression or activity is administered once or more daily. In some embodiments, the cancer is melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, the subject is a human.

In some embodiments, TVA, an active TVA derivative, or an inhibitor of GPR43 expression or activity is co-administered with one or more additional therapeutic and/or prophylactic agents. In some embodiments, the one or more additional therapeutic agents arc selected from chemotherapeutics and immunotherapeutics. In some embodiments, TVA, an active TVA derivative, or an inhibitor of GPR43 expression or activity is co-administered with a T-cell-based immunotherapeutic. In some embodiments, the T-cell-based immunotherapeutic is selected from a therapy comprising the administration of immune checkpoint inhibitor, CAR-T cell therapy, monoclonal antibody therapy, and bispecific T-cell engager therapy. In some embodiments, the TVA, an active TVA derivative, or an inhibitor of GPR43 expression or activity is co-administered with an immune checkpoint inhibitor that binds to and inhibits the activity of an immune checkpoint protein is selected from the group consisting of CTLA4, PD-1, PD-L1, PD-L2, A2AR, B7-H3, B7-H4, BTLA, KIR, LAG3, TIM-3 or VISTA. In some embodiments, the TVA is co-administered with an immune checkpoint inhibitor selected from the group consisting of nivolumab, pembrolizumab, pidilizumab, AMP-224, AMP-514, STI-A1110, TSR-042, RG-7446, BMS-936559, BMS-936558, MK-3475, MPDL3280A, MEDI-4736, MSB-0020718C, AUR-012 and STI-A1010.

In some embodiments, provided herein is the use of an effective dose of trans-vaccenic acid (TVA) or an active TVA derivative for treating a subject suffering from cancer. In some embodiments, TVA is administered. In some embodiments, the TVA, an active TVA derivative, or an inhibitor of GPR43 expression or activity is co-administered with an immunotherapeutic selected from a therapy comprising the administration of immune checkpoint inhibitor, CAR-T cell therapy, monoclonal antibody therapy, and bispecific T-cell engager therapy.

In some embodiments, provided herein is the use of trans-vaccenic acid (TVA) or an active TVA derivative in the manufacture of a medicament for use in a method of treating a subject suffering from cancer. In some embodiments, the method further comprises an immune checkpoint inhibitor, CAR-T cell therapy, monoclonal antibody therapy, and bispecific T-cell engager therapy.

In some embodiments, provided herein are compositions or kits comprising trans-vaccenic acid (TVA) or an active TVA derivative and an additional therapeutic and/or prophylactic agent for the treatment of cancer. In some embodiments, the active TVA derivative is capable of enhancing CD8+ T cell activity and/or enhancing antitumor immunity. In some embodiments, the TVA or active TVA derivative is a compound of formula (I):

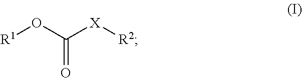

wherein: R¹ is selected from hydrogen and C₁-C₄ alkyl; X is selected from C₁-C₂₄ alkylene, C₂-C₂₄ alkenylene, and C₂-C₂₄ alkynylene; and R² is selected from hydrogen, optionally substituted aryl, optionally substituted heteroaryl, C₁-C₄ alkoxy, hydroxy, and halo. In some embodiments, R¹ is selected from hydrogen and methyl. In some embodiments, R¹ is hydrogen. In some embodiments, X is selected from C₈-C₂₄ alkylene, C₈-C₂₄ alkenylene, and C₈-C₂₄ alkynylene. In some embodiments, X is selected from C₁₂-C₂₄ alkylene, C₁₂-C₂₄ alkenylene, and C₁₂-C₂₄ alkynylene. In some embodiments, X is C₁₂-C₂₄ alkylene. In some embodiments, X is C₁₂-C₂₄ alkenylene. In some embodiments, X is C₁₂-C₂₄ alkynylene. In some embodiments, R² is selected from hydrogen and optionally substituted aryl. In some embodiments, R² is selected from hydrogen and phenyl, wherein the phenyl is unsubstituted or substituted with one substituent selected from C₁-C₄ alkyl (e.g., methyl). In some embodiments, R² is hydrogen. In some embodiments, the compound is selected from:

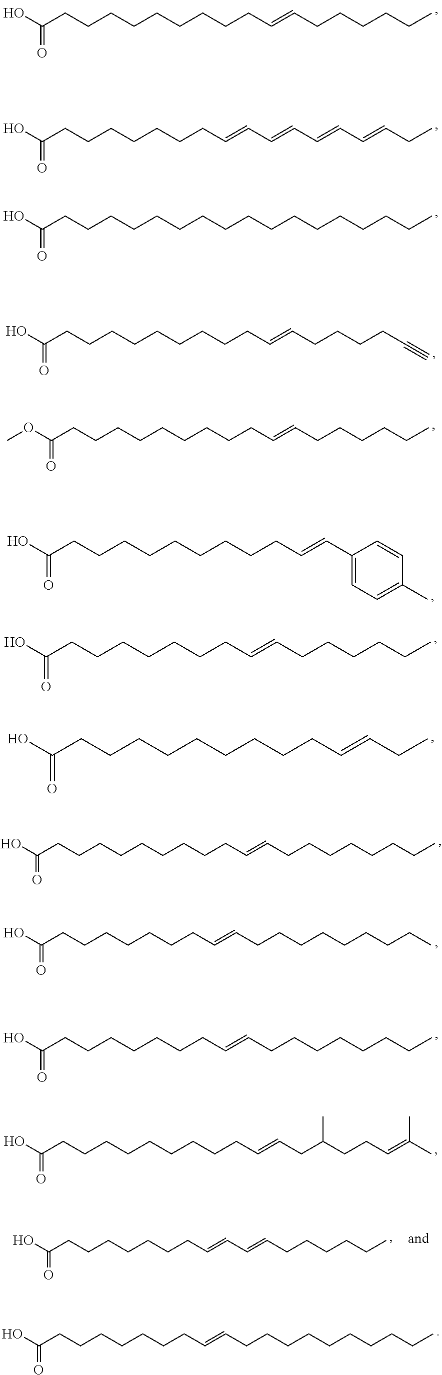

In some embodiments, the additional therapeutic agent is selected from a chemotherapeutic or immunotherapeutic. In some embodiments, the additional therapeutic agent is a T-cell-based immunotherapeutic. In some embodiments, the T-cell-based immunotherapeutic is selected from a therapy comprising the administration of immune checkpoint inhibitor, CAR-T cell therapy, monoclonal antibody therapy, and bispecific T-cell engager therapy.

In some embodiments, provided herein arc A TVA-containing or active-TVA-derivative-containing compositions for use in a method of treating cancer in a human subject. In some embodiments, the method comprises co-administering the TVA or active TVA derivative to the subject with an immunotherapeutic selected from a therapy comprising the administration of immune checkpoint inhibitor, CAR-T cell therapy, monoclonal antibody therapy, and bispecific T-cell engager therapy.

In some embodiments, provided herein are methods of preventing cancer in a subject in need thereof comprising administering an effective amount of trans-vaccenic acid (TVA) or an active TVA derivative to the subject.

In some embodiments, provided herein are methods of treating or preventing cancer in a subject comprising administering a composition that results in inhibition of GPR43 activity or expression. In some embodiments, the composition comprises trans-vaccenic acid (TVA) or active TVA derivative. In some embodiments, the composition comprises a nucleic acid inhibitor of GPR43 expression or activity. In some embodiments, the composition comprises a siRNA, shRNA, antisense RNA, or CRIPSR system to inhibit expression of GPR43. In some embodiments, the composition comprises a small molecule or peptide inhibitor of GPR43 activity. In some embodiments, the small molecule inhibitor of GPR43 activity is a GPR43 antagonist. In some embodiments, the GPR43 antagonist has the structure:

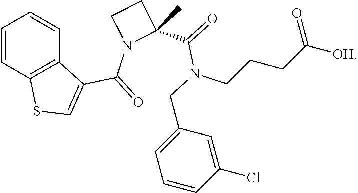 [SMILES: C[C@]1(C(=O)N(CCCC(=O)O)Cc2cccc(Cl)c2)CCN1C(=O)c1csc2ccccc12]

In some embodiments, the small molecule inhibitor of GPR43 activity is an inverse agonist of GPR43. In some embodiments, the inverse agonist of GPR43 has the structure:

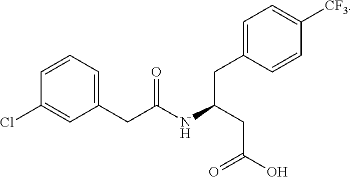 [SMILES: O=C(O)C[C@H](Cc1ccc(C(F)(F)F)cc1)NC(=O)Cc1cccc(Cl)c1]

### BRIEF DESCRIPTION OF THE DRAWINGS

FIG. 1. Dietary TVA enhances anti-tumor immunity. (A) First screen strategy to identify blood chemicals that enhance Jurkat T cell activation (upper) or reverse PD-L1/PD-1 mediated PD-1⁺ Jurkat T cell exhaustion induced by co-cultured H596 human lung cancer cells (lower). (B) Scatter plot showing result summary of first screens. Relative changed percentage was obtained by comparing IL-2 production level in treated group to that in control group. Candidates significantly enhanced (red) or reduced (blue) IL-2 levels were highlighted. (C) Second screen strategy: top 50 candidates of first screen 1a and 1b were combined. The resulting 6 overlapped candidates were subjected to analysis of IL-2 levels using murine primary T cells, revealing TVA as a top candidate that activates T cells. (D) Schematic depicting experimental design for in vivo mouse model (left). Effect of TVA-enriched diet (middle) or CVA-enriched diet (right) on B16F10 tumor growth in C57BL/6 mice. (E-G) Effect of TVA-enriched diet on colon cancer MC38 (E), breast cancer E0771 (F), and lung cancer LLC1 (G) tumor growth in C57BL/6 mice. (H-I) Effect of TVA-enriched diet on B16F10 tumor growth in nude mice (H) or TCR-α KO mice (I). (J) Effect of TVA-enriched diet on B16F10 tumor growth in C57BL/6 mice treated with isotype control (left) or depleting anti-CD8 (right) antibodies. Data were presented as mean±SEM (n≥8). p values were obtained by a two-way ANOVA test (ns, not significant; *0.01<p<0.05; ** 0.001<p<0.01; *** p<0.001). Also see FIGS. 17 and Table 1-5.

FIG. 2. Dietary TVA enhances tumor-infiltrating and cytotoxic function of effector CD8⁺ T cells. Schematic depicting experimental design for in vivo tumor-bearing mouse model fed with indicated diets and samples collection. TVA levels in B16F10 tumor bearing mice scrum (left) and TIF (right) were measured by ¹H nuclear magnetic resonance (NMR) spectroscopy. Quantification of the percentage of CD4⁺ and CD8⁺ T cells among intratumoral CD45⁺ cells. Quantification of the percentage of CD8⁺ T cells among spleen, dLN, and intratumoral CD45⁺ cells. (E-F) Quantification of PD-1 (E) and LAG-3 (F) expression among CD8⁺ T cells in spleen, dLN, and tumor. Flow cytometry and quantification of IL-2 (left), TNF-α (middle), and IFN-γ (right) expression among intratumoral CD8⁺ T cells after in vitro phorbol myristate acetate (PMA)/ionomycin stimulation. Quantification of Ki-67 (left), ICOS (middle), and GZMB (right) expression among intratumoral CD8⁺ T cells. Flow cytometry and quantification of TCF1 (left) and TOX (right) expression among intratumoral CD8⁺ T cells. Schematic depicting experimental setup for in vitro cell model. Mouse primary CD8⁺ T cells were isolated, activated, and treated with TVA, followed by analysis of TVA effect on cell functions. Relative cell number and Ki-67 expression among CD8⁺ T cells. Relative TNF-α (left) and IFN-γ (right) expression among CD8⁺ T cells after phorbol myristate acetate (PMA)/ionomycin stimulation. Percentage of apoptotic cells (left), Bcl2 level (middle), and active caspase-3 level (right) among CD8⁺ T cells. Data were presented as mean±SD (n≥3). p values were obtained by a two-tailed Student's t test (ns, not significant; * 0.01<p<0.05; ** 0.001<p<0.01; *** p<0.001). Also see FIGS. 18.

FIG. 3. TVA exhibits a nutrient-independent, signaling function through a GPCR-CREB axis. Schematic depicting experimental design for integrated temporal mechanistic studies. Human or mouse primary CD8⁺ T cells were isolated, activated, and treated with or without TVA for indicated time, followed by analysis through KAS-seq, phospho-antibody array and RNA-seq. Gene ontology (GO) enrichment graphs generated from KAS-seq differential analysis of H. sapiens (left) and M. musculus (right) CD8⁺ T cell gene bodies (treated with 20 μM TVA vs. untreated). Specifically, gene bodies exhibiting differential ssDNA levels for all timepoints (cutoff for individual timepoints of p<0.4 (H. sapiens) or p<0.5 (M. musculus)). Color indicates fold-enrichment and size of GO term circles denotes the number of differentially expressed genes from KAS-seq data for that term. GPCR-related terms in bold. Scatterplot of phospho antibody array representing Relative pixel density after TVA treatment for 40 minutes (left), 2 hours (middle), and 6 hours (right) versus corresponding-log 10 (P-value). Phospho proteins with relative pixel density >110% or <90%, meanwhile P<0.05 as significant change were highlighted. GSEA of upregulated effector CD8⁺ T cells (upper left), E2F targets (upper right), MYC targets V1 (lower left), and MYC target V2 (lower right) induced by TVA treatment in CD8⁺ T cells. NES, normalized enrichment score. Heatmap showing relative expression of PKA-CREB pathway genes in CD8⁺ T cells comparing TVA treatment group to the control ones. Date represent two or three independent biological replicates. Also see FIGS. 19-20 and Table 6.

FIG. 4. TVA signals through the GPCR-cAMP-PKA-CREB axis to enhance CD8⁺ T cell function. Schematic depicting experimental setup for in vitro T cell model treated with GPCR pathway modulators or inhibitors (left). Summary of TVA-GPCR downstream signaling changes induced by TVA in CD8⁺ T cells (right). The red fork means no change. Effects of treatment with GPCR modulator SCH on TVA-dependent CD8⁺ T cell activation assessed by IL-2 (left), TNF-α (middle left), IFN-γ (middle right), and p-STAT1 (right) level. Effects of TVA treatment on cAMP levels of CD8⁺ T cells. Effects of treatment with PKA inhibitor H89 cl on TVA-dependent CD8⁺ T cell activation assessed by IL-2 (left), TNF-α (middle), and IFN-γ (right) level. Effects of TVA treatment on p-CREB and p-LCK levels of CD8⁺ T cells. Effects of treatment with CREB inhibitor 666-15 on TVA-dependent CD8⁺ T cell activation assessed by IL-2 (left), TNF-α (middle), and IFN-γ (right) level. Effects of treatment with CREB inhibitor 666-15 on B16F10 cell proliferation in vitro. Schematic depicting experimental design for in vivo tumor-bearing mouse model fed with TVA diets and treated with CREB inhibitor (upper). Effects of treatment with CREB inhibitor 666-15 and/or TVA diet on B16F10 tumor growth in vivo (lower). Effects of treatment with different doses of cell permeable cAMP or TVA on p-CREB levels of CD8⁺ T cells. Data were presented as mean±SD except as mean±SEM in 4G-4H, and represent three independent biological replicates with n≥8 mice in 4H. p values were obtained by a two-tailed Student's t test except by a two-way ANOVA test in 4G-4H (ns, not significant; * 0.01<p<0.05; ** 0.001<p<0.01; *** p<0.001). Also see FIGS. 21.

FIG. 5. TVA-enhanced CD8⁺ T cell function is primarily mediated through CREB and its target gene sets. Schematic depicting experimental setup for in vitro T cell model for RNA-seq using CREB knockdown samples. Principal component analysis of the genes from RNA-seq in siRNA mediated Creb1 transient knockdown (siCreb) CD8⁺ T cells with or without TVA treatment comparing to that in non-targeting control siRNA (siNTC) group. GSEA of upregulated effector CD8⁺ T cells (upper left), E2F targets (upper right), MYC targets V1 (lower left), and MYC target V2 (lower right) in siNTC with TVA treatment compared to siCreb1 with TVA treatment. NES, normalized enrichment score. Heatmap of differentially expressed genes from RNA-seq in siCreb CD8⁺ T cells comparing to siNTC group with or without TVA treatment. The up- or down-regulated genes only in siNTC+TVA group compared to the other 3 groups were gated yellow box (left) and enriched for GO analysis (right). TVA-Creb1 target genes validation: Log 2 fold changes of cell number (upper left), apoptosis (upper middle), Ki-67 (upper right), IL-2 (lower left), TNF-α (lower middle), and IFN-γ (lower right) after TVA treatment in CD8⁺ T cells with individually transient knockdown of Creb1, Il18, Tbx21, Ilf2, Bcl6, Foxo4, and Ebi3. Data were presented as mean±SD of three independent biological replicates. p values were obtained by a two-tailed Student's t test in 5D (ns, not significant; * 0.01<p<0.05; ** 0.001<p<0.01; *** p<0.001). Also see Table 7.

FIG. 6. TVA antagonizes immunosuppressive SCFA-binding GPR43. Schematic depicting experimental setup for in vitro T cell model for flow cytometry using GPRs knockdown samples (upper). Effects of transient knockdown of known fatty acid related GPCRs (Gpr40, Gpr41, Gpr120, Gpr84, Gpr119, Gpr43) on TNF-α level in mouse CD8⁺ T cells treated with or without TVA (lower). Effects of Gpr43 transient knockdown on cAMP (left), p-CREB (middle), and TNF-α (right) levels in mouse CD8⁺ T cells treated with or without TVA. Effects of individually knockout of Gpr43 with three sgRNA on cAMP (left), p-CREB (middle), and TNF-α (right) levels in mouse Cas9; OT-I cells treated with or without TVA. Chemical structures of the three synthetic designed photo-affinity probes of TVA. Effects of treatment with 20, 40, or 100 μM TVA probes on TNF-α level in mouse CD8⁺ T cells. Schematic depicting experimental setup for pulldown assay (left). Western blot showing Gpr43 level in TVA probe 3 pulldown proteins by Gpr43 antibody (right upper), and TVA probe 3 pulldown proteins ladder by biotin antibody (right lower). Effect of treatment with 10 mM short chain fatty acids mix (mix of acetate, propionate, and butyrate) with or without 20 μM TVA on TNF-α level in mouse CD8⁺ T cells. (H-I) Effects of synchronized treatment with different doses of acetate with or without 20 μM TVA on TNF-α (H) and IFN-γ (I) level in mouse CD8⁺ T cell. Effects of synchronized treatment with different doses of TVA with or without 20 mM acetate TNF-α level in mouse CD8⁺ T cell. (K-L) Effects of subsequent treatment with 20 mM acetate and 20 μM TVA (acetate first: acetate for 12 hours first and then TVA added for another 12 hours; TVA first: TVA for 12 hours first and then acetate added for another 12 hours) on TNF-α (K) and IFN-γ (L) levels in CD8⁺ T cells. (M) Relative Gpr43 mRNA level in CD8⁺ T cells and CD4⁺ T cells with or without anti-CD3/CD28 stimulation. Data were presented as mean±SD of three independent biological replicates. p values were obtained by a two-tailed Student's t test (ns, not significant; *0.01<p<0.05; ** 0.001<p<0.01; *** p<0.001). Also see FIGS. 22.

FIG. 7. TVA augments effectiveness of multiple T cell-based anti-cancer therapies. Schematic depicting experimental design for in vivo tumor-bearing mouse model fed with TVA diets and treated with anti-PD-1 antibody (upper). Effect of anti-PD-1 antibody on B16F10 tumor growth in C57BL/6 mice fed with TVA-enriched diet or control diet (lower). Schematic depicting experimental design for in vitro blinatumomab-mediated cytotoxicity (upper). Box plots representing effect of combined indicated concentration of blinatumomab and TVA on RS4;11 target cells specific lysis percentage in the presence of PBMC assessed by flow cytometry (lower). Schematic depicting experimental design for in vitro CAR-T cells expansion (left). Effects of treatment with 20 μM TVA on anti-CD19-CD28z CAR-T cells expansion of four cases of lymphoma patients in vitro (right). (D) Schematic depicting experimental design for serum collection from patients with CAR-T cell therapy (left). Violin plots showing serum TVA levels of 10 lymphoma patients that have undergone commercial CAR-T cell therapy. Each patient has blood collection at 4 different timepoints (detailed information can be found in Table 8). Red violin plots represent the patients who have complete response to CAR-T cell therapy. Blue violin plots represent the patients who have progressive disease to CAR-T cell therapy. (E) Working model. Data were presented as mean±SD except as mean±SEM in 7A, and represent at least three independent biological replicates with n≥8 mice in 7A except technical replicates in 7C-7F. p values were obtained by a two-tailed Student's t test except by a two-way ANOVA test in 7A (*0.01<p<0.05; ** 0.001<p<0.01; *** p<0.001). Also see FIGS. 23 and Table 8.

FIG. 8. TVA suppresses tumorigenesis and tumor progression. Representative colons showing tumors in situ (images on left labeled “Ctl” and “TVA”). Tumor multiplicity and tumor pathology (CIS carcinoma in situ; Inv Ca invasive cancer; n=11 per group, Kruskal Wallis statistic) (charts on right).

FIG. 9. TVA effect on macrophages.

FIG. 10. TVA effect on CD8 cells.

FIG. 11. TVA effect on Tregs and YTHDF2.

FIG. 12. TVA effect on DCs and YTHDF1.

FIG. 13. Oral gavage of TVA to mice ameliorates immune response to infection by influenza virus (by Zhong Zheng and Hao Fan).

FIG. 14. TVA enhances immune response to infection by influenza virus with sex preference to male (by Zhong Zheng and Hao Fan).

FIG. 15. TVA derivative #203 enhances CD8+ T cell function in vitro.

FIG. 16. Oral gavage of TVA derivative #203 enhances anti-tumor immunity in syngeneic mice.

FIG. 17. Related to FIG. 1. TVA functions directly through T cells but not tumor cells. (A-B) Effect of treatment with 20 μM TVA on IL-2 levels in mouse primary T cell (A) and human primary T cell (B). Effect of treatment with 20 μM TVA on PD-L1/PD-1 mediated Jurkat T cell exhaustion induced by co-cultured H596 (left) and H460 (middle) human lung cancer cells, or A375 human melanoma cells (right), assessed by IL-2 production levels. Effect of treatment with 20 μM TVA on cytotoxicity of B16F10 cells by co-cultured Pmel cells in vitro. Effects of treatment with 20 μM TVA on B16F10 cell proliferation (left) or apoptosis (right). Body weight changes of B16F10 tumor-bearing C57BL/6 mice fed with control diet or TVA diet. Body weight changes of B16F10 tumor-bearing C57BL/6 mice fed with control diet or CVA diet. Effects of treatment with 20 μM TVA on MC38 cell proliferation. CD8⁺ T cell depletion efficiency checked by flow cytometry. Data were presented as mean±SD except as mean±SEM for the cell proliferation assay, and represent three independent biological replicates with n≥8 mice in FIG. 17F and FIG. 171G. p values were obtained by a two-tailed Student's t test except by a two-way ANOVA test for cell proliferation assay (ns, not significant; *0.01<p<0.05; ** 0.001<p<0.01; *** p<0.001).

FIG. 18. Related to FIG. 2. TVA shows distinct effect among leukocytes populations specific to tumor context. Box-and-whisker plot showing alpha diversity of gut feces microbiota in B16F10 tumor bearing mice fed with control diet or TVA-enriched diet. The heatmap shows the microbial composition of the samples at the species level with the top fifty most abundant species identified. Each row represents the abundance for each taxon, with the taxonomy ID shown on the right. Each column represents the abundance for each sample, with the sample ID shown at the bottom. Group information is indicated by the colored bar located on the top of each column. Hierarchical clustering was performed on samples based on Bray-Curtis dissimilarity. Hierarchical clustering was also performed on the taxa so that taxa with similar distributions are grouped together. Quantification of the percentage of dendritic cells, macrophages, neutrophils, and monocytes among tumor CD45⁺ cells. (D-E) Quantification of the percentage of dendritic cells, macrophages, neutrophils, and monocytes among spleen (D) or dLN (E) CD45⁺ cells. Gating strategies for flow cytometry analysis of TILs in FIGS. 2C-2I. Gating strategies for flow cytometry analysis of spleen and dLN lymphocytes in FIGS. 2C-2F. Quantification of the percentage of CD4⁺ T cells among spleen, dLN, and intratumoral CD45⁺ cells. Quantification of the percentage of CD4⁺Foxp3⁺ Treg cells among spleen, dLN, and intratumoral CD4⁺ cells. Quantification of the percentage of CD8⁺ and CD4⁺ T cells among intratumoral CD45⁺ cells (left two), and quantification of PD-1, TNF-α, TCF1 expression among CD8⁺ TILs (right three) in MC38 tumors. Quantification of the percentage of CD4⁺ and CD8⁺ T cells among dLN CD45⁺ cells (left), and quantification of IL-2, TNF-α, IFN-γ expression among dLN CD45⁺ cells (right three) in non-tumor bearing mice. Relative IL-2, TNF-α, and IFN-γ expression among CD4⁺ cells after phorbol myristate acetate (PMA)/ionomycin stimulation in vitro (left three), and apoptotic cell percentage among CD4⁺ cells (right). Data were presented as mean±SD of three independent biological replicates. p values were obtained by a two-tailed Student's t test (ns, not significant; * 0.01<p<0.05; ** 0.001<p<0.01).

FIG. 19. Related to FIG. 3. TVA functions in an extracellular manner. Schematic depicting experimental setup for in vitro T cell model for ¹³C1-TVA metabolic flux analysis by GC-MS (left). Detection of intracellular ¹³C-labeled TVA level in mouse CD8⁺ T cells by GC-MS (middle). Effect of CD36 inhibitor SSO on ¹³C-labeled TVA uptake in mouse CD8⁺ T cells was accessed by GC-MS (right). Schematic depicting experimental setup for in vitro T cell model for CD36 inhibitor SSO treatment (left). Effect of TVA treatment with or without CD36 inhibitor SSO on production of TNF-α (middle) or INF-y (right) of mouse CD8⁺ T cells. Schematic depicting experimental setup for in vitro T cell model for TVA “wash-off” assay (TVAc: CD8⁺ T cells were treated with 20 μM TVA for 48 hours; TVAw: CD8⁺ T cells were treated with 20 μM TVA for 24 hours and then changed to culture medium without TVA for another 24 hours) (left). Quantification of TNF-α (middle) and IFN-γ (right) expression after phorbol myristate acetate (PMA)/ionomycin stimulation among CD8⁺ T cells for TVA “wash-off” assay. Data were presented as mean±SD of three independent biological replicates. p values were obtained by a two-tailed Student's t test (ns, not significant; ** 0.001<p<0.01; *** p<0.001).

FIG. 20. Related to FIG. 3. TVA signals through a GPCR-CREB axis to enhance CD8⁺ T cell function. (A-B) Human (A) and mouse (B) Volcano plots depicting differential promoter and gene body ssDNA levels measured by KAS-scq in CD8⁺ T cell treated with 20 μM TVA for indicated timepoint (relative to untreated cells). Heavy horizontal lines correspond to p-values of 0.2 (M. musculus only), 0.1, and 0.05, while heavy vertical lines correspond to a log 2|fold-change| value of 0.5. GO enrichment graphs for all differentially expressed gene bodies (at any timepoint) for the corresponding p-value cutoffs and species. Color and GO term circle size correspond to fold-enrichment and number of differentially-expressed gene bodies for that term. Scatterplot of phospho antibody array representing Relative pixel density after TVA treatment for 24 hours versus corresponding-log 10 (P-value). Phospho proteins with relative pixel density >110% or <90%, meanwhile P<0.05 as significant change were highlighted. Flow cytometry (left) and quantification (right) of p-CREB (S133) expression among CD8⁺ T cells treated with 20 μM TVA for 2 hours. Quantification of p-CREB (left), and p-STAT1 (right) expression among mouse tumor-infiltrating CD8⁺ T cells. GO enrichment analysis of up-regulated genes (upper) and down-regulated genes (lower) by RNA-seq in CD8⁺ T cells treated with TVA comparing to control. Validation of some differentially expressed genes (DEGs) of RNA-seq by RT-PCR. Data were presented as mean±SD of two or three independent biological replicates. p values were obtained by a two-tailed Student's t test (*0.01<p<0.05; ** 0.001<p<0.01; *** p<0.001).

FIG. 21. Related to FIG. 4. TVA signals through the cAMP-PKA-CREB axis but not other downstream pathway(s) of GPCR. (A-C) Effect of treatment with ERK inhibitor U0126 (A), NFAT inhibitor (B), or RhoA inhibitor Rhosin cl (C) on IL-2 (left), TNF-α (middle), and IFN-γ (right) level in mouse CD8⁺ T cells treated with or without TVA. Ebi3, Foxo4, Bcl6, Ilf2, Tbx21, IL18, and Creb1 knockdown efficiency checked by RT-PCR. Data were presented as mean±SD of three independent biological replicates. p values were obtained by a two-tailed Student's t test (*** p<0.001).

FIG. 22. Related to FIG. 6. Gpr43 knockdown efficiency in CD8⁺ T cells was checked by RT-PCR (left). Effects of Gpr43 transient knockdown on IFN-γ (middle) and p-STAT1 (right) levels in mouse CD8⁺ T cells treated with or without TVA were shown. Gpr43 knockdown efficiency in OT-I cells was checked by RT-PCR (left). Effect of Gpr43 transient knockdown on TNF-α (right) level in OT-I cells treated with or without TVA was shown. Knockout efficiency of Gpr43 in Cas9; OT-I cells was checked by RT-PCR (left). Effects of individually knockout of Gpr43 with three sgRNAs on apoptosis in mouse Cas9; OT-I cells treated with or without TVA were shown (right). Chemical structures of TVA and its designed 15 TVA derivatives. Effects of treatment with 20 μM TVA or TVA derivatives on TNF-α (upper) and IFN-γ (lower) in CD8⁺ T cells. Effects of subsequent treatment with 20 mM propionate (left) or 20 mM butyrate (right) and 20 μM TVA (propionate or butyrate first: propionate or butyrate for 12 hours first and then TVA added for another 12 hours; TVA first: TVA for 12 hours first and then propionate or butyrate added for another 12 hours) on TNF-α level in CD8⁺ T cells. Data were presented as mean±SD of three independent biological replicates. p values were obtained by a two-tailed Student's t test (ns, not significant; * 0.01<p<0.05; ** 0.001<p<0.01; *** p<0.001).

FIG. 23. Related to FIG. 7. TVA significantly reverses PD-1/PD-L1-induced exhaustion of primary CD8⁺ T cells. (A-B) Effect of treatment with 20 μM TVA on human bulk T cells HD501 (A) and HD505 (B) exhaustion induced by purified PD-L1, assessed by IL-2 production levels. (C-D) Effect of treatment with 20 μM TVA on human CD8⁺ T cells exhaustion induced by purified PD-L1, assessed by IL-2 (C) and TNF-α (D) levels. (E-F) Effect of treatment with 20 μM TVA on mouse primary T cells exhaustion induced by co-cultured B16F10 cells expressing PD-L1 (B16F10 PD-L1⁺), assessed by IL-2 (E) and TNF-α (F) level. (G-H) Effect of treatment with 20 μM TVA on mouse CD8⁺ T cells exhaustion induced by co-cultured B16F10 cells expressing PD-L1 (B16F10 PD-L1⁺), assessed by IL-2 (G) and TNF-α (H) level. Data were presented as mean±SD of three independent biological replicates. p values were obtained by a two-tailed Student's t test (*0.01<p<0.05; ** 0.001<p<0.01; *** p<0.001).

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the terms “trans-vaccenic acid” or “TVA” refer to a compound of the formula:

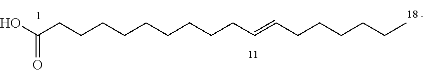 [SMILES: CCCCCC/C=C/CCCCCCCCCC(=O)O]

As used herein, the term “active TVA derivative” refers to a TVA derivative is a compound of formula (I):

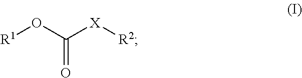

wherein: R¹ is selected from hydrogen and C₁-C₄ alkyl; X is selected from C₁-C₂₄ alkylene, C₂-C₂₄ alkenylene, and C₂-C₂₄ alkynylene; and R² is selected from hydrogen, optionally substituted aryl, optionally substituted heteroaryl, C₁-C₄ alkoxy, hydroxy, and halo; wherein the compound is capable of enhancing CD8+ T cell activity and/or enhancing antitumor immunity via the mechanisms and/or interactions described herein for TVA. In some embodiments, R¹ is selected from hydrogen and methyl. In some embodiments, R¹ is hydrogen. In some embodiments, X is selected from C₈-C₂₄ alkylene, C₈-C₂₄ alkenylene, and C₈-C₂₄ alkynylene. In some embodiments, X is selected from C₁₂-C₂₄ alkylene, C₁₂-C₂₄ alkenylene, and C₁₂-C₂₄ alkynylene. In some embodiments, X is C₁₂-C₂₄ alkylene. In some embodiments, X is C₁₂-C₂₄ alkenylene. In some embodiments, X is C₁₂-C₂₄ alkynylene. In some embodiments, R² is selected from hydrogen and optionally substituted aryl. In some embodiments, R² is selected from hydrogen and phenyl, wherein the phenyl is unsubstituted or substituted with one substituent selected from C₁-C₄ alkyl (e.g., methyl). In some embodiments, R² is hydrogen. In some embodiments, the compound is selected from:

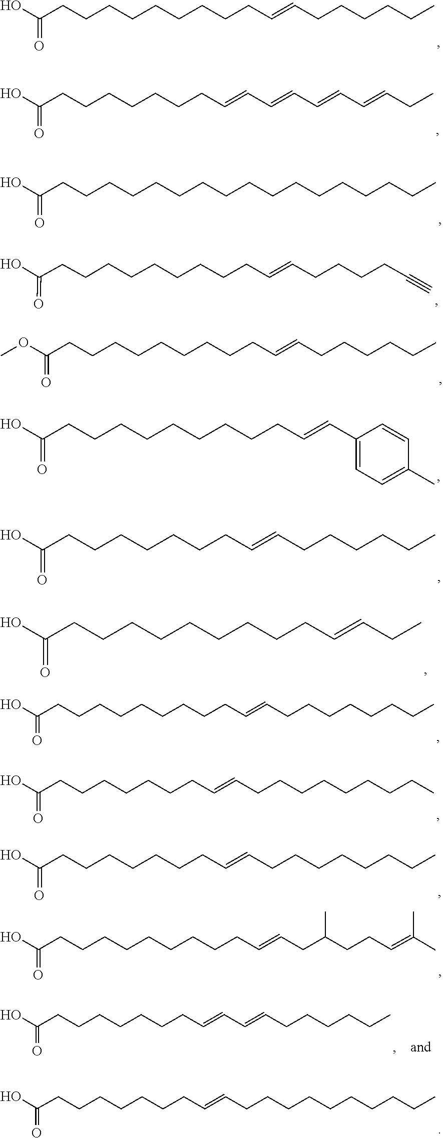

In some embodiments, a TVA derivative is not TVA.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkylene” refers to a divalent alkyl group.

As used herein, the term “alkenyl” refers to a radical of a straight or branched hydrocarbon chain containing at least one carbon-carbon double bond and no triple bonds. The double bond(s) may be located at any position(s) with the hydrocarbon chain. The alkenyl chain can include, e.g., from 2 to 24 carbon atoms (C₂-C₂₄ alkenyl), 2 to 16 carbon atoms (C₂-C₁₆ alkenyl), 2 to 14 carbon atoms (C₂-C₁₄ alkenyl), 2 to 12 carbon atoms (C₂-C₁₂ alkenyl), 2 to 10 carbon atoms (C₂-C₁₀ alkenyl), 2 to 8 carbon atoms (C₂-C₈ alkenyl), 2 to 6 carbon atoms (C₂-C₆ alkenyl), 2 to 4 carbon atoms (C₂-C₄ alkenyl), 2 to 3 carbon atoms (C₂-C₃ alkenyl), or 2 carbon atoms (C₂ alkenyl). Representative examples of alkenyl include, but are not limited to, ethenyl, 1-propenyl, 2-propenyl, 1-butenyl, 2-butenyl, butadienyl, 2-methyl-2-propenyl, 3-butenyl, pentenyl, pentadienyl, hexenyl, heptenyl, octenyl, octatrienyl, and the like.

As used herein, the term “alkenylene” refers to a divalent alkenyl group.

As used herein, the term “alkynyl” means a radical of a straight or branched hydrocarbon chain containing at least one carbon-carbon triple bond. The alkynyl chain can include, e.g., from 2 to 24 carbon atoms (C₂-C₂₄ alkynyl), 2 to 16 carbon atoms (C₂-C₁₆ alkynyl), 2 to 14 carbon atoms (C₂-C₁₄ alkynyl), 2 to 12 carbon atoms (C₂-C₁₂ alkynyl), 2 to 10 carbon atoms (C₂-C₁₀ alkynyl), 2 to 8 carbon atoms (C₂-C₈ alkynyl), 2 to 6 carbon atoms (C₂-C₆ alkynyl), 2 to 4 carbon atoms (C₂-C₄ alkynyl), 2 to 3 carbon atoms (C₂-C₃ alkynyl), or 2 carbon atoms (C₂ alkynyl). The triple bond(s) may be located at any position(s) with the hydrocarbon chain. Representative examples of alkynyl include, but are not limited to, ethynyl, 1-propynyl, 2-propynyl, 1-butynyl, 2-butynyl, and the like.

As used herein, the term “alkynylene” refers to a divalent alkynyl group.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl”; i.e., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracenyl and phenanthrenyl).

As used herein, the term “halogen” or “halo” refers to F, Cl, Br, or I.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein, the term “hydroxy” or “hydroxyl” refers to an —OH group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

The term “supplement” as used herein refers to a nutritional product that provides nutrients (e.g. vitamins, minerals, fatty acids (e.g., TVA)) to a subject that may otherwise not be consumed in sufficient quantities (e.g., to enhance cancer treatment) by the subject. Supplements may be, for example, provided in the form of a pill, a tablet, a lozenge, a chewy capsule or tablet, a capsule, or a powder supplement that can be, for example, dissolved in water or a beverage (e.g., milk), or sprinkled on food. Supplements typically provide one or more selected compounds (e.g., TVA) without providing a significant portion of the overall nutritional needs of a subject.

The term “pharmaceutical formulation” as used herein refers to a composition comprising at least one pharmaceutically-active agent, chemical substance or drug. The pharmaceutical formulation may be in solid or liquid form and can comprise at least one additional active agent, carrier, vehicle, excipient or auxiliary agent identifiable by the skilled person. The pharmaceutical formulation may be in the form of a tablet, capsule, granules, powder, liquid or syrup.

The term “effective dose” or “effective amount” refers to an amount of an agent, e.g., a neutralizing antibody, that results in the reduction of symptoms in a patient, treatment of prevention of a disease or condition, or results in a desired biological outcome.

As used herein, the terms “administration” and “administering” refer to the act of giving a drug, prodrug, or other agent, or therapeutic to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, an “immune response” refers to the action of a cell of the immune system (e.g., T lymphocytes, B lymphocytes, natural killer (NK) cells, macrophages, eosinophils, mast cells, dendritic cells, neutrophils, etc.) and soluble macromolecules produced by any of these cells or the liver (e.g., antibodies, cytokines, and complement) that results in selective targeting, binding to, damage to, destruction of, and/or elimination from a subject of invading pathogens, cells or tissues infected with pathogens, or cancerous cells or other abnormal/diseased-associated cells.

As used herein, the term “immunotherapy” refers to the treatment or prevention of a disease or condition by a method comprising inducing, enhancing, suppressing or otherwise modifying an immune response.

As used herein, the term “immunotherapeutic” refers to any agent (e.g., small molecule, peptide, antibody, engineered cell, etc.) capable of stimulating a host immune system to generate an immune response to a tumor or cancer in the subject.

As used herein, the term “T-cell-based therapy” refers to any immunotherapy that acts through T cells. T-cell-based therapies include the administration of exogenous T cells (e.g., CAR-T cell therapies) and therapies that act upon or through a subjects endogenous T cells (e.g., checkpoint inhibitors.

As used herein, the term “antibody” refers to a whole antibody molecule or a fragment thereof (e.g., fragments such as Fab, Fab′, and F(ab′)₂), unless specified otherwise; an antibody may be polyclonal or monoclonal antibody, a chimeric antibody, a humanized antibody, a human antibody, etc.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

As used herein, the term “single-chain bispecific antibody construct” refers to a polypeptide construct comprising two antibody-derived binding domains. In some embodiments herein the two antibody-derived binding domains are an antigen-recognition domain and an activation domain. The binding domains may comprise variable regions (or parts thereof) of an antibody, antibody fragment or derivative thereof, capable of specifically binding to/interacting with a target antigen and/or an activation molecule. In certain embodiments, a part of a variable region comprises at least one CDR (“Complementary determining region”), such as at least a CDR1, CDR2, or CDR3 region. The two domains/regions in the single chain antibody construct are preferably covalently connected to one another as a single chain. Illustrative examples of bispecific single chain molecules are known in the art and are described in WO 99/54440; Mack, J. Immunol. (1997), 158, 3965-3970; Mack, PNAS, (1995), 92, 7021-7025; Kufer, Cancer Immunol. Immunother., (1997), 45, 193-197; Loffler, Blood, (2000), 95, 6, 2098-2103; and Bruhl, J. Immunol., (2001), 166, 2420-2426; incorporated by reference in their entireties. As used herein, the term “engager” refers to a molecule that is secreted from a cell and activates immune cells with which it interacts. The engager activates specific immune cells according to the domains present in the engager. Illustrative examples of cells that secrete engagers, but are not limited to, include T-cells, NK cells, NKT cells, CAR T-cells, mesenchymal stem cells (MSCs), neuronal stem cells, hematopoietic stem cells, or a mixture thereof, in some cases.

As used herein, the term “bispecific” refers to any molecule or molecular complex that has two different binding specificities. The molecule or molecular complex may comprise two separate binding domains, each with the same specificity (“homobispecific”) or with specificity for different molecular entities (e.g., antigens) (“heterobispecific”). For example, a “bispecific engager” is an engager molecule capable of binding a target antigen (e.g., an antigen on a cancer cell) and a immunostimulatory element.

As used herein, the term “antigen-recognition domain” refers to a molecular moiety (e.g. part of an engager molecule or antibody) that recognizes an antigen. In particular embodiments, antigens can be of any nature including, but not limited to, proteins, carbohydrates, and/or synthetic molecules.

As used herein, the term “activation domain” refers to a molecular moiety (e.g. part of an engager molecule or antibody) that interacts with immune cells (e.g., T cell receptor (TCR)) and induces a positive or negative immunomodulatory signal. Illustrative examples of positive immunomodulatory signals include signals that induce cell proliferation, cytokine secretion, or cytolytic activity. Illustrative examples of negative immunomodulatory signals include signals that inhibit cell proliferation, inhibit the secretion of immunosuppressive factors, or induce cell death.

As used herein, the term “co-stimulatory domain” or “co-stimulatory signaling domain” refers to an intracellular signaling domain of a co-stimulatory molecule. In particular aspects, it refers to a domain that provides additional signals to the immune cell in conjunction with an activation domain. Co-stimulatory molecules are cell surface molecules other than antigen receptors or Fc receptors that provide a second signal required for efficient activation and function of T lymphocytes upon binding to antigen. Illustrative examples of such co-stimulatory molecules include CD27, CD28, 4-1BB (CD137), OX40 (CD134), CD30, CD40, ICOS (CD278), LFA-1, CD2, CD7, LIGHT, NKD2C, CD70, CD80, CD86, and CD83.

As used herein, the term “intracellular signaling domain,” when used in reference to a cell surface receptor or a CAR, is a moiety responsible for activation of at least one function of the cell upon which the receptor or CAR is displayed. The term “effector function” refers to a specialized function of a cell. For example, effector function of a T cell includes cytolytic activity or helper activity including the secretion of cytokines. Thus the term “intracellular signaling domain” refers to the portion of a protein which transduces the effector function signal and directs the cell to perform a specialized function. To the extent that a truncated portion or variant of a native intracellular signaling domain is active, such a polypeptide may be used in place of the full native chain, as long as it transduces the effector function signal. The term intracellular signaling domain includes any truncated or variant portion of a polypeptide sequence sufficient to transduce the effector function signal. Examples of intracellular signaling domains include the cytoplasmic sequences of the T cell receptor (TCR) and co-receptors that act in concert to initiate signal transduction following antigen receptor engagement, as well as any derivative or variant of these sequences and any synthetic sequence that has the same functional capability. Cytoplasmic signaling sequences that act in a stimulatory manner comprise signaling motifs which are known as immunoreceptor tyrosine-based activation motifs (ITAMs). Examples of ITAM containing cytoplasmic signaling sequences include those derived from TCR zeta, FcR gamma, FcR beta, CD3 gamma, CD3 delta, CD3 epsilon, CD3 zeta, CD5, CD22, CD79a, CD79b, and CD66d.

As used herein, the term “transmembrane domain,” when used in reference to a cell surface receptor or a CAR, is a moiety that spans the plasma membrane of the cell and is connected to both the intracellular signaling domain and the extracellular antigen-recognition domain. A transmembrane domain may be derived either from a natural or from a synthetic source. Where the source is natural, the domain may be derived from any membrane-bound or transmembrane protein, for example, the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, etc. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular signaling domain. A glycine-serine doublet provides a particularly suitable linker.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. See, e.g., Kohler and Milstein (1975) Nature 256:495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352:624-628; and Marks et al. (1991) J. Mol. Biol. 222:581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody). produced by recombinant DNA techniques, or chemical polypeptide synthesis.

As used herein, the term “native immune cell” refers to an immune cell that naturally occurs in the immune system of a subject. Illustrative examples include, but are not limited to, T-cells, NK cells, NKT cells, B cells, and dendritic cells.

As used herein, the term “engineered immune cell” refers to an immune cell (e.g., T-cell, NK cell, NKT cell, B cell, dendritic cell, etc.) that is genetically modified.

The term “chimeric antigen receptor” (“CAR”) refers to a recombinant polypeptide construct comprising at least an extracellular antigen-recognition domain, a transmembrane domain and an intracellular signaling domain. Upon binding to their target (e.g., displayed on a cancer cell), CARs typically modify the immune response of the immune cells they are displayed upon.

As used herein, the term “CAR-T cell” refers to a T cell that has been engineered to express a chimeric antigen receptor. In particular embodiments, T cells (e.g., from a subject) are engineered to express a CAR that binds to a cancer-specific antigen of cancer cells, thereby allowing CAR-T cells to effectively recognize and kill cancer cells,

As used herein, the term “adoptive cell transfer” (“ACT”) is the transfer of cells into a patient. The cells may have originated from the patient or from another individual or cell line.

The cells are most commonly derived from the immune system, with the goal of improving immune functionality or eliciting a desired immune response. In some embodiments, cells are extracted from a subject, genetically modified (e.g., to express a desired construct (e.g., CAR or endanger molecule)), cultured in vitro, and returned to the subject.

### DETAILED DESCRIPTION

Provided herein are compositions and methods for stimulating T-cell-based anti-tumor immunity by administration of trans-vaccenic acid (TVA), an active derivative thereof, or an inhibitor of GPR43 expression or activity. In particular embodiments, TVA, a TVA derivative, or an inhibitor of GPR43 expression or activity is administered to boost an endogenous T-cell response and/or is co-administered with a T-cell-based therapy, such as immune checkpoint blockade therapies, CAR-T therapies, monoclonal antibody therapies, bispecific T-cell engagers therapies, etc.

Provided herein arc compositions (e.g., pharmaceutical compositions, supplements, etc.) comprising TVA, an active TVA derivative, or an inhibitor of GPR43 expression or activity. In some embodiments, TVA-containing or active-TVA-derivative-containing compositions are provided for the treatment of cancer and/or as a supplement to enhance a cancer treatment. In some embodiments, TVA-containing or active-TVA-derivative-containing compositions are provided for the prevention of cancer. In some embodiments, TVA-containing or active-TVA-derivative-containing compositions are provided for reducing the likelihood or reducing the severity of cancer.

In some embodiments, provided herein is a compound of formula (I):

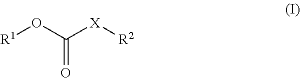

wherein:
R¹ is selected from hydrogen and C₁-C₄ alkyl;
X is selected from C₁-C₂₄ alkylene, C₂-C₂₄ alkenylene, and C₂-C₂₄ alkynylene; and
R² is selected from hydrogen, optionally substituted aryl, optionally substituted heteroaryl, C₁-C₄ alkoxy, hydroxy, and halo.

In some embodiments, R¹ is selected from hydrogen and methyl. In some embodiments, R¹ is hydrogen.

In some embodiments, X is selected from C₈-C₂₄ alkylene, C₈-C₂₄ alkenylene, and C₈-C₂₄ alkynylene. In some embodiments, X is selected from C₁₂-C₂₄ alkylene, C₁₂-C₂₄ alkenylene, and C₁₂-C₂₄ alkynylene. In some embodiments, X is C₁₂-C₂₄ alkylene. In some embodiments, X is C₁₂-C₂₄ alkenylene. In some embodiments, X is C₁₂-C₂₄ alkynylene.

In some embodiments, R² is selected from hydrogen and optionally substituted aryl. In some embodiments, R² is selected from hydrogen and phenyl, wherein the phenyl is unsubstituted or substituted with one substituent selected from C₁-C₄ alkyl (e.g., methyl). In some embodiments, R² is hydrogen.

In some embodiments, the compound is selected from:

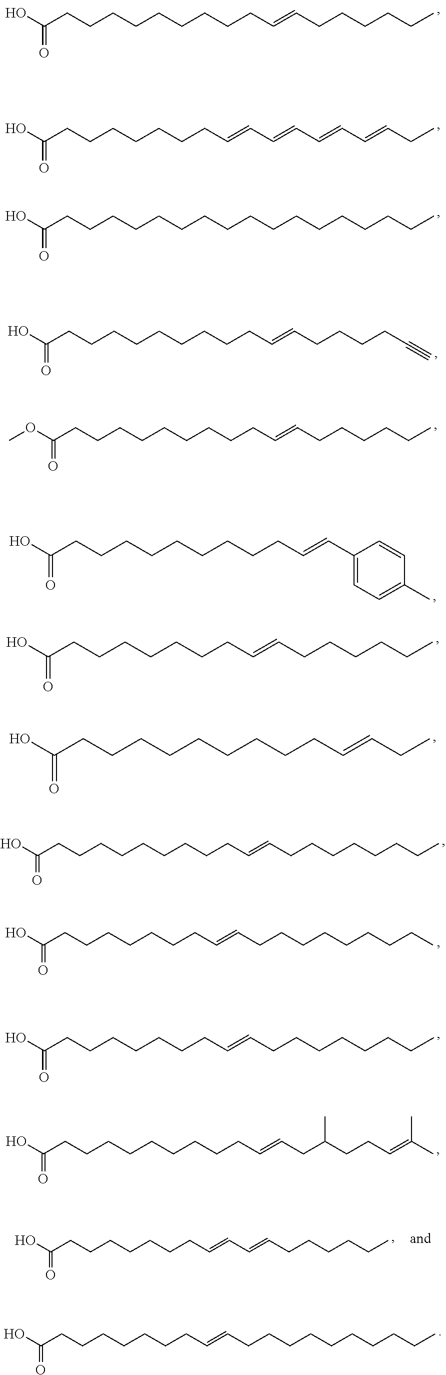

Experiments conducted during development of embodiments herein demonstrate that TVA exerts a therapeutic activity via attenuation of GPR43 activity. In some embodiments, provided herein are inhibitors of GPR43 activity or expression and methods of their use to boost an endogenous T-cell response and/or for use with a T-cell-based therapy as described herein.

In some embodiments, the composition comprises a nucleic acid inhibitor of GPR43 expression or activity. In some embodiments, the composition comprises a siRNA, shRNA, antisense RNA, or CRIPSR system to inhibit expression of GPR43.

In some embodiments, the composition comprises a small molecule or peptide inhibitor of GPR43 activity. In some embodiments, the small molecule inhibitor of GPR43 activity is a GPR43 antagonist. In some embodiments, the GPR43 antagonist has the structure:

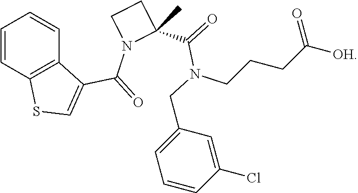 [SMILES: C[C@]1(C(=O)N(CCCC(=O)O)Cc2cccc(Cl)c2)CCN1C(=O)c1csc2ccccc12]

In some embodiments, the small molecule inhibitor of GPR43 activity is an inverse agonist of GPR43. In some embodiments, the inverse agonist of GPR43 has the structure:

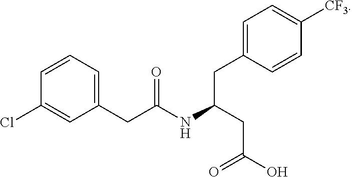 [SMILES: O=C(O)C[C@H](Cc1ccc(C(F)(F)F)cc1)NC(=O)Cc1cccc(Cl)c1]

In some embodiments, TVA, an active TVA derivative, or an inhibitor of GPR43 expression or activity is formulated for administration to a subject (e.g., human subject). In some embodiments, TVA, an active TVA derivative, or an inhibitor of GPR43 expression or activity is formulated as a supplement. In some embodiments, provided herein is a supplement consisting of TVA, an active TVA derivative, or an inhibitor of GPR43 expression or activity and suitable carriers. In some embodiments, the supplement comprises TVA (or an active TVA derivative or an inhibitor of GPR43 expression or activity) and other nutrients, vitamins, and/or minerals. In some embodiments, the other components of a supplement are present to assist or enable delivery of TVA, an active TVA derivative, or an inhibitor of GPR43 expression or activity. In some embodiments, other components of a supplement are present to enhance the health of a subject or to otherwise effect the treatment of cancer. Nutrients that may be provided with TVA, an active TVA derivative, or an inhibitor of GPR43 expression or activity include, but are not limited to minerals (e.g., iron, manganese, magnesium, copper, calcium, phosphorous, etc.), vitamins (e.g., biotin, choline, folate, niacin, pantothenic acid, riboflavin, thiamin, and vitamins A, B6, B12, C, D, E, K, etc.), and other fatty acids (e.g., omega-3 fatty acids (e.g., a-linolenic acid (ALA), eicosapentaenoic acid (EPA), docosahexaenoic acid (DHA), etc.), omega-6 fatty acids (e.g., linoleic acid (LA), etc.), trans fatty acids, saturated fatty acids, unsaturated fatty acids, polyunsaturated fatty acids (PUFA), etc.). In some embodiments, a supplement comprises TVA, an active TVA derivative, or an inhibitor of GPR43 expression or activity or activity and one or more active bacterial cultures, wherein the bacterial cultures comprise species of bacteria that are beneficial to human health or the treatment/prevention of cancer. In some embodiments, a TVA-containing, an active-TVA-derivative-containing, or GPR43-inhibitor-containing supplement is formulated according to standard supplement formulations that are understood in the art. In some embodiments, TVA, an active TVA derivative, or an inhibitor of GPR43 expression or activity is provided as a nutrient supplement to T cell activity when administered to, for example, an infant, an elderly subject, an immunocompromised patient, etc.

In some embodiments, TVA, an active TVA derivative, or an inhibitor of GPR43 expression or activity is formulated as a pharmaceutical composition. In some embodiments, TVA, an active TVA derivative, or an inhibitor of GPR43 expression or activity and any co-formulated agents (when present) are provided in pharmaceutical formulations for administration to a subject by a suitable route. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, TVA-containing or active-TVA-derivative-containing pharmaceutical compositions are formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragees, and capsules.

Pharmaceutical preparations comprising TVA, an active TVA derivative, or an inhibitor of GPR43 expression or activity provided for oral use can be obtained by mixing one or more solid excipients with TVA, an active TVA derivative, or an inhibitor of GPR43 expression or activity (and other therapeutic agents desired in the formulation) with any suitable substituents and functional groups disclosed herein, optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In some embodiments, routes of administration, formation of TVA, an active TVA derivative, or an inhibitor of GPR43 expression or activity, etc. are selected to provide efficient and effective delivery. In some embodiments, TVA, an active TVA derivative, or an inhibitor of GPR43 expression or activity is provided with a suitable carrier. In some embodiments, TVA, an active TVA derivative, or an inhibitor of GPR43 expression or activity is encapsulated of embedded into a carrier. In some embodiments, a carrier may comprise a liposome, nanoparticle, or other suitable system for delivery of TVA, an active TVA derivative, or an inhibitor of GPR43 expression or activity. In some embodiments, TVA, an active TVA derivative, or an inhibitor of GPR43 expression or activity is conjugated to a carrier molecule. Suitable carrier molecules for conjugation of TVA, an active TVA derivative, or an inhibitor of GPR43 expression or activity may include small molecules, peptides, proteins, polymers, etc. In some embodiments, the carrier and/or delivery system is selected to optimize the solubility, stability, bioavailability, targeting, etc. of the TVA, an active TVA derivative, or an inhibitor of GPR43 expression or activity.

The supplement or pharmaceutical compositions described herein may be in unit dosage forms suitable for single administration of precise dosages. In unit dosage form, the formulation is divided into unit doses containing appropriate quantities of TVA, an active TVA derivative, or an inhibitor of GPR43 expression or activity. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition.

Dosing and administration regimes are tailored by the clinician, or others skilled in supplements or the pharmacological arts, based upon well-known pharmacological and therapeutic considerations including, but not limited to, the desired level of therapeutic effect, and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy is desired, doses in excess of the average required dose are not uncommon. This approach to dosing is commonly referred to as the “maximal dose” strategy. In certain embodiments, TVA, an active TVA derivative, or an inhibitor of GPR43 expression or activity is administered to a subject at a dose of about 0.01 mg/kg to about 200 mg/kg (e.g., 0.01 mg/kg, 0.02 mg/kg, 0.05 mg/kg, 0.1 mg/kg, 0.2 mg/kg, 0.5 mg/kg, 1 mg/kg, 2 mg/kg, 5 mg/kg, 10 mg/kg, 20 mg/kg, 50 mg/kg, 100 mg/kg, 200 mg/kg, or ranges therebetween). Dosing may be once per day, multiple times per day (e.g., 2, 3, 4, etc.), once per week, or according to any suitable protocol. In some embodiments, TVA, an active TVA derivative, or an inhibitor of GPR43 expression or activity is administered a single time, each time a co-administered agent is delivered, or for a time period of days, weeks, months, indefinitely, etc.

In some embodiments, compositions comprising TVA, an active TVA derivative, or an inhibitor of GPR43 expression or activity are administered alone or in combination with any other cancer treatments (e.g., immunotherapies, chemotherapies, etc.) using standard delivery systems and methods, and in at least some aspects, together with a pharmaceutically acceptable carrier or excipient.

In some embodiments, methods are provided relating to the prevention, treatment or amelioration of a cancer comprising the step of administering to a subject in the need thereof an effective amount of TVA, an active TVA derivative, or an inhibitor of GPR43 expression or activity. In some embodiments, compositions comprising TVA, an active TVA derivative, or an inhibitor of GPR43 expression or activity are provided for the prevention, treatment or amelioration of a cancer. In some embodiments, the methods of administering TVA, an active TVA derivative, or an inhibitor of GPR43 expression or activity and compositions comprising TVA, an active TVA derivative, or an inhibitor of GPR43 expression or activity are provided in combination with other therapies and therapeutics for the treatment/prevention of cancer.

In some embodiments, indications for administration of the composition(s) herein are cancerous diseases. Examples of hematological (or hematogenous) cancers that are treated/prevented in embodiments herein include leukemias, including acute leukemias (such as acute lymphocytic leukemia, acute myelocytic leukemia, acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukemia and myelodysplasia. Examples of solid tumors that are treated/prevented in embodiments herein include, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medu!loblastoma, Schwannoma craniopharyogioma, ependymoma, pincaioma, hemangioblastoma, acoustic neuroma, oligodendroglioma, menangioma, neuroblastoma, retinoblastoma and brain metastases).

The disclosure further encompasses protocols for the co-administration of TVA-containing or active-TVA-derivative-containing compositions (e.g., alone, with a pharmaceutically-acceptable carrier, with other natural products, etc.) with other therapeutics, such as, bispecific antibody constructs, targeted toxins or other blocking or functional antibodies or compounds, immune cells (e.g., CAR-T cells), immune checkpoint blockade therapies, monoclonal antibody therapies, and other treatments described herein. The clinical regimen for co-administration may encompass co-administration at the same time, before or after the administration of the other component. Particular combination therapies include chemotherapy, radiation, surgery, hormone therapy, or other types of immunotherapy.

Many chemotherapeutics are presently known in the art and can be used in combination with the TVA or active-TVA-derivative therapy. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

In some embodiments, TVA, an active TVA derivative, or an inhibitor of GPR43 expression or activity is co-administered with one or more chemotherapeutics. Chemotherapies for use with TVA, an active TVA derivative, or an inhibitor of GPR43 expression or activity include all classes of chemotherapeutic agents, such as, alkylating agents, antimetabolites, plant alkaloids, antibiotics, hormonal agents, and miscellaneous anticancer drugs. Specific agents include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, fuldarabine, navelbine, farnesyl-protein tansferase inhibitors, transplatinum, 5-fluorouracil, vincristin, and vinblastin, or any analog or derivative variant of the foregoing and also combinations thereof. In some embodiments, chemotherapy is employed before, during and/or after administration of TVA, an active TVA derivative, or an inhibitor of GPR43 expression or activity.

In some embodiments, TVA, an active TVA derivative, or an inhibitor of GPR43 expression or activity is co-administered with radiotherapy, methods of which are understood in the field. In some embodiments, radiotherapy is employed before, during and/or after administration of TVA, an active TVA derivative, or an inhibitor of GPR43 expression or activity.

In some embodiments, TVA, an active TVA derivative, or an inhibitor of GPR43 expression or activity co-administered with non-immune based targeted therapies, such as, agents that inhibit signaling pathways such WNT, p53, and/or RB-signaling pathways. Other examples include agents that inhibit tyrosine kinases, BRAF, STAT3, c-met, regulate gene expression, induce cell death or block blood vessel formation. Examples of specific agents include imatinib mesylate, dasatinib, nilotinib, bosutinib, lapatinib, gefinitib, erlotinib, tensirolimus, everolimus, vemurafenib, crizotinib, vorinostat, romidepsin, bexarotene, alitrionin, tretionin, bortezomib, carfilzomib, pralatrexate, sorafenib, sunitinib, pazopanib, regorafenib, or cabozantinib. In some embodiments, non-immune based targeted therapy is employed before, during and/or after administration of TVA, an active TVA derivative, or an inhibitor of GPR43 expression or activity.

In some embodiments, TVA, an active TVA derivative, or an inhibitor of GPR43 expression or activity is co-administered with a gene therapy in which a therapeutic polynucleotide is administered before, after, or at the same time as the composition comprising TVA. A variety of expression products are encompassed, including inducers of cellular proliferation, inhibitors of cellular proliferation, or regulators of programmed cell death.

In some embodiments, TVA, an active TVA derivative, or an inhibitor of GPR43 expression or activity is administered before, during, and/or after surgery. Surgeries include resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that embodiments herein may be used in conjunction with removal of superficial cancers, precancers, or incidental amounts of normal tissue.

In some embodiments, TVA, an active TVA derivative, or an inhibitor of GPR43 expression or activity is co-administered with other agents to improve the therapeutic efficacy of treatment.

In some embodiments, TVA, an active TVA derivative, or an inhibitor of GPR43 expression or activity is provided as part of a kit or system along with one or more additional components, such as instructions, devices for administration, additional therapeutic agents, diagnostic agents, research agents, etc.

In particular embodiments, TVA, an active TVA derivative, or an inhibitor of GPR43 expression or activity is co-administered with an immunotherapy. Immunotherapeutics generally rely on the use of immune effector cells (e.g., NK cells, T cells (e.g., CAR-T cells), etc.) and/or molecules (e.g., checkpoint inhibitors, bispecific engager molecules, monoclonal antibodies, etc.) to target and destroy cancer cells.

A immune effector for co-administration with TVA, an active TVA derivative, or an inhibitor of GPR43 expression or activity may be, for example, an antibody, antibody fragment, bispecific engager, etc. that is specific (e.g., binds to) a marker on the surface of a cancer cell, tumor cell, cancer stem cell, etc. The immune effector alone may serve as a therapy or it may recruit cells to effect cell killing. The immune effector may also prevent cancer immunoevasion or immunosuppression. The immune effector also may be conjugated to a drug or toxin (e.g., chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T-cells, NKT cells, and NK cells. In some embodiments, immunotherapy is employed before, during and/or after administration of TVA. In some embodiments, TVA is co-administered with an immune checkpoint inhibitor (e.g., anti-PD1, anti-PDL1, anti-CTLA-4, etc.).

In some embodiments, methods comprise co-administering TVA, an active TVA derivative, or an inhibitor of GPR43 expression or activity to a subject with an immune checkpoint inhibitor. In some embodiments, the immune checkpoint inhibitor is a small molecule, protein, or peptide that specifically binds to an immune checkpoint protein. In some embodiments, the immune checkpoint protein is selected from the group consisting of CTLA4, PD-1, PD-L1, PD-L2, A2AR, B7-H3, B7-H4, BTLA, KIR, LAG3, TIM-3 or VISTA. In some embodiments, the immune checkpoint inhibitor is an antibody or antigen-binding fragment thereof. In some embodiments, the immune checkpoint inhibitor is an interfering nucleic acid molecule. In some embodiments, the interfering nucleic acid molecule is an siRNA molecule, an shRNA molecule or an antisense RNA molecule. In some embodiments, the immune checkpoint inhibitor is selected from the group consisting of nivolumab, pembrolizumab, pidilizumab, AMP-224, AMP-514, STI-A1110, TSR-042, RG-7446, BMS-936559, BMS-936558, MK-3475, CT O1 1, MPDL3280A, MEDI-4736, MSB-0020718C, AUR-012 and STI-A1010. In some embodiments, the immune checkpoint inhibitor is administered before the TVA, an active TVA derivative, or an inhibitor of GPR43 expression or activity. In some embodiments, the immune checkpoint inhibitor is administered at least one day before the TVA, an active TVA derivative, or an inhibitor of GPR43 expression or activity. In some embodiments, the immune checkpoint is administered at about the same time as the TVA, an active TVA derivative, or an inhibitor of

GPR43 expression or activity. In some embodiments, the immune checkpoint inhibitor is administered on the same day as the TVA, an active TVA derivative, or an inhibitor of GPR43 expression or activity. In some embodiments, the immune checkpoint inhibitor is administered after the TVA, an active TVA derivative, or an inhibitor of GPR43 expression or activity. In some embodiments, the immune checkpoint inhibitor is administered at least one day after the TVA, an active TVA derivative, or an inhibitor of GPR43 expression or activity. In some embodiments, the immune checkpoint inhibitor is administered by injection. In some embodiments, the injection is an intravenous, intramuscular, intratumoral or subcutaneous injection.

In some embodiments, methods comprise co-administering TVA, an active TVA derivative, or an inhibitor of GPR43 expression or activity to a subject with a bispecific agent (e.g., bispecific antibody, bispecific engager, etc.). In some embodiments, TVA is co-administered with bispecific antibodies that bind with one “arm” to a surface antigen on a target cells (e.g., a tumor antigen), and with the second “arm” to an activating, invariant component of the T cell receptor (TCR) complex. The simultaneous binding of such a bispecific agent to both of its targets forces an interaction between target cell (e.g., cancer cell) and T cell, causing activation of any cytotoxic T cell and subsequent lysis of the target cell. Bispecific T cell engager (BiTE) molecules are tandem scFv molecules wherein two scFv molecules are fused by a flexible linker. BiTEs have been very well characterized and already shown some promise in the clinic (reviewed in Nagorsen and Bauerle, Exp Cell Res 317, 1255-1260 (2011); incorporated by reference in its entirety). Further bispecific engager formats for T cell engagement and targeting to cancer cells that find use with TVA co-administration include diabodies (Holliger et al, Prot Eng 9, 299-305 (1996); incorporated by reference in its entirety); derivatives thereof, such as tandem diabodies (Kipriyanov et al, J Mol Biol 293, 41-66 (1999); incorporated by reference in its entirety); dual affinity retargeting (DART) molecules, which are based on the diabody format but feature a C-terminal disulfide bridge for additional stabilization (Moore et al, Blood 117, 4542-51 (2011); incorporated by reference in its entirety); triomabs, which are whole hybrid mouse/rat IgG molecules (reviewed in Seimetz et al, Cancer Treat Rev 36, 458-467 (2010); incorporated by reference in its entirety). In some embodiments, the bispecific engager comprises an activation domain that binds to a T cell activating antigen, such as CD3. In some embodiments, the bispecific engager comprises an antigen-binding domain capable of binding is a tumor or cancer cell antigen. In some embodiments, antigen-binding domain binds a tumor associated antigen (TAA). In some embodiments, antigen-binding domain binds a tumor specific antigen (TSA). Non-limiting examples of TSAs or TAAs include the following: Differentiation antigens such as MART-1/MelanA (MART-I), gp100 (Pmel 17), tyrosinase, TRP-1, TRP-2 and tumor-specific multi-lineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In some embodiments, methods comprise co-administering TVA, an active TVA derivative, or an inhibitor of GPR43 expression or activity to a subject with a CAR or CAR-T cell. CARs are hybrid molecules comprising three essential units: (1) an extracellular antigen-binding motif, (2) linking/transmembrane motifs, and (3) intracellular T-cell signaling motifs (Long A H, Haso W M, Orentas R J. Oncoimmunology. 2013; 2 (4): e23621; incorporated by reference in its entirety). The antigen-binding motif of a CAR is commonly fashioned after an single chain Fragment variable (ScFv), the minimal binding domain of an immunoglobulin (Ig) molecule. The linking motifs of a CAR can be a relatively stable structural domain, such as the constant domain of IgG, or designed to be an extended flexible linker. Structural motifs, such as those derived from IgG constant domains, can be used to extend the ScFv binding domain away from the T-cell plasma membrane surface. Signaling motifs used in CARs typically comprise the CD3-zeta chain because this core motif is the key signal for T cell activation. CARs and CAR-T cells capable of interacting (binding) any antigens described herein are provided for co-administration with TVA, an active TVA derivative, or an inhibitor of GPR43 expression or activity.

In some embodiments, methods comprise co-administering TVA, an active TVA derivative, or an inhibitor of GPR43 expression or activity to a subject with a monoclonal antibody (mAb). Examples of mAb for co-administration with TVA, an active TVA derivative, or an inhibitor of GPR43 expression or activity include known antibodies such as brentuximab, trastuzumab, inotuzumab, gemtuzumab, glembatumumab, labetuzumab, sacituzumab, lifastuzumab, indusatumab, polatuzumab, pinatuzumab, coltuximab, indatuximab, milatuzumab, rovalpituzumab, anetumab, tisotumab, mirvetuximab, lorvotuzumab, rituximab, depatuxizumab, denintuzumab, enfortumab, telisotuzumab, vandortuzumab, sofituzumab, vorsetuzumab, mirvetuximab, naratuximab, cantuzumab, laprituximab, bivatuzumab, vadastuximab, lupartumab, aprutumab, abagovomab, abciximab, abituzumab, abrilumab, actoxumab, adalimumab, adecatumumab, aducanumab, afasevikumab, afelimomab, alacizumab, alemtuzumab, alirocumab, altumomab, amatuximab, anatumomab, anifrolumab, anrukinzumab, apolizumab, arcitumomab, ascrinvacumab, aselizumab, atezolizumab, atinumab, atorolimumab, avelumab, azintuxizumab, bapineuzumab, basiliximab, bavituximab, bectumomab, begelomab, belimumab, benralizumab, bertilimumab, besilesomab, bevacizumab, bezlotoxumab, biciromab, bimagrumab, bimekizumab, bleselumab, blinatumomab, blontuvetmab, blosozumab, bococizumab, brazikumab, briakinumab, brodalumab, brolucizumab, brontictuzumab, burosumab, cabiralizumab, camrelizumab, caplacizumab, capromab, carlumab, carotuximab, catumaxomab, cedelizumab, certolizumab, cetuximab, citatuzumab, cixutumumab, clenoliximab, clivatuzumab, codrituzumab, conatumumab, concizumab, cosfroviximab, crenczumab, crizanlizumab, crotedumab, dacetuzumab, daclizumab, dalotuzumab, dapirolizumab, daratumumab, dectrekumab, demcizumab, denosumab, detumomab, dezamizumab, dinutuximab, diridavumab, domagrozumab, dorlimomab, drozitumab, duligotuzumab, dupilumab, durvalumab, dusigitumab, duvortuxizumab, ecromeximab, eculizumab, edobacomab, edrecolomab, efalizumab, efungumab, eldelumab, elezanumab, elotuzumab, elsilimomab, emactuzumab, emapalumab, emibetuzumab, emicizumab, enavatuzumab, enlimomab, enoblituzumab, enokizumab, enoticumab, ensituximab, epitumomab, epratuzumab, eptinezumab, erenumab, erlizumab, ertumaxomab, etaracizumab, etrolizumab, evinacumab, evolocumab, exbivirumab, faralimomab, farletuzumab, fasinumab, felvizumab, fezakinumab, ficlatuzumab, figitumumab, firivumab, flanvotumab, fletikumab, fontolizumab, foralumab, foravirumab, fremanezumab, fresolimumab, firunevetmab, fulranumab, futuximab, galcanezumab, galiximab, ganitumab, gantenerumab, gatipotuzumab, gavilimomab, gedivumab, gevokizumab, gilvetmab, girentuximab, golimumab, guselkumab, ibalizumab, ibritumomab, icrucumab, idarucizumab, ifabotuzumab, igovomab, imalumab, imciromab, imgatuzumab, inclacumab, inebilizumab, infliximab, inolimomab, intetumumab, ipilimumab, iratumumab, isatuximab, itolizumab, ixekizumab, keliximab, lacnotuzumab, lampalizumab, lanadelumab, landogrozumab, larcaviximab, lebrikizumab, lemalesomab, lenzilumab, lerdelimumab, lesofavumab, letolizumab, lexatumumab, libivirumab, lifatuzumab, ligelizumab, lilotomab, lintuzumab, lirilumab, lodelcizumab, lokivetmab, lorvotuzumab, losatuximab, lucatumumab, lulizumab, lumretuzumab, lutikizumab, mapatumumab, margetuximab, maslimomab, matuzumab, mavrilimumab, mepolizumab, metelimumab, minretumomab, mitumomab, modotuximab, mogamulizumab, monalizumab, morolimumab, motavizumab, moxetumomab, muromonab, nacolomab, namilumab, naptumomab, narnatumab, natalizumab, navicixizumab, navivumab, nebacumab, necitumumab, nemolizumab, nerelimomab, nesvacumab, nimotuzumab, nivolumab, obiltoxaximab, obinutuzumab, ocaratuzumab, ocrelizumab, odulimomab, ofatumumab, olaratumab, oleclumab, olendalizumab, olokizumab, omalizumab, onartuzumab, ontuxizumab, opicinumab, oportuzumab, oregovomab, oreticumab, orticumab, otelixizumab, otlertuzumab, oxelumab, ozanezumab, ozoralizumab, pagibaximab, palivizumab, pamrevlumab, panitumumab, panobacumab, parsatuzumab, pascolizumab, pasotuxizumab, pateclizumab, patritumab, pembrolizumab, perakizumab, pertuzumab, pexelizumab, pidilizumab, placulumab, plozalizumab, ponczumab, porgaviximab, prczalumab, priliximab, pritoxaximab, pritumumab, quilizumab, racotumomab, radretumab, rafivirumab, ralpancizumab, ramucirumab, ranevetmab, ranibizumab, raxibacumab, refanezumab, regavirumab, remtolumab, reslizumab, rilotumumab, rinucumab, risankizumab, rivabazumab, robatumumab, roledumab, romosozumab, rontalizumab, rosmantuzumab, rovelizumab, rozanolixizumab, ruplizumab, samalizumab, sarilumab, satralizumab, satumomab, secukinumab, selicrelumab, seribantumab, setoxaximab, sevirumab, sibrotuzumab, sifalimumab, siltuximab, simtuzumab, siplizumab, sirukumab, solanezumab, solitomab, sontuzumab, stamulumab, sulesomab, suptavumab, suvizumab, suvratoxumab, tabalumab, tadocizumab, talizumab, tamtuvetmab, tanezumab, taplitumomab, tarextumab, tavolixizumab, fanolesomab, nofetumomab, pintumomab, tefibazumab, telimomab, telisotuzumab, tenatumomab, teneliximab, teplizumab, teprotumumab, tesidolumab, tezepelumab, tigatuzumab, tildrakizumab, timigutuzumab, timolumab, tocilizumab, tomuzotuximab, toralizumab, tosatoxumab, tositumomab, tovetumab, tralokinumab, tregalizumab, tremelimumab, trevogrumab, tucotuzumab, tuvirumab, ublituximab, ulocuplumab, urelumab, urtoxazumab, ustekinumab, utomilumab, vantictumab, vanucizumab, vapaliximab, varisakumab, varlilumab, vatelizumab, vedolizumab, veltuzumab, vepalimomab, vesencumab, visilizumab, vobarilizumab, volociximab, vonlerolizumab, votumumab, vunakizumab, tacatuzumab, zalutumumab, zanolimumab, ziralimumab, zolimomab or anti-embigin antibody; examples of another aspect thereof include brentuximab, trastuzumab, inotuzumab, gemtuzumab, labetuzumab, polatuzumab, coltuximab, indatuximab, anetumab, rituximab, denintuzumab, laprituximab, vadastuximab, glembatumumab, cetuximab, alemtuzumab or depatuxizumab; examples of another aspect thereof include brentuximab, trastuzumab, rituximab or anti-embigin antibody; and examples of another aspect thereof include brentuximab or trastuzumab, and preferably, examples of another aspect include brentuximab.

Some embodiments described herein find use with TVA. Some embodiments herein find use with the full genus of TVA derivatives described herein. Some embodiments herein find use with specific TVA derivatives described herein.

### EXPERIMENTAL

### Example 1

### Dietary Trans-Vaccenic Acid Promotes Anti-Tumor Immunity

### Results

### Dietary TVA Enhances Anti-Tumor Immunity

A blood chemical compound library was constructed for the screening purpose. There are roughly 633 circulating “blood chemicals” (Table 1), including inorganics (e.g., minerals), organic metabolites (e.g., carbohydrates and amino acids), lipids (e.g., unsaturated and saturated fatty acids), dietary supplements (e.g., vitamins and antioxidants), and proteins (e.g., hormones and cytokines). This summary combines the United States Department of Agriculture (USDA) list of defined diet-derived nutrients (www.nal.usda.gov/legacy/fnic/nutrient-lists-standard-reference-legacy-2018), GNC list of dietary supplements (www.gnc.com/vitamins-supplements), and Wikipedia list of human blood components (www.wikipedia.org/wiki/List_of_human_blood_components). The library used for cell-based screens contains 255 compounds (Table 2) that are commercially available. Proteins that have been extensively studied and some supplements that only function at the organismal level such as fish oils and herbs were excluded. The reported physiological range of serum levels and working levels (˜2× of highest reported serum level) in the screens of individual blood chemicals are included in Table 2.

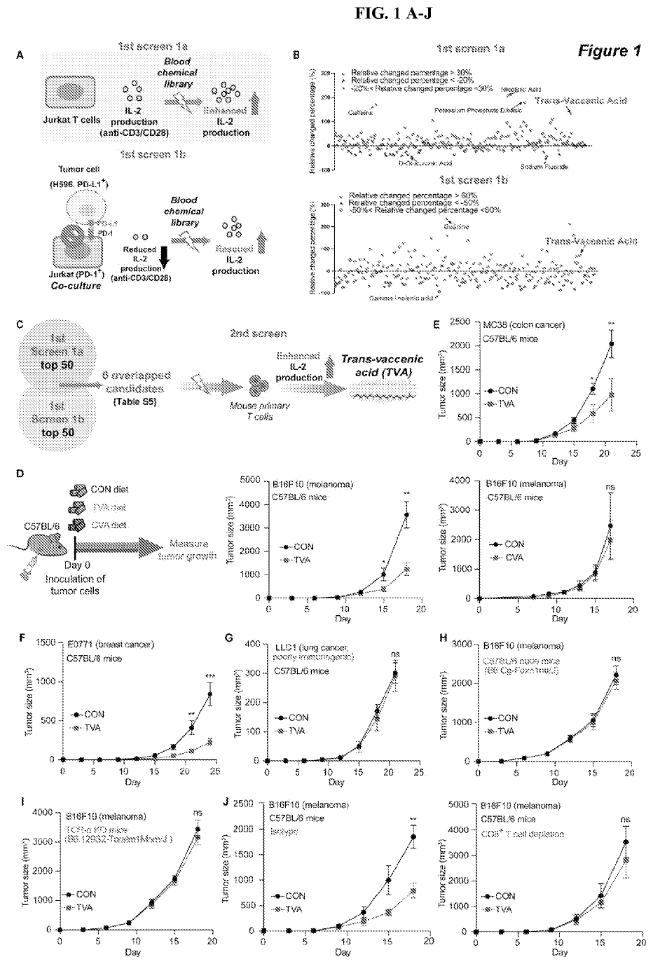

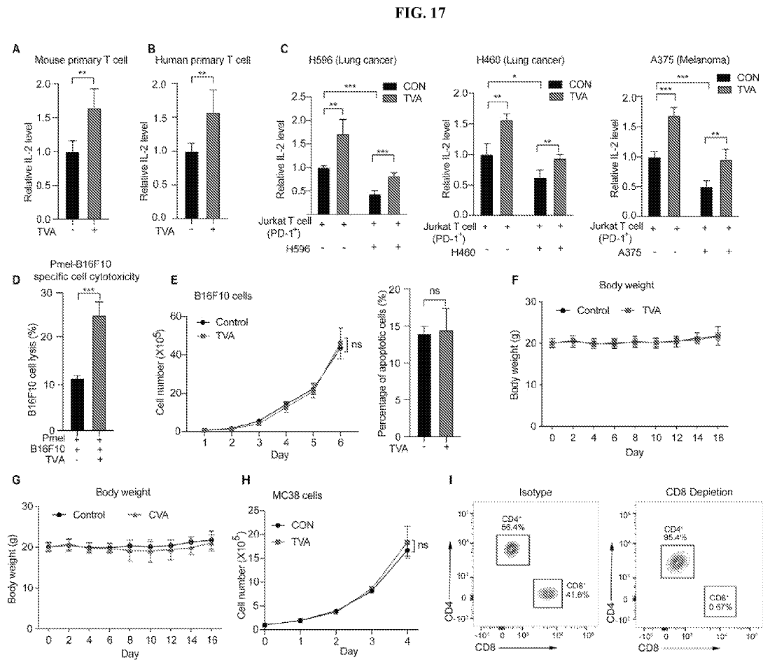

Two preliminary screens were designed and performed. The 1^{st} screen 1a (Table 3) was to identify blood chemicals that enhance activation of Jurkat T cells stimulated by anti-CD3/CD28 antibodies (FIG. 1A, upper). The 1^{st} screen 1b (Table 4) was to identify blood chemicals that rescue PD-L1/PD-1 dependent exhaustion of Jurkat T cells stably expressing PD-1 induced by co-cultured PD-L1-expressing human H596 lung cancer cells (FIG. 1A, lower). The results of the two screens are summarized in FIG. 1B and Tables 3-4. Six overlapping top candidates identified from these screens (FIG. 1C), when comparing top 50 candidates from each screen, were further examined for their effects on activation of murine primary T cells, and CD4⁺ or CD8⁺ T cells (Table 5). Trans-vaccenic acid (TVA) was top ranked in the secondary screen (Table 5) and was focused on. It was confirmed that TVA treatment enhances IL-2 production from mouse and human primary T cells (FIGS. 17A and 17B, respectively). TVA treatment also rescued PD-L1/PD-1-dependent exhaustion of Jurkat T cells induced by co-cultured PD-L1-expressing human H596 and H460 lung cancer cells, or A375 human melanoma cells (FIGS. 17C), assessed by reversed inhibition of IL-2 production. These results together suggest that TVA may not only enhance T cell activity but also rescue PD-1-dependent T cell exhaustion.

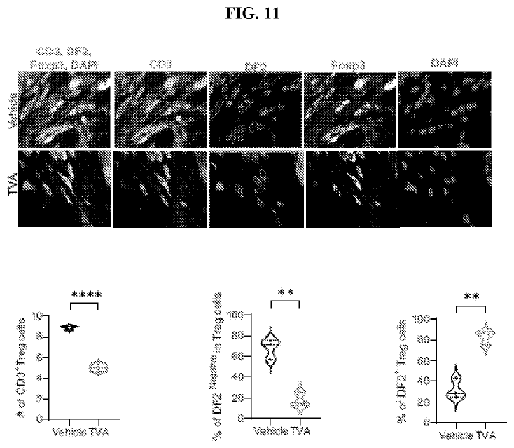

To determine whether TVA enhances cytotoxic T cell function, TVA's effects on co-cultured mouse melanoma B16F10 cells and pmel-1-specific mouse T cells was tested. It was found that TVA treatment enhances cytotoxicity of B16F10 cells induced by co-cultured pmel-1-specific T cells (FIG. 17D). TVA treatment did not alter B16F10 cell proliferation or apoptosis (FIG. 17E), suggesting that TVA functions through T cells, not B16F10 tumor cells. In addition, it was observed that tumor growth potential of immunogenic B16F10 cells was significantly attenuated in syngeneic mice fed with TVA-enriched diet (10 kcal % fat with 1% TVA), compared to mice fed with control diet (10 kcal % fat) (FIG. 1D, middle). In contrast, the control group of B16F10 syngeneic mice fed with either CVA-enriched diet or control diet did not show difference in tumor growth potential of B16F10 cells (FIG. 1D, right). Both TVA- or CVA-enriched diets did not alter body weights of mice (FIG. 17F-17G, respectively). Consistent with this finding, dietary TVA significantly attenuated tumor growth potential of mouse immunogenic MC38 colon cancer (FIG. 1E) without altering MC38 cell proliferation (FIG. 17H). Dietary TVA also reduced tumor growth potential of E0771 breast cancer cells in syngeneic mice (FIG. 1F), but not in mice inoculated with poorly immunogenic mouse LLC1 lung cancer cells (FIG. 1G). Furthermore, dietary TVA did not affect B16F10 tumor growth in syngeneic immune-deficient nude mice (FIG. 1H), nor in TCR-a knockout (KO) mice (FIG. 11), or mice with depletion of CD8⁺ T cells by anti-CD8a antibodies (FIG. 1J; depletion efficiency shown in FIG. 17I). These data together indicate that TVA likely promotes anti-tumor immunity through regulation of cytotoxic CD8⁺ T cells.

### Dietary TVA Enhances Tumor-Infiltrating and Cytotoxic Function of Effector CD8⁺ T Cells

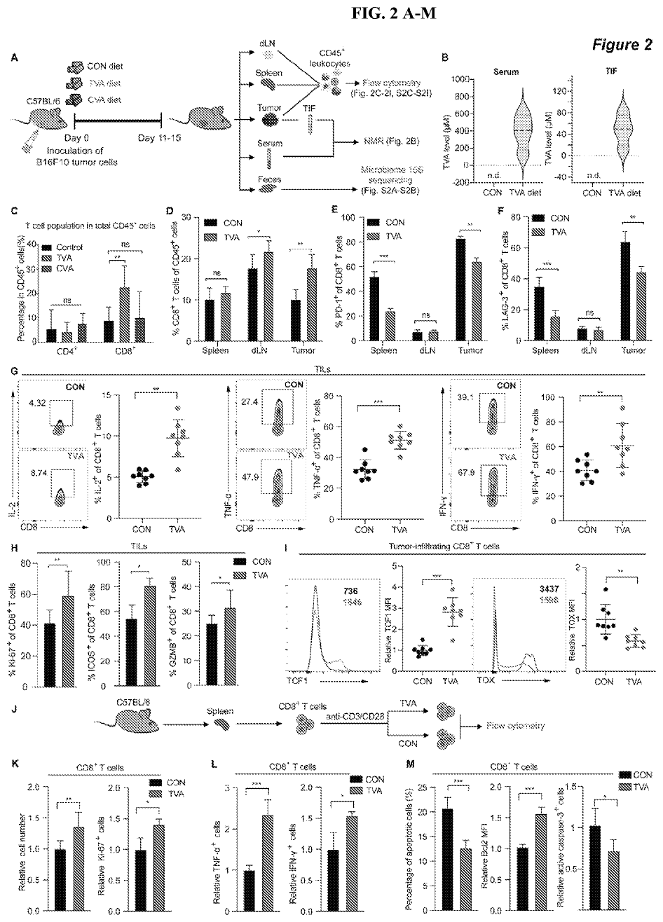

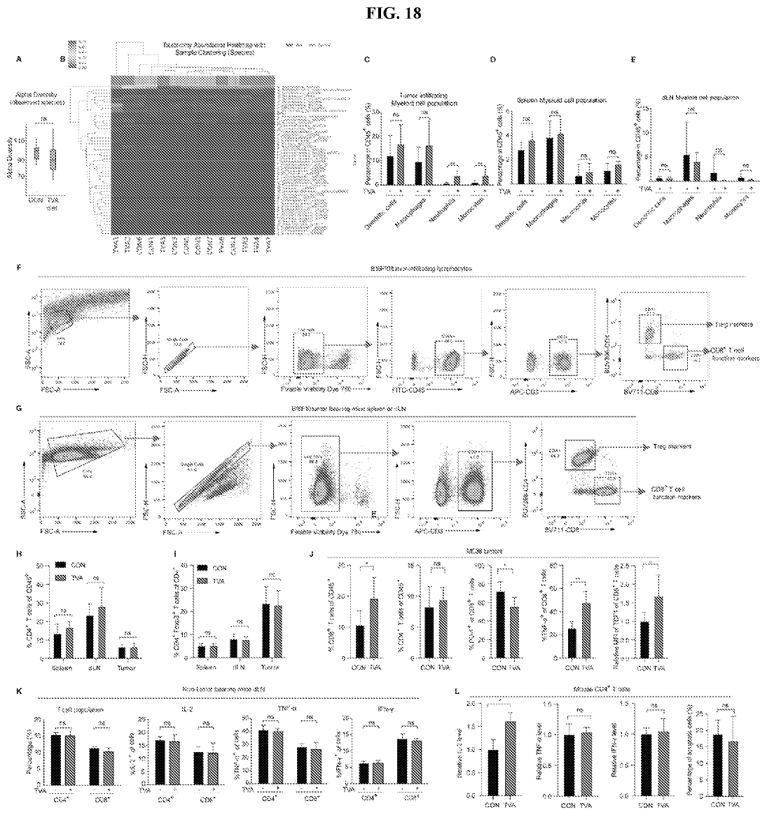

To understand how dietary TVA influences tumor bearing mice, gut microbiota was first examined. The results suggest that TVA-enriched diet does not significantly alter the diversity and patterns of gut microbial distribution in mice (FIGS. 18A-18B). B16F10 tumors were analyzed 11-15 days after implantation, when tumors were similar in volume (FIG. 2A). Quantitative nuclear magnetic resonance (NMR)-based measurement revealed that the average serum TVA level is ˜386 μM, and the average TVA level in tumor interstitial fluid (TIF) is ˜48 μM (FIG. 2B). Flow cytometry analyses revealed that TVA diet, but not control CVA diet, results in an increased CD8⁺ T cell population in tumor-infiltrating lymphocytes (TILs). In contrast, CD4⁺ T cell population in TILs is not altered (FIG. 2C). TVA diet also markedly increases tumor-infiltrating populations of neutrophils and monocytes, but not dendritic cells or macrophages (FIG. 18C), nor does TVA diet alter populations of these myeloid cells in spleens (FIG. 18D) or draining lymph nodes (dLNs; FIG. 18E).

Dietary TVA effects on different T cell populations were next examined. It was found that TVA diet results in a larger percentage of CD8⁺ T cells as a fraction of the CD45⁺ leukocytes infiltrated in B16F10 tumors and dLNs but not spleens (FIGS. 2D, 18F-18G). Dietary TVA reduces exhaustion of CD8⁺ T cells in tumors and spleens, not in dLNs, which is assessed by decreased expression levels of PD-1 and LAG-3 as exhaustion markers in CD8⁺ T cells (FIGS. 2E-2F, respectively). In contrast, TVA diet has no effects on conventional CD4⁺ T cell (FIG. 18H) or CD4⁺Foxp3⁺ Treg cell (FIG. 18I) populations in spleens, dLNs and tumors from B16F10 tumor-bearing mice. Further analysis of tumor-infiltrating CD8⁺ T cells with additional representative markers revealed that dietary TVA promotes CD8⁺ T cell function with increased expression levels of cytokines including IL-2, TNF-α, and IFN-γ (FIG. 2G), proliferation marker Ki-67, co-stimulatory receptor ICOS and cytolytic molecule granzyme B (GZMB) (FIG. 2H), as well as increased TCF1 expression for stem-like CD8⁺ T cell survival (Chen et al., 2019) but reduced expression level of exhaustion marker TOX (FIG. 2I). Similar results were obtained using tumor-infiltrating CD8⁺ T cells isolated from MC38 tumors (FIG. 18J). In contrast, TVA diet has no effects on CD4⁺ and CD8⁺ T cell population, nor activation assessed by expression levels of IL-2, TNF-α, and IFN-γ in non-tumor bearing mice (FIG. 18K), suggesting that dietary TVA's effects on T cells are specific to tumor context. These results together suggest that dietary TVA promotes accumulation and function of tumor-infiltrating CD8⁺ T cells.

Consistent with these findings, anti-CD3/CD28-stimulated primary CD8⁺ T cells with TVA treatment in vitro (FIG. 2J) show an increased cell number with elevated expression of proliferation marker Ki-67 (FIG. 2K), increased expression of TNF-α and IFN-γ (FIG. 2L), but reduced apoptosis assessed by decreased annexin V staining, increased Bcl-2 with decreased active caspases-3 levels (FIG. 2M). In contrast, primary CD4⁺ T cells with TVA treatment in vitro show increased IL-2 production but non-altered expression of TNF-α and IFN-γ or apoptosis assessed by annexin V staining (FIG. 18L). These results collectively suggest that TVA selectively enhances function of stimulated CD8⁺ T cells.

### TVA Exhibits a Nutrient-Independent, Signaling Function Through a GPCR-CREB Axis

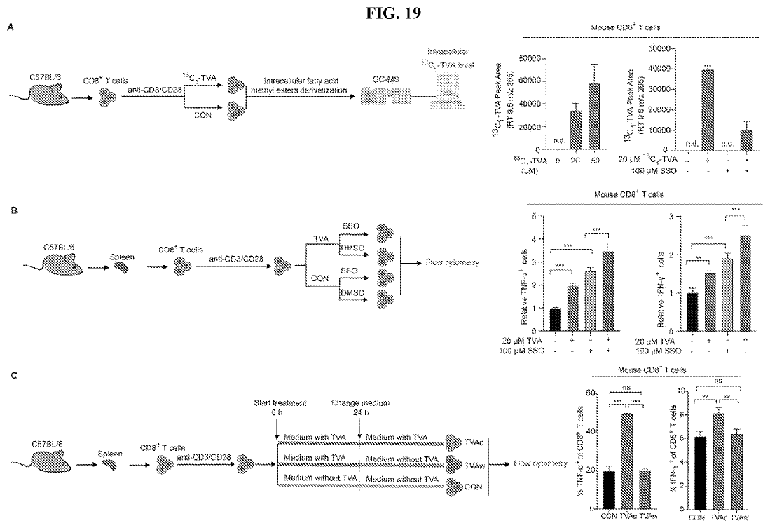

To determine the underlying mechanisms by which TVA activates CD8⁺ T cells, it was first determined whether TVA functions in an extracellular or intracellular manner. Quantitative mass spectrometry detected isotope-labeled ¹³C-TVA imported from media to primary murine CD8⁺ T cells in a dose-dependent manner, which is CD36-dependent because treatment with CD36 inhibitor sulfosuccinimidyl oleate (SSO) drastically reduced import of ¹³C-TVA (FIG. 19A). However, SSO treatment does not affect TVA-dependent CD8⁺ T cell activation (FIG. 19B). In addition, continuous TVA treatment (TVAc) enhances activation of mouse CD8⁺ T cells, whereas withdrawal of TVA from culture media (TVAw) abolishes TVA-enhanced CD8⁺ T cell activation, suggesting that TVA's effects are reversible (FIG. 19C). These data together indicate that TVA functions outside of CD8⁺ T cells without being metabolized to provide nourishment. Thus, TVA may have a nutrient-independent effect on CD8⁺ T cells.

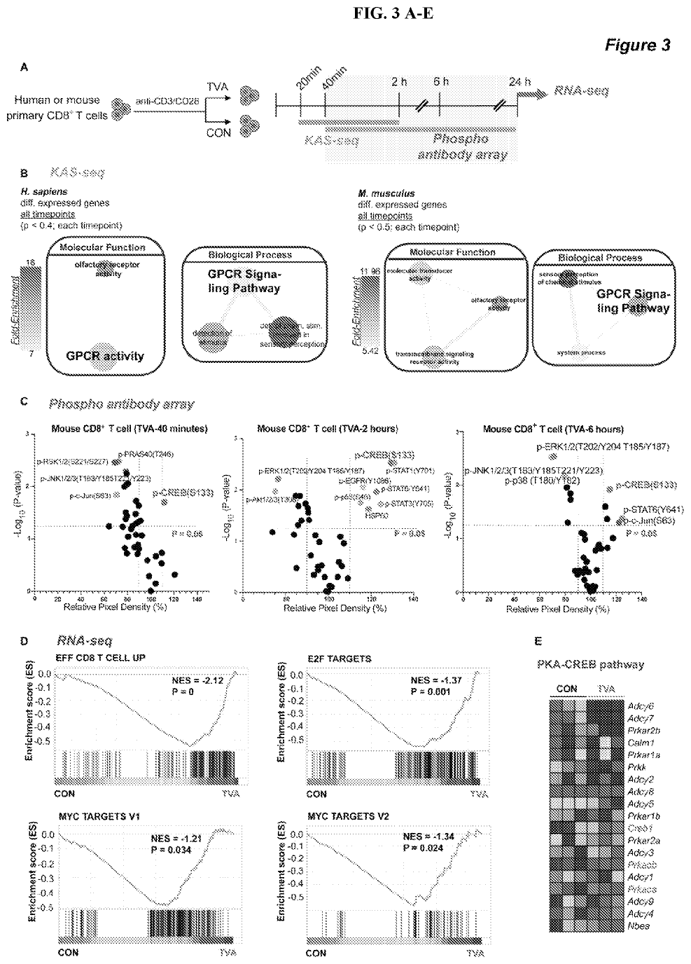

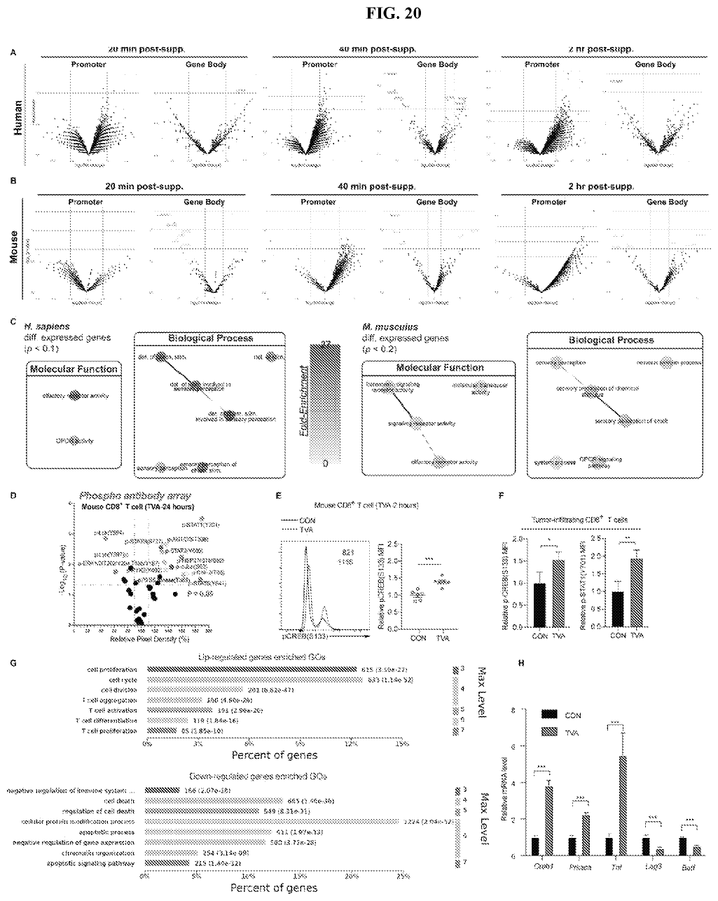

To elucidate the underlying mechanisms, integrated temporal mechanistic investigations on TVA's effects using primary human or murine CD8⁺ T cells (FIG. 3A) were designed and performed. These include (1) kethoxal-assisted single-stranded DNA sequencing (KAS-seq) approach (20 min-2 h) (Wu et al., 2020b) for initial influences of TVA treatment on cells by capturing global transcription dynamics at different time intervals post-TVA treatment; (2) phospho-antibody array (40 min-24 h) for early cellular signaling changes; and (3) RNA-seq approach (24 h) whole transcriptome analysis. Functional enrichment of the top ranking altered gene bodies from the genome-scale KAS-seq results (FIGS. 20A-20B) revealed G-protein coupled receptor (GPCR) activities among the top-enriched ontologies in CD8⁺ T cells at an early stage of post-TVA treatment (20 min-2 h) (FIGS. 3B and 20C). Consistent with this finding, an increased phosphorylation level of the transcription factor cAMP response element-binding protein (CREB) was observed, which is a common downstream effector of GPCR signaling (Zhang et al., 2020). Increased p-CREB level was detected at as early as 40 min, and at 2 h and 6 h post-TVA treatment by phospho-antibody array analysis, which reduces at 24 h post-TVA treatment (FIGS. 3C, 20D) (full phospho-antibody array data could be found in Table 6). The increased p-CREB level in mouse primary CD8⁺ T cells treated with TVA for 2 h was confirmed by flow cytometry analysis (FIG. 20E). Phosphorylation levels of signal transducer and activator of transcription 1 (p-STAT1) is also elevated upon TVA treatment at 2 h and 24 h (FIGS. 3C and 20D), which is important for efficient expansion of CD8⁺ T cells (Quigley et al., 2008). Increased p-CREB and p-STAT1 levels in tumor-infiltrating CD8⁺ T cells in B16F10 tumors from syngeneic mice fed on TVA were confirmed by flow cytometry analysis (FIG. 20F).

Consistent with these findings, Gene Ontology (GO) enrichment analysis (Ashburner et al., 2000; Carbon et al., 2021) and global Gene Set Enrichment Analysis (GSEA) (Subramanian et al., 2005) identified a set of increased signal pathways which are responsible for the effects of TVA treatment on CD8⁺ T cells at 24 h including T cell proliferation and activation, and a set of decreased signal pathways including apoptosis (FIG. 20G). Notably, TVA treatment enhances expression of the genes enriched in effector CD8⁺ T cell function, E2F and MYC pathways, correlating with enhanced CD8⁺ T cell function and proliferation (FIG. 3D). In particular, TVA treatment upregulates expression of Creb1 encoding CREB and Prkach and Prkaca encoding catalytic subunit alpha and beta, respectively, of cAMP-dependent protein kinase A (PKA) (FIGS. 3E and 20H), as well as cytokine gene Tnf, but downregulates T cell exhaustion-related Lag3 and Batf gene (FIG. 20H). Collectively, these results suggest that enhanced CD8⁺ T cell function induced by TVA is mediated through a GPCR-CREB pathway with a positive feedback to augment PKA and CREB gene expression.

### TVA Signals Through the GPCR-cAMP-PKA-CREB Axis to Enhance CD8⁺ T Cell Function

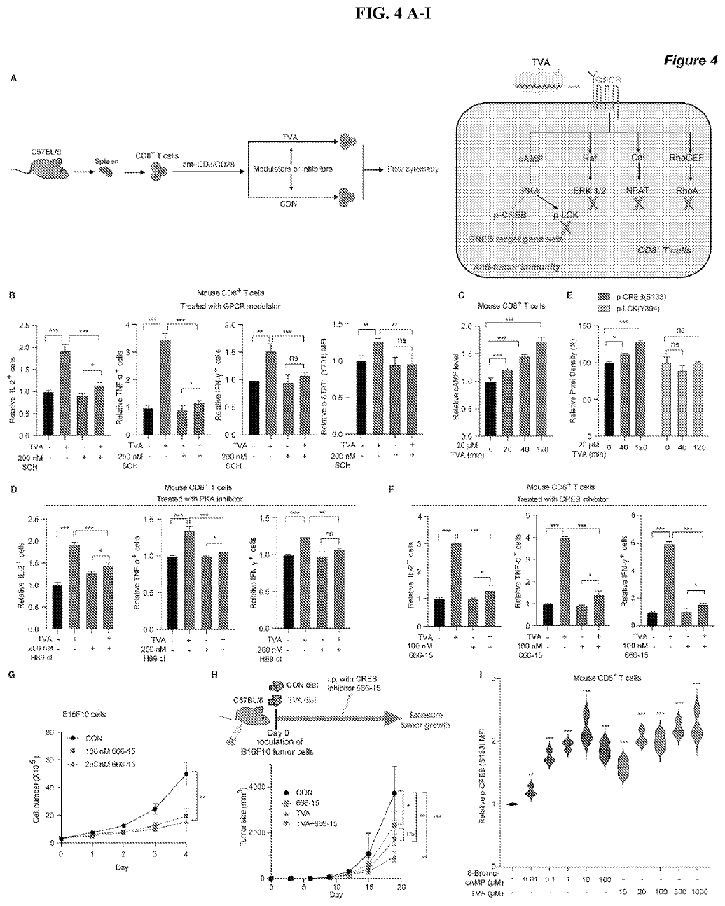

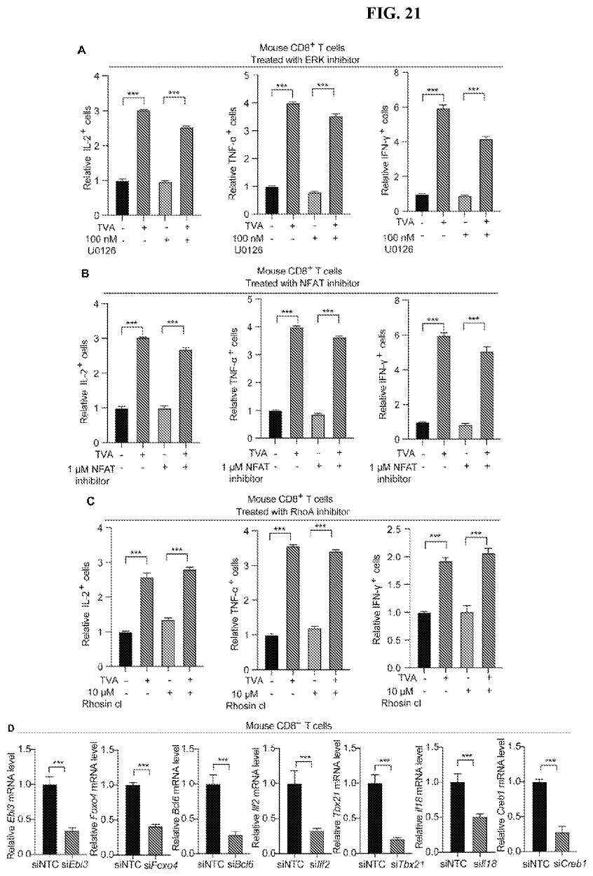

Next, which downstream pathway(s) of GPCR is required for TVA function (results summarized in FIG. 4A) was determined. It was confirmed that treatment with SCH 202676 hydrobromide, a sulphydryl-reactive compound that blocks both agonist and antagonist binding to GPCRs, abolishes TVA-enhanced activation of CD8⁺ T cells assessed by expression levels of IL-2, TNF-α and IFN-γ and p-STAT1 level (FIG. 4B). Consistent with this finding, TVA treatment elevates cAMP levels in CD8⁺ T cells (FIG. 4C), whereas treatment with PKA inhibitor H89 similarly abolishes TVA-enhanced activation of CD8⁺ T cells (FIG. 4D). In contrast, specific inhibitors targeting alternative downstream effectors of GPCR including MEK1 (FIG. 21A), NFAT (FIG. 21B) or RhoA (FIG. 21C) do not alter TVA-dependent CD8⁺ T cell activation. PKA downstream effectors include CREB (Wen et al., 2010) and lymphocyte-specific tyrosine kinase Lck (Tasken and Ruppelt, 2006) that are reported to positively and negatively regulate T cell activation, respectively. It was found that TVA treatment promotes phosphorylation of CREB but does not alter phosphorylation level of Lck (p-Lck) (FIG. 4E). In addition, treatment with CREB inhibitor 666-15 drastically eliminates TVA-enhanced activation of CD8⁺ T cells (FIG. 4F). It was next found that treatment with 666-15 as a single agent results in reduced B16F10 cell proliferation (FIG. 4G) and tumor growth (FIG. 4H). Consistent with previous finding, dietary TVA drastically reduces B16F10 tumor growth in syngeneic mice (FIG. 4H). However, combined treatment with 666-15 and dietary TVA resulted in a significantly rescued tumor growth potential of B16F10 syngeneic mice compared to mice treated with dietary TVA alone, back to a comparable level to that in mice treated with 666-15 alone (FIG. 4H). These results suggest that inhibition of CREB overshadows dietary TVA's effects on anti-tumor immunity. Lastly, treatment with increasing concentrations of cell permeable 8-Bromo-cAMP similarly increase p-CREB level in CD8⁺ T cells in a dose-dependent manner as observed in cells treated with increasing concentrations of TVA (FIG. 4I)

### TVA-Enhanced CD8⁺ T Cell Function is Primarily Mediated Through CREB and its Target Gene Sets

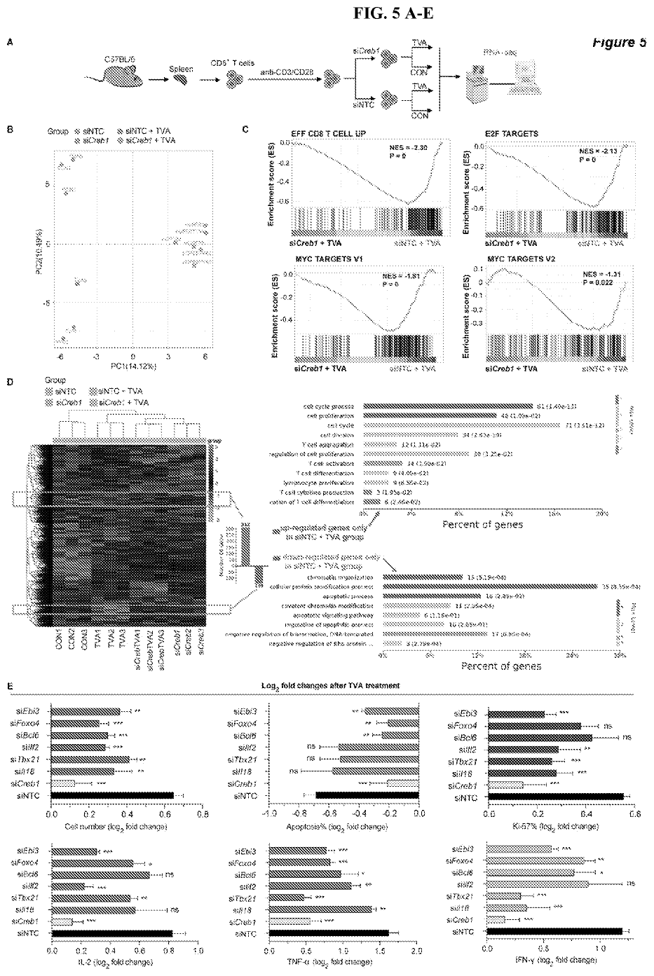

Next, the importance of CREB and its gene targets was explored. Transcriptome-wide RNA-seq was performed using CD8⁺ T cells with siRNA-mediated knockdown of CREB compared to control CD8⁺ T cells treated with non-targeting control siRNA (siNTC) in the presence and absence of TVA treatment (FIG. 5A). Principal component analysis (PCA) revealed that cells treated with siCreb in the presence and absence of TVA can be grouped together and are separated from the siNTC control cell group, or the group of control cells treated with TVA (FIG. 5B). GSEA analysis identified a set of upregulated and downregulated signaling pathways which are responsible for TVA-enhanced CD8⁺ T cell function that is mediated by CREB. Notably, consistent with RNA-seq results shown in FIG. 3D, knockdown of CREB reverses TVA-dependent upregulation of gene sets related to effector CD8⁺ T cell function, and cell proliferation including E2F and MYC pathways (FIG. 5C).

To further identify the TVA-CREB downstream targets, genes were characterized that were upregulated or downregulated only in the siNTC+TVA group, compared to other 3 groups (marked by yellow boxes in FIG. 5D, left; full gene list can be found in Table 7). These represent the CREB target genes which respond to TVA treatment, and knockdown of CREB reverses TVA-dependent alteration of these genes. In total, 312 upregulated genes are enriched in 11 GOs including, for example, cell cycle, cell proliferation, cell division, T cell aggregation, T cell activation, T cell differentiation, and cytokine production (FIG. 5D, upper right). In contrast, a total of 139 downregulated genes are enriched in 8 GOs including, for example, apoptosis, and chromatin organization (FIG. 5D, lower right). For functional validation, four upregulated genes were chosen that are critical in cell proliferation and T cell function. These genes include Il18 (GOs of cell proliferation, T cell activation, and cytokine production), Ebi3 (GOs of cell proliferation and T cell activation), Tbx21 (GOs of T cell activation and cytokine production), and Ilf2 (GOs of cell proliferation, T cell activation, and cytokine production). Two downregulated genes were also tested including Foxo4 and Bcl6 that are critical in apoptosis-related GOs. Each of these 6 TVA-CREB target genes was knocked down by distinct siRNA in mouse CD8⁺ T cells (knockdown efficiency shown in FIG. 21E), followed by functional studies upon TVA treatment.

As shown in FIG. 5E, knockdown of Creb1 by siCreb1 as a positive control effectively reverses TVA-dependent changes of cell number, apoptosis, and levels of Ki-67, IL-2, TNF-α, and IFN-γ in CD8⁺ T cells, compared to control cells treated with siNTC. (1) Knockdown of each of the 6 genes all partially reverses TVA-dependent change of cell number (FIG. 5E, upper left). (2) Knockdown of Bcl6, Foxo4, or Ebi3 partially reverses TVA-dependent change of apoptotic cell population of CD8⁺ T cells, whereas knockdown of Il18, Tbx21, or Ilf2 has no effects (FIG. 5E; upper middle). (3) Knockdown of Il18, Tbx21, Ilf2, or Ebi3 partially reverses TVA-dependent change of Ki-67 level, whereas knockdown of Bcl6 or Foxo4 has no effects (FIG. 5E, upper right). (4) Knockdown of Foxo4, Tbx21, Ilf2, or Ebi3 partially reverses TVA-dependent change of IL-2 level, whereas knockdown of Bcl6 or Il18 has no effects (FIG. 5E, lower left). (5) Knockdown of each of the 6 genes all partially reverses TVA-dependent change of TNF-α level (FIG. 5E, lower middle). (6) Knockdown of Bcl6, Il18, Foxo4, Tbx21, or Ebi3 partially reverses TVA-dependent changes of IFN-γ level, whereas knockdown of Ilf2 has no effect (FIG. 5E, lower right). These results together establish differentially functional contributions of diverse CREB target genes to distinct TVA-dependent property changes of CD8⁺ T cells.

### TVA Antagonizes Immunosuppressive SCFA-Binding GPR43

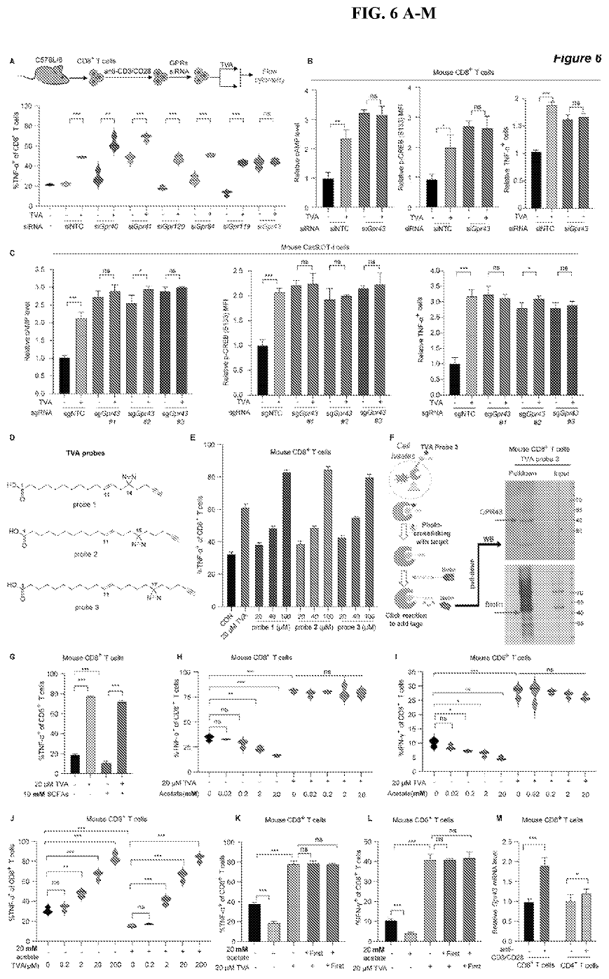

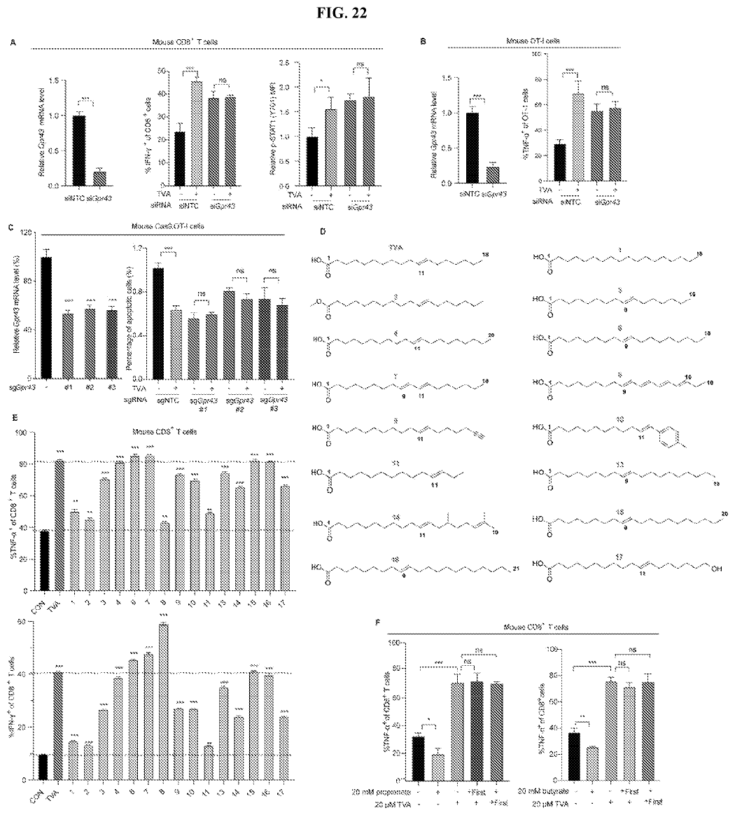

To identify the GPCR target of TVA, six of the known fatty acid-binding GPCRs were screened including GPR40, GPR41, GPR43, GPR84, GPR119, GPR120 (Swaminath, 2008) (FIG. 6A). It was found that only knockdown of GPR43 (FIG. 22A, left) abolishcs TVA-enhanced TNF-α level of primary mouse CD8⁺ T cells (FIG. 6A). Further analysis confirmed that knockdown of GPR43 in primary mouse CD8⁺ T cells leads to increased cAMP, p-CREB and TNF-α levels (FIG. 6B) as well as IFN-γ and p-STAT1 levels (FIG. 22A) in mouse primary CD8⁺ T cells that cannot be further activated by TVA treatment. These data suggest that GPR43 plays a suppressive role in CD8⁺ T cell activation, and that TVA likely attenuates GPR43 function. These results are also consistent with previous reports that GPR43 is an immunosuppressive GPCR (Ang and Ding, 2016), and that activation of GPR43 is linked with Gai and decreases cAMP levels (Brown et al., 2003). Similar results were obtained using OT-IT cells with siRNA-mediated GPR43 knockdown (FIG. 22B), and OT-I; Cas9 T cells with knockout of GPR43 by CRISPR/Cas9 (FIGS. 6C and 22C).

To explore the underlying mechanism by which TVA antagonizes GPR43, structure-activity relationship (SAR) studies were performed on TVA (FIG. 22D-22E). Fifteen TVA derivatives (FIG. 22D) were designed and compared with TVA for bioactivity to enhance CD8⁺ T cell activation (FIG. 22E). SAR results revealed that the double bond (1), acid group (2) or maintaining length of at least 20 carbon (3, 4, 13, 15, 16) are crucial for maintaining TVA bioactivity. In addition, shifting double bond position from C₁₁-C₁₂ to C₉-C₁₀ (6), or adding a C₉-C₁₀ double bond (7) results in enhanced TVA bioactivity, suggesting that a C₉-C₁₀ double bone may optimize TVA bioactivity. The SAR results of 9, 10, 14 and 17 also suggest that the terminal chain can be modified and the resulting TVA derivatives remain 70-80% bioactivity. Thus, photo-affinity probes were designed using TVA as a parental molecule to bear a photo-reactive diazirine group and a tail alkynyl group (FIG. 6D). All three TVA probes are able to enhance CD8⁺ T cells in a dose-dependent manner (FIG. 6E). Next, a photo-affinity labeling (PAL) study (Mackinnon and Taunton, 2009) was performed using mouse CD8⁺ T cells treated with the TVA probe 3. The diazirine allows covalent photo-crosslinking to contacting protein residues and the alkynyl group for click chemistry to link the reporter (biotin) and probe TVA-binding protein target(s). Western blot results demonstrate binding between the TVA probe and GPR43 (FIG. 6F), supporting their direct interaction.

Next, whether TVA may function like a “ligand-like” antagonist of GPR43 by competing off its SCFA agonists was examined. Indeed, it was found that GPR43 SCFA agonists (20 mM) significantly decrease mouse CD8⁺ T cell activation assessed by TNF-α level, while TVA at a 1,000× lower level (20 μM) is able to reverse SCFA-dependent suppression on CD8⁺ T cells (FIG. 6G). In addition, increasing concentrations of SCFA acetate up to 20 mM attenuate CD8⁺ T cell activation assessed by reduced TNF-α (FIG. 6H) and IFN-γ (FIG. 6I) levels, which is effectively reversed by TVA at 20 μM. In contrast, TVA level as low as 2 μM is sufficient to reverse suppression of CD8⁺ T cells by acetate at 20 mM (FIG. 6J). Consistent with these findings, suppression of CD8⁺ T cells with reduced TNF-α (FIG. 6K) and IFN-γ (FIG. 6L) levels by pre-treatment with 20 mM acetate (+First) can be reversed by TVA at 20 μM. In contrast, enhanced CD8⁺ T cell activation by pre-treatment with 20 μM TVA (+First) cannot be reversed by 20 mM acetate. Similar results were obtained using additional SCFA agonists including propionate and butyrate (FIG. 22F).

Lastly, it was found that stimulation by anti-CD3/CD28 antibodies results in a much higher increased GPR43 expression level in CD8⁺ T cells than that in CD4⁺ T cells (FIG. 6M). This may partly explain why activated CD8⁺ T cells are sensitive to TVA treatment. These results collectively suggest that TVA binds to and antagonizes GPR43 by competing off its SCFA agonists

### TVA Augments Effectiveness of Multiple T Cell-Based Anti-Cancer Therapies

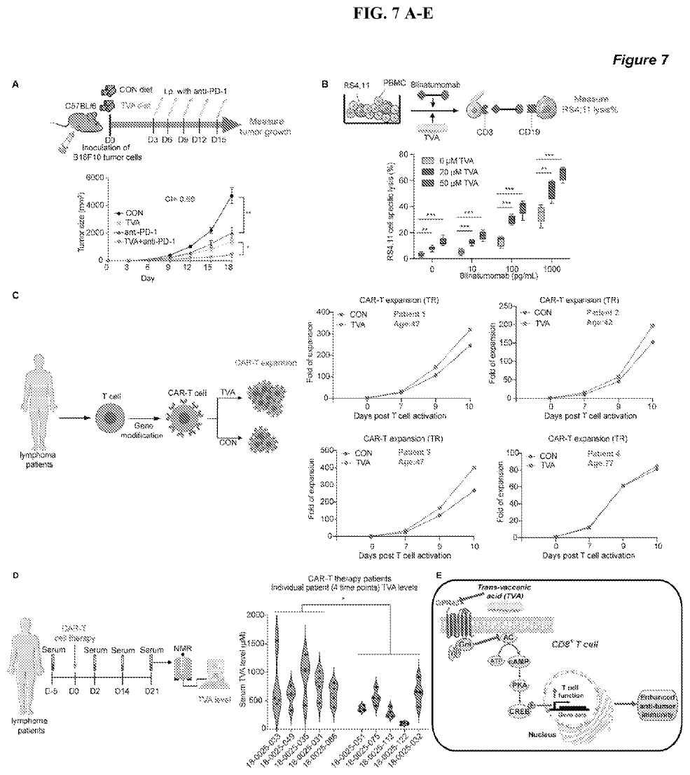

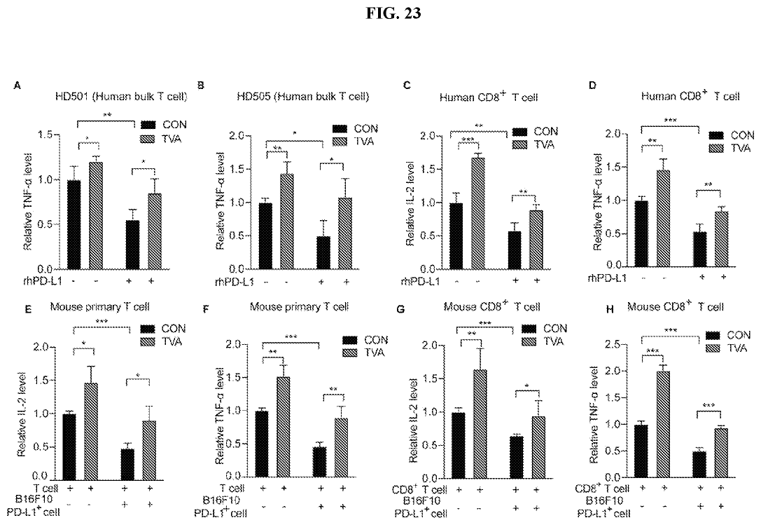

To explore translational potential of these findings, TVA-dependent effects on multiple T cell-based immunotherapies were examined. Immune checkpoint inhibitors (ICIs) including antibodies targeting PD-1 and PD-L1 have changed the treatment landscape of many tumors (Dall′Olio et al., 2021). However, immunological treatment of “cold tumors” due to lack of tumor-infiltrating T cells or not eliciting an immune response remains a great challenge (Jenkins et al., 2018; Vukadin et al., 2021). It was found that TVA treatment significantly reverses exhaustion of primary human bulk T cells (FIG. 23A-23B) and CD8⁺ T cells (FIG. 23C-23D) treated with purified recombinant PD-L1. Similar results were obtained using mouse primary bulk T cells (FIG. 23E-23F) or CD8⁺ T cells (FIG. 23G-23H) co-cultured B16F10 cells overexpressing PD-L1. Moreover, dietary TVA combined with anti-PD-1 antibody result in synergistic attenuation of B16F10 tumor growth (FIG. 7A).

Next, the effects of TVA on efficacy of Blinatumomab were tested. Blinatumomab is a bi-specific T-cell engager (BiTE) designed as a constructed monoclonal antibody targeting CD19 antigen on B cells and CD3 on T cells for treatment of human B cell acute lymphoblastic leukemia (B-ALL). Clinically, Blinatumomab is effective for minimal residual disease, but the response rate in relapsed disease is <50% due to lack of T-cell activation (Kantarjian et al., 2017). It was found that TVA treatment significantly enhances in vitro killing efficiency of human peripheral blood mononuclear cells (PBMCs) on human B-ALL RS4;11 cells in the presence of Blinatumomab in a dose-dependent manner (FIG. 7B).

Furthermore, TVA treatment improves in vitro expansion of chimeric antigen receptor (CAR) T cells derived from primary T cells from 3 lymphoma patients with age between 42 and 47 (FIG. 7C). In contrast, cells from an elder patient (age 77) do not respond to TVA treatment (FIG. 7C, lower right), which might be due to a lower starting cell number compared to those of younger patients. More importantly, in a retrospective clinical study, it was found that serum TVA levels are higher in a group of lymphoma patients that respond to CAR T-cell therapy compared to non-responders (detailed timepoint information can be found in Table 8) (FIG. 7D). These findings together align with the concept that dietary TVA may improve clinical responsiveness to T cell-based immunotherapies.

This study demonstrates that TVA as a diet-derived nutrient exhibits a nutrient-independent, extracellular signaling function to enhance CD8⁺ T cell function and consequent anti-tumor immunity. Thus, as a natural food component, TVA has translational potential to be used as a dietary element in therapeutic approaches to ameliorate clinical outcomes. For example, since TVA signals through the GPR43-CREB pathway that is independent of the PD-L1/PD-1 axis, the finding experimentally provides a mechanism-based rationale supporting a combination of TVA and immune checkpoint inhibitors for improved immunotherapies to treat cancer patients with immunologically cold tumors. In addition, TVA can be combined with specific T-cell engagers such as Blinatumomab to treat B-ALL patients. TVA can also be used to help expand and prime CAR T cells for improved efficacy of treating cancer patients. Lastly, serum level of TVA may be a useful biomarker for prediction of responsiveness to T cell-based immunotherapies such as CAR T-cell therapy and T cell receptor (TCR)-T cell therapy. Epidemiological studies suggest that the circulating levels of TVA in humans are associated with lower adiposity, diabetes risk and systemic inflammation, but the effects of dietary TVA on risk of cancer and cardiovascular diseases are unclear, despite that, in rodent models of dyslipidemia, TVA-enriched diet has hypolipidemic effects by lowering circulating triglyceride (Gebauer et al., 2011; Pranger et al., 2019; Wang et al., 2008). The reported normal ranges of blood TVA levels in human adults vary widely from 10.7+/−5.0 μM (Psychogios et al., 2011) to 186.5+/−45.8 μM (Dercksen et al., 2016), which are lower than these measurements using serum samples from lymphoma patients. This may be due to variations of sample collections and measurement approaches. Regardless, these SAR studies reveal that the bioactivity of TVA could be improved through chemical modifications including, for example, shifting the double bond to a more optimized position or changing the chain length. Thus, the next generation of TVA-derivatives could be designed and developed with improved effectiveness to target GPR43 and consequently enhance CD8⁺ T cell function and anti-tumor immunity.

This study also validates a new dimension through a comprehensive evaluation of blood chemicals in order to uncover and understand mechanistic links between diet and cancer or other human diseases. Circulating diet-derived blood chemicals have been inextricably linked to human physiology during evolution, which not only provide energy and precursors for biosynthesis for growth but also function as signaling molecules (Rodriguez et al., 2017). Accordingly, despite the vast diversity of food and diet origins, studies with a focus on blood chemicals get to the heart of the molecular mechanisms underlying nutritional influences on cancer and effectiveness of anti-cancer therapies. This evaluation of blood chemicals, as a representative, also indicates that the approach may have broad implications for future elucidation of previously unknown physiological and pathological roles of the circulating blood chemicals in human health and disease, respectively.

The integrated mechanistic studies identified cell-surface immunosuppressive GPR43, a SCFA-binding GPCR, as the target of TVA. It was shown that GPR43 contributes to immunosuppression through regulation of CD8⁺ T cell activation and exhaustion by signaling through the cAMP-PKA-CREB axis. This signaling mechanism may be cell type-specific for CD8⁺ T cells because GPR43 agonism was reported to promote colonic expansion and function of Group 3 innate lymphoid cells (ILC3s) and IL-22 production through AKT and STAT3 signaling pathways (Chun et al., 2019). However, TVA-dependent antagonism of GPR43 results in an increased phosphorylation level of STAT3 (p-STAT3) with a reduced phosphorylation level of AKT (p-AKT) in CD8⁺ T cells at 2 h post-TVA treatment (FIG. 3C) and eventually an increased p-AKT level at 24 h (FIG. 20D). These observations are different from those reported in ILC3 cells with GPR43 agonism. Future studies are warranted to comprehensively determine how GPR43 switches its downstream effector signaling pathways in different immune cells, whether TVA regulates functions of other immune cells with high levels of GPR43 expression such as monocytes, neutrophils, and marginal zone B cells (Brown et al., 2003; Le Poul et al., 2003; Nilsson et al., 2003; Rohrbeck et al., 2021), and whether TVA similarly antagonizes GPR43 to activate the PKA-CREB pathway in these cells.

Also observed was a much higher increase in GPR43 expression upon activation of CD8⁺ T cells than that in CD4⁺ T cells (FIG. 6M). This not only partly explains why activated CD8⁺ T cells are more sensitive to TVA treatment, but also may represent a previously unknown self-checking mechanism to avoid overactivation of cytotoxic CD8⁺ T cells that might lead to auto-immune issues. Future studies are warranted to fully elucidate the underlying signaling and molecular mechanisms associated with physiological function of GPR43 regulation.

### Materials and Methods

### Data and Code Availability

16S amplicon sequencing data have been deposited in the GEO repository with the accession number GSE202266 and are publicly available as of the date of publication. The KAS-seq data have been deposited in the GEO repository with the accession number GSE202730. The RNA-sequencing data have been deposited in the GEO repository with the accession number GSE202276 and GSE202274. Project number and accession links are listed in the Resources Table. Original western blot images have been deposited on Mendeley and are publicly available as of the date of publication. The DOI is also listed in the Resources Table. This paper does not report original code

### Animal Study

5-8 weeks old C57BL/6 mice, C57BL/6 nude mice, TCR-a KO, and Pmel-1 mice were purchased from the Jackson Laboratory. Please also refer to Resources Table for more details

### Primary Cells

Cas9; OT-I cells were isolated from the spleen and peripheral lymph nodes (PLN) (provided by Dr. Hongbo Chi's lab) of Cas9; OT-I mice (Wei et al., 2019) by magnetic bead purification using Easy Sep™ Mouse naïve CD8⁺ T Cell Isolation Kit according to the manufacturer's instructions (Stem Cell Technologies). Cas9; OT-I cells were activated in vitro for 18 hours with plate-bound anti-CD3 (10 μg/mL; Biolegend) and anti-CD28 (5 μg/mL; Biolegend) antibodies in Click's media at 37° C. and 5% CO₂ incubator for further experiments

### Cell Line

All cell lines were authenticated by genomic short tandem repeat (STR) profiling in University of Chicago Integrated Genomics Core (EIGC) upon purchase and at least annually as appropriate. Human T lymphocyte cell line Jurkat T was purchased from American Type Culture Collection (ATCC). Human Plat-E cells were provided by Dr. Hongbo Chi's lab. Human RS4;11 cells were provided by Dr. Wendy Stock lab. Mouse melanoma cancer cell line B16F10, breast cancer cell line E0771, and Lewis lung carcinoma cell line LLC1 were purchased from ATCC. Mouse colorectal adenocarcinoma cell line MC38 was purchased from Kerafast. Plat-E, B16F10, E0771, and LLC1 cells were cultured in Dulbecco Modified Eagle Medium (DMEM) with 10% fetal bovine serum (FBS) (Sigma, F2442) and penicillin/streptomycin. Jurkat T cells were cultured in RPMI 1640 medium with 10% FBS and penicillin/streptomycin. All the cells were cultured at 37° C. and 5% CO₂. Please also refer to Resources Table for detailed information of each cell line.

### Human Samples Collection

Serums of patients that have undergone commercial CAR T cell therapy were from University of Chicago cell therapy biobank. Please also refer to Resources Table and Table 8 for more details.

### Construction of PD-1⁺ Jurkat T Cell Line

Jurkat T cells were infected with pre-made lentivirus expresses human PD-1 (Gen Target Inc, Cat #LVP1076-PBS) according to the manufacturer's instructions. After infection for 24 hours, cells were selected with 2 μg/mL puromycin to obtain PD-1⁺ Jurkat T cells. The PD-1 expression level was checked using western blot

### Blood Chemicals Library Screens

To construct “blood chemical compound library for cell-based screening purposes, components such as antibodies that are difficult to distinguish due to a wide variety, and some supplements that only function at a whole organism level including fish oils and herbs were excluded. Physiological ranges of serum levels of individual blood chemicals are available in the human metabolome database (https://hmdb.ca/) and applied in the experimental design. For first screen 1a, Jurkat T cells were treated with blood chemical library for 48 hours and then activated with 2.5 μg/mL anti-CD3 and 0.5 μg/mL anti-CD28 antibodies for another 12 hours, followed by measurement of IL-2 level in medium supernatant using ELISA kit (Biolegend). For first screen 1b, 1×10⁵ PD-1⁺ Jurkat T cells were co-cultured with 2×10⁴ H596 (PD-L1⁺) cells in a well of 96-well plate for 60 hours, and then activated with 2.5 μg/mL anti-CD3 and 0.5 μg/mL anti-CD28 antibodies for another 12 hours, followed by measurement of IL-2 level in medium supernatant using ELISA kit. Please also refer to Resources Table and Table 1-2 for more details.

### Mouse Tumor Models

For C57BL/6 mice and TCR-a KO mice tumor model, mice were anesthetized with isoflurane, shaved the injection site, and then injected subcutaneously in the abdominal flank with 1×10⁵ B16F10, MC38, or LLC1 cells, or in the mammary gland with 2×10⁵ E0771 cells. C57BL/6 nude mice were injected subcutaneously in the abdominal flank with 1×10⁵B16F10 cells. The tumor bearing mice were assigned to TVA enriched diet (1% TVA, special order from Research Diets Inc), CVA enriched diet (1% CVA, special order from Research Diets Inc) or control diet (Research Diets Inc) as of the day of tumor inoculation, with mice body weight monitored. Tumors were measured the tumors using a caliper every 2-3 days. Tumor volumes were calculated using the following formula: length×width×[(length×width)^{0.5}]×π/6. Mice were euthanized at humane endpoints or day 11-15 for tissue collection.

For CREB inhibitor 666-15 treatment mouse model, 6-8 weeks old C57BL/6 mice were anesthetized with isoflurane, shaved the injection site, and then injected subcutaneously in the abdominal flank with 1×10⁵ B16F10 for tumor development. The tumor bearing mice were assigned to TVA enriched diet or control diet as of the day of tumor inoculation, when the tumor volume reached approximately 100 mm³, the mice were treated with either vehicle or 666-15 at 20 mg/kg. 666-15 was dissolved in 1% N-methylpyrrolidone (NMP), 5% Tween-80 in H₂O. The mice were treated once a day for 5 consecutive days per week for 2 weeks. Tumors were measured the tumors using a caliper every 2-3 days. Tumor volumes were calculated using the following formula: length×width×[(length×width)^{0.5}]×π/6. Mice were euthanized at humane endpoints.

For anti-PD-1 treatment mouse model, 6-8 weeks old C57BL/6 mice were anesthetized with isoflurane, shaved the injection site, and then injected subcutaneously in the abdominal flank with 1×10⁵ B16F10 for tumor development. The tumor bearing mice were assigned to TVA enriched diet or control diet as of the day of tumor inoculation. In day 3, 6, 9, 12, and 15, 250 μg anti-PD-1 (BioXCell) or IgG control (BioXCell) was injected intraperitoneally per mouse. Tumors were measured the tumors using a caliper every 2-3 days. Tumor volumes were calculated using the following formula: length×width×[(length×width)^{0.5}]×π/6. Mice were euthanized at humane endpoints. Please also refer to Resources Table for more details.

### Antibody-Mediated T Cell Depletions

5-8 weeks old C57BL/6 mice were injected subcutaneously in the abdominal flank with 1×10⁵ B16F10 cells, and then injected intraperitoneally with six doses of depleting antibodies (anti-CD8a, BioXCell, Clone #2.43) or isotype control (rat IgG2b isotype control, BioXCell, Clone #LTF-2) on days 1 (200 μg), 2 (200 μg), 4 (200 μg), 8 (200 μg), 12 (200 μg), and 16 (200 μg) relative to tumor injection (day 0). Cheek bleeds were collected and subjected to flow cytometry to check CD8⁺ T cell depletion efficiency on days 3, 12, and 18 using antibodies targeting non-competing CD8 epitopes (BV711 anti-mouse CD8a).

### Secreted Cytokines Level

Human or mouse T cell secreted IL-2, TNF-α, and IFN-γ levels were detected by ELISA MAX™ Standard Set (Biolegend) as the manufacturer's instructions. Please also refer to Resources Table for more details.

### Pmel-1 Killing Assay

Pmel-1 cells were isolated from Pmel mouse and seeded at density of 1×10⁶ cells/well in 6-well plate (pre-coated with anti-CD3 and anti-CD28) for 18 hours. 1×10⁶ activated Pmel-1 cells were then co-cultured with 2×10⁵ B16F10 cells for 24 hours with or without 20 μM TVA. All cells in suspension were collected, stained with anti-mouse CD45 and PI, and analyzed by flow cytometry. Please also refer to Resources Table for more details.

### Cell Proliferation Assay

Cell proliferation assays were conducted by seeding 5×10⁴ cells in a 6-well plate. Cell numbers were recorded daily within 5 days using TC20 Automated Cell Counter (BioRad).

### Extraction and Quantification of TVA Levels by NMR

Tumor interstitial fluid (TIF) and serum from tumor-bearing animals were isolated as described (Sullivan et al., 2019). In brief, tumors were dissected away from the euthanized animal, rinsed in the saline containing Petri dish, and then placed on top of the 20 μm mesh nylon filter (Spectrum Labs, Cat #148134) that was affixed to the conical tube (Falcon, Cat #1495949A). After the conical tube lid was placed on top without screwing and taped using laboratory tape in place, the tumor containing conical tube was subject to centrifugation at 4° C. for 10 minutes at 106×g using a refrigerated clinical centrifuge. 10-50 μL of fluid were obtained in the bottom of the conical tube. To prepare the mouse serum, once the tumors were dissected, blood was isolated from the mouse heart by cardiac puncture using 1 mL 25G TB syringe, allowed to clot by leaving it undisturbed at room temperature, and then centrifuged at 1,500×g for 10 min in a refrigerated centrifuge. The resulting supernatant was designated serum. The human serum samples were obtained from University of Chicago cell therapy biobank. The TIL and serum samples are lyophilized and subjected to quantification of TVA levels by ¹H nuclear magnetic resonance (NMR) spectroscopy using a Bruker Ascend™ 600 spectrometer equipped with CryoProbeProdigy™. In a typical procedure, 350 μL deuterated methanol (Methanol-d⁴) with 0.03% tetramethylsilane (TMS) purchased from Oakwood Chemical (Estill, SC, USA) was added to a lyophilized sample to dissolve TVA. After being vortexed, the sample was then centrifuged at 10000×g for 5 min to collect the supernatant. ¹H-NMR spectrum of the supernatant (250 μL) in a 335-pp Precision 3 mm NMR tube (Wilmad-Lab Glass, Vineland, NJ, USA) was acquired with delay time (d¹) set to 2 s for 3072 scans. TVA concentration was calculated based on the integral of peak at 1.97 ppm and the TMS internal standard

### CD45⁺ Tumor-Infiltrating Leukocytes Isolation

Tumor tissue were dissected from euthanized tumor-bearing mice, minced into small pieces (≤2 mm) using a scalpel in a dish, and then transferred to a 14 mL round-bottom tube containing 5 mL tumor digestion medium (500 μL Collagenase/Hyaluronidase Solution, 750 μL 1 mg/mL DNase I Solution, and 3.75 mL RPMI 1640 Medium). After incubation at 37° C. for 25 min on a shaking platform, the digested tumor tissues were transferred into a 70 μm mesh nylon strainer on a 50 mL conical tube, pushed through the strainer using the rubber end of a syringe plunger, and rinsed with the recommended medium. After centrifugation at 300×g for 10 min at room temperature with the brake on low, the resulting cell pellets were added 10 mL of ammonium chloride solution for incubation at room temperature for 5 minutes, followed by centrifugation at 300×g for 10 minutes at room temperature with the brake on low. The resulting cell pellets were re-suspended at 1-10×10⁶ cells/mL in PBS and then subjected to CD45⁺ tumor-infiltrating leukocytes isolation by magnetic bead purification using EasySep™ Mouse TIL (CD45) Positive Selection Kit according to the manufacturer's instructions (Stemcell Technologies). Please also refer to Resources Table for more details.

### Mouse Tumor-Infiltrating Lymphocytes Isolation

Freshly excised mouse tumor tissues were minced into small pieces (volume smaller than 1 mm³) by scissors in PBS, digested by Collagenase IV (1 mg/mL) and DNase I (200 U/mL) at 37° C. for 20 minutes in PBS with gentle rocking. The digested tumor tissues were added 5 times volume 0.01M EDTA, filtered into new tube through 200 mesh screen (100 μm strainer), and then centrifuged at 300×g for 5 minutes at room temperature. The resulting cell pellets were re-suspended with 3 mL PBS, laid gently on 3 mL lymphocytes isolation liquid (pre-warmed to room temperature), and subjected to density gradient centrifugation (300×g, 30 minutes, room temperature, accelerate 6, decelerate 2). The middle layer of cells was moved carefully to a new tube, added PBS to 15 mL, and centrifuged (300×g, 10 minutes, room temperature). The cell pellets (lyse red cells if necessary) were designated tumor-infiltrating lymphocytes and used for following experiments. Please also refer to Resources Table for more details.

### Mouse Spleen Lymphocytes Isolation

Mouse spleens were disrupted with syringe plunger in 1 mL PBS in a 40 μm strainer filtered to a 15 mL tube, washed with PBS and centrifuged at 300×g for 5 minutes. The resulting cell pellets were re-suspended the with 2 mL red cell lysis buffer (Invitrogen), incubated at room temperature for 10 minutes, and centrifuged at 300×g for 5 minutes after adding 13 mL PBS. The resulting cell pellets was designated splenocytes and used for following experiments.

### Mouse Draining Lymph Nodes Lymphocytes Isolation

Draining lymph nodes were disrupted with syringe plunger in 1 mL PBS in a 40 μm strainer filtered to a 15 mL tube, washed with PBS, and centrifuged at 300×g for 5 minutes. The resulting cell pellets was designated LN Lymphocytes and used for following experiments.

Primary CD8⁺ or CD4⁺ T Cell Isolation and Activation

Mouse primary CD8⁺ or CD4⁺ T cells were isolated from the spleen and peripheral lymph nodes (PLN) of C57BL/6 mice by magnetic bead purification using EasySep™ Mouse CD8⁺ or CD4⁺ T Cell Isolation Kit according to the manufacturer's instructions (Stemcell Technologies). Human primary CD8⁺ T cells were isolated from PBMC (Zen-Bio) by human CD8⁺ T Cell Isolation Kit according to the manufacturer's instructions (Stemcell Technologies). Isolated primary CD8⁺ or CD4⁺ T cells were activated in vitro for 18 hours with plate-bound anti-CD3 (10 μg/mL; Biolegend) and anti-CD28 (5 μg/mL; Biolegend) antibodies in click's media at 37° C. and 5% CO₂ incubator. Activated CD8⁺ or CD4⁺ T cells were ready for further experiments. A naive CD8⁺ T cells control was maintained in Click's media containing 10 ng/ml IL-7 (BioLegend). Please also refer to Resources Table for more details.

### Flow Cytometry and Staining

Mouse primary cells isolated from tumor, spleen, draining lymph node were stained with fluorescent antibodies and analyzed by flow cytometry.

For experiments with live/dead criteria, cells were first stained with Fixable Viability Dyes (FVD) (Thermo Fisher Scientific) according to the manufacturer's instructions. Subsequent surface marker staining was performed in Flow Cytometry Staining Buffer (Thermo Fisher Scientific). Intracellular staining for flow panels containing nuclear proteins was performed using the eBioscience FoxP3/Transcription Factor Staining Buffer Set (Thermo Fisher Scientific) according to the manufacturer's instructions. For intracellular staining of cytoplasmic proteins, the Fixation/Permeabilization Solution Kit (BD Biosciences) was used according to the manufacturer's instructions.

For phospho antibody staining, cells were incubated with FVD (cell viability dye) for 15 minutes at room temperature in a tube, re-suspended with 200 μL pre-warmed 1×BD Phosflow Lyse/Fix buffer directly into the tube, and incubated at 37° C. for 10-15 minutes, followed by centrifugation at 300×g for 5 minutes. The resulting cell pellets were washed once with FACS buffer, permeabilized with 200 μL of BD Phosflow Perm Buffer III for 45 minutes on ice, and centrifuged at 300×g for 5 minutes. The cell pellets were washed again with FACS buffer, centrifuged at 300×g for 5 minutes, and incubated with antibodies in FACS buffer for 45 minutes-1 hour at room temperature.

Mouse anti-CD11b antibody was used for myeloid cell (CD11b⁺) marker. After gated with CD11b⁺ cells, anti-F4/80 and Gr1 antibodies were used for macrophage (Gr1⁻ F4/80⁺) markers, anti-CD11c antibody was used for dendritic cells (CD11c⁺) marker, anti-CD16 and CD63 antibodies were used for neutrophils (CD16⁻CD63⁺) markers, anti-CD14 antibody was used for monocytes (CD14⁺) marker.

Data was collected on LSR-Fortessa 4-15 flow cytometer or Attune NxT 4-14 and analyzed using FlowJo v10.4. Please also refer to Resources Table for more details.

### Microbiome 16S Sequencing

Gut feces of CON and TVA group (7 samples per group) B16F10 tumor bearing mice were collected at day 16, and then subjected to microbiome 16S sequencing by Zymo Research (Irvine, CA). In brief, The ZymoBIOMICS®-96 MagBead DNA Kit (Zymo Research) was used to extract DNA using an automated platform. Bacterial 16S ribosomal RNA gene targeted sequencing was performed using the Quick-16S™ NGS Library Prep Kit (Zymo Research). The bacterial 16S primers amplified the V3-V4 region of the 16S rRNA gene. The sequencing library was prepared using an innovative library preparation process in which PCR reactions were performed in real-time PCR machines to control cycles and therefore limit PCR chimera formation. The final PCR products were quantified with qPCR fluorescence readings and pooled together based on equal molarity. The final pooled library was cleaned with the Select-a-Size DNA Clean & Concentrator™ (Zymo Research), then quantified with TapeStation® (Agilent Technologies) and Qubit® (Thermo Fisher Scientific). The ZymoBIOMICS® Microbial Community Standard (Zymo Research) was used as a positive control for each DNA extraction, if performed. The final library was sequenced on Illumina® MiSeq™ with a v3 reagent kit (600 cycles). The sequencing was performed with 10% PhiX spike-in. For Bioinformatics Analysis, unique amplicon sequences variants were inferred from raw reads using the DADA2 pipeline (Callahan et al., 2016). Potential sequencing errors and chimeric sequences were also removed with the Dada2 pipeline. Chimeric sequences were also removed with the DADA2 pipeline. Taxonomy assignment was performed using Uclust from Qiime v.1.9.1 with the Zymo Research Database, a 16S database that is internally designed and curated, as reference. Composition visualization, alpha-diversity, and beta-diversity analyses were performed with Qiime v.1.9.1.

### ¹³C1-TVA Metabolic Flux Analysis by GC-MS

1×10⁶ activated mouse primary CD8⁺ T cells were cultured for 24 hours in RPMI-1640 medium containing 0, 20, 50 UM ¹³C1-TVA (Sigma), rinsed with 0.9% saline solution, and lysed with lysis buffer (400 μL of cold MeOH, 300 μL of 0.88% (w/v) KCl). Cell lysis were scraped off the plate into a glass vial, added 800 μL of cold dichloromethane, and vortexed for 15 minutes at 4° C., followed by centrifugation at max speed for 10 minutes at 4° C. Samples were kept at room temperature after centrifugation to form two distinct phases. 650 μL of the bottom dichloromethane fraction was transferred into a new glass tube and dried with nitrogen gas to obtain a lipid fraction pellet. FAME Derivatization was performed as previously described (Lien et al., 2020). In brief, the dried lipid pellet was dissolved in 100 μL of toluene, added 200 μL of 2% sulfuric acid in MeOH for incubation at 50° C. overnight and then added 500 μL of 5% NaCl to extract twice with 500 μL hexane. FAME cleanup was applied if analyzing animal tissues (FAME Cleanup: a Florisil column was pre-conditioned with 3 mL of hexane, added methyl ester, and eluted twice with 1 mL isohexane-diethyl ether (95:5 v/v), with drying down in between elutions). Otherwise, samples were dried down under nitrogen and re-suspended in 50 μL of hexane to load onto gas chromatography (GC) mass spectrometry (MS). Derivatized samples were injected into a GC-MS using DB-FastFAME column (Agilent Technologies, Cat #G3903-63011) installed in an Agilent GC system. TVA-FAME has retention time of 9.6 minutes and m/z of 264 and 292, ¹³C1-TVA-FAME has retention time of 9.6 minutes and m/z 265. Metabolite levels and mass isotopomer distributions of derivatized fragments were analyzed with an in-house MATLAB script, which integrated the metabolite fragment ions and corrected for natural isotope abundances.

### Cell Culture Treatment

Mouse primary CD8⁺ T cells were isolated, activated, and subjected to further treatment. Treatment with sulfosuccinimidyl oleate (SSO) was performed by incubating cells with 100 μM SSO for 24 hours. For inhibitors and modulators treatments, SCH-202676 (200 nM), 666-15 (100 nM), H-89 dihydrochloride (200 nM), Rhosin HCl (10 μM), NFAT inhibitor (1 μM), U0126 (100 nM), Ruxolitinib (100 nM), short chain fatty acid mix (10 mM), acetate (0.02, 0.2, 2, 20 mM), butyrate (0.02, 0.2, 2, 20 mM), propionate (0.02, 0.2, 2, 20 mM) were added to medium synchronized with 20 μM TVA for 24 hours. For TVA washing experiment, mouse CD8⁺ T cells were treated with TVA for 24 hours, washed off, and then cultured for another 24 hours in media with or without TVA. For 8-Bromo-cAMP and TVA different doses treatment experiment, activated mouse CD8⁺ T cells were treated with 8-Bromo-cAMP (0.01, 0.1, 1, 10, 100 μM) or TVA (10, 20, 100, 500, 1000 μM) for 24 hours, cells were collected for p-CREB level detection. For human T cell and CD8⁺ T cell rhPD-L1 assay, activated cells were treated with 0.5 μg/mL rhPD-L1 (R&D) for 72 hours in the presence or absence of 20 μM TVA, followed by ELISA detection of IL-2 and TNF-α level. Please also refer to Resources Table for more details.

### Kethoxal-Assisted Single-Stranded DNA Sequencing (KAS-Seq) and Data Analysis

Mouse and human CD8⁺ T cells were isolated, activated, and treated with or without 20 μM TVA for 20 minutes, 40 minutes, and 2 hours. 500 mM N3-kethoxal was then supplemented into the media followed by brief, vigorous shaking to fully homogenize the solution. The 6-well plates were then moved into the cell incubator (37° C., 5% CO2) for 10 minutes to promote labeling of genomic single-stranded DNA (ssDNA). Cell suspensions were then transferred to 15 mL conical tubes and centrifuged at 300×g for 5 minutes at 4° C. The supernatant media was discarded, and genomic DNA was then extracted. The ssDNA with N3-kethoxal label was biotinylated, enriched, and fragmented following the established KAS-seq protocol (Lyu et al., 2022; Wu et al., 2020a). Dual index libraries were constructed for high throughput sequencing using an Accel-NGS Methyl-seq DNA library kit and then sequenced at the Genomics Facility (University of Chicago) via Illumina NovaSeq 6000 (SP flowcell, 100 bp cassette) in two separate runs. Raw sequencing data under each condition was then catenated from the two runs, and adapter trimming; read deduplication and mapping; and differential KAS-seq analysis (comparing TVA-treated to untreated cells) was performed following the KAS-pipe analysis pipeline (Lyu et al., 2022; Wu et al., 2020a) Volcano plots were subsequently generated in RStudio. For Gene Ontology (GO) enrichment analysis, a list of gene bodies exhibiting differential ssDNA levels following TVA treatment (p-values as indicated in FIG. 20A-20B) was generated for each timepoint. M. musculus and H. sapiens CD8⁺ T cell KAS-seq data were assessed separately. The list of differentially-expressed gene bodies was then submitted for GO enrichment and visualization, which was performed via the Gene Ontology project (Ashburner et al., 2000; Carbon et al., 2021) and REVIGO (Supek et al., 2011)/Cytoscape (Shannon et al., 2003), respectively.

### Phospho Antibody Array

To analyze signaling pathways, mouse primary CD8⁺ T cells were isolated, activated, and treated with TVA for indicated time, followed by Phospho Antibody Array (R&D Systems) according to the manufacturer's instructions. The quantification for pixel density in each spot of the array was carried out by subtracting background signals from the spot intensity using software ImageJ (ImageJ, RRID: SCR_003070). Please also refer to Resources Table and Table 6 for more details.

### Quantitative Real-Time PCR (RT-PCR)

Total RNA was extracted with TRIzol Reagent (Invitrogen) and then used for synthesizing the first strand cDNA with PrimeScript™ 1st strand cDNA Synthesis Kit (Takara) according to the manufacturer's instructions. Quantitative RT-PCR was conducted with iTaq Universal SYBR Green Supermix (Bio-Rad). Please also refer to Resources Table for more details of reagents and RT-PCR primers.

RNA Interference (RNAi) with Accell siRNA

Mouse primary CD8⁺ T cells were isolated and cultured in replete media (RPMI 1640 medium or Click's medium containing 15% FBS, 55 μM 2-mercaptoethanol, 2 mM glutamine, penicillin/streptomycin, and either PHA, CD3 or IL-2) for 24 hours, followed by incubation with Accell delivery mix (Accell siRNA Delivery Media (Horizon Discovery) with 1 μM siRNA, 20 IU/mL IL-2 and 1% FBS) for 72 hours. Cells were collected for subsequent function analysis as well as depletion efficiency validation using RT-PCR. Please also refer to Resources Table for more details.

### RNA-Sequencing

To check effect of TVA treatment on gene expression of mouse CD8⁺ T cells, primary mouse CD8⁺ T cells were isolated, activated, and then treated with or without 20 μM TVA (TVA group vs. CON group) for 24 hours, followed by RNA extraction using the PureLink RNA Mini Kit as the manufacturer's instructions. RNA samples in triplicate were used for Illumina Next Generation Sequencing. To check effect of Creb1 knockdown on TVA dependent CD8⁺ T cell gene expression changes, mouse primary CD8⁺ T cells were isolated, activated, and then transfected with siNTC or siCreb1 using Accell siRNA method, followed by treatment with or without 20 μM TVA for 24 hours. RNA samples from four groups (siNTC, siNTC+TVA, siCreb1, siCreb1+TVA) were extracted and used for Illumina Next Generation Sequencing. Data processing and analysis were performed as previous described (Su et al., 2018). Please also refer to Resources Table and Table 7 for more details.

cAMP Level

Mouse primary CD8⁺ T cells were isolated, activated, and then treated with 20 μM TVA for 0, 20 minutes, 40 minutes, and 2 hours. cAMP-Screen Cyclic AMP Immunoassay System (Fisher Scientific Company) was used to check cAMP level according to the manufacturer's instructions. Please also refer to Resources Table for more details.

### CRISPR Editing of Mouse OT-I Cells

CRISPR editing of mouse primary CD8⁺ T cells was performed as previously described. In brief, LMPd-sgNTC-mPGK-Ametrine, LMPd-sgGPR43 #1-mPGK-Ametrine, LMPd-sgGPR43 #2-mPGK-Ametrine, and LMPd-sgGPR43 #3-mPGK-Ametrine were generated and co-transfected with pCL-Eco (Addgene, Cat #12371) into Plat-E cells using TransIT®-LT1 Transfection Reagent to produce retrovirus. Virus harvest media was changed 18 hours after transfection and then collected 48 hours later. Primary Cas9-expressing OT-I (Cas9; OT-I) cells were derived from the spleen and peripheral lymph nodes (PLN) of Cas9; OT-I mice (Wei et al., 2019) by magnetic bead purification using EasySep™ Mouse T Cell Isolation Kit according to the manufacturer's instructions (Stemcell Technologies). 2-5 million Cas9; OT-I cells were activated, placed into one well of 24-well plate, and supplemented with retrovirus supernatant containing 10 μg/mL polybrene, followed by spin infection (800×g, brake 3) for 3 hours. After infection, cells were moved to 37° C. cell culture incubator for another 2 hours and then changed with new complete Click's medium (containing 20 IU/mL human IL-2, 2.5 ng/ml mIL-7, 25 ng/ml mIL-15) for 72 hours. Cells were harvested, sorted with flow cytometry to enrich virus-transduced cells (Ametrine positive), and subjected to subsequent function analysis as well as knockout efficiency validation using RT-PCR. Please also refer to Resources Table for more details.

### Pull Down Assay to Identify the Crosslinked Protein-TVA Complexes

Mouse primary CD8⁺ T cells were isolated and activated. 10 million cells were collected, pelleted, and re-suspended in 0.3 mL ice-cold PBS containing EDTA-free protease and phosphatase inhibitor Cocktail (Thermo Fisher Scientific, Cat #A32961), followed by sonication on ice. TVA Probe 3 was added and incubated at 37° C. for 2 hours under dark conditions. After probe labeling, the sample was irradiated under 365 nm UV light for 6 minutes on ice, diluted with 1% SDS and sonicated on ice. The proteome concentration was determined using Pierce™ BCA Protein Assay Kit (Thermo Fisher Scientific) on a microplate reader (Bio-Rad) and normalized to 1.5 mg/mL. 700 μL protein sample was conjugated with a biotin tag by “click chemistry” (100 μM biotin-azide, 100 μM TBTA, 1 mM CuSO4 and 1 mM TCEP) in dark at room temperature for 1 hour. The sample was added 4 volumes of acetone, chilled to −20° C., vortexed, and then incubated for 3 hours at −20° C. to completely precipitate the proteins and remove unreacted biotin-azide. The sample was centrifuged at 17,000×g for 15 minutes at 4° C. to pellet the precipitated proteins. The resulting protein pellets were resolved in 1% SDS by sonication, added PBS to dilute the concentration of SDS to 0.2% and then added 60 μl pre-washed high-capacity streptavidin C₁ beads, followed by incubation overnight at 4° C. with rotation. The beads were washed 3 times with 0.1% SDS in PBS and once with PBS, added 2×SDS sample buffer, and boiled for western blot analysis of TVA binding proteins. Please also refer to Resources Table for more details.

Co-Culture Assay with Blinatumomab

The RS4;11 target cells were stained using the CellTrace™ Far Red Cell Proliferation Kit (Invitrogen, Cat #C₃₄₅₆₄), and then co-cultured for 24 hours with PBMC in flat bottom 24 well-plate at a 1:5 ratio (2×10⁵ target cells to 10⁶ PBMC) at indicated concentrations of Blinatumomab (0, 10, 100, 1000 μg/mL). derived from the leftover of infusions (Provided by Dr. Wendy Stock's lab). The cell mixture was resuspended in a flow cytometry staining buffer, stained using a Fixable Viability Dye780 (R&D), and then analyzed by flow cytometry. Please also refer to Resources Table for more details.

### CAR-T Cell Expansion Assay

63,000 anti-human CD19-CD28z-GFP CAR T cells (Provided by Justin Kline lab) were cultured in T cell expansion medium (StemCell) with IL-7 (5 ng/mL) and IL-15 (5 ng/ml) in the presence or absence of 20 μM TVA. Medium was changed to fresh medium (with IL-7 and IL-15 in the presence or absence of 20 μM TVA) in Day 2, 5, 7, and 9. Cell number was counted in day 7, 9, and 10.

### Quantification and Statistical Analysis

A two-tailed Student's t test was subjected to data statistical analysis, except a two-way ANOVA test was performed for cell proliferation assay and tumor growth analysis. p values less than 0.05 were considered significant. Data with error bars represent mean±SD, except for cell proliferation and tumor growth curves which represent mean±SEM. Statistical analysis and graphical presentation was performed using Prism 5.0 (GraphPad).

Key Resources Table

| | | |
|---|---|---|
| REAGENT or RESOURCE | SOURCE | IDENTIFIER |

Antibodies

| | | |
|---|---|---|
| Rat anti-IgG2b isotype | BioXCell | Cat#BE0090; |
| | | Clone#LTF-2; |
| | | RRID: AB_1107780. |
| Mouse anti-CD8α | BioXCell | Cat#BE0061; |
| | | Clone#2.43; |
| | | RRID: AB_1125541. |
| Mouse anti-PD-1(CD279) | BioXCell | Cat#BE0146; |
| | | Clone#RMP1-14; |
| | | RRID: AB_10949053. |
| Mouse PerCP/Cyanine5.5 anti-Ki-67 | Biolegend | Cat#652423; |
| Antibody | | Clone#16A8; |
| | | RRID: AB_2629530. |
| Brilliant Violet 605 ™ anti-T-bet | Biolegend | Cat#644817; |
| Antibody | | Clone#4B10; |
| | | RRID: AB_11219388. |
| Mouse APC anti-CD223(LAG-3) Antibody | Biolegend | Cat#125209; |
| | | Clone#C9B7W; |
| | | RRID: AB_10639935. |
| Mouse Brilliant Violet 650 ™ anti-CD223 | Biolegend | Cat#125227; |
| (LAG-3) Antibody | | Clone#C9B7W; |
| | | RRID: AB_2687209. |
| Mouse APC anti-Ly108 Antibody | Biolegend | Cat#134609; |
| | | Clone#330-AJ; |
| | | RRID: AB_2728154. |
| Mouse PerCP/Cyanine5.5 anti-CD366(Tim-3) | Biolegend | Cat#134012; |
| Antibody | | Clone#B8.2C12; |
| | | RRID: AB_2632736. |
| PE anti-TCF1 (TCF7) Antibody | Biolegend | Cat#655207; |
| | | Clone#7F11A10; |
| | | RRID: AB_2728491. |
| Human/mouse/rat FITC anti-CD278(ICOS) | Biolegend | Cat#313505; |
| Antibody | | Clone#C398.4A; |
| | | RRID: AB_416329. |
| Human/mouse FITC anti-Granzyme B | Biolegend | Cat#372205; |
| Recombinant Antibody | | Clone#QA16A02; |
| | | RRID: AB_2687029. |
| Mouse PE/Cyanine5 anti-CD69 Antibody | Biolegend | Cat#104509; |
| | | Clone#H1.2F3; |
| | | RRID: AB_313112. |
| Mouse APC anti-CD152 Antibody | Biolegend | Cat#106309; |
| | | Clone#UC10-4B9; |
| | | RRID: AB_2230158. |
| Mouse APC anti-CD279(PD-1) Antibody | Biolegend | Cat#135209; |
| | | Clone#29F.1A12; |
| | | RRID: AB_2251944. |
| Mouse PE/Cyanine5 anti-CD4 Antibody | Biolegend | Cat#100409; |
| | | Clone#GK1.5; |
| | | RRID: AB_312694. |
| Mouse Brilliant Violet 421 ™ anti-IL-2 | Biolegend | Cat#503825; |
| Antibody | | Clone#JES6-5H4; |
| | | RRID: AB_10895901. |
| Mouse APC anti-CD45.2 Antibody | Biolegend | Cat#109813; |
| | | Clone#104; |
| | | RRID: AB_389210. |
| Mouse APC anti-IFN-γ Antibody | Biolegend | Cat#505810; |
| | | Clone#XMG1.2; |
| | | RRID: AB_315404. |
| Human/mouse PE/Cyanine7 anti-Granzyme B | Biolegend | Cat#372213; |
| Recombinant Antibody | | Clone#QA16A02; |
| | | RRID: AB_2728380. |
| Mouse PerCP/Cyanine5.5 anti-TNF-α | Biolegend | Cat#506321; |
| Antibody | | Clone#MP6-XT22; |
| | | RRID: AB_961435. |
| Mouse Brilliant Violet 711 ™ anti-CD8a | Biolegend | Cat#100747; |
| Antibody | | Clone#53-6.7; |
| | | RRID: AB_11219594. |
| Mouse Brilliant Violet 421 ™ anti-FOXP3 | Biolegend | Cat#126419; |
| Antibody | | Clone#MF-14; |
| | | RRID: AB_2565933. |
| Mouse APC anti-CD3 Antibody | Biolegend | Cat#100235; |
| | | Clone#17A2; |
| | | RRID: AB_2561455. |
| FITC anti-Bcl-2 | Biolegend | Cat#633503; |
| | | Clone#BCL/10C4; |
| | | RRID: AB_2028392. |
| Mouse PE anti-CD71 | Biolegend | Cat#113807; |
| | | Clone#RI7217; |
| | | RRID: AB_313568. |
| Mouse APC anti-CD98(4F2) | Biolegend | Cat#128211; |
| | | Clone#RL388; |
| | | RRID: AB_2750544. |
| PE anti-RPS6 Phospho(Ser235/Ser236) | Biolegend | Cat#608603; |
| | | Clone#A17020B; |
| | | RRID: AB_2750251. |
| PE anti-NFATc1 | Biolegend | Cat#649605; |
| | | Clone#7A6; |
| | | RRID: AB_2562546. |
| Mouse FITC anti-F4/80 Recombinant | Biolegend | Cat#157309; |
| Antibody | | Clone#QA17A29; |
| | | RRID: AB_2876535. |
| Mouse APC anti-Ly-6G(Gr1) Antibody | Biolegend | Cat#127613; |
| | | Clone#1A8; |
| | | RRID: AB_1877163. |
| Mouse/human APC anti-CD11b Antibody | Biolegend | Cat#101211; |
| | | Clone#M1/70; |
| | | RRID: AB_312794. |
| Mouse PerCP anti-CD11c Antibody | Biolegend | Cat#117325; |
| | | Clone#N418; |
| | | RRID: AB_893236. |
| Alexa Fluor ® 647 anti-mouse CD16 Antibody | Biolegend | Cat#158021; |
| | | Clone#S17014E; |
| | | RRID: AB_2904300. |
| PE/Cyanine5 anti-mouse CD28 Antibody | Biolegend | Cat#102108; |
| | | Clone#37.51; |
| | | RRID: AB_312873. |
| Mouse PE/Cyanine7 anti-CD14 Antibody | Biolegend | Cat#123315; |
| | | Clone#Sa14-2; |
| | | RRID: AB_10641133. |
| Mouse/human PE anti-Ki-67 Antibody | Biolegend | Cat#151210; |
| | | Clone#11F6; |
| | | RRID: AB_2716008. |
| PE anti-Lck Phospho(Tyr394) | Biolegend | Cat#933103; |
| | | Clone#A18002D; |
| | | RRID: AB_2820203. |
| PE TOX Monoclonal Antibody(TXRX10) | Thermo Fisher Scientific | Cat#12-6502-82; |
| | | Clone#TXRX10; |
| | | RRID: AB_10855034. |
| APC Phospho-CREB(Ser133) Recombinant | Thermo Fisher Scientific | Cat#MA5-36992; |
| Rabbit Monoclonal Antibody | | Clone#CREBS133- |
| | | 4D11; |
| | | RRID: AB_2896927. |
| Rabbit PE Active Caspase-3 | Thermo Fisher Scientific | Cat#BDB561011; |
| | | Clone#C92-605; |
| | | RRID: AB_2033931. |
| Phospho-NFATC1(Ser172) Polyclonal | Thermo Fisher Scientific | Cat#PA564696; |
| Antibody | | Clone#N/A; |
| | | RRID: AB_2662248. |
| Rabbit PE Phospho-Stat1(Tyr701) | Thermo Fisher Scientific | Cat#MA5-37039; |
| Recombinant Monoclonal Antibody | | Clone#Stat1Y701- |
| | | 3E6; |
| | | RRID: AB_2896974. |
| GPR43 Polyclonal Antibody | Thermo Fisher Scientific | Cat#PA5-111780; |
| | | Clone#N/A; |
| | | RRID: AB_2857189. |
| Biotin Monoclonal Antibody(Z021) | Thermo Fisher Scientific | Cat#03-3700; |
| | | Clone#Z021; |
| | | RRID: AB_2532265. |
| PKA C-α Antibody | Cell Signaling | Cat#4782S; |
| | Technology | Clone#N/A; |
| | | RRID: AB_2170170. |
| Phospho-PKA C(Thr197) Antibody | Cell Signaling | Cat#4781S; |
| | Technology | Clone#N/A; |
| | | RRID: AB_2300165. |
| Rabbit Stat1(D1K9Y) mAb | Cell Signaling | Cat#14994S; |
| | Technology | Clone#D1K9Y; |
| | | RRID: AB_2737027. |
| Mouse monoclonal anti-β-actin antibody | Sigma-Aldrich | Cat#A1978; |
| | | Clone#AC-15; |
| | | RRID: AB_476692. |
| Goat anti-Mouse IgG (H + L) Secondary | Thermo Fisher Scientific | Cat#31430; |
| Antibody, HRP | | Clone#N/A; |
| | | RRID: AB_228307. |
| Goat anti-Rabbit IgG (H + L) Secondary | Thermo Fisher Scientific | Cat#31460; |
| Antibody, HRP | | Clone#N/A; |
| | | RRID: AB_228341. |
| Goat Polyclonal IFN-alpha/beta R1 Antibody | Novus biologicals Inc. | Cat#AF3039-SP; |
| | | Clone#N/A; |
| | | RRID: AB_664107. |
| Hamster Monoclonal TNF RI/TNFRSF1A | Novus biologicals Inc. | Cat#MAB430-SP; |
| Antibody | | Clone#55R170; |
| | | RRID: AB_2208782. |

Bacterial and virus strains

| | | |
|---|---|---|
| MAX Efficiency ™ Stbl2 ™ Competent Cells | Thermo Fisher Scientific | Cat#10268019 |
| h PDCD1(CD279) Expression, Concentrated | GenTarget Inc | Cat#: LVP1076-PBS |
| Lentivirus | | |

Biological samples

| | | |
|---|---|---|
| Serum of patient that have undergone | University of Chicago cell | Cat#18-0025-035, |
| commercial CAR T cell therapy | therapy biobank | RF |
| Serum of patient that have undergone | University of Chicago cell | Cat#18-0025-033, |
| commercial CAR T cell therapy | therapy biobank | PB |
| Serum of patient that have undergone | University of Chicago cell | Cat#18-0025-031, |
| commercial CAR T cell therapy | therapy biobank | EG |
| Serum of patient that have undergone | University of Chicago cell | Cat#18-0025-049, |
| commercial CAR T cell therapy | therapy biobank | MC |
| Serum of patient that have undergone | University of Chicago cell | Cat#18-0025-086, |
| commercial CAR T cell therapy | therapy biobank | LT |
| Serum of patient that have undergone | University of Chicago cell | Cat#18-0025-032, |
| commercial CAR T cell therapy | therapy biobank | LB |
| Serum of patient that have undergone | University of Chicago cell | Cat#18-0025-051, |
| commercial CAR T cell therapy | therapy biobank | MH |
| Serum of patient that have undergone | University of Chicago cell | Cat#18-0025-075, |
| commercial CAR T cell therapy | therapy biobank | KY |
| Serum of patient that have undergone | University of Chicago cell | Cat#18-0025-112, |
| commercial CAR T cell therapy | therapy biobank | ND |
| Serum of patient that have undergone | University of Chicago cell | Cat#18-0025-122, |
| commercial CAR T cell therapy | therapy biobank | TM |
| Patient Peripheral Blood Mononuclear Cells | Justin Kline lab | N/A |
| (PBMCs) | | |
| Human Peripheral Blood Mononuclear Cells | Zen-Bio Inc | Cat#SER-PBMC- |
| (PBMCs) | | 200-F |
| CAS9; OT-I mouse spleen and lymph node | Hongbo Chi lab | N/A |

Chemicals, peptides, and recombinant proteins

| | | |
|---|---|---|
| Human IL-2 IS | Miltenyi Biotec | Cat#130-097-743 |
| Mouse IL-2 IS | Miltenyi Biotec | Cat#130-120-662 |
| Mouse IL-7 | Fisher Scientific Company | Cat#50813299 |
| Mouse IL-15 | Fisher Scientific Company | Cat#50813273 |
| Recombinant Human PD-L1/B7-H1 Fc | R&D | Cat#156-B7-100 |
| Chimera Protein | | |
| Fixable viability dye 780 | R&D | Cat#65-0865-14 |
| DNase I | Millipore | Cat#EN0521 |
| N-methylpyrrolidone | Sigma-Aldrich | Cat#PHR1352 |
| | | CAS:872-50-4 |
| TWEEN-80 | Sigma-Aldrich | Cat#P1754 |
| H 89 Dihydrochloride | Fisher Scientific Company | Cat#29101 |
| | | CAS: 130964-39-5 |
| 666-15 | Fisher Scientific Company | 501871791 |
| | | CAS: 1433286-70-4 |
| Rhosin hydrochloride | Fisher Scientific Company | 500310R |
| | | CAS: 1281870-42-5 |
| Rho inhibitor I | Fisher Scientific Company | Cat#NC9588154 |
| XL019 | Fisher Scientific Company | Cat#501364339 |
| 8-Bromo-cAMP | Fisher Scientific Company | Cat#11-401-0 |
| Acetate Solution | Sigma-Aldrich | Cat#3863 |
| NFAT Inhibitor | Cayman Chemical | Cat#13855-1 |
| SCH 202676 | Cayman Chemical | Cat#16945-10 |
| cis-Vaccenic Acid | Cayman Chemical | Cat#20023-500 |
| Trans-Vaccenic acid | Sigma-Aldrich | Cat#V1131 |
| | | CAS: 693-72-1 |
| Trans-Vaccenic acid-1-13C | Sigma-Aldrich | Cat#645516 |
| Sulfo-N-succinimidyl Oleate sodium | Sigma-Aldrich | Cat#SML2148 |
| | | CAS: 135661-44-8 |
| GLPG0974 | Sigma-Aldrich | Cat#SML2443 |
| | | CAS: 1391076-61-1 |
| AH-7614 | Sigma-Aldrich | Cat#SML2025 |
| | | CAS: 6326-06-3 |
| DL-β-Hydroxybutyric acid sodium salt | Sigma-Aldrich | Cat#H6501 |
| | | CAS: 150-83-4 |
| Collagenase/Hyaluronidase | Stemcell Technologies Inc | Cat#07912 |
| Ammonium Chloride Solution | Stemcell Technologies Inc | Cat#07800 |
| T4 DNA Ligase Reaction Buffer | New England Biolabs Inc | Cat#B0202S |
| NEBuffer Set (r1.1, r2.1, r3.1 & rCutSmart) | New England Biolabs Inc | Cat#B7030S |

Critical commercial assays

| | | |
|---|---|---|
| ELISA MAX ™ Deluxe Set Human IL-2 | Biolegend | Cat#431804 |
| ELISA MAX ™ Deluxe Set Human TNF-α | Biolegend | Cat#430204 |
| ELISA MAX ™ Deluxe Set Human IFN-γ | Biolegend | Cat#430104 |
| ELISA MAX ™ Deluxe Set Mouse IL-2 | Biolegend | Cat#431004 |
| ELISA MAX ™ Deluxe Set Mouse TNF-α | Biolegend | Cat#430904 |
| ELISA MAX ™ Deluxe Set Mouse IFN-γ | Biolegend | Cat#430804 |
| Fitc annexin V apoptosis detection kit I | BD Biosciences | Cat#556547 |
| CellTrace Violet Cell Proliferation Kit | Fisher Scientific Company | Cat#C34571_372 |
| EasySep ™ Mouse T Cell Isolation Kit | Stemcell Technologies Inc | Cat#19851 |
| EasySep ™ Mouse TIL (CD45) Positive | Stemcell Technologies Inc | Cat#100-0350 |
| Selection Kit | | |
| EasySep ™ Mouse CD8+ T Cell Isolation Kit | Stemcell Technologies Inc | Cat#19853 |
| EasySep ™ Mouse CD4+ T Cell Isolation Kit | Stemcell Technologies Inc | Cat#19852 |
| EasySep ™ Human T Cell Isolation Kit | Stemcell Technologies Inc | Cat#17951 |
| EasySep ™ Human CD8+ T Cell Isolation Kit | Stemcell Technologies Inc | Cat#17953 |
| EasySep ™ Human CD4+ T Cell Isolation Kit | Stemcell Technologies Inc | Cat#17952 |
| CaspaseGlo 3/7 | Promega | Cat#G8090 |
| Cell Activation Cocktail | Biolegend | Cat#423304 |
| (with Brefeldin A) | | |
| eBioscience 7-AAD Viability Staining | Fisher Scientific Company | Cat#00-6993-50 |
| Solution | | |
| eBioscience IC Fixation Buffer | Fisher Scientific Company | Cat#00-8222-49 |
| eBioscience Intracellular Fixation & | Fisher Scientific Company | Cat#G762684 |
| Permeabilization Buffer Set | | |
| FoxP3/Transcription Factor Staining | Thermo Fisher Scientific | Cat#00-5523-00 |
| Buffer Set | | |
| Phosflow Perm Buffer III | BD Biocience | Cat# 558050 |
| Phosflow Lyse/Fix buffer | BD Biocience | Cat#558049 |
| CAMP-Screen Cyclic AMP Immunoassay | Fisher Scientific Company | Cat#4412182 |
| System | | |
| Purelink RNA Mini Kit | Fisher Scientific Company | Cat#12183018A |
| Pierce ™ Rapid Gold BCA Protein Assay | Thermo Fisher Scientific | Cat#A53225 |
| Kit | | |
| QIAprep Spin Miniprep Kit | Qiagen | Cat#27106 |
| RBC lysis buffer | Invitrogrn | Cat#00-4300-54 |
| TRIzol Reagent | Invitrogen | Cat#15596026 |
| Accell siRNA Delivery Media | Horizon Discovery | Cat#B-005000-500 |
| QIAquick Gel Extraction Kit | Qiagen Inc | Cat#28706 |
| Lymphocytes isolation liquid | GE | Cat# 17144002 |
| Proteome Profiler Human Phospho-Kinase | R&D Systems | Cat#ARY003C |
| Array Kit | | |

Deposited data

| | | |
|---|---|---|
| RNA-seq data: effect of TVA treatment on | This paper | GEO: GSE202276 |
| gene expression of mouse CD8+ T cells | | |
| RNA-seq data: effect of Creb1 KD on TVA | This paper | GEO: GSE202274 |
| dependent CD8+ T cell gene expression | | |
| changes | | |
| KAS-seq data | This paper | GEO: GSE202730 |
| Microbiome 16S rRNA sequencing data | This paper | GEO: GSE202266 |
| Original images were deposited to Mendeley | This paper | https://data. |
| data | | mendeley.com/ |
| | | datasets/ |
| | | 89j7ytsgn5/ |
| | | draft?a=b922 |
| | | 746d-9ce5-4775- |
| | | 8e9c-55dfda2e6f90 |

Experimental models: Cell lines

| | | |
|---|---|---|
| Human: Jurkat T cells | ATCC | Cat#TIB-152; |
| | | RRID: CVCL_0367 |
| Human: Jurkat T PD-1 + cells | This paper | N/A |
| Human: Plat-E cells | (Wei et al., 2019) | N/A |
| Human: RS4;11 cells | Dr. Wendy Stock lab | N/A |
| Mouse: B16F10 cells | ATCC | Cat#CRL-6475; |
| | | RRID: CVCL_0159 |
| Mouse: MC38 cells | Kerafast | Cat#ENH204-FP; |
| | | RRID: CVCL_B288 |
| Mouse: E0771 cells | ATCC | Cat#CRL-3461; |
| | | RRID: CVCL_GR23 |
| Mouse: LLC1 cells | ATCC | Cat#CRL-1642; |
| | | RRID: CVCL_4358 |

Experimental models: Organisms/strains

| | | |
|---|---|---|
| Mouse: C57BL/6J | The Jackson Laboratory | JAX: 000664: |
| | | RRID: IMSR_JAX: |
| | | 000664 |
| Mouse: C57BL/6 nude (B6.Cg-Foxn1nu/J) | The Jackson Laboratory | JAX: 000819; |
| | | RRID: IMSR_JAX: |
| | | 000819 |
| Mouse: TCRα Knock-out (B6.129S2- | The Jackson Laboratory | JAX:002116; |
| Tcratm1Mom/J) | | RRID:IMSR_JAX:00 |
| | | 2116 |
| Mouse: PmeI-1 (B6.Cg- | The Jackson Laboratory | JAX: 005023; |
| Thy1a/Cy Tg(TcraTcrb)8Rest/J) | | RRID: IMSR_JAX: |
| | | 005023 |
| Mouse: OT-I (C57BL/6- | The Jackson Laboratory | JAX:003831; |
| Tg(TcraTcrb)1100Mjb/J) | | RRID: IMSR_JAX: |
| | | 003831 |

Oligonucleotides

| | | |
|---|---|---|
| Accell Mouse Ffar1 (233081) siRNA- | Horizon Discovery Ltd. | E-051167-00-0005 |
| SMARTpool | | |
| Accell Mouse Ffar3 (233080) siRNA- | Horizon Discovery Ltd. | E-058296-00-0005 |
| SMARTpool | | |
| Accell Mouse Gpr84 (80910) siRNA- | Horizon Discovery Ltd. | E-047398-00-0005 |
| SMARTpool | | |
| Accell Mouse Gpr119 (236781) siRNA- | Horizon Discovery Ltd. | E-042791-00-0005 |
| SMARTpool | | |
| Accell Non-targeting Pool | Horizon Discovery Ltd. | D-001910-10-05 |
| Accell Mouse Ffar2 (233079) siRNA- | Horizon Discovery Ltd. | E-061251-00-0020 |
| SMARTpool | | |
| Accell Mouse Ffar4 (107221) siRNA- | Horizon Discovery Ltd. | E-042801-00-0005 |
| SMARTpool | | |
| Accell Mouse Creb1 (12912) siRNA- | Horizon Discovery Ltd. | E-040959-00-0020 |
| SMARTpool, | | |
| Accell Non-targeting siRNA #1 | Horizon Discovery Ltd. | D-001910-01-20 |
| Accell GAPD siRNA - Mouse | Horizon Discovery Ltd. | D-001930-02-05 |
| Accell Mouse II18 (16173) siRNA- | Horizon Discovery Ltd. | E-046634-00-0005 |
| SMARTpool | | |
| Accell Mouse Tbx21 (57765) siRNA- | Horizon Discovery Ltd. | E-040669-00-0005 |
| SMARTpool | | |
| Accell Mouse Ilf2 (67781) siRNA-SMARTpool | Horizon Discovery Ltd. | E-060911-00-0005 |
| Accell Mouse Foxo4 (54601) siRNA- | Horizon Discovery Ltd. | E-045377-00-0005 |
| SMARTpool | | |
| Accell Mouse Bcl6 (12053) siRNA- | Horizon Discovery Ltd. | E-057495-00-0005 |
| SMARTpool | | |
| Accell Mouse Dffb (13368) siRNA- | Horizon Discovery Ltd. | E-058666-00-0005 |
| SMARTpool | | |
| Accell Mouse Ebi3 (50498) siRNA- | Horizon Discovery Ltd. | E-047002-00-0005 |
| SMARTpool | | |
| sgRNA targeting GPR43 sequence #1, | This paper | N/A |
| Forward: 5′- | | |
| CACCGGCGCGCACACGATCTTTGGT-3′ | | |
| (SEQ ID NO: 1) | | |
| sgRNA targeting Gpr43 sequence #1, | This paper | N/A |
| Reverse: 5′- | | |
| AAACACCAAAGATCGTGTGCGCGCC-3′ | | |
| (SEQ ID NO: 2) | | |
| sgRNA targeting Gpr43 sequence #2, | This paper | N/A |
| Forward: 5′- | | |
| CACCGGCAGCGATCACTCCATACAG-3′ | | |
| (SEQ ID NO: 3) | | |
| sgRNA targeting Gpr43 sequence #2, | This paper | N/A |
| Reverse: 5′- | | |
| AAACCTGTATGGAGTGATCGCTGCC-3′ | | |
| (SEQ ID NO: 4) | | |
| sgRNA targeting Gpr43 sequence #3, | This paper | N/A |
| Forward: 5′- | | |
| CACCGCTGAACTCAACTGAGCAGGT-3′ | | |
| (SEQ ID NO: 5) | | |
| sgRNA targeting Gpr43 sequence #3, | This paper | N/A |
| Reverse: 5′- | | |
| AAACACCTGCTCAGTTGAGTTCAGC-3′ | | |
| (SEQ ID NO: 6) | | |
| RT-PCR Primer: Dffb, Forward: | This paper | N/A |
| 5′-GGATGGGAGGGAGGTGAACTGG-3′ | | |
| (SEQ ID NO: 7) | | |
| RT-PCR Primer: Dffb, Reverse: | This paper | N/A |
| 5′-GGTCTTCTTGTGGCAGGCGATG-3′ | | |
| (SEQ ID NO: 8) | | |
| RT-PCR Primer: Bcl6, Forward: | This paper | N/A |
| 5′-AGGTCGTGAGGTCGTGGAGAAC-3′ | | |
| (SEQ ID NO: 9) | | |
| RT-PCR Primer: Bcl6, Reverse: | This paper | N/A |
| 5′-GGATAAGAGGCTGGTGGTGTTGAC-3′ | | |
| (SEQ ID NO: 10) | | |
| RT-PCR Primer: Tnf, Forward: | This paper | N/A |
| 5′-GCCTCTTCTCATTCCTGCTTGTGG-3′ | | |
| (SEQ ID NO: 11) | | |
| RT-PCR Primer: Tnf, Reverse: | This paper | N/A |
| 5′-GTGGTTTGTGAGTGTGAGGGTCTG-3′ | | |
| (SEQ ID NO: 12) | | |
| RT-PCR Primer: Foxo4, Forward: | This paper | N/A |
| 5′-GATGGGTCTTTGTCAGCAGGAGAAG-3′ | | |
| (SEQ ID NO: 13) | | |
| RT-PCR Primer: Foxo4, Reverse: | This paper | N/A |
| 5′-AGCAGGAGGTGGTGGTGTATCAG-3′ | | |
| (SEQ ID NO: 14) | | |
| RT-PCR Primer: Ilf2, Forward: | This paper | N/A |
| 5′-AGCCAGCACCTGATGAGACCTC-3′ | | |
| (SEQ ID NO: 15) | | |
| RT-PCR Primer: Ilf2, Reverse: | This paper | N/A |
| 5′-TGTCACCAAAGAAAGGATGGAAGCC-3′ | | |
| (SEQ ID NO: 16) | | |
| RT-PCR Primer: Tbx21, Forward: | This paper | N/A |
| 5′-ATCACTAAGCAAGGACGGCGAATG-3′ | | |
| (SEQ ID NO: 17) | | |
| RT-PCR Primer: Tbx21, Reverse: | This paper | N/A |
| 5′-ACCAAGACCACATCCACAAACATCC-3′ | | |
| (SEQ ID NO: 18) | | |
| RT-PCR Primer: Il18, Forward: | This paper | N/A |
| 5′-CAAAGTGCCAGTGAACCCCAGAC-3′ | | |
| (SEQ ID NO: 19) | | |
| RT-PCR Primer: Il18, Reverse: | This paper | N/A |
| 5′-ACAGAGAGGGTCACAGCCAGTC-3′ | | |
| (SEQ ID NO: 20) | | |
| RT-PCR Primer: Gpr41, Forward: | This paper | N/A |
| 5′-CCACACTGCTCATCTTCTTCGTCTG-3′ | | |
| (SEQ ID NO: 21) | | |
| RT-PCR Primer: Gpr41, Reverse: | This paper | N/A |
| 5′-ACGGACTCTCACGGCTGACATAG-3′ | | |
| (SEQ ID NO: 22) | | |
| RT-PCR Primer: Gpr43, Forward: | This paper | N/A |
| 5′-CTGTATGGAGTGATCGCTGCTCTG-3′ | | |
| (SEQ ID NO: 23) | | |
| RT-PCR Primer: Gpr43, Reverse: | This paper | N/A |
| 5′-CTGCTCTTGGGTGAAGTTCTCGTAG-3′ | | |
| (SEQ ID NO: 24) | | |
| RT-PCR Primer: Il2, Forward: | This paper | N/A |
| 5′-GCAGCTCGCATCCTGTGTCAC-3′ | | |
| (SEQ ID NO: 25) | | |
| RT-PCR Primer: Il2, Reverse: | This paper | N/A |
| 5′-CTGCTGTGCTTCCGCTGTAGAG-3′ | | |
| (SEQ ID NO: 26) | | |
| RT-PCR Primer: Creb1, Forward: | This paper | N/A |
| 5′-GCTGGCTAACAATGGTACGGATGG-3′ | | |
| (SEQ ID NO: 27) | | |
| RT-PCR Primer: Creb1, Reverse: | This paper | N/A |
| 5′-AACTTGGTTGCTGGGCACTAGAATC-3′ | | |
| (SEQ ID NO: 28) | | |
| RT-PCR Primer: Prkaca, Forward: | This paper | N/A |
| 5′-GGCTCTCGGAGTCCTCATCTACG-3′ | | |
| (SEQ ID NO: 29) | | |
| RT-PCR Primer: Prkaca, Reverse: | This paper | N/A |
| 5′-CGCAGCAGGTCCTTCAAGTCAG-3′ | | |
| (SEQ ID NO: 30) | | |
| RT-PCR Primer: Lag3, Forward: | This paper | N/A |
| 5′-GCCATCTCGTTCTCGTTCTCATCC-3′ | | |
| (SEQ ID NO: 31) | | |
| RT-PCR Primer: Lag3, Reverse: | This paper | N/A |
| 5′-TTCTCCACCAGTGAAAGCCAAAGG-3′ | | |
| (SEQ ID NO: 32) | | |
| RT-PCR Primer: Batf, Forward: | This paper | N/A |
| 5′-AGAGCCGACAGAGACAGACACAG-3′ | | |
| (SEQ ID NO: 33) | | |
| RT-PCR Primer: Batf, Reverse: | This paper | N/A |
| 5′-TCGGTGAGCTGTTTGATCTCTTTGC-3′ | | |
| (SEQ ID NO: 34) | | |
| RT-PCR Primer: Ebi3, Forward: | This paper | N/A |
| 5′-TCAAGTACCGACTCCGCTACCG-3′ | | |
| (SEQ ID NO: 35) | | |
| RT-PCR Primer: Ebi3, Reverse: | This paper | N/A |
| 5′-CTGAGCTGACACCTGGATGCAATAC-3′ | | |
| (SEQ ID NO: 36) | | |
| RT-PCR Primer: Gpr40, Forward: | This paper | N/A |
| 5′-GAAGCTGGCTGGACAACAGTACC-3′ | | |
| (SEQ ID NO: 37) | | |
| RT-PCR Primer: Gpr40, Reverse: | This paper | N/A |
| 5′-AAGGGCAGAAAGAAGAGCAGAATGG-3′ | | |
| (SEQ ID NO: 38) | | |
| RT-PCR Primer: Gpr120, Forward: | This paper | N/A |
| 5′-AATCGCACCCACTTCCCTTTCTTC-3′ | | |
| (SEQ ID NO: 39) | | |
| RT-PCR Primer: Gpr120, Reverse: | This paper | N/A |
| 5′-GCCCAGCAGTGAGACGACAAAG-3′ | | |
| (SEQ ID NO: 40) | | |
| RT-PCR Primer: Gpr84, Forward: | This paper | N/A |
| 5′-CAGCCTTTCTCCGTGGACACATAC-3′ | | |
| (SEQ ID NO: 41) | | |
| RT-PCR Primer: Gpr84, Reverse: | This paper | N/A |
| 5′-TAGAGCAATGAGACAGAGGGTGAGG-3′ | | |
| (SEQ ID NO: 42) | | |
| RT-PCR Primer: Gpr119, Forward: | This paper | N/A |
| 5′-TCCATATTCCAGCAGACCACCTACC-3′ | | |
| (SEQ ID NO: 43) | | |
| RT-PCR Primer: Gpr119, Reverse: | This paper | N/A |
| 5′-AGAGGCAATCTTGAGCATGTCACAG-3′ | | |
| (SEQ ID NO: 44) | | |

Recombinant DNA

| | | |
|---|---|---|
| LMPd-gRNA-mPGK-Ametrine | (Wei et al., 2019) | N/A |
| LMPd-sgNTC-mPGK-Ametrine | (Wei et al., 2019) | N/A |
| LMPd-sgGPR43#1-mPGK-Ametrine | This paper | N/A |
| LMPd-sgGPR43#2-mPGK-Ametrine | This paper | N/A |
| LMPd-sgGPR43#3-mPGK-Ametrine | This paper | N/A |
| anti-humanCD19-CD28z-GFP | This paper | N/A |
| Software and algorithms | | |
| GraphPad Prism 9 software | GraphPad Software | https://www. |
| | | graphpad.com/ |
| ImageJ | https://imagej.nih.gov/ij/ | RRID: SCR_003070 |
| FlowJo 10.4 | FlowJo Software | https://www. |
| | | flowjo.com/ |
| | | solutions/flowjo/ |

Other

| | | |
|---|---|---|
| Control Rodent Diets | Research Diets, Inc. | D12450J |
| Rodent Diets with 1% Trans-Vaccenic Acid | Research Diets, Inc. | D19110701 |
| Rodent Diets with 1% Cis-Vaccenic Acid | Research Diets, Inc. | D19110582 |

### Example 2

### TVA Suppresses Tumorigenesis and Tumor Progression

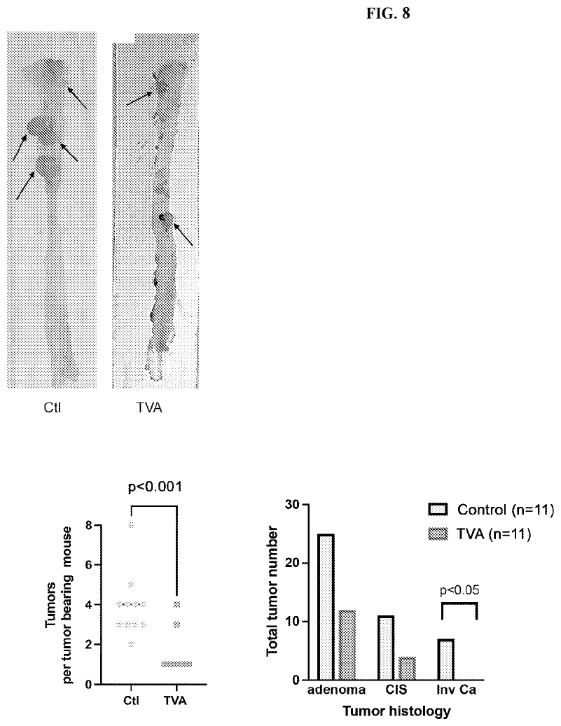

Apc^{+/fl} and Cdx-2P-Cre-NLS mice were interbred to generate Apc^{+/fl}; Cdx-2P-Cre-NLS 5 mice. Genotypes were verified using PCR protocols recommended by Jackson Laboratories. At 12 wks age, mice were started on lipid rich diet (20% mixed lipids) that mimics Western diet. After 2 wks, mice were divided into two cohorts balanced for males and females and gavaged with TVA 35 mg/kg bw or vehicle (ETOH/DMSO 2:1 v/v) daily×5 days for 4 wks. After 4 weeks, mice were killed, and samples collected. Left panel. Representative colons showing 10 tumors in situ (see FIG. 8).

### Example 3

### Enhanced Anti-Tumor Immunity Function of TVA

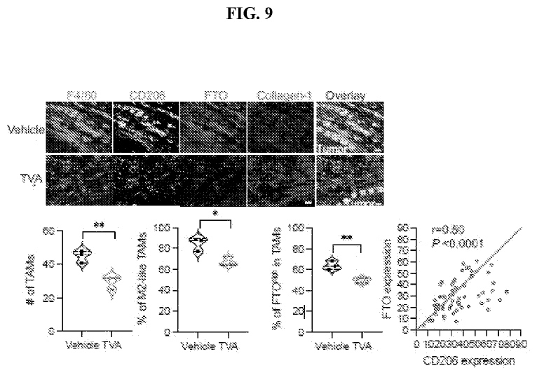

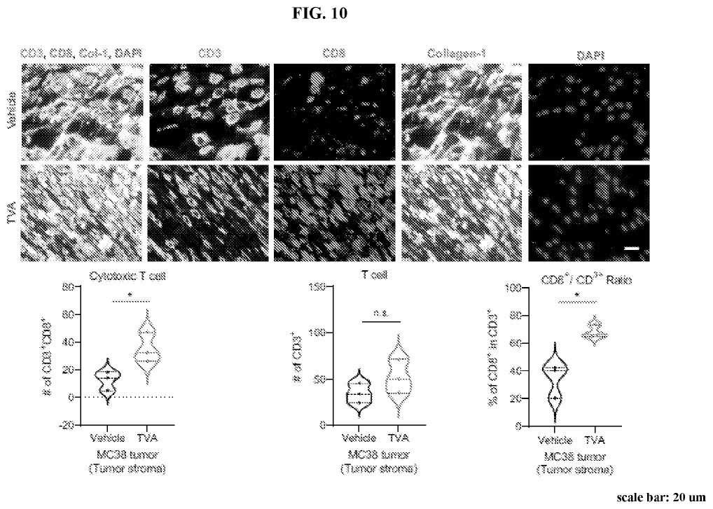

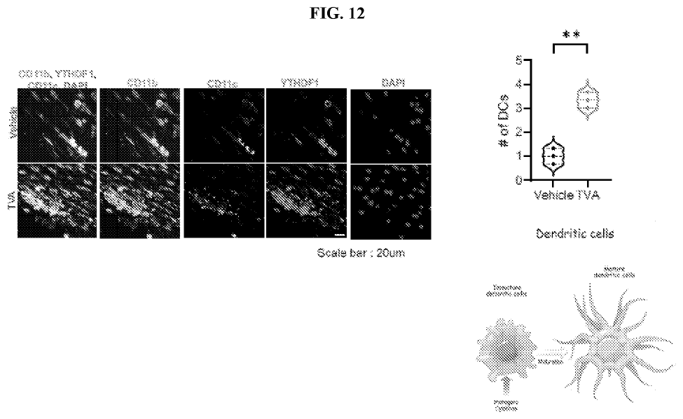

TVA treatment enhances anti-tumor immunity by coupling tumor infiltrating CD8+ T cells with increased pro-immune dendritic cells and by reducing anti-immune Treg and tumor associated macrophages (TAMs) (see FIGS. 9-12).

### Example 4

### Oral Gavage of TVA to Mice Ameliorates Immune Response to Infection by Influenza Virus (by Zhong Zheng and Hao Fan)

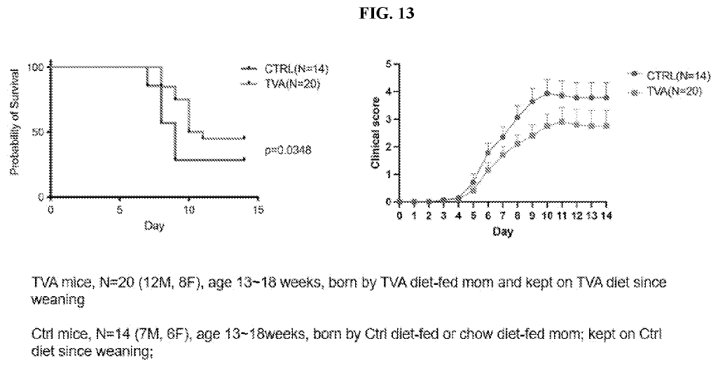

Zhong Zheng in Chuan's lab helped Hao study the effects of TVA exposure during fetal and infant stages on mouse immune response to viral challenge using influenza virus. The TVA mice were born by mothers on TVA enriched diet and fed on TVA-containing milk from mothers, followed with TVA diet since weaning. Control mice were born by mothers on normal diet, followed by normal milk feeding and normal diet since weaning. It was found that TVA mice showed improved immune responses to influenza virus with significantly increased survival (see FIG. 13).

### Example 5

### TVA Enhances Immune Response to Infection by Influenza Virus with Sex Preference to Male (by Zhong Zheng and Hao Fan)

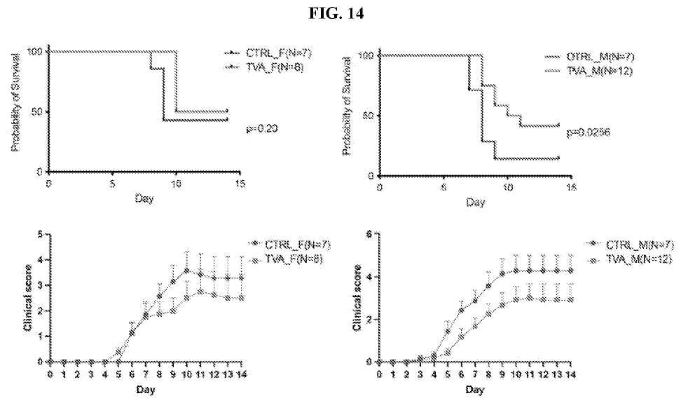

Intriguingly, it appears that male TVA mice showed better immune responses to viral exposure compared to female TVA mice, suggesting that TVA might enhance immune response in mice with sex preference to male (see FIG. 14).

### Example 6

### TVA Derivative #203 Enhances CD8+ T Cell Function In Vitro

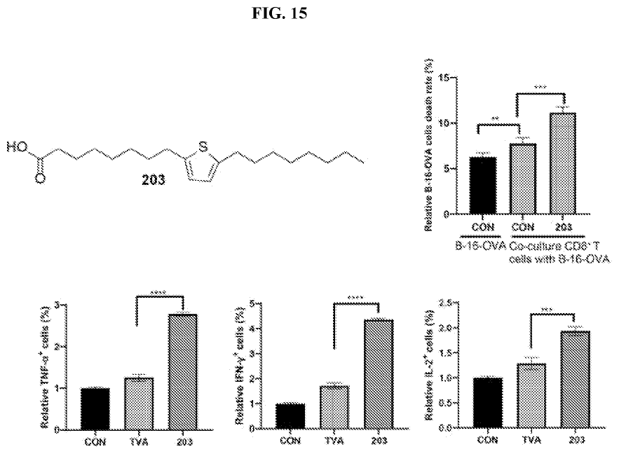

Also tested was a spectrum of TVA derivatives. #203 (top left) also enhances CD8+ T cells mediated killing of mouse melanoma B16 cells (top right) through enhanced CD8 T cell function (lower 3 panels) better than cells treated with TVA in vitro (see FIG. 15).

### Example 7

### Oral Gavage of TVA Derivative #203 Enhances Anti-Tumor Immunity in Syngeneic Mice

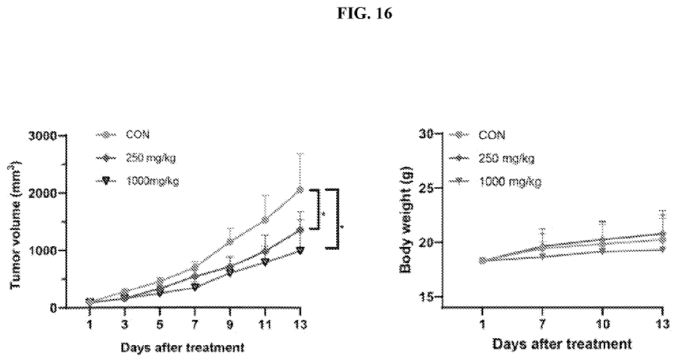

Oral gavage of #203 resulted in increased anti-tumor immunity in a dose dependent manner in syngeneic mice inoculated with murine melanoma B16 cells (see FIG. 16).

### Example 8

### TVA Derivatives and Probe Synthetic Procedures

### Synthesis of Compound 9

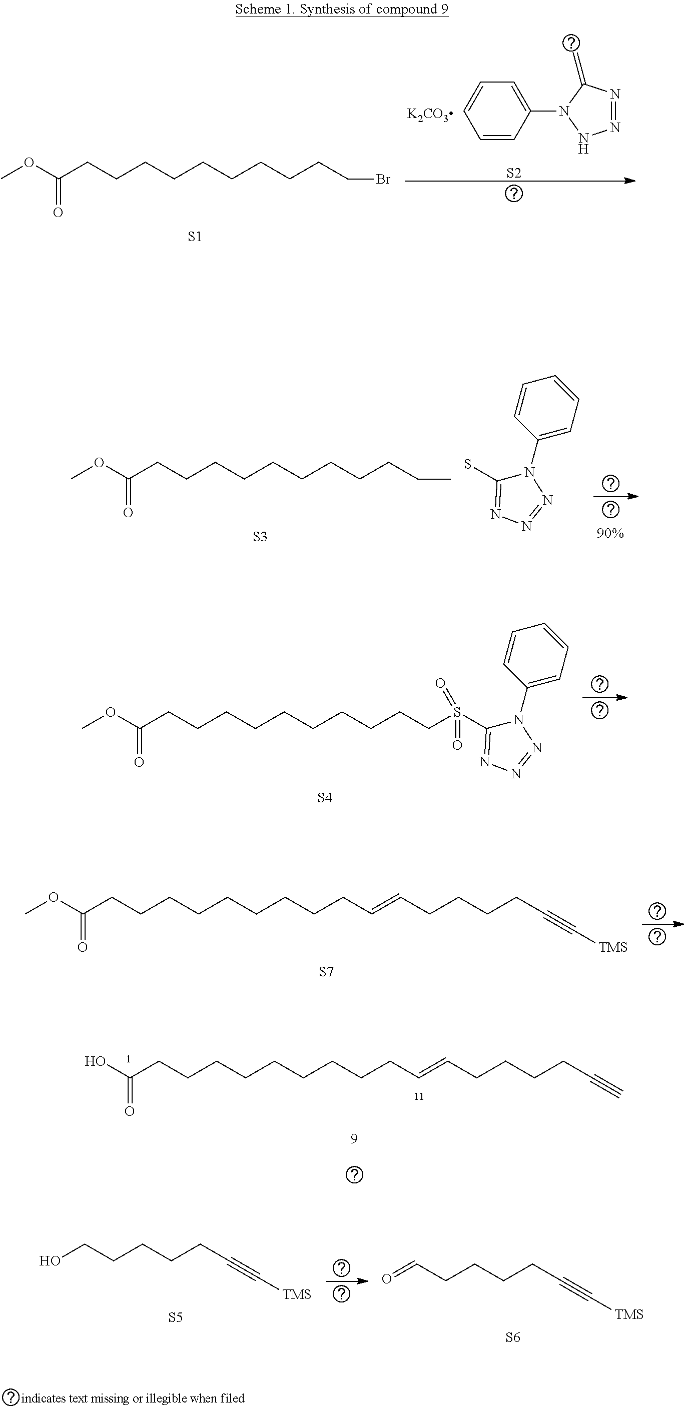

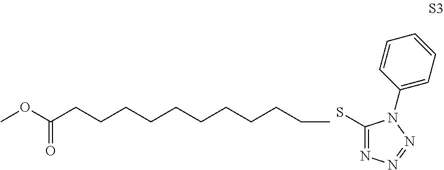

The bromide S1 (1.0 g, 3.6 mmol) was added to a stirred solution of 1-phenyl-1H-tetrazol-5-thiol S2 (1.14 g, 6.4 mmol) and potassium carbonate (1.0 g, 7.2 mmol) in acetone (20 mL) at room temperature. The mixture was heated to 65° C. and stirred for 2.5 h, then cooled to room temperature, the mixture was filtered, then the solvent was evaporated and the residue was diluted with a mixture of DCM (100 mL) and water (20 mL). The organic layer was separated and the aqueous layer was extracted with the DCM (2×50 mL). The combined organic layers were dried over NaSO₄ and evaporated. Silica gel column chromatography (EtOAc:hexane=1:6) gave a white solid (1.4 g, 96%). Obtained characterization data were in agreement with those published in the literature.

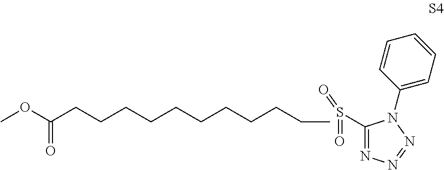

To a solution of sulfide S3 (500 mg, 1.2 mmol) in EtOH/THF=5:1 (12 mL) was added a solution of (NH₄)₆Mo7O₂₄·4H₂O (293.8 mg, 0.23 mmol) in H₂O₂ (2.6 mL, 30%) at 0° C. The reaction was stirred at room temperature overnight and quenched with H₂O (5.0 mL). The mixture was extracted with DCM (3×20 mL). The combined organic phases were dried over Na₂SO₄ and evaporated. Purification by silica gel column chromatography (EtOAc:hexane=1:5) gave a white solid (448 mg, 90%). Obtained characterization data were in agreement with those published in the literature.

To a stirred solution of the S4 (100 mg, 0.25 mmol) in DME (1.6 mL) at −78° C. under

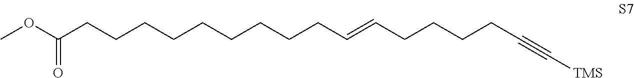 [SMILES: COC(=O)CCCCCCCCC/C=C/CCCCC#C[Si](C)(C)C]

nitrogen was added dropwise the KHMDS (0.5 M in toluene, 0.54 mL, 0.27 mmol) The mixture was then stirred for 30 min before addition of the aldehyde S6 (prepared as reported, 45 mg, 0.25 mmol). After stirring for a further 1 h at −78° C. the reaction mixture was quenched with sat. NH₄Cl (1.0 mL), then the mixture was extracted with EtOAc (3×10 mL). The combined organic phases were dried over Na₂SO₄ and evaporated. Purification by silica gel column chromatography (EtOAc:hexane=100:1) gave a colourless oil (65 mg, 73%). ¹H NMR (400 MHz, CDCl₃) δ 5.38 (q, J=5.1 Hz, 2H), 3.66 (s, 3H), 2.29 (t, J=7.5 Hz, 2H), 2.21 (t, J=6.9 Hz, 2H), 1.97 (h, J=5.7 Hz, 4H), 1.60 (m, 2H), 1.56-1.37 (m, 4H), 1.28 (m, 12H), 0.14 (s, 9H). ¹³C NMR (101 MHz, CDCl₃) δ 174.5, 130.9, 129.9, 107.8, 84.4, 51.6, 34.3, 32.7, 32.1, 29.7, 29.6, 29.5, 29.4, 29.3, 29.3, 28.9, 28.2, 25.1, 19.9, 0.3.

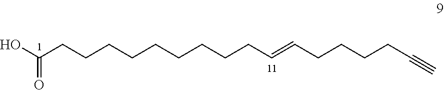 [SMILES: C#CCCCC/C=C/CCCCCCCCCC(=O)O]

To a solution of sulfide S7 (21 mg, 0.057 mmol) in THF (2.0 mL) was added a solution of TBAF (1.0 M in THF, 86 μL, 0.086 mmol) at room temperature. The reaction was stirred at room temperature for 30 min then quenched with H₂O (2.0 mL). The mixture was extracted with EtOAc (3×10 mL). The combined organic phases were dried over Na₂SO₄ and concentrated in vacuo to yield the product which was used directly in the follow reaction. The above product was dissolved in THF:H₂O=3:1 (0.8 mL), then LiOH (14 mg, 0.34 mmol) was added into the above solution, heated the mixture to 66° C. and stirred for 3 h, cooled to room temperature, 2M HCl was added to the mixture to adjust the pH to 2, then the mixture was extracted with EtOAc (3×5.0 mL). The combined organic phases were dried over Na₂SO₄ and evaporated. Purification by silica gel column chromatography (EtOAc:hexane=1:5) gave a white solid (15 mg, 93% for 2 steps). ¹H NMR (400 MHZ, CDCl₃) δ 5.48-5.27 (m, 2H), 2.35 (t, J=7.5 Hz, 2H), 2.18 (td, J=6.9, 2.7 Hz, 2H), 2.08-1.91 (m, 5H), 1.69-1.58 (m, 2H), 1.57-1.40 (m, 4H), 1.39-1.27 (m, 12H). ¹³C NMR (101 MHz, CDCl₃) δ 180.2, 131.0, 129.8, 84.8, 68.3, 34.2, 32.7, 32.1, 29.8, 29.7, 29.6, 29.5, 29.4, 29.3, 29.2, 28.8, 28.1, 24.8, 18.4. HRMS ESI (m/z): calcd. for C₁₈H₃₀O₂H⁺ [M+H]⁺: 279.2319, found 279.2317.

### Synthesis of Compound 10

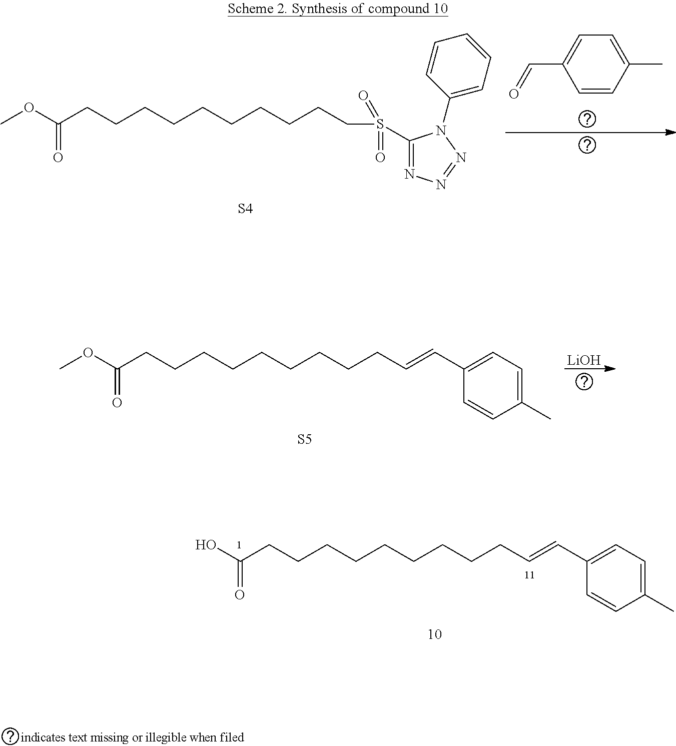

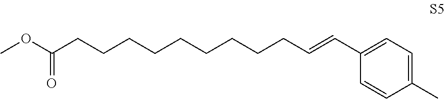

To a stirred solution of the S4 (50 mg, 0.12 mmol) in DME (1.6 mL) at −78° C. under nitrogen was added dropwise the KHMDS (0.5 M in toluene, 0.27 mL, 0.14 mmol) The mixture was then stirred for 30 min before addition of the p-tolualdehyde (16 mg, 0.27 mmol). After stirring for a further 1 h at −78° C. the reaction mixture was quenched with sat. NH₄Cl (1.0 mL), then the mixture was extracted with EtOAc (3×10 mL). The combined organic phases were dried over Na₂SO₄ and evaporated. Purification by silica gel column chromatography (EtOAc:hexane=60:1) gave a colorless oil (22 mg, 60%).

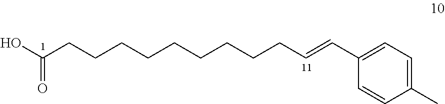 [SMILES: Cc1ccc(/C=C/CCCCCCCCCC(=O)O)cc1]

The above product S5 (12 mg, 0.039 mmol) was dissolved in THF:H₂O=3:1 (0.8 mL), then LiOH (7.0 mg, 0.16 mmol) was added into the above solution, heated the mixture to 66° C. and stirred for 3 h, cooled to room temperature, 2M HCl was added to the mixture to adjust the pH to 2, then the mixture was extracted with EtOAc (3×5.0 mL). The combined organic phases were dried over Na₂SO₄ and evaporated. Purification by silica gel column chromatography (EtOAc:hexane=1:6) gave a white solid (10 mg, 90%). ¹H NMR (400 MHZ, CDCl₃) δ 7.23 (d, J=7.9 Hz, 2H), 7.09 (d, J=7.8 Hz, 2H), 6.34 (d, J=15.8 Hz, 1H), 6.16 (dt, J=15.8, 6.9 Hz, 1H), 2.36 (d, J=7.6 Hz, 2H), 2.32 (d, J=2.9 Hz, 3H), 2.18 (q, J=6.5 Hz, 2H), 1.63 (p, J=7.4 Hz, 2H), 1.45 (p, J=7.0 Hz, 2H), 1.30 (s, 10H). ¹³C NMR (101 MHZ, CDCl₃) δ 179.6, 136.6, 135.3, 130.3, 129.7, 129.3, 125.9, 34.1, 33.2, 29.9, 29.6, 29.6, 29.4, 29.3, 29.2, 24.8, 21.3. HRMS-ESI (m/z): calcd. for C₁₉H₂₈O₂H⁺ [M+H]⁺: 289.2162, found 289.2149.

### Synthesis of Compound 11

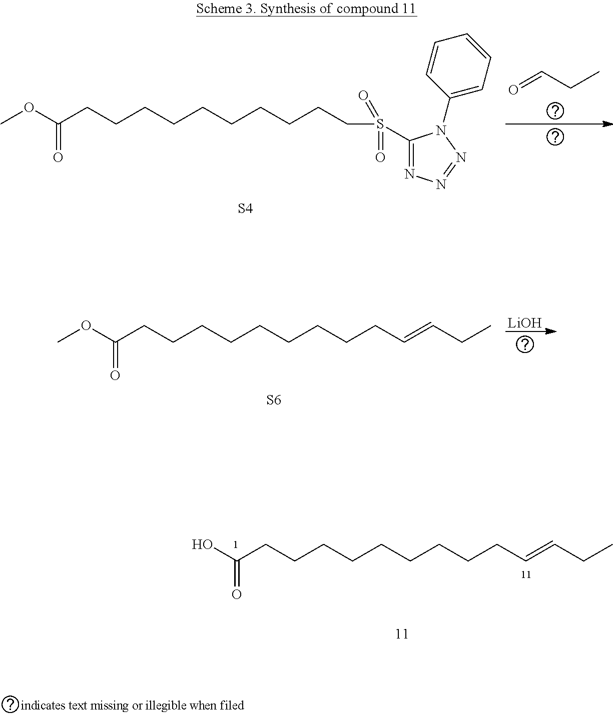

To a stirred solution of the S4 (100 mg, 0.25 mmol) in DME (1.6 mL) at −78° C. under

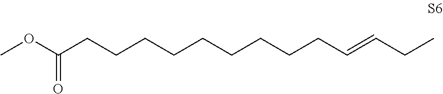 [SMILES: CC/C=C/CCCCCCCCCC(=O)OC]

nitrogen was added dropwise the KHMDS (0.5 M in toluene, 0.54 mL, 0.28 mmol) The mixture was then stirred for 30 min before addition of the propionaldehyde (16 mg, 0.27 mmol). After stirring for a further 1 h at −78° C. the reaction mixture was quenched with sat. NH₄Cl (1.0 mL), then the mixture was extracted with EtOAc (3×10 mL). The combined organic phases were dried over Na₂SO₄ and evaporated. Purification by silica gel column chromatography (EtOAc:hexane=1:60) gave a colorless oil (43 mg, 73%). ¹H NMR (400 MHZ, CDCl₃) δ 5.49-5.30 (m, 2H), 3.66 (s, 3H), 2.29 (t, J=7.5 Hz, 2H), 1.98 (dqd, J=14.8, 6.9, 5.1 Hz, 4H), 1.61 (h, J=7.5 Hz, 2H), 1.38-1.20 (m, 12H), 1.02-0.85 (m, 3H). ¹³C NMR (101 MHz, CDCl₃) δ 174.5, 132.0, 129.5, 51.6, 34.3, 32.7, 29.8, 29.6, 29.5, 29.4, 29.3, 29.3, 25.7, 25.1, 14.1. HRMS-ESI (m/z): calcd. for C₁₅H₂₈O₂Na⁺ [M+Na]⁺: 263.1982, found 263.1978.

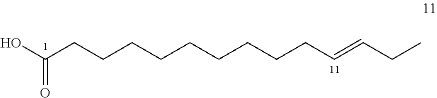 [SMILES: CC/C=C/CCCCCCCCCC(=O)O]

The above product S6 (28 mg, 0.12 mmol) was dissolved in THF:H₂O=3:1 (1.2 mL), then LiOH (20 mg, 0.46 mmol) was added into the above solution, heated the mixture to 66° C. and stirred for 3 h, cooled to room temperature, 2M HCl was added to the mixture to adjust the pH to 2, then the mixture was extracted with EtOAc (3×5.0 mL). The combined organic phases were dried over Na₂SO₄ and evaporated. Purification by silica gel column chromatography (EtOAc:hexane=1:10-1:5-1:1) gave a white solid (20 mg, 72%). ¹H NMR (400 MHZ, CDCl₃) δ 5.50-5.31 (m, 2H), 2.35 (t, J=7.5 Hz, 2H), 1.98 (dq, J=13.9, 7.2 Hz, 4H), 1.63 (p, J=7.3 Hz, 2H), 1.40-1.26 (m, 12H), 0.96 (t, J=7.4 Hz, 3H). ¹³C NMR (101 MHz, CDCl₃) δ 180.4, 132.0, 129.5, 34.2, 32.7, 29.9, 29.8, 29.6, 29.5, 29.4, 29.3, 29.2, 25.7, 24.8, 14.1. HRMS-ESI (m/z): calcd. for C₁₄H₂₆O₂H⁺ [M+H]⁺: 249.1825, found 249.1821.

### Synthesis of Compound 13

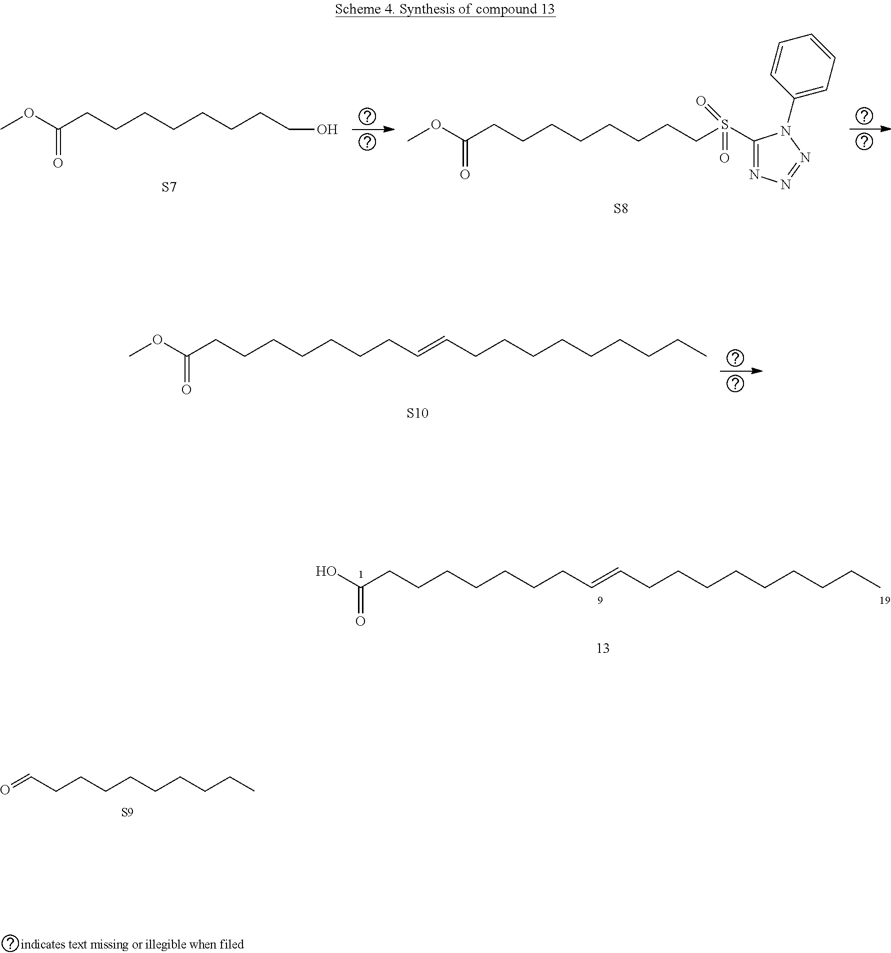

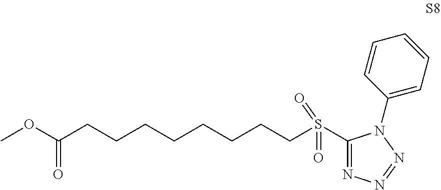 [SMILES: COC(=O)CCCCCCCCS(=O)(=O)c1nnnn1-c1ccccc1]

A solution of PT-SH (568 mg, 3.2 mmol), PPh₃ (836 mg, 3.2 mmol) and alcohol S7 (500 mg, 2.7 mmol) in THF (13 mL) and DEAD (644 mg, 3.2 mmol) was added. The resulting solution was stirred for 3 h at room temperature. The resulting solution was diluted with EtOH (20 mL), cooled to 0° C. and (NH₄)₆Mo7O₂₄·4H₂O (641 mg, 0.51 mmol) in H₂O₂ (5.8 mL, 30%) were added. The resulting yellowish solution was allowed to warm to room temperature and stirred for 10 h. Water (20 mL) was added and the whole mixture was extracted with EtOAc (3×100 mL). The combined organic layers were washed with brine (20 mL), dried over Na₂SO₄, filtered and the solvents were evaporated under reduced pressure. Purification by silica gel column chromatography (EtOAc:hexane=1:5) gave a white solid (900 mg, 83% for 2 steps). ¹H NMR (400 MHZ, CDCl₃) δ 7.70 (ddt, J=7.2, 3.4, 2.5 Hz, 2H), 7.65-7.56 (m, 3H), 3.81-3.70 (m, 2H), 3.66 (s, 3H), 2.30 (t, J=7.5 Hz, 2H), 2.01-1.89 (m, 2H), 1.68-1.44 (m, 4H), 1.33 (qd, J=8.8, 4.5 Hz, 6H). ¹³C NMR (101 MHz, CDCl₃) δ 174.3, 153.6, 133.2, 131.6, 129.8, 125.2, 56.1, 51.6, 34.1, 29.0, 28.9, 28.8, 28.2, 24.9, 22.1. HRMS-ESI (m/z): calcd. for C₁₇H₂₄N₄O₄SH⁺ [M+H]⁺: 381.1591, found 381.1600.

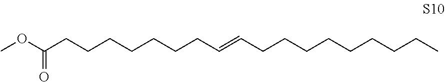 [SMILES: CCCCCCCCC/C=C/CCCCCCCC(=O)OC]

Compounds S10 was synthesized following a similar procedure described for S6. Without purified for the next step.

Compounds 13 was synthesized following a similar procedure described for 11. White

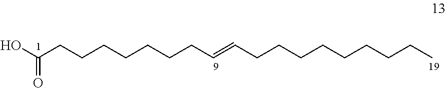 [SMILES: CCCCCCCCC/C=C/CCCCCCCC(=O)O]

solid, yield: 70%. ¹H NMR (400 MHZ, CDCl₃) δ 5.41-5.34 (m, 2H), 2.34 (t, J=7.5 Hz, 2H), 1.96 (q, J=6.2 Hz, 4H), 1.63 (p, J=7.2 Hz, 2H), 1.44-1.15 (m, 22H), 0.88 (t, J=6.7 Hz, 3H). ¹³C NMR (101 MHz, CDCl₃) δ 180.0, 130.5, 130.2, 34.0, 32.6, 32.6, 31.9, 29.7, 29.6, 29.6, 29.6, 29.4, 29.2, 29.1, 29.0, 28.9, 24.7, 22.7, 14.1. HRMS-ESI (m/z): calcd. for C₁₉H₃₆O₂H⁺

[M+H]⁺: 297.2788, found 297.2777.

### Synthesis of Compound 14

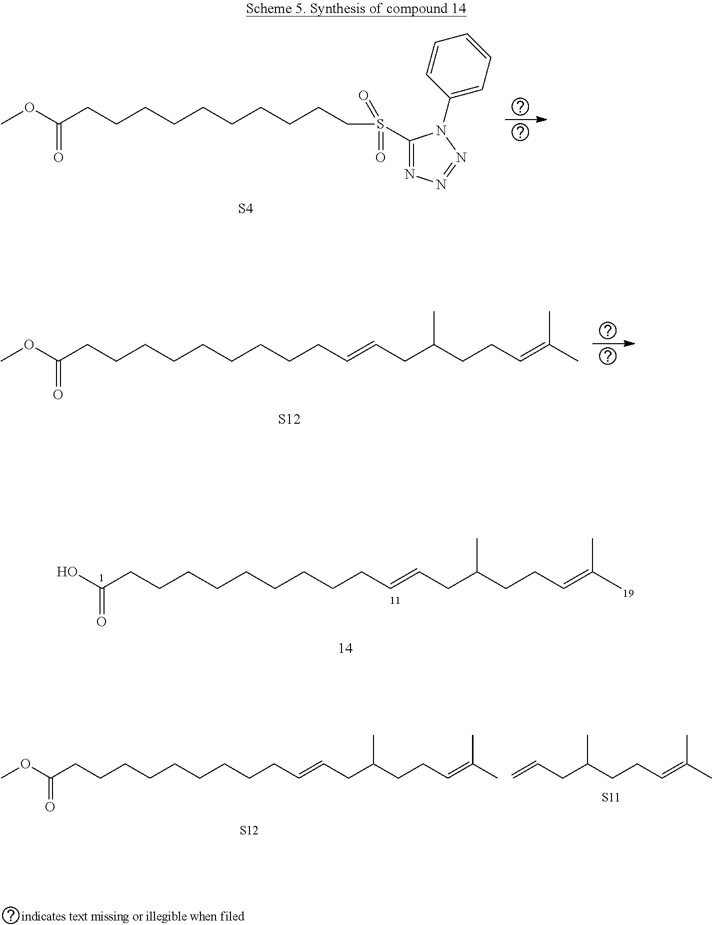

Compounds S12 was synthesized following a similar procedure described for S6. Without purified for the next step.

Compounds 14 was synthesized following a similar procedure described for 11. White

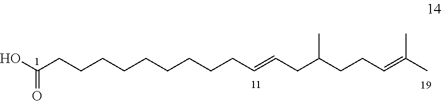 [SMILES: CC(C)=CCCC(C)C/C=C/CCCCCCCCCC(=O)O]

solid, yield: 65%, ¹H NMR (400 MHZ, CDCl₃) δ 5.44-5.29 (m, 2H), 5.10 (ddt, J=8.6, 7.1, 1.4 Hz, 1H), 2.35 (t, J=7.5 Hz, 2H), 1.97 (dh, J=10.2, 4.4 Hz, 5H), 1.87-1.77 (m, 1H), 1.68 (s, 3H), 1.65-1.62 (m, 1H), 1.60 (s, 3H), 1.49-1.40 (m, 1H), 1.37-1.24 (m, 16H), 0.86 (d, J=6.6 Hz, 3H). ¹³C NMR (101 MHz, CDCl₃) δ 180.0, 131.7, 131.0, 128.7, 125.0, 40.0, 36.6, 34.0, 32.8, 32.6, 29.7, 29.6, 29.4, 29.4, 29.2, 29.1, 29.1, 25.7, 25.6, 24.7, 19.4, 17.6. HRMS-ESI (m/z): calcd. for C₂₁H₃₈O₂H⁺ [M+H]⁺: 323.2945, found 323.2947.

### Synthesis of Compound 15

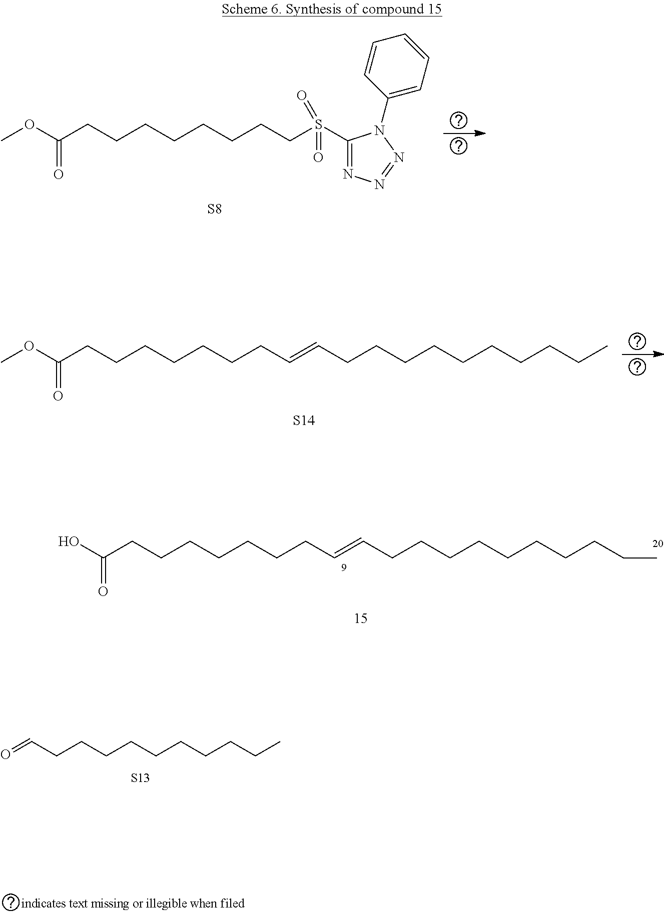

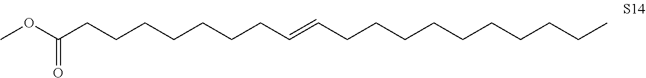 [SMILES: CCCCCCCCCC/C=C/CCCCCCCC(=O)OC]

Compounds S14 was synthesized following a similar procedure described for S6. Without purified for the next step.

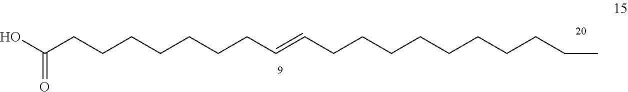 [SMILES: CCCCCCCCCC/C=C/CCCCCCCC(=O)O]

Compounds 15 was synthesized following a similar procedure described for 11. White solid, yield: 73%. ¹H NMR (400 MHZ, CDCl₃) δ 5.42-5.34 (m, 2H), 2.40-2.30 (m, 2H), 2.01-1.92 (m, 4H), 1.63 (p, J=7.4 Hz, 2H), 1.38-1.21 (m, 24H), 0.88 (t, J=1.9 Hz, 3H). ¹³C NMR (101 MHz, CDCl₃) δ 180.0, 130.5, 130.2, 34.0, 32.6, 32.6, 31.9, 29.7, 29.6, 29.5, 29.4, 29.2, 29.1, 29.0, 28.9, 24.7, 22.7, 14.1. HRMS-ESI (m/z): calcd. for C₂₀H₃₈O₂H⁺ [M+H]⁺: 311.2945, found 311.2938.

### Synthesis of Compound 16

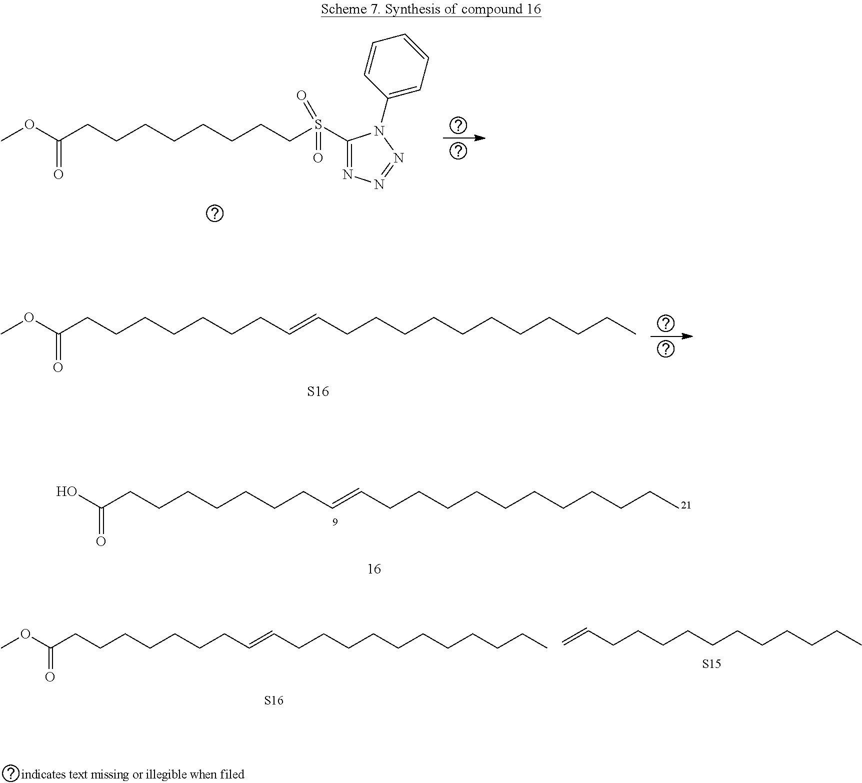

Compounds S16 was synthesized following a similar procedure described for S6. Without purified for the next step.

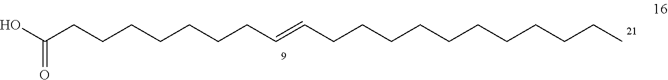 [SMILES: CCCCCCCCCCC/C=C/CCCCCCCC(=O)O]

Compounds 16 was synthesized following a similar procedure described for 11. White, solid yield: 78%. ¹H NMR (400 MHZ, CDCl₃) δ 5.42-5.34 (m, 2H), 2.35 (ddd, J=9.2, 6.7, 1.5 Hz, 2H), 2.01-1.92 (m, 4H), 1.63 (p, J=7.4 Hz, 2H), 1.37-1.25 (m, 26H), 0.92-0.84 (m, 3H). ¹³C NMR (101 MHz, CDCl₃) δ 179.8, 130.5, 130.2, 34.0, 32.6, 32.6, 31.9, 29.7, 29.7, 29.6, 29.5, 29.4, 29.2, 29.1, 29.0, 28.9, 24.7, 22.7, 14.1. HRMS-ESI (m/z): calcd. for C₂₁H₄₀O₂H⁺ [M+H]⁺: 325.3101, found 325.3097.

### Synthesis of Compound 17

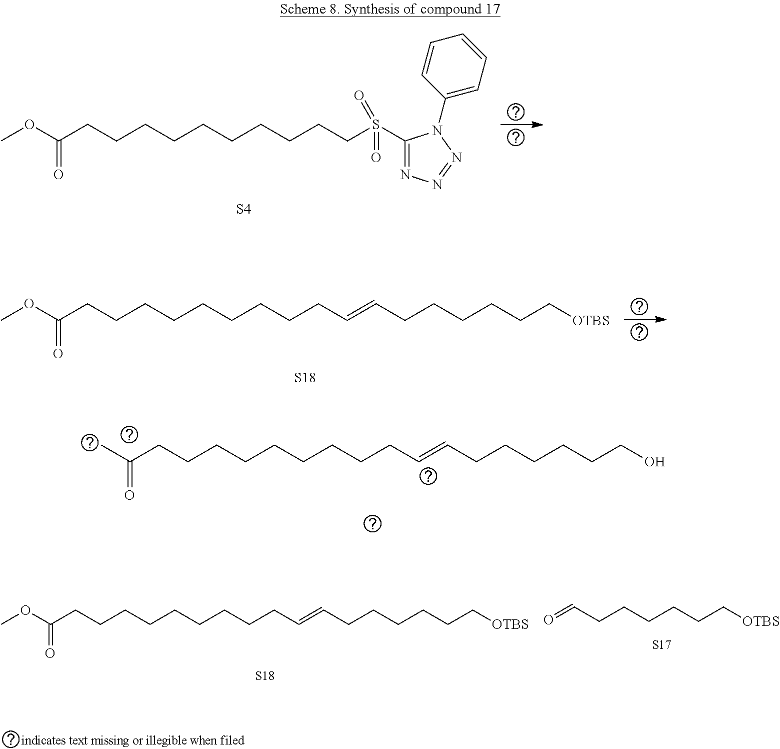

To a stirred solution of the S4 (80 mg, 0.20 mmol) in DME (1.3 mL) at −78° C. under nitrogen was added dropwise the KHMDS (0.5 M in toluene, 0.43 mL, 0.22 mmol) The mixture was then stirred for 30 min before addition of the aldehyde S17 (prepared as reported, 53 mg, 0.22 mmol). After stirring for a further 1 h at −78° C. the reaction mixture was quenched with sat. NH₄Cl (1.0 mL), then the mixture was extracted with EtOAc (3×10 mL). The combined organic phases were dried over Na₂SO₄ and evaporated. Without purified for the next step.

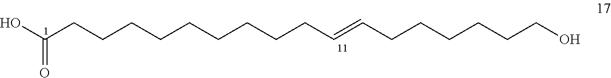 [SMILES: O=C(O)CCCCCCCCC/C=C/CCCCCCO]

To a solution of sulfide S18 (60 mg, 0.15 mmol) in THF (1.5 mL) was added a solution of TBAF (1.0 M in THF, 181 μL, 0.18 mmol) at room temperature. The reaction was stirred at room temperature for 30 min then quenched with H₂O (2.0 mL). The mixture was extracted with EtOAc (3×10 mL). The combined organic phases were dried over Na₂SO₄ and concentrated in vacuo to yield the product which was used directly in the follow reaction. The above product was dissolved in THF:H₂O=3:1 (2.4 mL), then LiOH (25 mg, 0.60 mmol) was added into the above solution, heated the mixture to 66° C. and stirred for 3 h, cooled to room temperature, 2M HCl was added to the mixture to adjust the pH to 2, then the mixture was extracted with EtOAc (3×5.0 mL). The combined organic phases were dried over Na₂SO₄ and evaporated. Purification by silica gel column chromatography (EtOAc:hexane=1:4-1:1) gave a white solid (31 mg, 70% for 2 steps). ¹H NMR (400 MHZ, CDCl₃) δ 5.43-5.32 (m, 2H), 3.64 (t, J=6.6 Hz, 2H), 2.34 (t, J=7.5 Hz, 2H), 2.04-1.92 (m, 4H), 1.69-1.51 (m, 5H), 1.41-1.20 (m, 19H). ¹³C NMR (101 MHz, CDCl₃) & 179.3, 130.5, 130.2, 63.0, 34.0, 32.7, 32.5, 32.5, 29.6, 29.5, 29.4, 29.2, 29.0, 29.02, 29.0, 25.6, 24.7. HRMS-ESI (m/z): calcd. for C₁₈H₃₄O₃H⁺ [M+H]⁺: 299.2581, found 299.2582.

### Synthesis of Probe-1

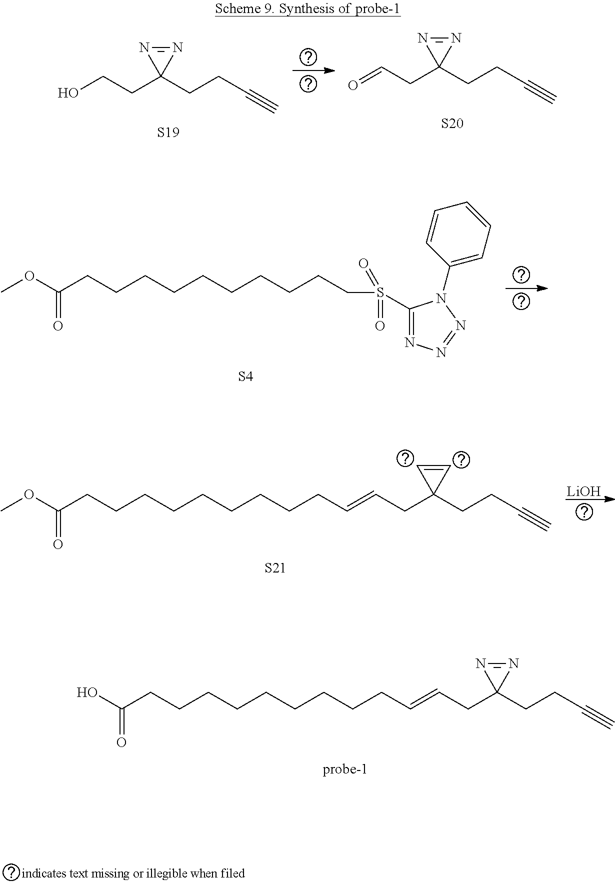

Oxalyl chloride (46 μL, 0.54 mmol) was dissolved in 2.0 mL DCM and brought to −78

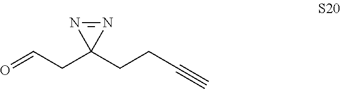

° C. A solution of DMSO (78 μL, 1.08 mmol) in DCM (1.0 mL) was added dropwise, and the reaction was allowed to stir for 15 min. A solution of the alcohol S19 (50 mg, 0.36 mmol) in DCM (1.0 mL) was added dropwise, and the reaction was allowed to stir an additional 15 min. Then Et₃N (300 μL, 2.2 mmol) was added dropwise. After 15 min, the reaction was allowed to warm to room temperature. The reaction mixture was transferred to a separatory funnel and washed with H₂O (2 mL). The phases were separated, and the aqueous phase was extracted with DCM (3×5.0 mL). The combined organic extracts were dried over Na₂SO₄, and concentrated. Purification by silica gel column chromatography (EtOAc:hexane=1:6) gave a brown oil (20 mg, 40%). ¹H NMR (400 MHZ, CDCl₃) δ 9.74 (s, 1H), 2.46 (d, J=1.6 Hz, 2H), 2.06 (td, J=7.3, 2.7 Hz, 2H), 2.01 (d, J=2.6 Hz, 1H), 1.73 (t, J=7.2 Hz, 2H). ¹³C NMR (101 MHZ, CDCl₃) δ 197.5, 82.5, 69.8, 48.4, 32.4, 24.5, 13.3.

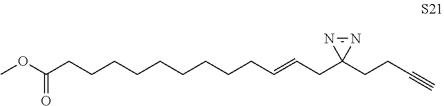 [SMILES: C#CCCC1(C/C=C/CCCCCCCCCC(=O)OC)N=N1]

To a stirred solution of the S4 (40 mg, 0.10 mmol) in DME (0.50 mL) at −78° C. under nitrogen was added dropwise the KHMDS (0.5 M in toluene, 0.19 mL, 0.10 mmol) The mixture was then stirred for 30 min before addition of the aldehyde S20 (12 mg, 0.09 mmol). After stirring for a further 1 h at −78° C. the reaction mixture was quenched with sat. NH₄Cl (1.0 mL), then the mixture was extracted with EtOAc (3×10 mL). The combined organic phases were dried over Na₂SO₄ and evaporated. Purification by silica gel column chromatography (EtOAc:hexane=1:30) gave a colorless oil (10 mg, 37%). ¹H NMR (400 MHZ, CDCl₃) δ 5.56-5.42 (m, 1H), 5.25-5.12 (m, 1H), 3.66 (s, 3H), 2.30 (t, J=7.6 Hz, 2H), 2.08-1.89 (m, 6H), 1.68-1.54 (m, 4H), 1.44-1.12 (m, 13H). ¹³C NMR (101 MHZ, CDCl₃) δ 174.5, 135.5, 122.1, 83.0, 69.1, 51.6, 37.0, 34.3, 32.7, 31.9, 29.5, 29.4, 29.3, 29.2, 28.3, 25.1, 13.4. HRMS-ESI (m/z): calcd. for C₁₉H₃₀N₂O₂H⁺ [M+H]⁺: 319.2380, found 319.2381.

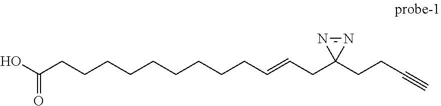 [SMILES: C#CCCC1(C/C=C/CCCCCCCCCC(=O)O)N=N1]

The above product S21 (10 mg, 0.031 mmol) was dissolved in THF:H₂O=3:1 (0.40 mL), then LiOH (5.4 mg, 0.13 mmol) was added into the above solution, heated the mixture to 66° C. and stirred for 3 h, cooled to room temperature, 2M HCl was added to the mixture to adjust the pH to 2, then the mixture was extracted with EtOAc (3×5.0 mL). The combined organic phases were dried over Na₂SO₄ and evaporated. Purification by silica gel column chromatography (EtOAc:hexane=1:3) gave a white solid (7.6 mg, 76%). ¹H NMR (400 MHZ, CDCl₃) δ 5.49 (dt, J=15.3, 6.6 Hz, 1H), 5.25-5.13 (m, 1H), 2.35 (t, J=7.5 Hz, 2H), 2.09-1.89 (m, 6H), 1.63 (td, J=7.5, 3.9 Hz, 4H), 1.42-1.25 (m, 13H). ¹³C NMR (101 MHz, CDCl₃) δ 179.7, 135.5, 122.1, 83.0, 69.1, 37.0, 32.7, 31.9, 29.8, 29.5, 29.5, 29.4, 29.3, 29.20, 28.3, 24.8, 13.4. HRMS-ESI (m/z): calcd. for C₁₈H₂₈N₂O₂H⁺ [M+H]⁺: 305.2224, found 305.2220.

### Synthesis of Probe-2

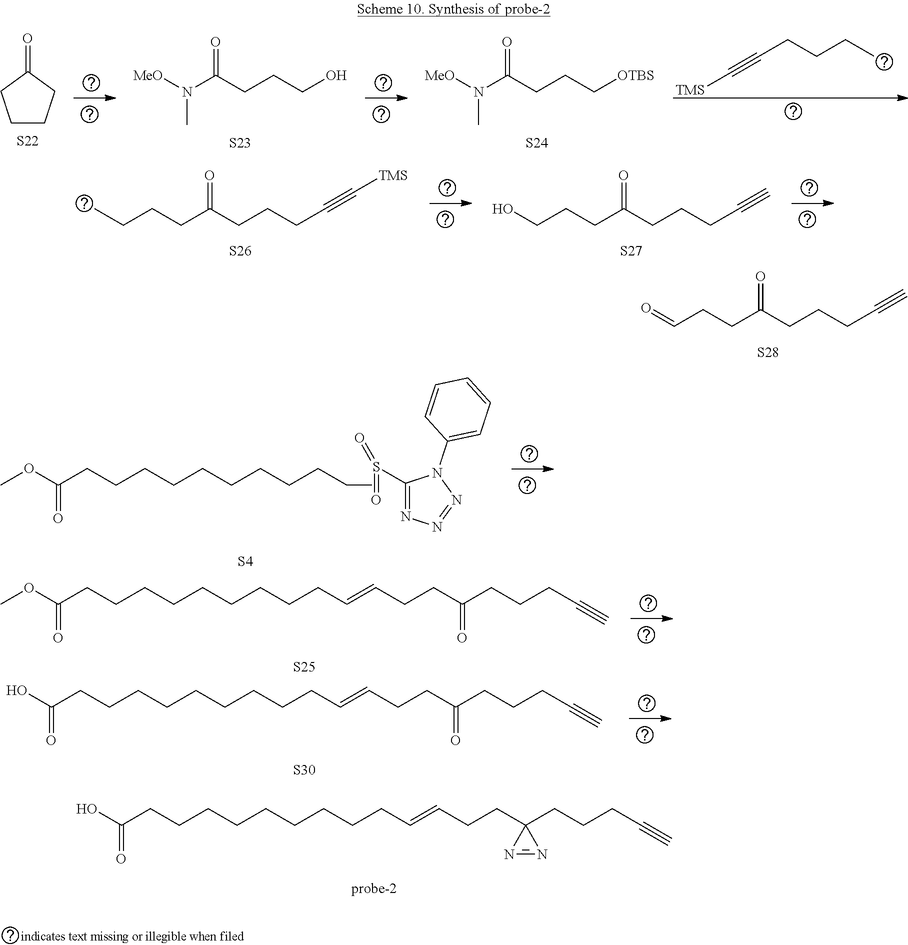

Trimethylaluminium (66 mL, 132 mmol, 2.0 M in toluene) was added over 1 h to a

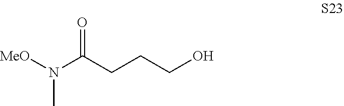

solution of N,O-dimethylhydroxylamine hydrochloride (13.6 g, 139.4 mmol) in DCM (50 mL) at −78° C. The solution was warmed to room temperature and stirred for 4 h. The solution was then cooled to −5° C., y-butyrolactone S22 (4.4 ml, 57.2 mmol) was added and the resulting mixture stirred for a further 1.5 h. After this time, the solution was carefully quenched at 0° C. by addition of a solution of potassium sodium L-tartrate tetrahydrate (16 g) in water (20 mL) and stirred overnight. The resulting precipitate was filtered through a plug of Celite and washed with DCM. The organic phase was dried over Na₂SO₄, filtered, and the solvent removed in vacuo to give a light yellow oil (8.31 g, 99%). Obtained characterization data were in agreement with those published in the literature.

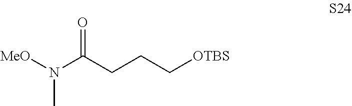 [SMILES: CON(C)C(=O)CCCO[Si](C)(C)C(C)(C)C]

To a solution of S23 (3.4 g, 23.2 mmol) and imidazole (2.4 g, 34.8 mmol) in DCM (70 mL) was added TBSCl (3.8 g, 25.5 mmol) at 0° C. The mixture was stirred at rt for 3 h. The mixture was then quenched by addition of H₂O (15 mL), and extracted with DCM (3× 30 mL). The combined organic layers were washed with brine, dried over Na₂SO₄, and concentrated. The residue was purified by silica gel chromatography (EtOAc:hexane=1:3) gave a colorless oil (5.6 g, 92%). Obtained characterization data were in agreement with those published in the literature.

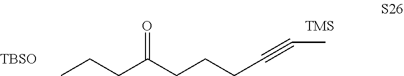

### Synthesis of (5-(trimethylsilyl) pent-4-yn-1-yl) magnesium chloride

This compound was prepared by following the reported procedure. Magnesium turnings (206 mg, 8.6 mmol) were etched with the back of a glass pipette and added to a flame-dried, two-neck RBF containing a stir bar and fitted with an oven-dried reflux condenser. After purging the reaction vessel with argon, a small bead of 12 was added to the magnesium turnings followed by anhydrous THF (3.0 mL) and the resulting mixture was stirred for 15 min at room temperature. A few drops of (5-chloropent-1-yn-1yl)trimethylsilane (1.0 g, 5.7 mmol) dissolved in anhydrous THF (7.0 mL) was then added to the mixture and the mixture was heated to reflux. The remaining (5-chloropent-1yn-1-yl)trimethylsilane solution was then slowly added to the refluxing reaction mixture over 30 min. When the addition was complete, the reaction was refluxed for an additional 3 h before cooling to room temperature.

Compound S24 (1.5 g, 5.7 mmol) was dissolved in anhydrous THF (30 mL) and cooled to 0° C. under N₂. The above fresh prepared (5-(Trimethylsilyl) pent-4-yn-1-yl) magnesium chloride S25 was then added dropwise and stirring for an additional 1 h at room temperature. After stirring for 1 h, the reaction was quenched with the addition of sat. NH₄Cl (aq.) (10 mL) and the product was extracted with EtOAc (3×30 mL). The combined organic layers were washed with brine, then dried over Na₂SO₄ and concentrated under reduced pressure. The residue was purified by silica gel chromatography (EtOAc:hexane=1:20) gave a yellow oil (1.2 g, 63%). ¹H NMR (400 MHZ, CDCl₃) δ 3.60 (td, J=5.7, 2.3 Hz, 2H), 2.59-2.45 (m, 4H), 2.25 (tt, J=6.9, 1.2 Hz, 2H), 1.84-1.71 (m, 4H), 0.88 (s, 9H), 0.14 (s, 9H), 0.03 (s, 6H). ¹³C NMR (101 MHz, CDCl₃) δ 210.2, 106.2, 85.2, 62.0, 41.1, 39.1, 26.7, 25.8, 22.3, 19.1, 18.2, −0.1, −5.5. HRMS-ESI (m/z): calcd. for C₁₈H₃₆O₂Si₂H⁺ [M+H]⁺: 341.2327, found 341.2331.

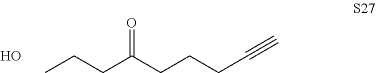

To a solution of S26 (300 mg, 0.88 mmol) in THF (5.0 mL) was added a solution of TBAF (1.0 M in THF, 2.2 mL, 2.2 mmol) at room temperature. The reaction was stirred at room temperature for 30 min then quenched with H₂O (5.0 mL). The mixture was extracted with EtOAc (3×20 mL). The combined organic phases were washed with brine, dried over Na₂SO₄ and concentrated in vacuo. The residue was purified by silica gel chromatography (EtOAc:hexane=1:1) gave a yellow oil (130 mg, 96%). ¹H NMR (400 MHZ, CDCl₃) δ 3.64 (td, J=6.1, 1.8 Hz, 2H), 2.59 (dtd, J=8.9, 7.1, 1.7 Hz, 4H), 2.24 (ddd, J=7.0, 4.7, 2.2 Hz, 2H), 2.16 (s, 1H), 1.97 (q, J=2.1 Hz, 1H), 1.91-1.74 (m, 4H). ¹³C NMR (101 MHZ, CDCl₃) δ 210.9, 83.6, 69.1, 62.2, 41.2, 39.6, 26.5, 22.2, 17.7. HRMS-ESI (m/z): calcd. for C₉H₁₄O₂H⁺ [M+H]⁺: 155.1067, found 155.1067.

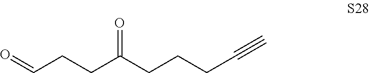

Dess-Martin reagent (99 mg, 0.23 mmol) was added to the solution of S27 (30 g, 0.2 mmol) in DCM (2.0 mL) at 0° C., and the mixture was stirred at room temperature for 4 hours. The reaction mixture was quenched with saturated Na₂S₂O₃:NaHCO₃=1:1 (5 mL), and the product was extracted with DCM (3×10 mL). The organic layer was washed with brine and dried over Na₂SO₄, concentrated in vacuo. The residue was purified by silica gel chromatography (EtOAc:hexane=1:4) gave a yellow oil (20 mg, 70%). ¹H NMR (400 MHZ, CDCl₃) δ 9.80 (s, 1H), 2.76 (p, J=2.8 Hz, 4H), 2.64 (td, J=7.2, 2.8 Hz, 2H), 2.24 (tt, J=6.9, 2.3 Hz, 2H), 2.01-1.93 (m, 1H), 1.88-1.75 (m, 2H). ¹³C NMR (101 MHz, CDCl₃) δ 208.0, 200.4, 83.5, 69.1, 41.0, 37.5, 34.8, 22.3, 17.7. HRMS-ESI (m/z): calcd. for C₉H₁₂O₂Na⁺ [M+Na]⁺: 175.0730, found 175.0735.

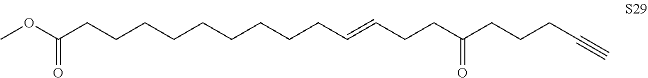 [SMILES: C#CCCCC(=O)CC/C=C/CCCCCCCCCC(=O)OC]

To a stirred solution of the S4 (54 mg, 0.13 mmol) in DME (2.0 mL) at −78° C. under nitrogen was added dropwise the KHMDS (0.5 M in toluene, 0.26 mL, 0.13 mmol) The mixture was then stirred for 30 min before addition of the aldehyde S28 (20 mg, 0.13 mmol). After stirring for a further 1 h at −78° C. the reaction mixture was quenched with sat. NH₄Cl (2.0 mL), then the mixture was extracted with EtOAc (3×10 mL). The combined organic phases were washed with brine, dried over Na₂SO₄ and evaporated. Purification by silica gel column chromatography (EtOAc:hexane=1:10) gave a yellow oil (16 mg, 37%). ¹H NMR (400 MHZ, CDCl₃) δ 5.49-5.29 (m, 2H), 3.66 (s, 3H), 2.63-2.52 (m, 2H), 2.47 (t, J=7.4 Hz, 2H), 2.34-2.16 (m, 6H), 2.06-1.90 (m, 3H), 1.79 (pd, J=7.0, 1.0 Hz, 2H), 1.62 (p, J=6.5 Hz, 2H), 1.37-1.23 (m, 12H). ¹³C NMR (101 MHZ, CDCl₃) δ 209.9, 174.3, 131.6, 128.2, 83.6, 69.0, 51.4, 42.8, 41.1, 34.1, 32.5, 29.5, 29.4, 29.4, 29.3, 29.1, 29.1, 26.9, 25.0, 22.2, 17.8. HRMS-ESI (m/z): calcd. for C₂₁H₃₄O₃H⁺ [M+H]⁺: 335.2581, found 335.2584.

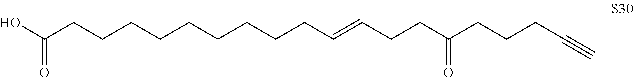 [SMILES: C#CCCCC(=O)CC/C=C/CCCCCCCCCC(=O)O]

The above product S29 (16 mg, 0.048 mmol) was dissolved in THF:H₂O=3:1 (0.40 mL), then LiOH (8.0 mg, 0.19 mmol) was added into the above solution, heated the mixture to 66° C. and stirred for 3 h, cooled to room temperature, 2M HCl was added to the mixture to adjust the pH to 2, then the mixture was extracted with EtOAc (3×5.0 mL). The combined organic phases were washed with brine, dried over Na₂SO₄ and evaporated. Purification by silica gel column chromatography (EtOAc:hexane=1:10) gave a white solid (15 mg, 96%). ¹H NMR (400 MHZ, CDCl₃) δ 5.49-5.31 (m, 2H), 2.55 (t, J=7.3 Hz, 2H), 2.48 (t, J=7.5 Hz, 2H), 2.35 (t, J=5.8 Hz, 2H), 2.30-2.16 (m, 4H), 2.06-1.90 (m, 3H), 1.85-1.73 (m, 2H), 1.63 (p, J=7.6 Hz, 2H), 1.41-1.24 (m, 12H). ¹³C NMR (101 MHZ, CDCl₃) δ 210.0, 179.9, 131.6, 128.2, 83.6, 69.0, 42.8, 41.1, 34.0, 32.5, 29.4, 29.4, 29.4, 29.2, 29.1, 29.1, 26.9, 24.7, 22.2, 17.8. HRMS-ESI (m/z): calcd. for C₁₈H₃₂O₃H⁺ [M+H]⁺: 321.2424, found 321.2424.

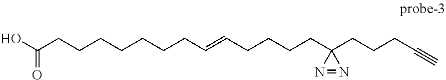 [SMILES: C#CCCCC1(CCCC/C=C/CCCCCCCC(=O)O)N=N1]

Ketone S30 (16 mg, 0.050 mmol) was dissolved in a solution of NH₃ (7.0 N in MeOH, 0.19 mL, 1.3 mmol) at 0° C. under N₂. After stirring for 3 h at 0° C., a solution of hydroxylamine-O-sulfonic acid (6.5 mg, 0.058 mmol) in MeOH (0.1 mL) was added dropwise. The reaction mixture was allowed to slowly warm to room temperature while stirring overnight. The reaction was then concentrated and the remaining residue was redissolved in anhydrous DCM (1.0 mL) and pyridine (0.1 mL) under the protection of N2. PCC (11 mg, 0.05 mmol) was then added in small portions while the reaction mixture was cooled to 0° C. The reaction was then allowed to warm to room temperature and stirred for an additional 1 h, then 2 M HCl (1.0 mL) was added into above solution. The resulting solution was extracted with DCM (3×10 mL).

The combined organic phases were washed with brine, dried over Na₂SO₄ and evaporated. Purification by silica gel column chromatography (EtOAc:hexane=1:3) gave a oil (4.0 mg, 30%). ¹H NMR (400 MHZ, CDCl₃) δ 5.50-5.24 (m, 2H), 2.35 (t, J=7.5 Hz, 2H), 2.16 (td, J=7.0, 2.7 Hz, 2H), 2.02-1.91 (m, 3H), 1.79 (p, J=6.6 Hz, 2H), 1.63 (p, J=7.3 Hz, 2H), 1.56-1.34 (m, 2H), 1.34-1.17 (m, 16H). ¹³C NMR (101 MHZ, CDCl₃) δ 179.7, 131.7, 128.3, 83.4, 68.9, 34.0, 33.1, 32.6, 32.5, 31.8, 29.7, 29.6, 29.5, 29.5, 29.4, 29.4, 29.2, 29.1, 29.1, 28.3, 26.8, 24.7, 22.7, 18.0. HRMS-mixed (m/z): calcd. for C₂₀H₃₂N₂O₂H⁺ [M+H]⁺: 333.2537, found 333.2535.

### Synthesis of Probe-3

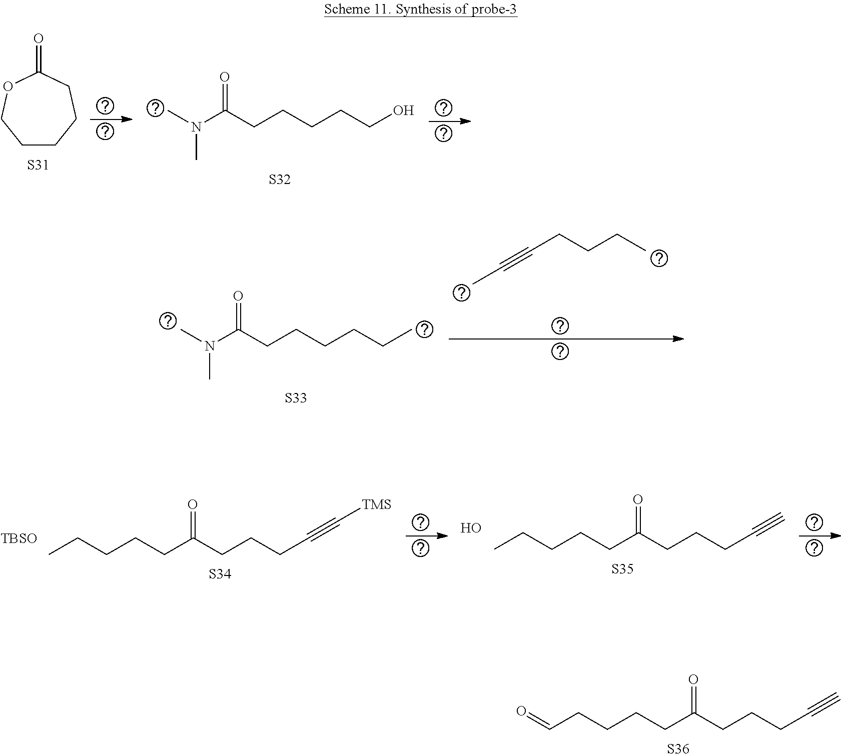

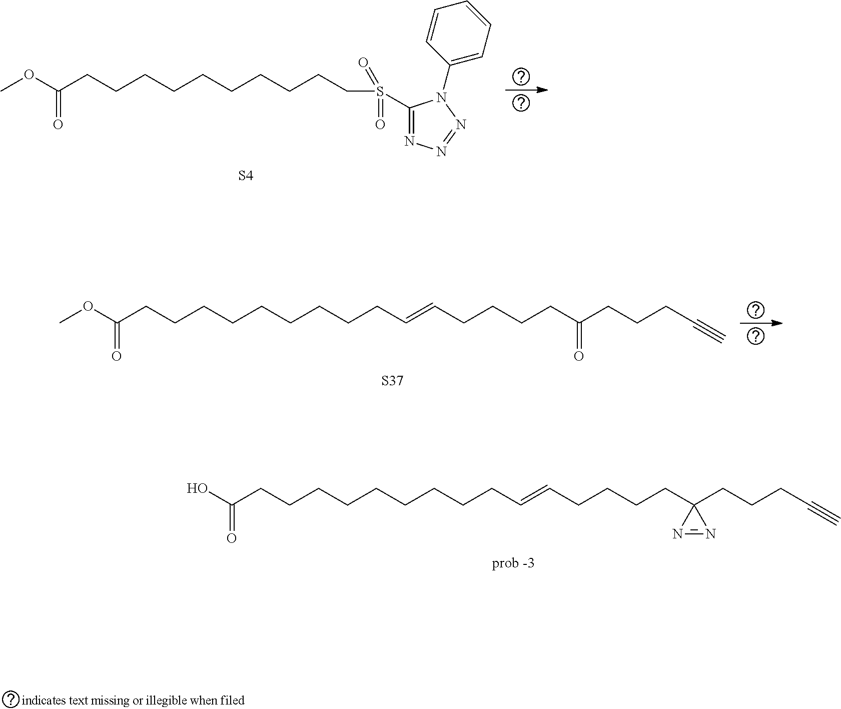 [SMILES: COC(=O)CCCCCCCCCCS(=O)(=O)c1nnnn1-c1ccccc1 ; "S4" ; C#CCCCC(=O)CCCC/C=C/CCCCCCCCCC(=O)OC ; "S37" ; C#CCCCC1(CCCC/C=C/CCCCCCCCCC(=O)O)N=N1 ; "prob -3"]

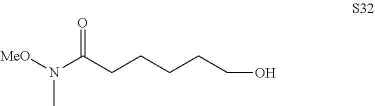

Trimethylaluminium (2.0 M in toluene, 7.5 mL, 35 mmol) was added dropwise to a solution of N,O-dimethylhydroxylamine hydrochloride (3.4 g, 35 mmol) and S31 (2.0 g, 17.5 mmol) in DCM (60 mL) at 0° C. The solution stirred for 24 h at 0° C. After this time, the solution was carefully quenched at 0° C. by addition of a solution of potassium sodium L-tartrae tetrahydrate (3.9 g) in H₂O (5.9 mL). The resulting precipitate was filtered through a plug of Celite and washed with DCM. The organic phase was dried over Na₂SO₄, filtered, and the solvent removed in vacuo to give a light yellow oil (2.9 g, 99%). Obtained characterization data were in agreement with those published in the literature.

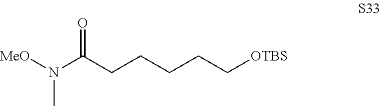 [SMILES: CON(C)C(=O)CCCCCO[Si](C)(C)C(C)(C)C]

To a solution of S32 (2.9 g, 16.6 mmol) and imidazole (1.8 g, 26.4 mmol) in DCM (50 mL) was added TBSCl (2.8 g, 18.6 mmol) at 0° C. The mixture was stirred at room temperature for 3 h. The mixture was then quenched by addition of H₂O (15 mL), and extracted with DCM (3×30 mL). The combined organic layers were washed with brine, dried over Na₂SO₄, and concentrated. The residue was purified by silica gel chromatography (EtOAc:hexane=1:3) gave a colorless oil (4.7 g, 98%). Obtained characterization data were in agreement with those published in the literature.

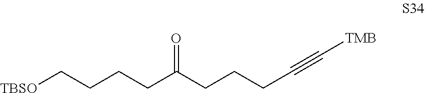

Compound S33 (1.6 g, 5.7 mmol) was dissolved in anhydrous THF (30 mL) and cooled to 0° C. under N₂. The fresh prepared (5-(Trimethylsilyl) pent-4-yn-1-yl) magnesium chloride S25 was then added dropwise and stirring for an additional 1 h at room temperature. After stirring for 1 h, the reaction was quenched with the addition of sat. NH₄Cl (aq.) (10 mL) and the product was extracted with EtOAc (3×30 mL). The combined organic layers were washed with brine, then dried over Na₂SO₄ and concentrated under reduced pressure. The residue was purified by silica gel chromatography (EtOAc:hexane=1:30) gave a yellow oil (0.7 g, 34%). ¹H NMR (400 MHZ, CDCl₃) δ 3.45 (td, J=6.5, 1.7 Hz, 2H), 2.39 (td, J=7.3, 1.7 Hz, 2H), 2.28 (td, J=7.5, 1.7 Hz, 2H), 2.11 (td, J=6.9, 1.7 Hz, 2H), 1.63 (pd, J=7.1, 1.7 Hz, 2H), 1.52-1.32 (m, 4H), 1.19 (ttd, J=8.7, 6.3, 3.3 Hz, 2H), 0.74 (d, J=1.7 Hz, 9H), 0.00 (d, J=1.7 Hz, 9H), −0.10 (d, J=1.6 Hz, 6H). ¹³C NMR (101 MHZ, CDCl₃) δ 210.4, 106.2, 85.2, 62.8, 42.8, 41.0, 32.5, 25.8, 25.4, 23.6, 22.3, 19.1, 18.2, −5.4. HRMS-ESI (m/z): calcd. for C₂₀H₄₀O₂Si₂H⁺ [M+H]⁺: 369.2640, found 369.2640.

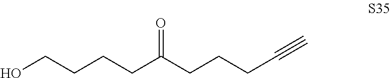

To a solution of S34 (710 mg, 1.9 mmol) in THF (8.0 mL) was added a solution of TBAF (1.0 M in THF, 4.8 mL, 4.8 mmol) at room temperature. The reaction was stirred at room temperature for 30 min then quenched with H₂O (5.0 mL). The mixture was extracted with EtOAc (3×30 mL). The combined organic phases were washed with brine, dried over Na₂SO₄ and concentrated in vacuo. The residue was purified by silica gel chromatography (EtOAc:hexane=1:1) gave a yellow oil (350 mg, 100%). ¹H NMR (400 MHZ, CDCl₃) δ 3.64 (td, J=6.5, 4.4 Hz, 2H), 2.56 (td, J=7.2, 3.1 Hz, 2H), 2.45 (td, J=7.2, 3.2 Hz, 2H), 2.23 (tdd, J=6.8, 4.2, 2.5 Hz, 2H), 1.97 (q, J=2.6 Hz, 1H), 1.86-1.71 (m, 2H), 1.68-1.51 (m, 4H), 1.44-1.30 (m, 2H). ¹³C NMR (101 MHz, CDCl₃) δ 210.6, 83.6, 69.1, 62.6, 42.8, 41.1, 32.4, 25.4, 23.5, 22.2, 17.8. HRMS-ESI (m/z): calcd. for C₁₁H₁₈O₂Na⁺ [M+Na]⁺: 205.1199, found 205.1202.

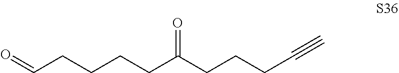

Dess-Martin reagent (413 mg, 0.97 mmol) was added to the solution of S35 (100 mg, 0.65 mmol) in DCM (6.5 mL) at 0° C., and the mixture was stirred at room temperature for 4 hours. The reaction mixture was quenched with saturated Na₂S₂O₃:NaHCO₃=1:1 (10 mL), and the product was extracted with DCM (3×20 mL). The organic layer was washed with brine and dried over Na₂SO₄, concentrated in vacuo. The residue was purified by silica gel chromatography (EtOAc:hexane=1:3) gave a yellow oil (70 mg, 70%). ¹H NMR (400 MHZ, CDCl₃) δ 9.77 (s, 1H), 2.56 (t, 2H), 2.48-2.41 (m, 4H), 2.23 (tdd, J=6.9, 2.7, 1.2 Hz, 2H), 1.97 (dq, J=2.7, 1.3 Hz, 1H), 1.79 (d, 2H), 1.70-1.55 (m, 4H). ¹³C NMR (101 MHZ, CDCl₃) δ 209.8, 202.2, 83.5, 77.4, 77.1, 76.8, 69.1, 43.7, 42.5, 41.1, 23.2, 22.2, 21.6, 17.7. HRMS-ESI (m/z): calcd. for C₁₁H₁₆O₂H⁺ [M+H]⁺: 181.1213, found 181.1218

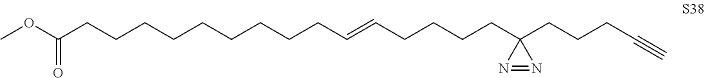 [SMILES: C#CCCCC1(CCCC/C=C/CCCCCCCCCC(=O)OC)N=N1]

To a stirred solution of the S4 (174 mg, 0.43 mmol) in DME (2.0 mL) at −78° C. under nitrogen was added dropwise the KHMDS (0.5 M in toluene, 0.85 mL, 0.43 mmol) The mixture was then stirred for 30 min before addition of the aldehyde S36 (70 mg, 0.39 mmol). After stirring for a further 1 h at −78° C. the reaction mixture was quenched with sat. NH₄Cl (3.0 mL), then the mixture was extracted with EtOAc (3×10 mL). The combined organic phases were washed with brine, dried over Na₂SO₄ and evaporated. Purification by silica gel column chromatography (EtOAc:hexane=1:10) gave a yellow oil (65 mg, 46%). ¹H NMR (400 MHZ, CDCl₃) δ 5.46-5.30 (m, 2H), 3.66 (s, 3H), 2.55 (t, J=7.2 Hz, 2H), 2.41 (t, J=7.4 Hz, 2H), 2.30 (t, J=7.5 Hz, 2H), 2.23 (td, J=6.9, 2.6 Hz, 2H), 2.08-1.91 (m, 5H), 1.79 (d, J=6.4 Hz, 2H), 1.69-1.52 (m, 4H), 1.40-1.24 (m, 15H). ¹³C NMR (101 MHZ, CDCl₃) δ 210.5, 174.3, 130.9, 129.6, 83.6, 69.0, 51.4, 42.8, 41.0, 34.1, 32.6, 32.3, 29.6, 29.4, 29.4, 29.3, 29.2, 29.2, 25.0, 23.4, 22.3, 17.8. HRMS-ESI (m/z): calcd. for C₂₃H₃₈O₃H⁺ [M+H]⁺: 363.2894, found 363.2890.

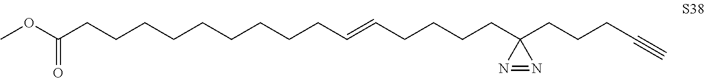 [SMILES: C#CCCCC1(CCCC/C=C/CCCCCCCCCC(=O)OC)N=N1]

To a solution of S37 (65 mg, 0.18 mmol) in MeOH (0.10 mL) was added an ammonia solution in MeOH (7 N, 2.0 mL) at 0° C. under Ar. The solution was stirred at that temperature for 1 h, then warmed to room temperature stirred for 2 h. Cooled to 0° C. again, to this solution was then added hydroxylamine-O-sulfonic acid (24.3 mg, 0.22 mmol) slowly at 0° C. The resulting mixture was warmed to rt and stirred for 16 h. The white precipitate was removed by filtration and the remaining solution was concentrated by vacuo. The residue was re-dissolved in DCM (1.0 mL). To this solution was added Et₃N (0.043 mL, 0.3 mmol) and a solution of 12 (82 mg, 0.32 mmol) in DCM (1.0 mL) dropwise until the solution stayed red-brown. The reaction mixture was quenched by saturated Na₂S₂O₃, and extracted with DCM (3×10 mL). The combined organic phases were washed with brine, dried over Na₂SO₄ and evaporated. Purification by silica gel column chromatography (EtOAc:hexane=1:30) gave a yellow oil (29 mg, 43%). ¹H NMR (400 MHz, CDCl₃) δ 5.46-5.27 (m, 2H), 3.66 (s, 3H), 2.30 (t, J=7.7 Hz, 3H), 2.16 (tdd, J=6.8, 2.6, 1.0 Hz, 2H), 2.02-1.89 (m, 5H), 1.67-1.56 (m, 2H), 1.56-1.44 (m, 2H), 1.41-1.20 (m, 18H), 1.15-1.03 (m, 1H). ¹³C NMR (101 MHz, CDCl₃) δ 174.3, 130.9, 129.6, 83.4, 68.9, 51.4, 34.1, 32.7, 32.6, 32.3, 31.8, 29.6, 29.4, 29.3, 29.2, 29.1, 29.1, 28.4, 25.0, 23.3, 22.8, 18.0. HRMS-mixed (m/z): calcd. for C₂₃H₃₈N₂O₂H⁺ [M+H]⁺: 375.3006, found 375.3018.

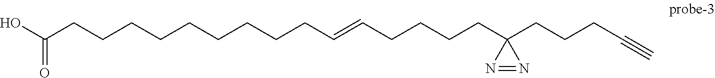 [SMILES: C#CCCCC1(CCCC/C=C/CCCCCCCCCC(=O)O)N=N1]

The above product S38 (28 mg, 0.076 mmol) was dissolved in THF:H₂O=3:1 (2.4 mL), then LiOH (13 mg, 0.30 mmol) was added into the above solution, heated the mixture to 66° C. and stirred for 3 h, cooled to room temperature, 2M HCl was added to the mixture to adjust the pH to 2, then the mixture was extracted with EtOAc (3×10 mL). The combined organic phases were washed with brine, dried over Na₂SO₄ and evaporated. Purification by silica gel column chromatography (EtOAc:hexane=1:6) gave a colorless oil (24 mg, 96%). ¹H NMR (400 MHZ, CDCl₃) δ 5.47-5.23 (m, 2H), 2.35 (t, J=7.5 Hz, 3H), 2.20-2.06 (m, 2H), 1.98-1.87 (m, 5H), 1.69-1.57 (m, 2H), 1.57-1.43 (m, 2H), 1.41-1.23 (m, 18H), 1.15-1.05 (m, 1H). ¹³C NMR (101 MHz, CDCl₃) δ 180.1, 130.9, 129.6, 83.5, 68.9, 34.1, 32.7, 32.6, 32.3, 31.8, 29.6, 29.4, 29.4, 29.2, 29.1, 29.1, 29.1, 28.4, 24.7, 23.3, 22.8, 18.0. HRMS-ESI (m/z): calcd. for C₂₂H₃₆N₂O₂H⁺ [M+H]⁺: 361.2850, found 361.2847.

TABLE 1
Human “blood chemicals” (633 in total)

| Organic metabolites & | | | | | | | |
|---|---|---|---|---|---|---|---|
| inorganics | | Lipids | | Supplements | | Proteins | |
| Inorganics | 26 | FA | 14 | Vitamins | 16 | Antibodies | 5 |
| | | (monounsaturated) | | | | | |
| Carbohydrates | 8 | FA | 11 | Fish oil & Omegas | 25 | Hormones | 49 |
| | | (polyunsaturated) | | | | | |
| Amino acids | 26 | FA | 37 | Minerals | 31 | Enzymes | 11 |
| | | (saturated) | | | | | |
| Glycosaminoglycans | 19 | FA | 3 | Antioxidants | 31 | Coagulation | 15 |
| | | (trans) | | | | Factors | |
| Nucleotides | 4 | FA | 18 | Herbs | 44 | Complement | 14 |
| | | (trans-monoenoic) | | | | system | |
| Organic chemicals | 45 | Other lipids | 5 | | | Peptides | 5 |
| | | | | | | Growth Factors | 4 |
| | | | | | | Cytokines/Chemokines | 96 |
| | | | | | | Other proteins | 29 |

TABLE 2

| | | | | | | Stock |
|---|---|---|---|---|---|---|
| | | Physiological | | Working | | concentration |
| Number | Name | concentration | | concentration | | 2000X |
| 1 | Alpha-linolenic | 18.5 +/− 7.9 | uM | 37 | uM | 20.6 ul in 2 ml |
| | acid (ALA) | | | | | |
| 2 | Arachidonic acid | 55.4 +/− 4.48 | uM | 110.8 | uM | 0.073 ul in 2 ml |
| 3 | Astaxanthin | 0.092 +/− 0.025 | uM | 0.184 | uM | 109.8186 ug in 1 ml |
| | | | | | | DMSO (1000X) |
| 4 | cis -Vaccenic acid | 10.7 +/− 5 | uM | 21.4 | uM | 0.013 ul in 2 ml |
| 5 | Coconut oil | — | | 800 | mg/L | 1.77 ul in 2 ml |
| 6 | Docosahexaenoic | 94.2 +/− 31.3 | uM | 188.4 | uM | 0.131 ul in 2 ml |
| | Acid (DHA) | | | | | |
| 7 | Docosapentaenoic | 9.0 (7.3-11.5) | uM | 18 | uM | 23.7954 ul in 2 ml |
| | acid (DPA) | | | | | |
| 8 | Eicosatrienoic Acid | 3.61 +/− 2.1 | uM | 7.22 | uM | |
| 9 | Eicosapentaenoic | 11.0 +/− 8.3 | uM | 22 | uM | 0.014 ul in 2 ml |
| | Acid (EPA) | | | | | |
| 10 | Erucic acid | 1.264 +/− 1.590 | uM | 2.58 | uM | 873.5106 ug in 1 ml |
| | | | | | | DMSO (1000X) |
| 11 | Evening | — | | 200 | mg/L | 0.43 ul in 2 ml |
| | primrose oil | | | | | |
| 12 | Flax seed oil | — | | 260 | mg/L | 0.56 ul in 2 ml |
| 13 | Gamma linolenic | 1.08 +/− 1.5 | uM | 2.16 | uM | 1.3 ul in 2 ml |
| | acid (GLA) | | | | | |
| 14 | linoleic acid (CLA) | 264 +/− 17.0 | uM | 528 | uM | 0.328 ul in 2 ml |
| 15 | Myristic Acid | 25.0 (8.0-70.0) | uM | 50 | uM | 11.4185 mg in 1 ml |
| | | | | | | DMSO (1000X) |
| 16 | Myristoleic acid | 2.016 +/− 0.733 | uM | 4.032 | uM | 0.002 ul in 2 ml |
| 17 | Nervonic acid | 0.900 +/− 0.974 | uM | 1.8 | uM | 659.916 ug mg in 1 |
| | | | | | | ml DMSO (1000X) |
| 18 | Oleic acid | 122 +/− 56 | uM | 244 | uM | 0.154 ul in 2 ml |
| 19 | Palmitic Acid | 122 +/− 48 | uM | 244 | uM | 0.146 ul in 2 ml |
| 20 | Palmitoleic acid | 31.0 (5.0-85.0) | uM | 62 | uM | 0.035 ul in 2 ml |
| 21 | Pumpkin seed oil | — | | 400 | mg/L | 0.87 ul in 2 ml |
| 22 | Stearic Acid | 85 (31-470) | uM | 170 | uM | 48.3616 mg |
| | | | | | | in 1 ml DMSO (1000X) |
| 23 | Stearidonic acid | 0.408 +/− 0.4 | uM | 0.816 | uM | |
| 24 | trans - | 10.7 +/− 5 | uM | 21.4 | uM | 6.0446 mg in 1 ml |
| | Vaccenic acid | | | | | DMSO (1000X) |
| 25 | alpha-lipoic acid | 0.077 +/− 0.017 | uM | 0.154 | uM | 31.7748 ug |
| | | | | | | in 1 ml DMSO (1000X) |
| 26 | CoQ-10 | 4.31 +/− 0.42 | uM | 8.62 | uM | 7.442 mg in 1 ml |
| | | | | | | DMSO (1000X) |
| 27 | Inositol | N/A | | 120 | mg/L | 120 mg in 1 ml |
| | | | | | | DMSO (1000X) |
| 28 | L-glutathione | 800.50 +/− 201.73 | uM | 1601 | uM | 492.0193 mg 1 ml |
| | | | | | | DMSO (1000X) |
| 29 | Lutein | 0.52 (0.24-0.79) | uM | 1.04 | uM | 591.624 ug 1 ml |
| | | | | | | DMSO (1000X) |
| 30 | Lycopene | 0.743 +/− 0.168 | uM | 1.486 | uM | 797.7888 ug 1 ml |
| | | | | | | DMSO (1000X) |
| 31 | L-α- | N/A | | 84 | mg/L | 25 mg in 298 ul |
| | Phosphatidylcholine | | | | | (1000X) |
| 32 | Pterostibene | N/A | | 10 | mg/L | 10 mg in 1 ml (1000X) |
| 33 | Quercetin | 0.06 +/− 0.03 | uM | 0.12 | uM | 37 ug in 1 ml (1000X) |
| 34 | Thymoquinone | N/A | | 200 | mg/L | 200 mg in 1 ml (1000X) |
| 35 | Trimethylglycine | N/A | | 130 | mg/L | 130 mg in 1 ml (1000X) |
| 36 | Ubiquinol | N/A | | 20 | mg/L | 50 mg in 2.5 ml (1000X) |
| 37 | Zeaxanthin | 0.04 +/− 0.04 | uM | 0.08 | uM | 46 ug in 1 ml (1000X) |
| 38 | 6-gingerol | N/A | | 5.5 | mg/L | 5.5 mg in 1 ml (1000X) |
| 39 | Boswellia Serrata | — | | 90 | mg/L | 90 mg in 1 ml (1000X) |
| | Extract | | | | | |
| 40 | Bromelain from | — | | 100 | mg/L | 100 mg in 1 ml (1000X) |
| | pineapple stem | | | | | |
| 41 | Caffeic acid | — | | 0.012 | mg/L | 0.012 mg in 1 ml (1000X) |
| | phenethyl ester | | | | | |
| 42 | capsaicin | N/A | | 100 | mg/L | 100 mg in 1 ml (1000X) |
| 43 | Chondroitin | 9.58 +/− 2.71 | uM | 300 | mg/L | 300 mg in 1 ml (1000X) |
| | sulfate sodium | | | | | |
| 44 | Collagen, type II, | — | | 0.2 | g/L | 200 mg in 1 ml (1000X) |
| | chicken-sternal cartilage | | | | | |
| 45 | Crucuminoids | — | | 95 | mg/L | 95 mg in 1 ml (1000X) |
| 46 | Curcumin | 0.17 +/− 0.013 | uM | 0.34 | uM | 133 mg in 1 ml (1000X) |
| | | | | 133 | mg/L | |
| 47 | D-glucosamine 3-sulfate | N/A | | 100 | mg/L | 100 mg in 1 ml (1000X) |
| 48 | Gelatin solution | — | | 115 | mg/L | |
| 49 | Glucosamine | 0.29 (0.0-0.6) | uM | 0.58 | uM | 125 ug in 1 ml (1000X) |
| | Hydrochloride | | | | | |
| 50 | Hyaluronic acid | 0.24 (0.12-0.36) | uM | 0.48 | uM | 20 mg/ml in 1 ml (1000X) |
| | sodium salt | | | | | |
| | | | | 20 | mg/L | |
| 51 | Methylsulfonylmethane | 8.8 +/− 7.3 | uM | 17.6 | uM | 1.65 mg in 1 ml (1000X) |
| 52 | N-acetyl D-glucosamine | N/A | | 140 | mg/L | 140 mg in 1 ml (1000X) |
| 53 | Phenolics, total | — | | 20 | mg/L | 20 mg in 1 ml (1000X) |
| 54 | Resveratrol | N/A | | 4 | mg/L | 4 mg in 1 ml (1000X) |
| 55 | Rutoside trihydrate | — | | 20 | mg/L | 20 mg in 1 ml (1000X) |
| 56 | Taurine | 162.0 +/− 60.0 | uM | 324 | uM | 40.54 mg in 1 ml (1000X) |
| 57 | Calcium citrate | Ca2+: 2420.0 | | 200 | mg/L | 200 mg in 1 ml (1000X) |
| | | (2250.0-2590.0) uM | | | | |
| | | Citrate: 190.0 | | | | |
| | | (30.0-400.0) uM | | | | |
| 58 | calcium d-glucarate | Ca2+: 2420.0 | | 200 | mg/L | 200 mg in 1 ml (1000X) |
| | | (2250.0-2590.0) uM | | | | |
| | | Glucarate: N/A in blood | | | | |
| 59 | chromium Chloride | Cr3+: | | 100 | ug/L | 100 ug in 1 ml (1000X) |
| | | 0.0075 +/− 0.0015 uM | | | | |
| | | Cl−: | | | | |
| | | 103700.0 +/− 1900.0 uM | | | | |
| 60 | Chromium picolinate | Cr3+: | | 100 | ug/L | 100 ug in 1 ml (1000X) |
| | | 0.0075 +/− 0.0015 uM | | | | |
| | | Picolinate: | | | | |
| | | 0.299 +/− 0.034 uM | | | | |
| 61 | Copper Gluconate | Cu2+: 37.6 | | 400 | ug/L | 400 ug in 1 ml (1000X) |
| | | (23.6-49.9) uM | | | | |
| | | Gluconate: | | | | |
| | | 3.295 +/− 0.534 uM | | | | |
| 62 | copper sulfate | Cu2+: 37.6 | | 600 | ug/L | |
| | | (23.6-49.9) uM | | | | |
| | | Sulfate: 490.0 | | | | |
| | | (310.0-580.0) uM | | | | |
| 63 | Dicalcium Phosphate | Ca2+: 2420.0 | | 50 | mg/L | 50 mg in 1 ml (1000X) |
| | | (2250.0-2590.0) uM | | | | |
| | | PO43−: 379.1 +/− 31.6 uM | | | | |
| 64 | Ferrous Fumarate | Fe2+: 9766 +/− 1246 uM | | 6 | mg/L | 6 mg in 1 ml (1000X) |
| | | Fumarate: 1.5 | | | | |
| | | (0.0-4.0) uM | | | | |
| 65 | Ferrous gluconate | Fe2+: 9766 +/− 1246 uM | | 3.6 | mg/L | 3.6 mg in 1 ml (1000X) |
| | | Gluconate: | | | | |
| | | 3.295 +/− 0.534 uM | | | | |
| 66 | Ferrous Sulfate | Fe2+: 9766 +/− 1246 uM | | 13 | mg/L | 13 mg in 1 ml (1000X) |
| | | Sulfate: 490.0 | | | | |
| | | (310.0-580.0) uM | | | | |
| 67 | Iron Gluconate | Fe3+: N/A | | 3.6 | mg/L | 3.6 mg in 1 ml (1000X) |
| | | Gluconate: | | | | |
| | | 3.295 +/− 0.534 uM | | | | |
| 68 | L-Aspartic acid | L-Aspartic acid: | | 50 | mg/L | 50 mg in 1 ml (1000X) |
| | potassium salt | 21.0 +/− 5.0 uM | | | | |
| | | K+: 4200.0 | | | | |
| | | (3600.0-4800.0) uM | | | | |
| 69 | Magnesium Aspartate | Mg2+: | | 50 | mg/L | 50 mg in 1 ml (1000X) |
| | | 833.0 +/− 208.0 uM | | | | |
| | | L-Aspartic acid: | | | | |
| | | 21.0 +/− 5.0 uM | | | | |
| 70 | Magnesium citrate | Mg2+: | | 50 | mg/L | 50 mg in 1 ml (1000X) |
| | | 833.0 +/− 208.0 uM | | | | |
| | | Citrate: 190.0 | | | | |
| | | (30.0-400.0) uM | | | | |
| 71 | Manganese sulfate | Mn2+: 0.17292 | | 400 | ug/L | 400 ug in 1 ml (1000X) |
| | | (0.16382-0.18202) uM | | | | |
| | | Sulfate: 490.0 | | | | |
| | | (310.0-580.0) uM | | | | |
| 72 | Malic acid | N/A | | 120 | mg/L | 120 mg in 1 ml (1000X) |
| 73 | Monopotassium | K+: 4200.0 | | 40 | mg/L | 40 mg in 1 ml (1000X) |
| | Phosphate | (3600.0-4800.0) uM | | | | |
| | | PO43−: 379.1 +/− 31.6 uM | | | | |
| 74 | Potassium Citrate | K+: 4200.0 | | 20 | mg/L | 20 mg in 1 mk (1000X) |
| | | (3600.0-4800.0) uM | | | | |
| | | Citrate: 190.0 | | | | |
| | | (30.0-400.0) uM | | | | |
| 75 | Potassium Gluconate | K+: 4200.0 | | 20 | mg/L | 20 mg in 1 ml (1000X) |
| | | (3600.0-4800.0) uM | | | | |
| | | Gluconate: | | | | |
| | | 3.295 +/− 0.534 uM | | | | |
| 76 | Potassium phosphate | K+: 4200.0 | | 10 | mg/L | 10 mg in 1 ml (1000X) |
| | dibasic | (3600.0-4800.0) uM | | | | |
| | | PO43−: | | | | |
| | | 379.1 +/− 31.6 uM | | | | |
| 77 | Potassium sorbate | N/A | | 2.4 | mg/L | 2.4 mg in 1 ml (1000X) |
| 78 | Silicic acid | N/A | | 1 | mg/L | 1 mg in 1 ml (1000X) |
| 79 | Sodium citrate | Na+: 142600.0 | | 5 | mg/L | 5 mg in 1 ml (1000X) |
| | | (138000.0-146000.0) uM | | | | |
| | | Citrate: 190.0 | | | | |
| | | (30.0-400.0) uM | | | | |
| 80 | Sodium Molybdate | Na+: 142600.0 | | 2.5 | ug/L | 2.5 ug in 1 ml (1000X) |
| | | (138000.0-146000.0) uM | | | | |
| | | Molybdate: N/A | | | | |
| 81 | sodium selenite | Na+: 142600.0 | | 60 | ug/L | 60 ug in 1 ml (1000X) |
| | | (138000.0-146000.0) uM | | | | |
| | | Selenite: 0.0013 | | | | |
| | | (0.00-0.0063) uM | | | | |
| 82 | strontium chloride | Sr2+: 0.44 +/− 0.17 uM | | 20 | mg/L | 20 mg in 1 ml (1000X) |
| | | Cl−: | | | | |
| | | 103700.0 +/− 1900.0 uM | | | | |
| 83 | Tricalcium phosphate | Ca2+: 2420.0 | | 100 | mg/L | 100 mg in 1 ml (1000X) |
| | hydrate | (2250.0-2590.0) uM | | | | |
| | | PO43−: | | | | |
| | | 379.1 +/− 31.6 uM | | | | |
| 84 | Vanadylsulfate | Sulfate: 490.0 | | 2 | mg/L | 2 mg in 1 ml (1000X) |
| | | (310.0-580.0) uM | | | | |
| 85 | Zinc Citrate | Zn2+: 16.8 +/− 6.3 uM | | 6 | mg/L | 6 mg in 1 ml (1000X) |
| | | Citrate: 190.0 | | | | |
| | | (30.0-400.0) uM | | | | |
| 86 | Zinc Oxide | | | 4 | mg/L | 4 mg in 1 ml (1000X) |
| 87 | Zinc Picolinate | Zn2+: 16.8 +/− 6.3 uM | | 10 | mg/L | 10 mg in 1 ml (1000X) |
| | | Picolinate: | | | | |
| | | 0.299 +/− 0.034 uM | | | | |
| 88 | p-Aminobenzoic Acid | 15.0 (5.01-32.0) | uM | 30 | uM | 4.1142 ug in 1 ml (1000X) |
| 89 | d-Biotin | 0.11-0.13 | uM | 0.26 | uM | 63.5206 ng in 1 ml (1000X) |
| 90 | Folic Acid | 0.021 (0.017-0.025) | uM | 0.042 | uM | 18.5388 ng in 1 ml (1000X) |
| 91 | Niacinamide | 0.44 +/− 0.0054 | uM | 0.88 | uM | 107.4656 ng in 1 ml (1000X) |
| 92 | D-Pantothenic Acid, | 4.91 +/− 0.38 | uM | 9.82 | uM | 2.3398 ug in 1 ml (1000X) |
| | hemicalcium salt | | | | | |
| 93 | Pyridoxal Hydrochloride | 0.251 +/− 0.051 | uM | 0.502 | uM | 102.2172 ng in 1 ml (1000X) |
| 94 | Pyridoxamine | 0.164 +/− 0.038 | uM | 0.328 | uM | 79.0841 ng in 1 ml (1000X) |
| | Dihydrochloride | | | | | |
| 95 | Pyridoxine | 0.025 (0.007-0.060) | uM | 0.05 | uM | 10.282 ng in 1 ml (1000X) |
| | Hydrochloride | | | | | |
| 96 | Riboflavin | 0.530 (0.265-1.30) | uM | 1.06 | uM | 398.9416 ng in 1 ml (1000X) |
| 97 | Thiamine Hydrochloride | 0.34 (0.14-0.79) | uM | 0.68 | uM | 229.3436 ng in 1 ml (1000X) |
| 98 | DL-6,8-Thioctic Acid | 0.077 +/− 0.017 | uM | 0.154 | uM | 31.7748 ng in 1 ml (1000X) |

| | |
|---|---|
| 99 | Tryptophan |
| 100 | Threonine |
| 101 | Isoleucine |
| 102 | Leucine |
| 103 | Lysine |

Blood Chemicals

| | | 2000* physilogical |
|---|---|---|
| | | concentration stock: |
| Number | Name | mass (mg) in 1 ml |
| | ALDOSTERONE | 0.04992094 |
| 105 | ALUMINUM | 0.2080104 |
| | CHLORIDE, ANHYDROUS | |
| 106 | ANDROSTERONE | 0.1277936 |
| | VETRANAL,/androsterone | |
| 107 | TIN(II) CHLORIDE/Tin | 0.3432122 |
| 108 | PHOSPHORUS | 300FIX |
| | PENTACHLORIDE | |
| 109 | FUROSEMIDE/LASIX | 800 |
| 110 | CALCIUM CHLORIDE/calcium | 77.1311 |
| 111 | CYTOSINE | 3.31078 |
| 112 | L-THYROXINE | 9.94394E−05 |
| 113 | (+)-DELTA-TOCOPHEROL | 22.22628 |
| 114 | THEOBROMINE | 2.34208 |
| 115 | O-METHYLGUANINE | 300FIX |
| 116 | 17A-HYDROXYPROGESTERONE | 0.17811794 |
| 117 | THYMIDINE | 0.823582 |
| 118 | | |
| 119 | CAFFEINE | 50.10102 |
| 120 | PREGNENOLONE | 2.5310806 |
| | ACETATE/pregnenolone | |
| 121 | FOLIC ACID, | 0.366362 |
| 122 | GUANINE | 29.31922 |
| 123 | ESTRIOL | 0.230704 |
| 124 | D-LACTOSE MONOHYDRATE | 307FIX |
| 125 | CADMIUM CHLORIDE/cadmium | 0.1246576 |
| 126 | SULFUR/sulfide | 0.009621 |
| 127 | PURINE | 305FIX |
| 128 | ESTRONE | 0.0324444 |
| 129 | LEAD(II) SULFIDE | 3.325853 |
| 130 | TETRAETHYLAMMONIUM | 0.4 |
| | HYDROGEN SULFATE, FOR IPC | |
| 131 | D-GLUCURONIC ACID | 76.49116 |
| 132 | CHOLINE CHLORIDE | 3.4905 |
| 133 | CREATININE, ANHYDROUS, | 24.614912 |
| 134 | L-CYSTEINE BIOULTRA, | 0.460408 |
| 135 | ACETYLCHOLINE | 0.0781138 |
| | CHLORIDE | |
| 136 | L-CITRULLINE | 16.11748 |
| 137 | GLYCEROL MOLECULAR | 8.84064 |
| | BIOLOGY REAGENT | |
| 138 | COPPER(II) SULFATE, | 4.859600979 |
| | ANHYDROUS,/copper | |
| 139 | | |
| 140 | CORTISONE | 0.25735416 |
| 141 | MERCURY(II) CHLORIDE | 0.192765 |
| 142 | URIC ACID | 121.71164 |
| 143 | POTASSIUM | 242.2875 |
| | CHLORIDE/potassium | |
| 144 | UREA | 270.27 |
| 145 | CHITIN | 302FIX |
| 146 | CELLULOSE FIBROUS | 304FIX |
| 147 | D(+)-GALACTOSE | 44.31936 |
| | ANHYDROUS | |
| 148 | SUCROSE | 2.0538 |
| 149 | SODIUM BICARBONATE/ | 1121.11345 |
| | hydrogen carbonate | |
| 150 | SODIUM SULFATE | 164.7664 |
| 151 | NICOTINIC ACID | 14.330004 |
| 152 | POTASSIUM | 297.5 |
| | BROMIDE/potassium | |
| 153 | 0% deoxycholic acid | 8.064 |
| | sodium salt + 50% cholic | |
| | acid sodium sa | |
| 154 | TAURINE | 42.551 |
| 155 | GUANIDINE | 0.19106 |
| | HYDROCHLORIDE, | |
| 156 | L-ALANINE | 72.51926 |
| 157 | THIAMINE | 2.664433 |
| | HYDROCHLORIDE/thiamine | |
| 158 | MANGANESE SULFATE | 3.09002364 |
| | MONOHYDRATE, | |
| 159 | MYO-INOSITOL | 6.161472 |
| 160 | L-ASCORBIC ACID | 40.15536 |
| | BIOXTRA, | |
| 161 | ZINC CHLORIDE | 6.29706 |
| 162 | L-ORNITHINE | 47.55084 |
| | MONOHYDROCHLORIDE | |
| 163 | DL-MALIC ACID | 5.63178 |
| 164 | ZINC SULFATE | 13.285272 |
| | HEPTAHYDRATE | |
| 165 | BETAINE | 66.7755 |
| 166 | MAGNESIUM | 1.73301242 |
| | CHLORIDE ANHYDROUS | |
| 167 | D-(+)-GLUCOSAMINE | 0.258756 |
| | HYDROCHLORIDE | |
| 168 | SUCCINIC ACID | 7.55776 |
| 169 | URACIL | 3.497208 |
| 170 | OXALIC ACID SODIUM SALT | 5.9496 |
| 171 | D-(+)-MALTOSE | 305.362725 |
| | MONOHYDRATE/D-Maltose | |
| 172 | SODIUM BROMIDE | 19.178696 |
| | BIOXTRA/Bromide | |
| 173 | D(−)FRUCTOSE | 12.25088 |
| 174 | PECTIN | 303FIX |
| 175 | SODIUM FLUORIDE/fluoride | 0.5450302 |
| 176 | SODIUM CHLORIDE | 4266.12 |
| | BIOXTRA/sodium | |
| 177 | LIPASE FROM | 1 |
| | PORCINE PANCREAS, | |
| 178 | D-PANTOTHENIC | 2.5208966 |
| | ACID HEMICALCIUM | |
| 179 | 2,3-DPG, D-Glycerate | 250.00675 |
| | 2,3-diphosphate sodium salt | |
| 180 | TRANS-B-CAROTENE, | 0.7140371 |
| | TYPE I,/all trans B carotene | |
| 181 | L-GLUTATHIONE REDUCED | 154.0026618 |
| 182 | VITAMIN K1 | 0.76619 |
| 183 | (+/−)-A-TOCOPHEROL/ | 49.96236 |
| | Alpha-Tocopherol | |
| 184 | 3-HYDROXYBUTYRIC ACID | 16.656 |
| 185 | L(+)LACTIC ACID SODIUM | 104.249418 |
| 186 | ADENOSINE FREE BASE | 1.7290428 |
| 187 | PYRUVIC ACID | 45.43896 |
| 188 | 3-HYDROXYTYRAMINE | 0.0123266 |
| | HCL/dopamine | |
| 189 | PHENOL | 1.6017522 |
| 190 | 4-AMINOBENZOIC ACID | 8.77696 |
| 191 | LYCOPENE | 4.8908857 |
| 192 | (+)-GAMMA-TOCOPHEROL | 55.3017696 |
| 193 | ALBUMIN HUMAN | 25000 |
| 194 | (−)-EPINEPHRINE | 0.161216 |
| 195 | ERGOCALCIFEROL | 0.277655 |
| 196 | BIOTIN, POWDER, | 0.2369807 |
| 197 | 2-DEOXY-L-RIBOSE/ | 0.67065 |
| | Deoxycytidine | |
| 198 | CHONDROITIN SULFATE, | 307.25 |
| | A SODIUM SALT | |
| 199 | CHOLECALCIFEROL | 300FIX |
| | CRYSTALLINE | |
| 200 | 5-HYDROXYTRYPTAMINE | 4.2536 |
| | HYDROCHLORIDE/Serotonin | |
| 201 | OXYTOCIN | 179.27982 |
| 202 | VASOACTIVE | 124.7175 |
| | INTESTINAL PEPTIDE | |
| 203 | C-PEPTIDE FRAGMENT | 0.13289144 |
| | 3-33, HUMAN/C-peptide | |
| 204 | OLECYSTOKININ | 1.6798718 |
| | FRAGMENT 26-33 | |
| | AMIDE NON SULFAT | |
| 205 | GLYCOGEN | 20FIX |
| 206 | ALPHA 1 ANTITRYPSIN, | 0.6 |
| | HUMAN PLASMA | |
| 207 | ,3′,5-TRIIODO-L-THYRONINE | 3.76858E−05 |
| | SODIUM SALT/Liothyronin | |
| 208 | HYALURONIC ACID | 100FIX |
| | SODIUM SALT | |
| 209 | ARG8-VASOPRESSIN | 0.000138781 |
| | ACETATE SALT/Vasopressin | |
| 210 | L-A- | 25FIX |
| | PHOSPHATIDYLETHANOLAMINE | |
| | TYPE I | |
| 211 | CHORIONIC | 50FIX |
| | GONADOTROPIN HUMAN, | |
| 212 | THYROCALCITONIN | 0.000002 |
| | SALMON/calcitonin | |
| 213 | BETA-CRYPTOXANTHIN | 1.1167974 |
| 214 | L-(+)- | 0.697072 |
| | ERGOTHIONEINE | |
| | INNER SALT | |
| 215 | FERRITIN TYPE IV | 0.6 |
| | FROM HUMAN LIVER | |
| 216 | HISTAMINE FREE | 0.122265 |
| | BASE CRYSTALLINE | |
| 217 | ANGIOTENSIN II HUMAN | 0.031594636 |
| 218 | L-A- | 27.81696 |
| | PHOSPHATIDYLCHOLINE | |
| | TYPE XVI-E | |
| 219 | MELATONIN | 0.00015795 |
| | CRYSTALLINE/melatonin | |
| 220 | INDOXYL beta-D- | 3.395835 |
| | GLUCOSIDE/Inodxylglucuronide | |
| 221 | SPHINGOMYELIN/ | 50FIX |
| | SM(d18:1/16:0) | |
| 222 | GUANOSINE 3′:5′-CYCLIC | 0.2129702 |
| | MONOPHOSPHATE SODIUM | |
| 223 | ARSENIC(III) CHLORIDE | 0.056613744 |
| 224 | SODIUM CYANIDE, | exclude |
| 225 | INIZING HORMONE | all prepared (fix) |
| | RELEASING HORMONE | |
| | HUMAN ACE | |
| 226 | 5alpha- | 300FIX |
| | Tetrahydrocorticosterone | |
| 227 | CHOLESTEROL | 2513.225 |
| 228 | ADENOSINE 5′- | 429.8892 |
| | TRIPHOSPHATE | |
| | DISODIUM SALT | |
| 229 | CITRIC ACID | 153.696 |
| 230 | CALCIUM | 77.1311 |
| | CHLORIDE/calcium | |
| 231 | BILIRUBIN MIXED ISOMERS | 24.55572 |
| 232 | ACETONE | 19.7472 |
| 233 | LITHIUM ACETOACETATE/ | 18.57944 |
| | Acetoacetic acid | |
| 234 | Carcinoembryonic | 10 |
| | Antigen (CEA) | |
| 235 | PHYTANIC ACID | 8.000768 |
| 236 | ETHYL ALCOHOL/Ethanol | 7.3712 |
| 237 | ALCOHOL | 7.3712 |
| 238 | A-CAROTENE | 6.012944 |
| 239 | PYRIMIDINE | 4.8054 |
| 240 | PROTOPORPHYRIN IX | 4.726344 |
| 241 | A-CRYPTOXANTHIN | 4.5114192 |
| 242 | LEPTIN | 4 |
| 243 | PROINSULIN C-PEPTIDE | 3.1829512 |
| | (55-89)/c-peptide | |
| 244 | SELENIUM TETRACHLORIDE/ | 1.175723881 |
| | Selenium ion (se2+) | |
| 245 | PYRIDOXINE | 1.01508 |
| 246 | XANTHOPHYLL FROM | 1.0012112 |
| | MARIGOLD/xanthophyll (Lutein) | |
| 247 | TESTOSTERONE--DEA | 1.0008174 |
| | SCHEDULE III | |
| 248 | IODINE | 0.925452 |
| | MONOCHLORIDE/IODINE | |
| 249 | IN ADENINE DINUCLEOTIDE | 0.8046247 |
| | DISODIUM SALT HYDRATE/ | |
| 250 | 5ALPHA- | 0.493748 |
| | DIHYDROTESTOSTERONE | |
| | (DHT)/DHT | |
| 251 | THIAMINE PYROPHOSPHATE | 0.3041082 |
| 252 | ADENOSINE 3′:5′- | 0.296289 |
| | CYCLIC MONOPHOSPHATE | |
| 253 | ANDROSTENEDIONE | 0.2823666 |
| 254 | NOREPINEPHRINE | 0.186098 |
| 255 | 11-DEOXYCORTISOL/Cortexolone | 0.1489778 |

TABLE 3

| Rank | Blood chemicals | Relative changed percentage (%) |
|---|---|---|
| 1 | Nicotinic Acid | 205.602475 |
| 2 | Potassium Phosphate Dibasic | 177.4846 |
| 3 | Caffeine | 170.0384 |
| 4 | Trans-Vaccenic Acid | 133.920675 |
| 5 | Stearidonic Acid | 109.251075 |
| 6 | Folic Acid | 105.595875 |
| 7 | Potassium Gluconate | 97.7133 |
| 8 | Sphingomyelin | 95.238125 |
| 9 | Niacinamide | 95.233175 |
| 10 | Pregnenolone Acetate | 82.906525 |
| 11 | POTASSIUM BROMIDE | 78.357625 |
| 12 | Oleic acid | 77.53305 |
| 13 | CYTOSINE | 74.6228 |
| 14 | SUCROSE | 70.452825 |
| 15 | PURINE | 63.4263 |
| 16 | Gelatin | 59.80065 |
| 17 | Potassium Citrate | 58.7511 |
| 18 | MELATONIN CRYSTALLINE/melatonin | 56.15455 |
| 19 | Palmitoleic acid | 54.625525 |
| 20 | alpha-lipoic acid | 54.6153775 |
| 21 | THIAMINE HYDROCHLORIDE | 53.320075 |
| 22 | (−)-EPINEPHRINE | 53.119875 |
| 23 | COPPER(II) SULFATE, ANHYDROUS,/copper | 51.653075 |
| 24 | Tricalcium phosphate hydrate | 51.487425 |
| 25 | SODIUM BICARBONATE | 50.3453675 |
| 26 | L-GLUTATHIONE REDUCED | 48.2820975 |
| 27 | p-Aminobenzoic Acid | 47.979275 |
| 28 | D-Glycerate 2,3-diphosphate sodium salt | 47.739595 |
| 29 | L-ALANINE | 44.49305 |
| 30 | Caffeic acid phenethyl ester | 43.8461725 |
| 31 | 6-gingerol | 42.62105 |
| 32 | GUANIDINE IIYDROCIILORIDE, | 41.8290325 |
| 33 | (+)-DELTA-TOCOPHEROL | 41.421275 |
| 34 | 17A-HYDROXYPROGESTERONE | 41.357225 |
| 35 | L-Alanine | 41.06955 |
| 36 | MYO-INOSITOL | 40.7157 |
| 37 | (+/−)-A-TOCOPHEROL/Alpha-Tocopherol | 40.654625 |
| 38 | NOREPINEPHRINE | 40.2469 |
| 39 | DL-MALIC ACID | 40.0104375 |
| 40 | THYMIDINE | 39.62865 |
| 41 | GLYCEROL MOLECULAR BIOLOGY REAGENT | 39.3621 |
| 42 | SODIUM SULFATE | 38.986945 |
| 43 | POTASSIUM CHLORIDE/potassium | 38.973175 |
| 44 | Stearic Acid | 38.766525 |
| 45 | Malic acid | 38.522425 |
| 46 | ALBUMIN HUMAN | 38.341525 |
| 47 | L-A-PHOSPHATIDYLCHOLINE TYPE XVI-E | 38.095225 |
| 48 | Nervonic acid | 37.681155 |
| 49 | 2-DEOXY-L-RIBOSE/Deoxycytidine | 36.6995175 |
| 50 | SODIUM CHLORIDE | 36.528025 |
| 51 | BIOTIN, POWDER, | 36.4532125 |
| 52 | D(+)-GALACTOSE ANHYDROUS | 35.840375 |
| 53 | TRANS-B-CAROTENE, TYPE I,/all trans B carotene | 35.08137 |
| 54 | L-Cysteine | 32.819475 |
| 55 | L-A-PHOSPHATIDYLETHANOLAMINE TYPE I | 32.242225 |
| 56 | CADMIUM CIILORIDE/cadmium | 32.11155 |
| 57 | Lutein | 31.92307 |
| 58 | D-Pantothenic Acid, hemicalcium salt | 31.9171 |
| 59 | MAGNESIUM CHLORIDE ANHYDROUS | 31.903775 |
| 60 | strontium chloride | 30.89245 |
| 61 | INDOXYL beta-D-GLUCOSIDE/Inodxyl glucuronide | 30.458225 |
| 62 | Lycopene | 30.0000125 |
| 63 | d-Biotin | 29.844575 |
| 64 | Manganese sulfate | 29.5514375 |
| 65 | A-CRYPTOXANTHIN | 29.100525 |
| 66 | FUROSEMIDE/LASIX | 29.012345 |
| 67 | Monopotassium Phosphate | 28.67195 |
| 68 | L-Asparagine | 28.4787 |
| 69 | LEPTIN | 27.08995 |
| 70 | PHENOL | 26.78725 |
| 71 | CHORIONIC GONADOTROPIN HUMAN, | 26.677575 |
| 72 | CoQ-10 | 26.5384625 |
| 73 | ANDROSTENEDIONE | 25.925925 |
| 74 | L-Glutamine | 25.51725 |
| 75 | CHOLECALCIFEROL CRYSTALLINE | 25.451545 |
| 76 | THEOBROMINE | 25.41615 |
| 77 | ESTRONE | 25.0199 |
| 78 | BETA-CRYPTOXANTHIN | 24.87725 |
| 79 | SULFUR/sulfide | 24.143425 |
| 80 | Riboflavin | 23.14715 |
| 81 | D-LACTOSE MONOHYDRATE | 22.470125 |
| 82 | Sodium Molybdate | 22.1968 |
| 83 | 11-DEOXYCORTISOL/Cortexolone | 21.85185 |
| 84 | BoswelliaSerrata Extract | 20.45585 |
| 85 | TIN(II) CHLORIDE/Tin | 18.86145 |
| 86 | 3-HYDROXYTYRAMINE HCL/dopamine | 18.776925 |
| 87 | L-THYROXINE | 18.7243 |
| 88 | PROINSULIN C-PEPTIDE (55-89)/c-peptide | 17.8836 |
| 89 | 4-AMINOBENZOIC ACID | 17.743325 |
| 90 | ANGIOTENSIN II HUMAN | 17.2506625 |
| 91 | Bromelain from pineapple stem | 16.0114 |
| 92 | ERGOCALCIFEROL | 16.00985 |
| 93 | ANDROSTERONE VETRANAL,/androsterone | 15.912225 |
| 94 | Inositol | 15.3846175 |
| 95 | Eicosatrienoic Acid | 15.3571425 |
| 96 | GUANINE | 15.13945 |
| 97 | Rutoside trihydrate | 14.9881 |
| 98 | L-ASCORBIC ACID | 14.4874425 |
| 99 | Sodium citrate | 13.8444075 |
| 100 | GLYCOGEN | 13.7470725 |
| 101 | L-glutathione | 13.4615325 |
| 102 | ZINC CHLORIDE | 13.3368125 |
| 103 | sodium selenite | 13.272315 |
| 104 | Resveratrol | 13.164175 |
| 105 | Evening primrose oil | 12.31885 |
| 106 | Astaxanthin | 12.261905 |
| 107 | THYROCALCITONIN SALMON/calcitonin | 11.374785 |
| 108 | PYRIDOXINE | 11.1111 |
| 109 | L-Arginine | 10.74767 |
| 110 | Erucic acid | 10.71428 |
| 111 | PYRUVIC ACID | 10.4220675 |
| 112 | VITAMIN K1 | 10.1265725 |
| 113 | ALCOHOL | 9.841275 |
| 114 | D-glucosamine 3-sulfate | 9.8006575 |
| 115 | TIIIAMINE PYROPIIOSPIIATE | 9.753075 |
| 116 | Trimethylglycine | 9.458665 |
| 117 | CHONDROITIN SULFATE, A SODIUM SALT | 9.1953975 |
| 118 | FERRITIN TYPE IV FROM HUMAN LIVER | 9.074575 |
| 119 | Gamma linolenic acid | 9.0579725 |
| 120 | Thiamine Hydrochloride | 8.9151325 |
| 121 | Collagen type II | 8.55482 |
| 122 | GUANOSINE 3′:5′-CYCLIC MONOPIIOSPIIATE | 8.265975 |
| | SODIUM | |
| 123 | L-ORNITHINE MONOHYDROCHLORIDE | 8.0543775 |
| 124 | Pumpkin seed oil | 7.9295 |
| 125 | Pyridoxal Hydrochloride | 7.787315 |
| 126 | XANTHOPHYLL FROM MARIGOLD/xanthophyll (Lutein | 7.53085 |
| 127 | Vanadylsulfate | 7.0938175 |
| 128 | Pterostibene | 6.87431 |
| 129 | FOLIC ACID, | 6.8525775 |
| 130 | A-CAROTENE | 6.56085 |
| 131 | Ferrous Fumarate | 6.27762 |
| 132 | O-METHYLGUANINE | 6.27401 |
| 133 | Curcumin | 5.9800925 |
| 134 | CHITIN | 5.909425 |
| 135 | Zinc Picolinate | 5.492225 |
| 136 | L-Valine | 5.29597 |
| 137 | trans-4-Hydroxy-L-proline | 5.2812025 |
| 138 | cis-Vaccenic acid | 5.238115 |
| 139 | L(+)LACTIC ACID SODIUM | 5.0818225 |
| 140 | 3-HYDROXYBUTYRIC ACID | 5.0818225 |
| 141 | Arachidonic acid | 4.583345 |
| 142 | Thymoquinone | 4.55838 |
| 143 | LECYSTOKININ FRAGMENT 26-33 AMIDE NON SULFA | 4.556175 |
| 144 | L-Aspartic acid potassium salt | 4.420875 |
| 145 | SUCCINIC ACID | 4.1401275 |
| 146 | PYRIMIDINE | 4.126995 |
| 147 | Copper Gluconate | 4.0672 |
| 148 | Magnesium citrate | 3.781875 |
| 149 | ADENOSINE FREE BASE | 3.703725 |
| 150 | Zinc Oxide | 3.6269575 |
| 151 | L-Histidine hydrochloride | 3.5306375 |
| 152 | Iron Gluconate | 3.27146 |
| 153 | Hyaluronic acid sodium salt | 3.09278 |
| 154 | ESTRIOL | 2.7888525 |
| 155 | ADENOSINE 3′:5′-CYCLIC MONOPHOSPHATE | 2.71604 |
| 156 | URACIL | 2.45679 |
| 157 | TAURINE | 2.3857 |
| 158 | L-Glutamic Acid | 2.2718 |
| 159 | Ubiquinol | 2.16524 |
| 160 | Dicalcium Phosphate | 1.94519 |
| 161 | Capsaicin | 1.661115 |
| 162 | (+)-GAMMA-TOCOPHEROL | 1.559935 |
| 163 | L-Tryptophan | 1.3580175 |
| 164 | ZINC SULFATE HEPTAHYDRATE | 1.046025 |
| 165 | VASOACTIVE INTESTINAL PEPTIDE | 1.0212 |
| 166 | UREA | −0.31116 |
| 167 | ADENINE DINUCLEOTIDE DISODIUM SALT HYDRAT | −0.3703625 |
| 168 | Silicic acid | −1.029765 |
| 169 | L-Lysine hydrochloride | −1.6049375 |
| 170 | Zcaxanthin | −1.6524075 |
| 171 | Zinc Citrate | −2.0725275 |
| 172 | Potassium sorbate | −2.28832 |
| 173 | Palmitic Acid | −2.643185 |
| 174 | MERCURY(II) CHLORIDE | −2.99494 |
| 175 | phenolics | −3.0134675 |
| 176 | L-CITRULLINE | −3.3839075 |
| 177 | 5ALPHA-DIHYDROTESTOSTERONE (DIIT)/DIIT | −3.4567975 |
| 178 | HISTAMINE FREE BASE CRYSTALLINE | −3.5938975 |
| 179 | L-Proline | −3.935085 |
| 180 | N-acetyl D-glucosamine | −4.044425 |
| 181 | Chromium picolinate | −4.0671975 |
| 182 | DL-6,8-Thioctic Acid | −4.135335 |
| 183 | Magnesium Aspartate | −4.155615 |
| 184 | % deoxycholic acid sodium salt + 50% cholic acid | −4.29424 |
| | sodium s | |
| 185 | Crucuminoids | −4.4019975 |
| 186 | Quercetin | −4.501425 |
| 187 | PECTIN | −4.9135575 |
| 188 | D-(+)-MALTOSE MONOHYDRATE | −4.9135625 |
| 189 | Dipotassium Phosphate | −5.3934625 |
| 190 | Pyridoxamine Dihydrochloride | −6.1761475 |
| 191 | L-Threonine | −6.543205 |
| 192 | URIC ACID | −6.65112 |
| 193 | Glucosamine Hydrochloride | −6.9785875 |
| 194 | OXYTOCIN | −6.99136 |
| 195 | L-Leucine | −7.16049 |
| 196 | ARG8-VASOPRESSIN ACETATE SALT/Vasopressin | −7.610475 |
| 197 | L-Isoleucine | −8.64197 |
| 198 | Calcium Carbonate | −8.7499975 |
| 199 | ACETYLCHOLINE CHLORIDE | −8.9394725 |
| 200 | Glycine | −8.9655275 |
| 201 | Flax seed oil | −9.0579825 |
| 202 | LIPASE FROM PORCINE PANCREAS | −9.4032675 |
| 203 | linoleic acid | −9.4202875 |
| 204 | L-Aspartic Acid | −9.6146025 |
| 205 | HYALURONIC ACID SODIUM SALT | −9.7381275 |
| 206 | L-(+)-ERGOTHIONEINE INNER SALT | −10.3927975 |
| 207 | ALPHA 1 ANTITRYPSIN, HUMAN PLASMA | −10.6048725 |
| 208 | Chondroitin sulfate sodium | −11.4617925 |
| 209 | C-PEPTIDE FRAGMENT 3-33, HUMAN/C-peptide | −11.7046425 |
| 210 | L-Methionine | −11.83801 |
| 211 | Calcium d-glucarate | −12.1428575 |
| 212 | CORTISONE | −12.2909425 |
| 213 | CELLULOSE FIBROUS | −13.200305 |
| 214 | ′,5-TRIIODO-L-THYRONINE SODIUM SALT/Liothyroni | −13.4206225 |
| 215 | L-Tyrosine | −13.4475575 |
| 216 | LEAD(II) SULFIDE | −13.6320975 |
| 217 | MANGANESE SULFATE MONOHYDRATE, | −14.2345875 |
| 218 | Calcium citrate | −14.5833325 |
| 219 | OXALIC ACID SODIUM SALT | −16.878975 |
| 220 | L-Phenylalanine | −17.2378 |
| 221 | Myristic Acid | −17.3913125 |
| 222 | Pyridoxine Hydrochloride | −17.45435 |
| 223 | Methylsulfonylmethane | −17.52577 |
| 224 | Taurine | −17.6843775 |
| 225 | PHYTANIC ACID | −18.606555 |
| 226 | BETAINE | −18.828455 |
| 227 | D-PANTOTHENIC ACID HEMICALCIUM | −18.9873425 |
| 228 | Docosapentaenoic acid | −19.39394 |
| 229 | IZING HORMONE RELEASING HORMONE HUMAN AC | −19.590155 |
| 230 | L-Cystine | −19.73001 |
| 231 | Myristoleic acid | −21.37681 |
| 232 | LITHIUM ACETOACETATE/Acetoacetic acid | −21.47541 |
| 233 | L-Serine | −22.1095325 |
| 234 | -HYDROXYTRYPTAMINE HYDROCHLORIDE/Serotoni | −22.38806 |
| 235 | L-CYSTEINE BIOULTRA, | −22.735805 |
| 236 | Alpha-linolenic acid | −24.529505 |
| 237 | D(−)FRUCTOSE | −25.5232025 |
| 238 | Copper sulfate | −26.31579 |
| 239 | SODIUM BROMIDE | −26.9790725 |
| 240 | Docosahexaenoic Acid | −29.8883575 |
| 241 | ACETONE | −31.065575 |
| 242 | CHOLESTEROL | −31.885245 |
| 243 | Chromium Chloride | −35.279105 |
| 244 | TETRAETHYLAMMONIUM HYDROGEN SULFATE | −35.945565 |
| 245 | Citric Acid | −36.80328 |
| 246 | L-a-Phosphatidylcholine | −37.161085 |
| 247 | Bilirubin Mixed Isomers | −37.6229525 |
| 248 | D-(+)-Glucosamine Hydrochloride | −38.232215 |
| 249 | Adenosine 5′-Triphosphate Disodium | −38.2786875 |
| 250 | Coconut Oil | −38.8516725 |
| 251 | Creatine | −40.990145 |
| 252 | Choline Chloride | −41.1074625 |
| 253 | Sodium Fluoride | −43.8125575 |
| 254 | D-Glucuronic Acid | −45.8000925 |

TABLE 4

| Rank | Blood chemicals | Relative changed percentage (%) |
|---|---|---|
| 1 | GUANINE | 236.028968 |
| 2 | THEOBROMINE | 211.643836 |
| 3 | THYMIDINE | 162.160531 |
| 4 | GUANOSINE 3′:5′-CYCLIC MONOPHOSPHATE SODIUM | 161.1621 |
| 5 | FERRITIN TYPE IV FROM HUMAN LIVER | 146.7316 |
| 6 | L-CITRULLINE | 137.20195 |
| 7 | CREATININE, ANHYDROUS, | 125.820975 |
| 8 | COPPER(II) SULFATE, ANHYDROUS,/copper | 108.412075 |
| 9 | BILIRUBIN MIXED ISOMERS | 98.6544475 |
| 10 | Pyridoxine Hydrochloride | 96.1931291 |
| 11 | FUROSEMIDE/LASIX | 93.8356164 |
| 12 | D-Glycerate 2,3-diphosphate sodium salt | 92.245325 |
| 13 | Riboflavin | 88.0222841 |
| 14 | D(+)-GALACTOSE ANHYDROUS | 87.926375 |
| 15 | CITRIC ACID | 82.446475 |
| 16 | L-Tryptophan | 76.3431151 |
| 17 | CELLULOSE FIBROUS | 72.1710725 |
| 18 | Trans-Vaccenic Acid | 69.862825 |
| 19 | p-Aminobenzoic Acid | 68.3070257 |
| 20 | D-Pantothenic Acid, hemicalcium salt | 67.4094708 |
| 21 | TIN(II) CHLORIDE/Tin | 66.3013699 |
| 22 | D-(+)-MALTOSE MONOHYDRATE | 63.1480275 |
| 23 | GUANIDINE HYDROCHLORIDE | 62.929575 |
| 24 | L-Methionine | 62.8893905 |
| 25 | ACETONE | 54.128425 |
| 26 | ANGIOTENSIN II HUMAN | 52.665725 |
| 27 | Alpha-linolenic acid (ALA) | 52.430375 |
| 28 | L-Threonine | 49.4808126 |
| 29 | ACETYLCHOLINE CHLORIDE | 46.018875 |
| 30 | Glycine | 43.9869281 |
| 31 | ADENOSINE 5′-TRIPHOSPHATE DISODIUM SALT | 42.629985 |
| 32 | Pyridoxal Hydrochloride | 42.3398329 |
| 33 | calcium d-glucarate | 41.6058394 |
| 34 | Pyridoxamine Dihydrochloride | 40.9470752 |
| 35 | THYROCALCITONIN SALMON/calcitonin | 39.4258125 |
| 36 | SODIUM CHLORIDE | 38.7733375 |
| 37 | BETA-CRYPTOXANTIIIN | 36.94945 |
| 38 | L-Aspartic acid potassium salt | 36.7029549 |
| 39 | L-A-PHOSPHATIDYLCHOLINE TYPE XVI-E | 36.39315 |
| 40 | INDOXYL beta-D-GLUCOSIDE/Inodxyl glucuronide | 34.0751 |
| 41 | Iron Gluconate | 33.437014 |
| 42 | Nervonic acid | 32.9066 |
| 43 | L-Glutamine | 32.6143791 |
| 44 | Evening primrose oil | 32.4109 |
| 45 | trans-4-Hydroxy-L-proline | 31.8954248 |
| 46 | L-Isoleucine | 31.5575621 |
| 47 | POTASSIUM BROMIDE | 30.326475 |
| 48 | Docosapentaenoic acid (DPA) | 29.35347 |
| 49 | D(−)FRUCTOSE | 28.93901 |
| 50 | Magnesium Aspartate | 27.8382582 |
| 51 | sodium selenite | 26.8647478 |
| 52 | CoQ-10 | 26.1655 |
| 53 | Monopotassium Phosphate | 26.1446164 |
| 54 | Malic acid | 25.6609642 |
| 55 | Inositol | 25.39915 |
| 56 | chromium Chloride | 25.3041363 |
| 57 | Calcium citrate | 24.6553122 |
| 58 | Chromium picolinate | 24.0064882 |
| 59 | GLYCEROL MOLECULAR BIOLOGY REAGENT | 23.6167425 |
| 60 | Glucosamine Hydrochloride | 23.4064125 |
| 61 | Bromelain from pineapple stem | 23.00595 |
| 62 | Stearidonic acid | 22.77165 |
| 63 | MERCURY(II) CHLORIDE | 22.68544 |
| 64 | CHITIN | 21.06117 |
| 65 | DL-6,8-Thioctic Acid | 20.3160271 |
| 66 | Pumpkin seed oil | 20.0438 |
| 67 | alpha-lipoic acid | 20.016425 |
| 68 | Erucic acid | 19.77587 |
| 69 | L-Serine | 18.7581699 |
| 70 | Dipotassium Phosphate | 17.7293935 |
| 71 | Tricalcium phosphate hydrate | 17.3630455 |
| 72 | L-a-Phosphatidylcholine | 17.214025 |
| 73 | CHOLINE CHLORIDE | 15.6995125 |
| 74 | Flax seed oil | 14.847725 |
| 75 | L-Cystine | 14.1327623 |
| 76 | Zcaxanthin | 14.04087 |
| 77 | PECTIN | 13.74691 |
| 78 | MELATONIN CRYSTALLINE/melatonin | 13.6300325 |
| 79 | Folic Acid | 13.5871247 |
| 80 | HISTAMINE FREE BASE CRYSTALLINE | 13.58368 |
| 81 | PYRUVIC ACID | 13.110525 |
| 82 | SODIUM SULFATE | 12.88661 |
| 83 | ZINC SULFATE HEPTAHYDRATE | 12.3672725 |
| 84 | NICOTINIC ACID | 12.242275 |
| 85 | L-Lysine hydrochloride | 11.2866817 |
| 86 | Myristoleic acid | 11.07895 |
| 87 | Astaxanthin | 10.193495 |
| 88 | SODIUM FLUORIDE | 9.5875875 |
| 89 | SODIUM BICARBONATE | 9.45016 |
| 90 | Zinc Citrate | 9.37790158 |
| 91 | OXALIC ACID SODIUM SALT | 7.9325475 |
| 92 | D-(+)-GLUCOSAMINE HYDROCHLORIDE | 7.3703925 |
| 93 | Copper Gluconate | 7.21816707 |
| 94 | ALBUMIN HUMAN | 6.7961275 |
| 95 | 3,3′,5-TRIIODO-L-THYRONINE SODIUM SALT/Liothyronin | 5.58025 |
| 96 | d-Biotin | 5.04487775 |
| 97 | D-GLUCURONIC ACID | 3.553765 |
| 98 | Ferrous Fumarate | 2.95489891 |
| 99 | Caffeic acid phenethyl ester | 2.439025 |
| 100 | Lutein | 2.386235 |
| 101 | Magnesium citrate | 2.33281493 |
| 102 | Gelatin | 1.98878123 |
| 103 | L-(+)-ERGOTHIONEINE INNER SALT | 1.8080625 |
| 104 | D-glucosamine 3-sulfate | 1.38734739 |
| 105 | Thiamine Hydrochloride | 0.72234763 |
| 106 | A-CRYPTOXANTHIN | 0.706805 |
| 107 | Hyaluronic acid sodium salt | 0.61193269 |
| 108 | TAURINE | 0.6013675 |
| 109 | L-Proline | 0.32679739 |
| 110 | Carcinoembryonic Antigen (CEA) | −0.3876075 |
| 111 | Niacinamide | −0.5261529 |
| 112 | Curcumin | −1.8868075 |
| 113 | L-Leucine | −2.6636569 |
| 114 | VITAMIN K1 | −3.1362375 |
| 115 | TRANS-B-CAROTENE, TYPE I,/all trans B carotene | −3.187675 |
| 116 | Capsaicin | −3.4406215 |
| 117 | L-glutathione | −4.0510575 |
| 118 | Potassium phosphate dibasic | −4.4995264 |
| 119 | Calcium Carbonate | −5.3527981 |
| 120 | ARG8-VASOPRESSIN ACETATE SALT/Vasopressin | −5.4185025 |
| 121 | ALPHA 1 ANTITRYPSIN, HUMAN PLASMA | −5.4185175 |
| 122 | PROINSULIN C-PEPTIDE (55-89)/c-peptide | −5.533555 |
| 123 | ALCOHOL | −5.9393525 |
| 124 | CAFFEINE | −6.2160531 |
| 125 | Sphingomyelin | −6.3420275 |
| 126 | D-LACTOSE MONOHYDRATE | −6.5769806 |
| 127 | LEPTIN | −6.6005475 |
| 128 | A-CAROTENE | −7.3985325 |
| 129 | PYRIMIDINE | −7.9799475 |
| 130 | LITHIUM ACETOACETATE/Acetoacetic acid | −8.413135 |
| 131 | ANDROSTENEDIONE | −8.442685 |
| 132 | LUTEINIZING HORMONE RELEASING HORMONE HUMAN | −8.4632925 |
| 133 | L-Phenylalanine | −8.7437545 |
| 134 | THIAMINE PYROPHOSPHATE | −9.50731 |
| 135 | ESTRIOL | −10.313901 |
| 136 | L-ALANINE | −10.442568 |
| 137 | L-Asparagine | −12.810458 |
| 138 | PHYTANIC ACID | −12.916098 |
| 139 | SUCROSE | −13.144325 |
| 140 | BoswelliaSerrata Extract | −13.71128 |
| 141 | TETRAETHYLAMMONIUM HYDROGEN SULFATE, FOR IPC | −14.050822 |
| 142 | Zinc Picolinate | −14.856082 |
| 143 | O-METHYLGUANINE | −16.535908 |
| 144 | UREA | −16.838108 |
| 145 | Zinc Oxide | −16.867843 |
| 146 | L-Glutamic Acid | −17.581699 |
| 147 | FLAVIN ADENINE DINUCLEOTIDE DISODIUM SALT HYDR | −17.863335 |
| 148 | Potassium Citrate | −17.950742 |
| 149 | ADENOSINE 3′:5′-CYCLIC MONOPHOSPHATE | −17.999503 |
| 150 | cis-Vaccenic acid | −18.40491 |
| 151 | copper sulfate | −19.545823 |
| 152 | Lycopene | −19.700335 |
| 153 | 50% deoxycholic acid sodium salt + 50% cholic acid sodium | −19.888333 |
| 154 | URIC ACID | −20.628048 |
| 155 | L-Valine | −20.770878 |
| 156 | HYALURONIC ACID SODIUM SALT | −21.593205 |
| 157 | strontium chloride | −23.150727 |
| 158 | CADMIUM CHLORIDE/cadmium | −23.318386 |
| 159 | L-CYSTEINE | −23.886633 |
| 160 | CHOLESTEROL | −24.097855 |
| 161 | BIOTIN, POWDER, | −24.341195 |
| 162 | SODIUM BROMIDE | −24.920693 |
| 163 | XANTHOPHYLL FROM MARIGOLD/xanthophyll (Lutein) | −24.981428 |
| 164 | Collagen type II | −25.471698 |
| 165 | phenolics | −25.522693 |
| 166 | Sodium Molybdate | −25.528892 |
| 167 | URACIL | −26.983125 |
| 168 | N-acetyl D-glucosamine | −27.205507 |
| 169 | Docosahexaenoic Acid (DHA) | −27.371385 |
| 170 | L-Tyrosine | −27.694504 |
| 171 | Nicotinic Acid | −27.82182 |
| 172 | 5ALPHA-DIHYDROTESTOSTERONE (DHT)/DHT | −28.076258 |
| 173 | Potassium sorbate | −28.702242 |
| 174 | 17A-HYDROXYPROGESTERONE | −28.907664 |
| 175 | L(+)LACTIC ACID SODIUM | −29.30591 |
| 176 | Coconut oil | −29.68382 |
| 177 | Manganese sulfate | −29.860031 |
| 178 | (+)-DELTA-TOCOPHEROL | −31.643836 |
| 179 | ANDROSTERONE VETRANAL,/androsterone | −32.054795 |
| 180 | Dicalcium Phosphate | −32.927818 |
| 181 | PYRIDOXINE | −33.17653 |
| 182 | Folic Acid | −33.308168 |
| 183 | Eicosatrienoic Acid | −33.55307 |
| 184 | Rutoside trihydrate | −33.860275 |
| 185 | CORTISONE | −34.650788 |
| 186 | ESTRONE | −34.828102 |
| 187 | CYTOSINE | −34.931507 |
| 188 | L-Arginine | −34.939329 |
| 189 | Arachidonic acid | −35.913165 |
| 190 | ERGOCALCIFEROL | −35.92233 |
| 191 | L-GLUTATHIONE REDUCED | −36.349615 |
| 192 | NOREPINEPHRINE | −37.505568 |
| 193 | 3-HYDROXYBUTYRIC ACID | −38.354755 |
| 194 | L-THYROXINE | −38.356164 |
| 195 | MAGNESIUM CHLORIDE ANHYDROUS | −38.600875 |
| 196 | VASOACTIVE INTESTINAL PEPTIDE | −38.677018 |
| 197 | LEAD(II) SULFIDE | −39.312407 |
| 198 | SULFUR/sulfide | −39.760837 |
| 199 | Methylsulfonylmethane | −40.489546 |
| 200 | GLYCOGEN | −40.51758 |
| 201 | ADENOSINE FREE BASE | −41.491003 |
| 202 | Potassium Gluconate | −41.506157 |
| 203 | L-Alanine | −43.361884 |
| 204 | POTASSIUM CHLORIDE/potassium | −43.638333 |
| 205 | Taurine | −44.212137 |
| 206 | L-Aspartic Acid | −45.503212 |
| 207 | Resveratrol | −46.175421 |
| 208 | L-Histidine hydrochloride | −46.680942 |
| 209 | 5-HYDROXYTRYPTAMINE HYDROCHLORIDE/Serotonin | −47.013163 |
| 210 | PURINE | −47.533632 |
| 211 | L-ORNITHINE MONOHYDROCHLORIDE | −48.433615 |
| 212 | D-PANTOTHENIC ACID HEMICALCIUM | −48.46669 |
| 213 | 6-gingerol | −49.505603 |
| 214 | Adenosine 5′-Triphosphate Disodium | −49.528125 |
| 215 | SPHINGOMYELIN/SM(d18:1/16:0) | −49.663608 |
| 216 | L-A-PHOSPHATIDYLETHANOLAMINE TYPE I | −49.979783 |
| 217 | Silicic acid | −50.078939 |
| 218 | 2-DEOXY-L-RIBOSE/Deoxycytidine | −50.556865 |
| 219 | CHOLECYSTOKININ FRAGMENT 26-33 AMIDE NON SULF | −50.691865 |
| 220 | ZINC CHLORIDE | −51.16857 |
| 221 | (−)-EPINEPHRINE | −51.595008 |
| 222 | Ubiquinol | −52.052658 |
| 223 | L-ASCORBIC ACID BIOXTRA, | −52.461463 |
| 224 | THIAMINE HYDROCHLORIDE | −52.66037 |
| 225 | Chondroitin sulfate sodium | −52.663708 |
| 226 | CHORIONIC GONADOTROPIN HUMAN, | −53.740398 |
| 227 | DL-MALIC ACID | −54.898063 |
| 228 | Quercetin | −55.667115 |
| 229 | Trimethylglycine | −55.823293 |
| 230 | Myristic Acid | −55.849253 |
| 231 | (+/−)-A-TOCOPHEROL/Alpha-Tocopherol | −56.143958 |
| 232 | 11-DEOXYCORTISOL/Cortexolone | −56.715753 |
| 233 | C-PEPTIDE FRAGMENT 3-33, HUMAN/C-peptide | −56.901788 |
| 234 | Niacinamide | −56.92714 |
| 235 | Sodium citrate | −60.767288 |
| 236 | Crucuminoids | −61.653718 |
| 237 | SUCCINIC ACID | −65.833855 |
| 238 | FOLIC ACID, | −65.962583 |
| 239 | LIPASE FROM PORCINE PANCREAS | −66.02044 |
| 240 | Pterostibene | −66.845158 |
| 241 | CHONDROITIN SULFATE, A SODIUM SALT | −67.16166 |
| 242 | L-a-Phosphatidylcholine | −67.469877 |
| 243 | (+)-GAMMA-TOCOPHEROL | −68.16921 |
| 244 | OXYTOCIN | −68.747893 |
| 245 | MYO-INOSITOL | −68.771755 |
| 246 | BETAINE | −69.206745 |
| 247 | PHENOL | −71.08183 |
| 248 | CHOLECALCIFEROL CRYSTALLINE | −71.751605 |
| 249 | linoleic acid | −73.48477 |
| 250 | 4-AMINOBENZOIC ACID | −73.578365 |
| 251 | 3-HYDROXYTYRAMINE HCL/dopamine | −73.994453 |
| 252 | PREGNENOLONE ACETATE/pregnenolone | −76.946288 |
| 253 | MANGANESE SULFATE MONOHYDRATE, | −79.462953 |
| 254 | Thymoquinone | −82.307006 |
| 255 | Palmitoleic acid | −82.70231 |
| 256 | Stearic Acid | −85.813338 |
| 257 | Palmitic Acid | −86.33945 |
| 258 | Gamma linolenic acid | −94.207538 |

TABLE 5

| | Mouse T cell IL-2 | Mouse CD4⁺ T cell IL-2 | Mouse CD8⁺ T cell IL-2 |
|---|---|---|---|
| | level changes after | level changes after | level changes after |
| Candidate name | treatment | treatment | treatment |
| Trans-Vaccenic Acid | 183.5% | 155.6% | 193.6% |
| Thymidine | 145.6% | 135.8% | 128.9% |
| D-Glycerate2,3- | 106.2% | 97.9% | 121.4% |
| diphosphate sodium salt | | | |
| p-Aminobenzoic Acid | 160.3% | 140.5% | 145.2% |
| D(+)-galactose anhydrous | 130.1% | 110.4% | 130.3% |
| GUANINE | 148.6% | 110.4% | 150.8% |

TABLE 6

| | Relative Pixel Density (%) | −Log10(P-value) |
|---|---|---|
| p-HSP27(S78/S82) | 120.689435 | 0.316964977 |
| p-CREB(S133) | 111.6374 | 1.702018135 |
| p-EGFR(Y1086) | 109.55535 | 0.733795502 |
| p-STATI(Y701) | 109.29665 | 0.533230814 |
| p-GSK-3?/?(S21/S9) | 104.3625 | 0.670840896 |
| p-STAT6(Y641) | 104.173575 | 0.148230402 |
| HSP60 | 99.96418 | 0.007185034 |
| ?-Catenin | 98.64661 | 0.288927992 |
| p-STAT5a/b(Y694/Y699) | 97.09414 | 0.591725263 |
| p-Yes(Y426) | 92.31604 | 1.066073081 |
| p-PDGF R?(Y751) | 91.23745 | 1.345515631 |
| p-Lck(Y394) | 89.85713 | 0.814107011 |
| p-MSK1/2(S376/S360) | 89.695615 | 1.637991908 |
| p-STAT3(Y705) | 89.61953 | 1.328125213 |
| p-PLC-?1(Y783) | 89.1635 | 0.89090845 |
| p-STAT2(Y689) | 88.72418 | 0.715027718 |
| p-GSK-3?(S9) | 87.430175 | 1.297616719 |
| p-Fgr(Y412) | 87.28216 | 1.724642219 |
| p-Lyn(Y397) | 87.24125 | 1.079569568 |
| p-PYK2(Y402) | 86.97589 | 1.477158775 |
| p-Src(Y419) | 85.88792 | 0.841504158 |
| p-eNOS(S1177) | 84.26072 | 1.245335838 |
| p-Akt1/2/3(S473) | 83.05442 | 1.193659351 |
| p-Chk-2(T68) | 80.693955 | 1.043199032 |
| p-p70 S6 Kinase(T421/S424) | 80.33929 | 2.061011185 |
| p-p38?(T180/Y182) | 79.60737 | 0.723801794 |
| p-p53(S15) | 79.533465 | 2.040065894 |
| p-STAT3(S727) | 79.499035 | 1.433119205 |
| p-ERK1/2(T202/Y204 T185/Y187) | 79.269935 | 2.250374315 |
| p-JNK1/2/3(T183/Y185T221/Y223) | 78.521155 | 2.28989768 |
| p-p53(S46) | 78.5116 | 1.501913129 |
| p-WNK1(T60) | 78.342385 | 1.379441613 |
| p-Akt1/2/3(T308) | 77.687955 | 0.826150604 |
| p-p70 S6 Kinase(T389) | 77.295155 | 2.001311438 |
| p-p53(S392) | 75.85709 | 1.231509636 |
| p-PRAS40(T246) | 72.29019 | 2.473862729 |
| p-c-Jun(S63) | 70.59788 | 1.84147218 |
| p-RSK1/2(S221/S227) | 69.16812 | 2.45867683 |
| p-RSK1/2/3(S380/S386/S377) | 64.02901 | 1.241087258 |

TABLE 7
Table 7: DEGs only in [symbol] NTC + TVA group

| | | | | | | gene_{—} | gene_{—} | gene_{—} | gene_{—} | gene_{—} | gene_{—} | gene_{—} | gene_{—} | tf_{—} |
|---|---|---|---|---|---|---|---|---|---|---|---|---|---|---|
| gene_id | [symbol] | [symbol] | FC | log2FoldCha | pvalue | name | chr | start | end | strand | length | biotype | descrip | family |
| ENSMUSG0 | [symbol] | 0 | [symbol] | [symbol] | [symbol] | [symbol] | 17 | [symbol] | [symbol] | + | 8514 | protein_codin | [symbol] | — |
| ENSMUSG0 | [symbol] | 0 | [symbol] | [symbol] | [symbol] | [symbol] | 5 | [symbol] | [symbol] | + | 1807 | protein_codin | [symbol] | — |
| ENSMUSG0 | [symbol] | 0 | [symbol] | [symbol] | [symbol] | [symbol] | 1 | [symbol] | [symbol] | − | 777 | protein_codin | [symbol] | — |
| ENSMUSG0 | [symbol] | 0 | [symbol] | [symbol] | [symbol] | [symbol] | 9 | [symbol] | [symbol] | + | 1031 | protein_codin | [symbol] | — |
| ENSMUSG0 | [symbol] | 0 | [symbol] | [symbol] | [symbol] | [symbol] | 11 | [symbol] | [symbol] | + | 4287 | protein_codin | [symbol] | — |
| ENSMUSG0 | [symbol] | 0 | [symbol] | [symbol] | [symbol] | [symbol] | 1 | [symbol] | [symbol] | − | 2759 | protein_codin | [symbol] | — |
| ENSMUSG0 | [symbol] | 0 | [symbol] | [symbol] | [symbol] | [symbol] | 4 | [symbol] | [symbol] | − | 13978 | protein_codin | [symbol] | — |
| ENSMUSG0 | [symbol] | 0 | [symbol] | [symbol] | [symbol] | [symbol] | 7 | [symbol] | [symbol] | − | 6120 | protein_codin | [symbol] | — |
| ENSMUSG0 | [symbol] | 0 | [symbol] | [symbol] | [symbol] | [symbol] | 1 | [symbol] | [symbol] | + | 2012 | protein_codin | [symbol] | — |
| ENSMUSG0 | [symbol] | 0 | [symbol] | [symbol] | [symbol] | [symbol] | 15 | [symbol] | [symbol] | − | 2435 | protein_codin | [symbol] | — |
| ENSMUSG0 | [symbol] | [symbol] | [symbol] | [symbol] | [symbol] | [symbol] | 9 | [symbol] | [symbol] | − | 3026 | protein_codin | [symbol] | — |
| ENSMUSG0 | [symbol] | [symbol] | [symbol] | [symbol] | [symbol] | [symbol] | 6 | [symbol] | [symbol] | + | 3335 | protein_codin | [symbol] | — |
| ENSMUSG0 | [symbol] | [symbol] | [symbol] | [symbol] | [symbol] | [symbol] | 7 | [symbol] | [symbol] | + | 2758 | protein_codin | [symbol] | — |
| ENSMUSG0 | [symbol] | [symbol] | [symbol] | [symbol] | [symbol] | [symbol] | X | [symbol] | [symbol] | + | [symbol] | protein_codin | [symbol] | — |
| ENSMUSG0 | [symbol] | [symbol] | [symbol] | [symbol] | [symbol] | [symbol] | 2 | [symbol] | [symbol] | + | 4194 | protein_codin | [symbol] | — |
| ENSMUSG0 | [symbol] | [symbol] | [symbol] | [symbol] | [symbol] | [symbol] | 5 | [symbol] | [symbol] | + | 10815 | protein_codin | [symbol] | — |
| ENSMUSG0 | [symbol] | [symbol] | [symbol] | [symbol] | [symbol] | [symbol] | 13 | [symbol] | [symbol] | − | 485 | protein_codin | [symbol] | — |
| ENSMUSG0 | [symbol] | [symbol] | [symbol] | [symbol] | [symbol] | [symbol] | 1 | [symbol] | [symbol] | + | 5313 | protein_codin | [symbol] | — |
| ENSMUSG0 | [symbol] | [symbol] | [symbol] | [symbol] | [symbol] | [symbol] | 15 | [symbol] | [symbol] | − | 2728 | protein_codin | [symbol] | — |
| ENSMUSG0 | [symbol] | [symbol] | [symbol] | [symbol] | [symbol] | [symbol] | X | [symbol] | [symbol] | − | 4165 | protein_codin | [symbol] | — |
| ENSMUSG0 | [symbol] | [symbol] | [symbol] | [symbol] | [symbol] | [symbol] | 9 | [symbol] | [symbol] | − | 1592 | protein_codin | [symbol] | — |
| ENSMUSG0 | [symbol] | [symbol] | [symbol] | [symbol] | [symbol] | [symbol] | 17 | [symbol] | [symbol] | − | 3872 | protein_codin | [symbol] | — |
| ENSMUSG0 | [symbol] | [symbol] | [symbol] | [symbol] | [symbol] | [symbol] | 2 | [symbol] | [symbol] | + | 1873 | protein_codin | [symbol] | — |
| ENSMUSG0 | [symbol] | [symbol] | [symbol] | [symbol] | [symbol] | [symbol] | 17 | [symbol] | [symbol] | + | 1154 | protein_codin | [symbol] | — |
| ENSMUSG0 | [symbol] | [symbol] | [symbol] | [symbol] | [symbol] | [symbol] | 5 | [symbol] | [symbol] | − | 5593 | protein_codin | [symbol] | — |
| ENSMUSG0 | [symbol] | [symbol] | [symbol] | [symbol] | [symbol] | [symbol] | 1 | [symbol] | [symbol] | − | 8467 | protein_codin | [symbol] | — |
| ENSMUSG0 | [symbol] | [symbol] | [symbol] | [symbol] | [symbol] | [symbol] | 12 | [symbol] | [symbol] | + | 1832 | protein_codin | [symbol] | — |
| ENSMUSG0 | [symbol] | [symbol] | [symbol] | [symbol] | [symbol] | [symbol] | 10 | [symbol] | [symbol] | − | [symbol] | protein_codin | [symbol] | — |
| ENSMUSG0 | [symbol] | [symbol] | [symbol] | [symbol] | [symbol] | [symbol] | 7 | [symbol] | [symbol] | + | 3747 | protein_codin | [symbol] | — |
| ENSMUSG0 | [symbol] | [symbol] | [symbol] | [symbol] | [symbol] | [symbol] | 14 | [symbol] | [symbol] | + | 5506 | protein_codin | [symbol] | — |
| ENSMUSG0 | [symbol] | [symbol] | [symbol] | [symbol] | [symbol] | [symbol] | 11 | [symbol] | [symbol] | − | 2089 | protein_codin | [symbol] | — |
| ENSMUSG0 | [symbol] | [symbol] | [symbol] | [symbol] | [symbol] | [symbol] | 13 | [symbol] | [symbol] | − | 2564 | protein_codin | [symbol] | — |
| ENSMUSG0 | [symbol] | [symbol] | [symbol] | [symbol] | [symbol] | [symbol] | 15 | [symbol] | [symbol] | − | 5170 | protein_codin | [symbol] | — |
| ENSMUSG0 | [symbol] | [symbol] | [symbol] | [symbol] | [symbol] | [symbol] | 11 | [symbol] | [symbol] | + | 2146 | protein_codin | [symbol] | — |
| ENSMUSG0 | [symbol] | [symbol] | [symbol] | [symbol] | [symbol] | [symbol] | 19 | [symbol] | [symbol] | − | 864 | protein_codin | [symbol] | — |
| ENSMUSG0 | [symbol] | [symbol] | [symbol] | [symbol] | [symbol] | [symbol] | 5 | [symbol] | [symbol] | + | 1037 | protein_codin | [symbol] | — |
| ENSMUSG0 | [symbol] | [symbol] | [symbol] | [symbol] | [symbol] | [symbol] | 1 | [symbol] | [symbol] | + | 16449 | protein_codin | [symbol] | — |
| ENSMUSG0 | [symbol] | [symbol] | [symbol] | [symbol] | [symbol] | [symbol] | 1 | [symbol] | [symbol] | − | 742 | protein_codin | [symbol] | — |
| ENSMUSG0 | [symbol] | [symbol] | [symbol] | [symbol] | [symbol] | [symbol] | 13 | [symbol] | [symbol] | + | 4936 | protein_codin | [symbol] | — |
| ENSMUSG0 | [symbol] | [symbol] | [symbol] | [symbol] | [symbol] | [symbol] | 18 | [symbol] | [symbol] | + | 2837 | protein_codin | [symbol] | — |
| ENSMUSG0 | [symbol] | [symbol] | [symbol] | [symbol] | [symbol] | [symbol] | 14 | [symbol] | [symbol] | + | 2413 | protein_codin | [symbol] | — |
| ENSMUSG0 | [symbol] | [symbol] | [symbol] | [symbol] | [symbol] | [symbol] | 17 | [symbol] | [symbol] | + | [symbol] | protein_codin | [symbol] | — |
| ENSMUSG0 | [symbol] | [symbol] | [symbol] | [symbol] | [symbol] | [symbol] | 17 | [symbol] | [symbol] | − | 1418 | protein_codin | [symbol] | — |
| ENSMUSG0 | [symbol] | [symbol] | [symbol] | [symbol] | [symbol] | [symbol] | X | [symbol] | [symbol] | + | 1255 | protein_codin | [symbol] | — |
| ENSMUSG0 | [symbol] | [symbol] | [symbol] | [symbol] | [symbol] | [symbol] | 7 | [symbol] | [symbol] | − | 2204 | protein_codin | [symbol] | — |
| ENSMUSG0 | [symbol] | [symbol] | [symbol] | [symbol] | [symbol] | [symbol] | 1 | [symbol] | [symbol] | + | [symbol] 422 | protein_codin | [symbol] | — |
| ENSMUSG0 | [symbol] | [symbol] | [symbol] | [symbol] | [symbol] | [symbol] | 4 | [symbol] | [symbol] | + | 1275 | protein_codin | [symbol] | — |
| ENSMUSG0 | [symbol] | [symbol] | [symbol] | [symbol] | [symbol] | [symbol] | 16 | [symbol] | [symbol] | + | 9141 | protein_codin | [symbol] | — |
| ENSMUSG0 | [symbol] | [symbol] | [symbol] | [symbol] | [symbol] | [symbol] | 7 | [symbol] | [symbol] | + | 5579 | protein_codin | [symbol] | — |
| ENSMUSG0 | [symbol] | [symbol] | [symbol] | [symbol] | [symbol] | [symbol] | 13 | [symbol] | [symbol] | − | 5773 | protein_codin | [symbol] | — |
| ENSMUSG0 | [symbol] | [symbol] | [symbol] | [symbol] | [symbol] | [symbol] | 7 | [symbol] | [symbol] | − | 4199 | protein_codin | [symbol] | zf- |
| | | | | | | | | | | | | | | C2H2 |
| ENSMUSG0 | [symbol] | [symbol] | [symbol] | [symbol] | [symbol] | [symbol] | 7 | [symbol] | [symbol] | + | 1164 | protein_codin | [symbol] | — |
| ENSMUSG0 | [symbol] | [symbol] | [symbol] | [symbol] | [symbol] | [symbol] | 10 | [symbol] | [symbol] | + | 3465 | protein_codin | [symbol] | — |
| ENSMUSG0 | [symbol] | [symbol] | [symbol] | [symbol] | [symbol] | [symbol] | 8 | [symbol] | [symbol] | − | 4141 | protein_codin | [symbol] | — |
| ENSMUSG0 | [symbol] | [symbol] | [symbol] | [symbol] | [symbol] | [symbol] | 2 | [symbol] | [symbol] | − | 177 [symbol] | protein_codin | [symbol] | — |
| ENSMUSG0 | [symbol] | [symbol] | [symbol] | [symbol] | [symbol] | [symbol] | 8 | [symbol] | [symbol] | + | 5528 | protein_codin | [symbol] | — |
| ENSMUSG0 | [symbol] | [symbol] | [symbol] | [symbol] | [symbol] | [symbol] | X | [symbol] | [symbol] | − | 2106 | protein_codin | [symbol] | — |
| ENSMUSG0 | [symbol] | [symbol] | [symbol] | [symbol] | [symbol] | [symbol] | 5 | [symbol] | [symbol] | − | 5850 | protein_codin | [symbol] | — |
| ENSMUSG0 | [symbol] | [symbol] | [symbol] | [symbol] | [symbol] | [symbol] | 19 | [symbol] | [symbol] | + | 2778 | protein_codin | [symbol] | — |
| ENSMUSG0 | [symbol] | [symbol] | [symbol] | [symbol] | [symbol] | [symbol] | 9 | [symbol] | [symbol] | − | 2000 | protein_codin | [symbol] | — |
| ENSMUSG0 | [symbol] | [symbol] | [symbol] | [symbol] | [symbol] | [symbol] | 19 | [symbol] | [symbol] | − | 6384 | protein_codin | [symbol] | — |
| ENSMUSG0 | [symbol] | [symbol] | [symbol] | [symbol] | [symbol] | [symbol] | 5 | [symbol] | [symbol] | + | 6433 | protein_codin | [symbol] | — |
| ENSMUSG0 | [symbol] | [symbol] | [symbol] | [symbol] | [symbol] | [symbol] | 4 | [symbol] | [symbol] | + | 4924 | protein_codin | [symbol] | — |
| ENSMUSG0 | [symbol] | [symbol] | [symbol] | [symbol] | [symbol] | [symbol] | 2 | [symbol] | [symbol] | − | 6679 | protein_codin | [symbol] | — |
| ENSMUSG0 | [symbol] | [symbol] | [symbol] | [symbol] | [symbol] | [symbol] | 5 | [symbol] | [symbol] | − | 5216 | protein_codin | [symbol] | — |
| ENSMUSG0 | [symbol] | [symbol] | [symbol] | [symbol] | [symbol] | [symbol] | 2 | [symbol] | [symbol] | + | 6558 | protein_codin | [symbol] | — |
| ENSMUSG0 | [symbol] | [symbol] | [symbol] | [symbol] | [symbol] | [symbol] | 16 | [symbol] | [symbol] | + | 3213 | protein_codin | [symbol] | — |
| ENSMUSG0 | [symbol] | [symbol] | [symbol] | [symbol] | [symbol] | [symbol] | 7 | [symbol] | [symbol] | − | 3455 | protein_codin | [symbol] | — |
| ENSMUSG0 | [symbol] | [symbol] | [symbol] | [symbol] | [symbol] | [symbol] | 9 | [symbol] | [symbol] | − | 3641 | protein_codin | [symbol] | — |
| ENSMUSG0 | [symbol] | [symbol] | [symbol] | [symbol] | [symbol] | [symbol] | 17 | [symbol] | [symbol] | − | 386 | protein_codin | [symbol] | — |
| ENSMUSG0 | [symbol] | [symbol] | [symbol] | [symbol] | [symbol] | [symbol] | 10 | [symbol] | [symbol] | − | 3594 | protein_codin | [symbol] | — |
| ENSMUSG0 | [symbol] | [symbol] | [symbol] | [symbol] | [symbol] | [symbol] | 1 | [symbol] | [symbol] | − | 4195 | protein_codin | [symbol] | — |
| ENSMUSG0 | [symbol] | [symbol] | [symbol] | [symbol] | [symbol] | [symbol] | 10 | [symbol] | [symbol] | − | 6016 | protein_codin | [symbol] | — |
| ENSMUSG0 | [symbol] | [symbol] | [symbol] | [symbol] | [symbol] | [symbol] | 15 | [symbol] | [symbol] | − | 1617 | protein_codin | [symbol] | — |
| ENSMUSG0 | [symbol] | [symbol] | [symbol] | [symbol] | [symbol] | [symbol] | 2 | [symbol] | [symbol] | + | 1000 | protein_codin | [symbol] | — |
| ENSMUSG0 | [symbol] | [symbol] | [symbol] | [symbol] | [symbol] | [symbol] | 2 | [symbol] | [symbol] | + | 9128 | protein_codin | [symbol] | — |
| ENSMUSG0 | [symbol] | [symbol] | [symbol] | [symbol] | [symbol] | [symbol] | 9 | [symbol] | [symbol] | + | 5392 | protein_codin | [symbol] | — |
| ENSMUSG0 | [symbol] | [symbol] | [symbol] | [symbol] | [symbol] | [symbol] | 2 | [symbol] | [symbol] | − | 5039 | protein_codin | [symbol] | — |
| ENSMUSG0 | [symbol] | [symbol] | [symbol] | [symbol] | [symbol] | [symbol] | 11 | [symbol] | [symbol] | − | 3460 | protein_codin | [symbol] | — |
| ENSMUSG0 | [symbol] | [symbol] | [symbol] | [symbol] | [symbol] | [symbol] | 15 | [symbol] | [symbol] | + | 2742 | protein_codin | [symbol] | — |
| ENSMUSG0 | [symbol] | [symbol] | [symbol] | [symbol] | [symbol] | [symbol] | 7 | [symbol] | [symbol] | − | 7170 | protein_codin | [symbol] | — |
| ENSMUSG0 | [symbol] | [symbol] | [symbol] | [symbol] | [symbol] | [symbol] | 2 | [symbol] | [symbol] | + | 9832 | protein_codin | [symbol] | — |
| ENSMUSG0 | [symbol] | [symbol] | [symbol] | [symbol] | [symbol] | [symbol] | 2 | [symbol] | [symbol] | − | 2531 | protein_codin | [symbol] | — |
| ENSMUSG0 | [symbol] | [symbol] | [symbol] | [symbol] | [symbol] | [symbol] | 2 | [symbol] | [symbol] | − | 2400 | protein_codin | [symbol] | — |
| ENSMUSG0 | [symbol] | [symbol] | [symbol] | [symbol] | [symbol] | [symbol] | 11 | [symbol] | [symbol] | − | 2762 | protein_codin | [symbol] | — |
| ENSMUSG0 | [symbol] | [symbol] | [symbol] | [symbol] | [symbol] | [symbol] | 6 | [symbol] | [symbol] | + | 3020 | protein_codin | [symbol] | — |
| ENSMUSG0 | [symbol] | [symbol] | [symbol] | [symbol] | [symbol] | [symbol] | 2 | [symbol] | [symbol] | − | 546 [symbol] | protein_codin | [symbol] | — |
| ENSMUSG0 | [symbol] | [symbol] | [symbol] | [symbol] | [symbol] | [symbol] | 3 | [symbol] | [symbol] | − | 12692 | protein_codin | [symbol] | — |
| ENSMUSG0 | [symbol] | [symbol] | [symbol] | [symbol] | [symbol] | [symbol] | 11 | [symbol] | [symbol] | − | 5965 | protein_codin | [symbol] | — |
| ENSMUSG0 | [symbol] | [symbol] | [symbol] | [symbol] | [symbol] | [symbol] | [symbol] | [symbol] | [symbol] | − | 4097 | protein_codin | [symbol] | — |
| ENSMUSG0 | [symbol] | [symbol] | [symbol] | [symbol] | [symbol] | [symbol] | 3 | [symbol] | [symbol] | + | 9831 | protein_codin | [symbol] | — |
| ENSMUSG0 | [symbol] | [symbol] | [symbol] | [symbol] | [symbol] | [symbol] | 1 | [symbol] | [symbol] | − | 9079 | protein_codin | [symbol] | — |
| ENSMUSG0 | [symbol] | [symbol] | [symbol] | [symbol] | [symbol] | [symbol] | 7 | [symbol] | [symbol] | − | 676 | protein_codin | [symbol] | — |
| ENSMUSG0 | [symbol] | [symbol] | [symbol] | [symbol] | [symbol] | [symbol] | 19 | [symbol] | [symbol] | − | 2053 | protein_codin | [symbol] | — |
| ENSMUSG0 | [symbol] | [symbol] | [symbol] | [symbol] | [symbol] | [symbol] | 5 | [symbol] | [symbol] | − | 3611 | protein_codin | [symbol] | — |
| ENSMUSG0 | [symbol] | [symbol] | [symbol] | [symbol] | [symbol] | [symbol] | 9 | [symbol] | [symbol] | + | 3512 | protein_codin | [symbol] | — |
| ENSMUSG0 | [symbol] | [symbol] | [symbol] | [symbol] | [symbol] | [symbol] | 6 | [symbol] | [symbol] | − | 1720 | protein_codin | [symbol] | — |
| ENSMUSG0 | [symbol] | [symbol] | [symbol] | [symbol] | [symbol] | [symbol] | 3 | [symbol] | [symbol] | − | 1126 | protein_codin | [symbol] | — |
| ENSMUSG0 | [symbol] | [symbol] | [symbol] | [symbol] | [symbol] | [symbol] | 11 | [symbol] | [symbol] | + | 4257 | protein_codin | [symbol] | — |
| ENSMUSG0 | [symbol] | [symbol] | [symbol] | [symbol] | [symbol] | [symbol] | 7 | [symbol] | [symbol] | − | 7342 | protein_codin | [symbol] | — |
| ENSMUSG0 | [symbol] | [symbol] | [symbol] | [symbol] | [symbol] | [symbol] | 8 | [symbol] | [symbol] | − | 2043 | protein_codin | [symbol] | — |
| ENSMUSG0 | [symbol] | [symbol] | [symbol] | [symbol] | [symbol] | [symbol] | 2 | [symbol] | [symbol] | + | 1893 | protein_codin | [symbol] | — |
| ENSMUSG0 | [symbol] | [symbol] | [symbol] | [symbol] | [symbol] | [symbol] | 13 | [symbol] | [symbol] | + | 2231 | protein_codin | [symbol] | — |
| ENSMUSG0 | [symbol] | [symbol] | [symbol] | [symbol] | [symbol] | [symbol] | 11 | [symbol] | [symbol] | + | 6797 | protein_codin | [symbol] | — |
| ENSMUSG0 | [symbol] | [symbol] | [symbol] | [symbol] | [symbol] | [symbol] | 3 | [symbol] | [symbol] | + | 4200 | protein_codin | [symbol] | — |
| ENSMUSG0 | [symbol] | [symbol] | [symbol] | [symbol] | [symbol] | [symbol] | 5 | [symbol] | [symbol] | − | 947 | protein_codin | [symbol] | — |
| ENSMUSG0 | [symbol] | [symbol] | [symbol] | [symbol] | [symbol] | [symbol] | 18 | [symbol] | [symbol] | + | 837 | protein_codin | [symbol] | — |
| ENSMUSG0 | [symbol] | [symbol] | [symbol] | [symbol] | [symbol] | [symbol] | X | [symbol] | [symbol] | + | 7996 | protein_codin | [symbol] | — |
| ENSMUSG0 | [symbol] | [symbol] | [symbol] | [symbol] | [symbol] | [symbol] | 2 | [symbol] | [symbol] | + | 3991 | protein_codin | [symbol] | — |
| ENSMUSG0 | [symbol] | [symbol] | [symbol] | [symbol] | [symbol] | [symbol] | 13 | [symbol] | [symbol] | − | 6180 | protein_codin | [symbol] | — |
| ENSMUSG0 | [symbol] | [symbol] | [symbol] | [symbol] | [symbol] | [symbol] | 13 | [symbol] | [symbol] | − | 11223 | protein_codin | [symbol] | — |
| ENSMUSG0 | [symbol] | [symbol] | [symbol] | [symbol] | [symbol] | [symbol] | 17 | [symbol] | [symbol] | − | 2283 | protein_codin | [symbol] | — |
| ENSMUSG0 | [symbol] | [symbol] | [symbol] | [symbol] | [symbol] | [symbol] | 4 | [symbol] | [symbol] | − | 4683 | protein_codin | [symbol] | — |
| ENSMUSG0 | [symbol] | [symbol] | [symbol] | [symbol] | [symbol] | [symbol] | 6 | [symbol] | [symbol] | − | 5796 | protein_codin | [symbol] | — |
| ENSMUSG0 | [symbol] | [symbol] | [symbol] | [symbol] | [symbol] | [symbol] | 19 | [symbol] | [symbol] | − | 1402 | protein_codin | [symbol] | — |
| ENSMUSG0 | [symbol] | [symbol] | [symbol] | [symbol] | [symbol] | [symbol] | 8 | [symbol] | [symbol] | + | 3140 | protein_codin | [symbol] | — |
| ENSMUSG0 | [symbol] | [symbol] | [symbol] | [symbol] | [symbol] | [symbol] | 17 | [symbol] | [symbol] | + | 4209 | protein_codin | [symbol] | zf- |
| | | | | | | | | | | | | | | C2H2 |
| ENSMUSG0 | [symbol] | [symbol] | [symbol] | [symbol] | [symbol] | [symbol] | 12 | [symbol] | [symbol] | − | 14093 | protein_codin | [symbol] | — |
| ENSMUSG0 | [symbol] | [symbol] | [symbol] | [symbol] | [symbol] | [symbol] | 17 | [symbol] | [symbol] | + | [symbol] | protein_codin | [symbol] | — |
| ENSMUSG0 | [symbol] | [symbol] | [symbol] | [symbol] | [symbol] | [symbol] | 9 | [symbol] | [symbol] | − | 6148 | protein_codin | [symbol] | — |
| ENSMUSG0 | [symbol] | [symbol] | [symbol] | [symbol] | [symbol] | [symbol] | 4 | [symbol] | [symbol] | − | 1441 | protein_codin | [symbol] | — |
| ENSMUSG0 | [symbol] | [symbol] | [symbol] | [symbol] | [symbol] | [symbol] | 7 | [symbol] | [symbol] | − | 4601 | protein_codin | [symbol] | — |
| ENSMUSG0 | [symbol] | [symbol] | [symbol] | [symbol] | [symbol] | [symbol] | 19 | [symbol] | [symbol] | − | 2548 | protein_codin | [symbol] | — |
| ENSMUSG0 | [symbol] | [symbol] | [symbol] | [symbol] | [symbol] | [symbol] | X | [symbol] | [symbol] | − | 1934 | protein_codin | [symbol] | — |
| ENSMUSG0 | [symbol] | [symbol] | [symbol] | [symbol] | [symbol] | [symbol] | 1 | [symbol] | [symbol] | − | 2878 | protein_codin | [symbol] | — |
| ENSMUSG0 | [symbol] | [symbol] | [symbol] | [symbol] | [symbol] | [symbol] | 2 | [symbol] | [symbol] | − | 1463 | protein_codin | [symbol] | — |
| ENSMUSG0 | [symbol] | [symbol] | [symbol] | [symbol] | [symbol] | [symbol] | 9 | [symbol] | [symbol] | + | 6540 | protein_codin | [symbol] | — |
| ENSMUSG0 | [symbol] | [symbol] | [symbol] | [symbol] | [symbol] | [symbol] | 14 | [symbol] | [symbol] | + | [symbol] 637 | protein_codin | [symbol] | — |
| ENSMUSG0 | [symbol] | [symbol] | [symbol] | [symbol] | [symbol] | [symbol] | 14 | [symbol] | [symbol] | − | 1623 | protein_codin | [symbol] | — |
| ENSMUSG0 | [symbol] | [symbol] | [symbol] | [symbol] | [symbol] | [symbol] | 7 | [symbol] | [symbol] | − | 1716 | protein_codin | [symbol] | — |
| ENSMUSG0 | [symbol] | [symbol] | [symbol] | [symbol] | [symbol] | [symbol] | 13 | [symbol] | [symbol] | − | 3968 | protein_codin | [symbol] | zf- |
| | | | | | | | | | | | | | | C2H2 |
| ENSMUSG0 | [symbol] | [symbol] | [symbol] | [symbol] | [symbol] | [symbol] | 19 | [symbol] | [symbol] | − | [symbol] | protein_codin | [symbol] | — |
| ENSMUSG0 | [symbol] | [symbol] | [symbol] | [symbol] | [symbol] | [symbol] | 6 | [symbol] | [symbol] | − | 5280 | protein_codin | [symbol] | MBD |
| ENSMUSG0 | [symbol] | [symbol] | [symbol] | [symbol] | [symbol] | [symbol] | 13 | [symbol] | [symbol] | + | 3350 | protein_codin | [symbol] | — |
| ENSMUSG0 | [symbol] | [symbol] | [symbol] | [symbol] | [symbol] | [symbol] | 16 | [symbol] | [symbol] | − | 5223 | protein_codin | [symbol] | — |
| ENSMUSG0 | [symbol] | [symbol] | [symbol] | [symbol] | [symbol] | [symbol] | 11 | [symbol] | [symbol] | − | 4461 | protein_codin | [symbol] | — |
| ENSMUSG0 | [symbol] | [symbol] | [symbol] | [symbol] | [symbol] | [symbol] | 7 | [symbol] | [symbol] | − | 5141 | protein_codin | [symbol] | — |
| ENSMUSG0 | [symbol] | [symbol] | [symbol] | [symbol] | [symbol] | [symbol] | 2 | [symbol] | [symbol] | − | 9154 | protein_codin | [symbol] | — |
| ENSMUSG0 | [symbol] | [symbol] | [symbol] | [symbol] | [symbol] | [symbol] | 14 | [symbol] | [symbol] | − | 2378 | protein_codin | [symbol] | — |
| ENSMUSG0 | [symbol] | [symbol] | [symbol] | [symbol] | [symbol] | [symbol] | 1 | [symbol] | [symbol] | − | 8287 | protein_codin | [symbol] | — |
| ENSMUSG0 | [symbol] | [symbol] | [symbol] | [symbol] | [symbol] | [symbol] | 15 | [symbol] | [symbol] | − | 4908 | protein_codin | [symbol] | — |
| ENSMUSG0 | [symbol] | [symbol] | [symbol] | [symbol] | [symbol] | [symbol] | 17 | [symbol] | [symbol] | − | 3530 | protein_codin | [symbol] | — |
| ENSMUSG0 | [symbol] | [symbol] | [symbol] | [symbol] | [symbol] | [symbol] | 2 | [symbol] | [symbol] | + | 2820 | protein_codin | [symbol] | — |
| ENSMUSG0 | [symbol] | [symbol] | [symbol] | [symbol] | [symbol] | [symbol] | 13 | [symbol] | [symbol] | − | 5600 | protein_codin | [symbol] | — |
| ENSMUSG0 | [symbol] | [symbol] | [symbol] | [symbol] | [symbol] | [symbol] | 13 | [symbol] | [symbol] | + | 692 | protein_codin | [symbol] | — |
| ENSMUSG0 | [symbol] | [symbol] | [symbol] | [symbol] | [symbol] | [symbol] | 17 | [symbol] | [symbol] | − | 2163 | protein_codin | [symbol] | — |
| ENSMUSG0 | [symbol] | [symbol] | [symbol] | [symbol] | [symbol] | [symbol] | 6 | [symbol] | [symbol] | − | 1136 | protein_codin | [symbol] | — |
| ENSMUSG0 | [symbol] | [symbol] | [symbol] | [symbol] | [symbol] | [symbol] | 14 | [symbol] | [symbol] | + | 6243 | protein_codin | [symbol] | — |
| ENSMUSG0 | [symbol] | [symbol] | [symbol] | [symbol] | [symbol] | [symbol] | 17 | [symbol] | [symbol] | − | 8466 | protein_codin | [symbol] | — |
| ENSMUSG0 | [symbol] | [symbol] | [symbol] | [symbol] | [symbol] | [symbol] | 18 | [symbol] | [symbol] | + | 3 [symbol] 67 | protein_codin | [symbol] | — |
| ENSMUSG0 | [symbol] | [symbol] | [symbol] | [symbol] | [symbol] | [symbol] | X | [symbol] | [symbol] | + | 1289 | protein_codin | [symbol] | — |
| ENSMUSG0 | [symbol] | [symbol] | [symbol] | [symbol] | [symbol] | [symbol] | 9 | [symbol] | [symbol] | − | 4645 | protein_codin | [symbol] | — |
| ENSMUSG0 | [symbol] | [symbol] | [symbol] | [symbol] | [symbol] | [symbol] | 5 | [symbol] | [symbol] | + | [symbol] | protein_codin | [symbol] | — |
| ENSMUSG0 | [symbol] | [symbol] | [symbol] | [symbol] | [symbol] | [symbol] | 17 | [symbol] | [symbol] | + | 2076 | protein_codin | [symbol] | — |
| ENSMUSG0 | [symbol] | [symbol] | [symbol] | [symbol] | [symbol] | [symbol] | 11 | [symbol] | [symbol] | − | 2873 | protein_codin | [symbol] | — |
| ENSMUSG0 | [symbol] | [symbol] | [symbol] | [symbol] | [symbol] | [symbol] | 9 | [symbol] | [symbol] | + | 5363 | protein_codin | [symbol] | — |
| ENSMUSG0 | [symbol] | [symbol] | [symbol] | [symbol] | [symbol] | [symbol] | 7 | [symbol] | [symbol] | − | 5210 | protein_codin | [symbol] | — |
| ENSMUSG0 | [symbol] | [symbol] | [symbol] | [symbol] | [symbol] | [symbol] | 5 | [symbol] | [symbol] | − | 5820 | protein_codin | [symbol] | — |
| ENSMUSG0 | [symbol] | [symbol] | [symbol] | [symbol] | [symbol] | [symbol] | 1 | [symbol] | [symbol] | + | 2921 | protein_codin | [symbol] | — |
| ENSMUSG0 | [symbol] | [symbol] | [symbol] | [symbol] | [symbol] | [symbol] | X | [symbol] | [symbol] | − | 5437 | protein_codin | [symbol] | — |
| ENSMUSG0 | [symbol] | [symbol] | [symbol] | [symbol] | [symbol] | [symbol] | 5 | [symbol] | [symbol] | + | 6303 | protein_codin | [symbol] | — |
| ENSMUSG0 | [symbol] | [symbol] | [symbol] | [symbol] | [symbol] | [symbol] | X | [symbol] | [symbol] | + | 2785 | protein_codin | [symbol] | — |
| ENSMUSG0 | [symbol] | [symbol] | [symbol] | [symbol] | [symbol] | [symbol] | 3 | [symbol] | [symbol] | + | 7286 | protein_codin | [symbol] | — |
| ENSMUSG0 | [symbol] | [symbol] | [symbol] | [symbol] | [symbol] | [symbol] | 2 | [symbol] | [symbol] | − | 1131 | protein_codin | [symbol] | — |
| ENSMUSG0 | [symbol] | [symbol] | [symbol] | [symbol] | [symbol] | [symbol] | 1 | [symbol] | [symbol] | − | 5195 | protein_codin | [symbol] | — |
| ENSMUSG0 | [symbol] | [symbol] | [symbol] | [symbol] | [symbol] | [symbol] | 16 | [symbol] | [symbol] | + | 1503 | protein_codin | [symbol] | — |
| ENSMUSG0 | [symbol] | [symbol] | [symbol] | [symbol] | [symbol] | [symbol] | 4 | [symbol] | [symbol] | − | 7857 | protein_codin | [symbol] | SAND |
| ENSMUSG0 | [symbol] | [symbol] | [symbol] | [symbol] | [symbol] | [symbol] | 6 | [symbol] | [symbol] | − | 6576 | protein_codin | [symbol] | — |
| ENSMUSG0 | [symbol] | [symbol] | [symbol] | [symbol] | [symbol] | [symbol] | 9 | [symbol] | [symbol] | + | 3731 | protein_codin | [symbol] | — |
| ENSMUSG0 | [symbol] | [symbol] | [symbol] | [symbol] | [symbol] | [symbol] | 4 | [symbol] | [symbol] | + | 1898 | protein_codin | [symbol] | — |
| ENSMUSG0 | [symbol] | [symbol] | [symbol] | [symbol] | [symbol] | [symbol] | 8 | [symbol] | [symbol] | + | 2709 | protein_codin | [symbol] | — |
| ENSMUSG0 | [symbol] | [symbol] | [symbol] | [symbol] | [symbol] | [symbol] | 4 | [symbol] | [symbol] | + | 2993 | protein_codin | [symbol] | — |
| ENSMUSG0 | [symbol] | [symbol] | [symbol] | [symbol] | [symbol] | [symbol] | 15 | [symbol] | [symbol] | − | 6256 | protein_codin | [symbol] | — |
| ENSMUSG0 | [symbol] | [symbol] | [symbol] | [symbol] | [symbol] | [symbol] | 8 | [symbol] | [symbol] | + | 5227 | protein_codin | [symbol] | HMG |
| ENSMUSG0 | [symbol] | [symbol] | [symbol] | [symbol] | [symbol] | [symbol] | 5 | [symbol] | [symbol] | + | 5860 | protein_codin | [symbol] | — |
| ENSMUSG0 | [symbol] | [symbol] | [symbol] | [symbol] | [symbol] | [symbol] | 4 | [symbol] | [symbol] | + | 17627 | protein_codin | [symbol] | zf- |
| | | | | | | | | | | | | | | C2H2 |
| ENSMUSG0 | [symbol] | [symbol] | [symbol] | [symbol] | [symbol] | [symbol] | 14 | [symbol] | [symbol] | + | [symbol] 526 | protein_codin | [symbol] | — |
| ENSMUSG0 | [symbol] | [symbol] | [symbol] | [symbol] | [symbol] | [symbol] | 16 | [symbol] | [symbol] | − | [symbol] | protein_codin | [symbol] | — |
| ENSMUSG0 | [symbol] | [symbol] | [symbol] | [symbol] | [symbol] | [symbol] | 9 | [symbol] | [symbol] | + | [symbol] | protein_codin | [symbol] | — |
| ENSMUSG0 | [symbol] | [symbol] | [symbol] | [symbol] | [symbol] | [symbol] | 3 | [symbol] | [symbol] | − | 2344 | protein_codin | [symbol] | — |
| ENSMUSG0 | [symbol] | [symbol] | [symbol] | [symbol] | [symbol] | [symbol] | 4 | [symbol] | [symbol] | + | [symbol] | protein_codin | [symbol] | — |
| ENSMUSG0 | [symbol] | [symbol] | [symbol] | [symbol] | [symbol] | [symbol] | 11 | [symbol] | [symbol] | − | 2464 | protein_codin | [symbol] | — |
| ENSMUSG0 | [symbol] | [symbol] | [symbol] | [symbol] | [symbol] | [symbol] | 17 | [symbol] | [symbol] | − | 4840 | protein_codin | [symbol] | — |
| ENSMUSG0 | [symbol] | [symbol] | [symbol] | [symbol] | [symbol] | [symbol] | 14 | [symbol] | [symbol] | − | 2563 | protein_codin | [symbol] | — |
| ENSMUSG0 | [symbol] | [symbol] | [symbol] | [symbol] | [symbol] | [symbol] | 2 | [symbol] | [symbol] | + | 2330 | protein_codin | [symbol] | — |
| ENSMUSG0 | [symbol] | [symbol] | [symbol] | [symbol] | [symbol] | [symbol] | 15 | [symbol] | [symbol] | − | 3738 | protein_codin | [symbol] | — |
| ENSMUSG0 | [symbol] | [symbol] | [symbol] | [symbol] | [symbol] | [symbol] | 7 | [symbol] | [symbol] | + | 4176 | protein_codin | [symbol] | — |
| ENSMUSG0 | [symbol] | [symbol] | [symbol] | [symbol] | [symbol] | [symbol] | 14 | [symbol] | [symbol] | − | 4298 | protein_codin | [symbol] | — |
| ENSMUSG0 | [symbol] | [symbol] | [symbol] | [symbol] | [symbol] | [symbol] | X | [symbol] | [symbol] | + | 6098 | protein_codin | [symbol] | — |
| ENSMUSG0 | [symbol] | [symbol] | [symbol] | [symbol] | [symbol] | [symbol] | 12 | [symbol] | [symbol] | − | 3931 | protein_codin | [symbol] | — |
| ENSMUSG0 | [symbol] | [symbol] | [symbol] | [symbol] | [symbol] | [symbol] | 3 | [symbol] | [symbol] | − | 2366 | protein_codin | [symbol] | — |
| ENSMUSG0 | [symbol] | [symbol] | [symbol] | [symbol] | [symbol] | [symbol] | 4 | [symbol] | [symbol] | − | 2766 | protein_codin | [symbol] | — |
| ENSMUSG0 | [symbol] | [symbol] | [symbol] | [symbol] | [symbol] | [symbol] | 17 | [symbol] | [symbol] | − | 5950 | protein_codin | [symbol] | — |
| ENSMUSG0 | [symbol] | [symbol] | [symbol] | [symbol] | [symbol] | [symbol] | 14 | [symbol] | [symbol] | − | 11570 | protein_codin | [symbol] | — |
| ENSMUSG0 | [symbol] | [symbol] | [symbol] | [symbol] | [symbol] | [symbol] | 6 | [symbol] | [symbol] | − | 4171 | protein_codin | [symbol] | — |
| ENSMUSG0 | [symbol] | [symbol] | [symbol] | [symbol] | [symbol] | [symbol] | 10 | [symbol] | [symbol] | − | 4091 | protein_codin | [symbol] | — |
| ENSMUSG0 | [symbol] | [symbol] | [symbol] | [symbol] | [symbol] | [symbol] | 19 | [symbol] | [symbol] | − | 3798 | protein_codin | [symbol] | — |
| ENSMUSG0 | [symbol] | [symbol] | [symbol] | [symbol] | [symbol] | [symbol] | 13 | [symbol] | [symbol] | − | 5553 | protein_codin | [symbol] | — |
| ENSMUSG0 | [symbol] | [symbol] | [symbol] | [symbol] | [symbol] | [symbol] | 6 | [symbol] | [symbol] | + | 2818 | protein_codin | [symbol] | — |
| ENSMUSG0 | [symbol] | [symbol] | [symbol] | [symbol] | [symbol] | [symbol] | 12 | [symbol] | [symbol] | + | 4599 | protein_codin | [symbol] | — |
| ENSMUSG0 | [symbol] | [symbol] | [symbol] | [symbol] | [symbol] | [symbol] | 1 | [symbol] | [symbol] | + | 8400 | protein_codin | [symbol] | ZBTB |
| ENSMUSG0 | [symbol] | [symbol] | [symbol] | [symbol] | [symbol] | [symbol] | 11 | [symbol] | [symbol] | − | 2488 | protein_codin | [symbol] | T-box |
| ENSMUSG0 | [symbol] | [symbol] | [symbol] | [symbol] | [symbol] | [symbol] | 13 | [symbol] | [symbol] | − | 5429 | protein_codin | [symbol] | E2F |
| ENSMUSG0 | [symbol] | [symbol] | [symbol] | [symbol] | [symbol] | [symbol] | 12 | [symbol] | [symbol] | − | 20676 | protein_codin | [symbol] | — |
| ENSMUSG0 | [symbol] | [symbol] | [symbol] | [symbol] | [symbol] | [symbol] | 14 | [symbol] | [symbol] | − | 6015 | protein_codin | [symbol] | — |
| ENSMUSG0 | [symbol] | [symbol] | [symbol] | [symbol] | [symbol] | [symbol] | 5 | [symbol] | [symbol] | + | 2590 | protein_codin | [symbol] | — |
| ENSMUSG0 | [symbol] | [symbol] | [symbol] | [symbol] | [symbol] | [symbol] | 1 | [symbol] | [symbol] | − | 2005 | protein_codin | [symbol] | — |
| ENSMUSG0 | [symbol] | [symbol] | [symbol] | [symbol] | [symbol] | [symbol] | 2 | [symbol] | [symbol] | + | 6195 | protein_codin | [symbol] | — |
| ENSMUSG0 | [symbol] | [symbol] | [symbol] | [symbol] | [symbol] | [symbol] | 10 | [symbol] | [symbol] | − | 9028 | protein_codin | [symbol] | — |
| ENSMUSG0 | [symbol] | [symbol] | [symbol] | [symbol] | [symbol] | [symbol] | 16 | [symbol] | [symbol] | − | 3221 | protein_codin | [symbol] | — |
| ENSMUSG0 | [symbol] | [symbol] | [symbol] | [symbol] | [symbol] | [symbol] | 15 | [symbol] | [symbol] | + | 4198 | protein_codin | [symbol] | — |
| ENSMUSG0 | [symbol] | [symbol] | [symbol] | [symbol] | [symbol] | [symbol] | 11 | [symbol] | [symbol] | + | 872 | protein_codin | [symbol] | — |
| ENSMUSG0 | [symbol] | [symbol] | [symbol] | [symbol] | [symbol] | [symbol] | X | [symbol] | [symbol] | − | 419 | protein_codin | [symbol] | — |
| ENSMUSG0 | [symbol] | [symbol] | [symbol] | [symbol] | [symbol] | [symbol] | 4 | [symbol] | [symbol] | + | 5093 | protein_codin | [symbol] | — |
| ENSMUSG0 | [symbol] | [symbol] | [symbol] | [symbol] | [symbol] | [symbol] | 11 | [symbol] | [symbol] | − | 6248 | protein_codin | [symbol] | — |
| ENSMUSG0 | [symbol] | [symbol] | [symbol] | [symbol] | [symbol] | [symbol] | 4 | [symbol] | [symbol] | − | 3088 | protein_codin | [symbol] | — |
| ENSMUSG0 | [symbol] | [symbol] | [symbol] | [symbol] | [symbol] | [symbol] | 13 | [symbol] | [symbol] | − | 21824 | protein_codin | [symbol] | — |
| ENSMUSG0 | [symbol] | [symbol] | [symbol] | [symbol] | [symbol] | [symbol] | 4 | [symbol] | [symbol] | − | 2040 | protein_codin | [symbol] | — |
| ENSMUSG0 | [symbol] | [symbol] | [symbol] | [symbol] | [symbol] | [symbol] | 8 | [symbol] | [symbol] | − | 5253 | protein_codin | [symbol] | — |
| ENSMUSG0 | [symbol] | [symbol] | [symbol] | [symbol] | [symbol] | [symbol] | 2 | [symbol] | [symbol] | − | 1032 | protein_codin | [symbol] | — |
| ENSMUSG0 | [symbol] | [symbol] | [symbol] | [symbol] | [symbol] | [symbol] | 5 | [symbol] | [symbol] | + | 13135 | protein_codin | [symbol] | — |
| ENSMUSG0 | [symbol] | [symbol] | [symbol] | [symbol] | [symbol] | [symbol] | 11 | [symbol] | [symbol] | − | 3454 | protein_codin | [symbol] | — |
| ENSMUSG0 | [symbol] | [symbol] | [symbol] | [symbol] | [symbol] | [symbol] | 2 | [symbol] | [symbol] | + | 2703 | protein_codin | [symbol] | — |
| ENSMUSG0 | [symbol] | [symbol] | [symbol] | [symbol] | [symbol] | [symbol] | 7 | [symbol] | [symbol] | − | 2684 | protein_codin | [symbol] | — |
| ENSMUSG0 | [symbol] | [symbol] | [symbol] | [symbol] | [symbol] | [symbol] | 2 | [symbol] | [symbol] | − | 2088 | protein_codin | [symbol] | — |
| ENSMUSG0 | [symbol] | [symbol] | [symbol] | [symbol] | [symbol] | [symbol] | 10 | [symbol] | [symbol] | − | 3850 | protein_codin | [symbol] | — |
| ENSMUSG0 | [symbol] | [symbol] | [symbol] | [symbol] | [symbol] | [symbol] | 17 | [symbol] | [symbol] | − | 7315 | protein_codin | [symbol] | NF-YA |
| ENSMUSG0 | [symbol] | [symbol] | [symbol] | [symbol] | [symbol] | [symbol] | 2 | [symbol] | [symbol] | + | 3728 | protein_codin | [symbol] | — |
| ENSMUSG0 | [symbol] | [symbol] | [symbol] | [symbol] | [symbol] | [symbol] | 2 | [symbol] | [symbol] | + | 5707 | protein_codin | [symbol] | — |
| ENSMUSG0 | [symbol] | [symbol] | [symbol] | [symbol] | [symbol] | [symbol] | 14 | [symbol] | [symbol] | − | 5039 | protein_codin | [symbol] | — |
| ENSMUSG0 | [symbol] | [symbol] | [symbol] | [symbol] | [symbol] | [symbol] | 11 | [symbol] | [symbol] | − | 3420 | protein_codin | [symbol] | — |
| ENSMUSG0 | [symbol] | [symbol] | [symbol] | [symbol] | [symbol] | [symbol] | 10 | [symbol] | [symbol] | − | 3210 | protein_codin | [symbol] | — |
| ENSMUSG0 | [symbol] | [symbol] | [symbol] | [symbol] | [symbol] | [symbol] | 11 | [symbol] | [symbol] | − | 1633 | protein_codin | [symbol] | — |
| ENSMUSG0 | [symbol] | [symbol] | [symbol] | [symbol] | [symbol] | [symbol] | 10 | [symbol] | [symbol] | + | 1740 | protein_codin | [symbol] | — |
| ENSMUSG0 | [symbol] | [symbol] | [symbol] | [symbol] | [symbol] | [symbol] | 5 | [symbol] | [symbol] | + | 3709 | protein_codin | [symbol] | — |
| ENSMUSG0 | [symbol] | [symbol] | [symbol] | [symbol] | [symbol] | [symbol] | 12 | [symbol] | [symbol] | − | 1518 | protein_codin | [symbol] | — |
| ENSMUSG0 | [symbol] | [symbol] | [symbol] | [symbol] | [symbol] | [symbol] | 18 | [symbol] | [symbol] | + | 3698 | protein_codin | [symbol] | — |
| ENSMUSG0 | [symbol] | [symbol] | [symbol] | [symbol] | [symbol] | [symbol] | 12 | [symbol] | [symbol] | − | 5080 | protein_codin | [symbol] | — |
| ENSMUSG0 | [symbol] | [symbol] | [symbol] | [symbol] | [symbol] | [symbol] | 5 | [symbol] | [symbol] | − | 9664 | protein_codin | [symbol] | — |
| ENSMUSG0 | [symbol] | [symbol] | [symbol] | [symbol] | [symbol] | [symbol] | X | [symbol] | [symbol] | − | 5570 | protein_codin | [symbol] | — |
| ENSMUSG0 | [symbol] | [symbol] | [symbol] | [symbol] | [symbol] | [symbol] | 17 | [symbol] | [symbol] | + | 1799 | protein_codin | [symbol] | — |
| ENSMUSG0 | [symbol] | [symbol] | [symbol] | [symbol] | [symbol] | [symbol] | 19 | [symbol] | [symbol] | − | 2813 | protein_codin | [symbol] | — |
| ENSMUSG0 | [symbol] | [symbol] | [symbol] | [symbol] | [symbol] | [symbol] | 18 | [symbol] | [symbol] | + | 4257 | protein_codin | [symbol] | — |
| ENSMUSG0 | [symbol] | [symbol] | [symbol] | [symbol] | [symbol] | [symbol] | 13 | [symbol] | [symbol] | − | 1131 | protein_codin | [symbol] | — |
| ENSMUSG0 | [symbol] | [symbol] | [symbol] | [symbol] | [symbol] | [symbol] | 7 | [symbol] | [symbol] | − | 3828 | protein_codin | [symbol] | — |
| ENSMUSG0 | [symbol] | [symbol] | [symbol] | [symbol] | [symbol] | [symbol] | 15 | [symbol] | [symbol] | + | 2287 | protein_codin | [symbol] | — |
| ENSMUSG0 | [symbol] | [symbol] | [symbol] | [symbol] | [symbol] | [symbol] | 15 | [symbol] | [symbol] | − | 2731 | protein_codin | [symbol] | — |
| ENSMUSG0 | [symbol] | [symbol] | [symbol] | [symbol] | [symbol] | [symbol] | 8 | [symbol] | [symbol] | − | 1178 | protein_codin | [symbol] | — |
| ENSMUSG0 | [symbol] | [symbol] | [symbol] | [symbol] | [symbol] | [symbol] | 39 | [symbol] | [symbol] | + | 1751 | protein_codin | [symbol] | — |
| ENSMUSG0 | [symbol] | [symbol] | [symbol] | [symbol] | [symbol] | [symbol] | 4 | [symbol] | [symbol] | + | 4500 | protein_codin | [symbol] | — |
| ENSMUSG0 | [symbol] | [symbol] | [symbol] | [symbol] | [symbol] | [symbol] | 17 | [symbol] | [symbol] | − | 1467 | protein_codin | [symbol] | — |
| ENSMUSG0 | [symbol] | [symbol] | [symbol] | [symbol] | [symbol] | [symbol] | 9 | [symbol] | [symbol] | + | 836 | protein_codin | [symbol] | — |
| ENSMUSG0 | [symbol] | [symbol] | [symbol] | [symbol] | [symbol] | [symbol] | 5 | [symbol] | [symbol] | − | 7721 | protein_codin | [symbol] | — |
| ENSMUSG0 | [symbol] | [symbol] | [symbol] | [symbol] | [symbol] | [symbol] | 12 | [symbol] | [symbol] | − | 7590 | protein_codin | [symbol] | — |
| ENSMUSG0 | [symbol] | [symbol] | [symbol] | [symbol] | [symbol] | [symbol] | 10 | [symbol] | [symbol] | + | 7267 | protein_codin | [symbol] | — |
| ENSMUSG0 | [symbol] | [symbol] | [symbol] | [symbol] | [symbol] | [symbol] | 7 | [symbol] | [symbol] | + | 2854 | protein_codin | [symbol] | — |
| ENSMUSG0 | [symbol] | [symbol] | [symbol] | [symbol] | [symbol] | [symbol] | 3 | [symbol] | [symbol] | + | 8580 | protein_codin | [symbol] | — |
| ENSMUSG0 | [symbol] | [symbol] | [symbol] | [symbol] | [symbol] | [symbol] | 16 | [symbol] | [symbol] | + | 3970 | protein_codin | [symbol] | — |
| ENSMUSG0 | [symbol] | [symbol] | [symbol] | [symbol] | [symbol] | [symbol] | 4 | [symbol] | [symbol] | + | 5314 | protein_codin | [symbol] | — |
| ENSMUSG0 | [symbol] | [symbol] | [symbol] | [symbol] | [symbol] | [symbol] | 2 | [symbol] | [symbol] | + | 5373 | protein_codin | [symbol] | — |
| ENSMUSG0 | [symbol] | [symbol] | [symbol] | [symbol] | [symbol] | [symbol] | 6 | [symbol] | [symbol] | − | 5347 | protein_codin | [symbol] | — |
| ENSMUSG0 | [symbol] | [symbol] | [symbol] | [symbol] | [symbol] | [symbol] | 4 | [symbol] | [symbol] | − | 8744 | protein_codin | [symbol] | — |
| ENSMUSG0 | [symbol] | [symbol] | [symbol] | [symbol] | [symbol] | [symbol] | 3 | [symbol] | [symbol] | + | 6008 | protein_codin | [symbol] | — |
| ENSMUSG0 | [symbol] | [symbol] | [symbol] | [symbol] | [symbol] | [symbol] | 14 | [symbol] | [symbol] | + | 2377 | protein_codin | [symbol] | — |
| ENSMUSG0 | [symbol] | [symbol] | [symbol] | [symbol] | [symbol] | [symbol] | 10 | [symbol] | [symbol] | − | 863 | protein_codin | [symbol] | — |
| ENSMUSG0 | [symbol] | [symbol] | [symbol] | [symbol] | [symbol] | [symbol] | 9 | [symbol] | [symbol] | + | 3077 | protein_codin | [symbol] | — |
| ENSMUSG0 | [symbol] | [symbol] | [symbol] | [symbol] | [symbol] | [symbol] | 15 | [symbol] | [symbol] | − | 3567 | protein_codin | [symbol] | — |
| ENSMUSG0 | [symbol] | [symbol] | [symbol] | [symbol] | [symbol] | [symbol] | 1 | [symbol] | [symbol] | − | 3392 | protein_codin | [symbol] | — |
| ENSMUSG0 | [symbol] | [symbol] | [symbol] | [symbol] | [symbol] | [symbol] | X | [symbol] | [symbol] | + | 6851 | protein_codin | [symbol] | — |
| ENSMUSG0 | [symbol] | [symbol] | [symbol] | [symbol] | [symbol] | [symbol] | 19 | [symbol] | [symbol] | − | 820 | protein_codin | [symbol] | — |
| ENSMUSG0 | [symbol] | [symbol] | [symbol] | [symbol] | [symbol] | [symbol] | X | [symbol] | [symbol] | + | 2517 | protein_codin | [symbol] | — |
| ENSMUSG0 | [symbol] | [symbol] | [symbol] | [symbol] | [symbol] | [symbol] | 6 | [symbol] | [symbol] | − | 2509 | protein_codin | [symbol] | — |
| ENSMUSG0 | [symbol] | [symbol] | [symbol] | [symbol] | [symbol] | [symbol] | 11 | [symbol] | [symbol] | + | 3332 | protein_codin | [symbol] | — |
| ENSMUSG0 | [symbol] | [symbol] | [symbol] | [symbol] | [symbol] | [symbol] | 4 | [symbol] | [symbol] | + | 770 | protein_codin | [symbol] | — |
| ENSMUSG0 | [symbol] | [symbol] | [symbol] | [symbol] | [symbol] | [symbol] | 9 | [symbol] | [symbol] | + | 3827 | protein_codin | [symbol] | — |
| ENSMUSG0 | [symbol] | [symbol] | [symbol] | [symbol] | [symbol] | [symbol] | 19 | [symbol] | [symbol] | − | 5030 | protein_codin | [symbol] | — |
| ENSMUSG0 | [symbol] | [symbol] | [symbol] | [symbol] | [symbol] | [symbol] | 17 | [symbol] | [symbol] | − | 7060 | protein_codin | [symbol] | — |
| ENSMUSG0 | [symbol] | [symbol] | [symbol] | [symbol] | [symbol] | [symbol] | 12 | [symbol] | [symbol] | + | 3299 | protein_codin | [symbol] | — |
| ENSMUSG0 | [symbol] | [symbol] | [symbol] | [symbol] | [symbol] | [symbol] | 2 | [symbol] | [symbol] | + | 8490 | protein_codin | [symbol] | — |
| ENSMUSG0 | [symbol] | [symbol] | [symbol] | [symbol] | [symbol] | [symbol] | 7 | [symbol] | [symbol] | + | 11369 | protein_codin | [symbol] | — |
| ENSMUSG0 | [symbol] | [symbol] | [symbol] | [symbol] | [symbol] | [symbol] | 12 | [symbol] | [symbol] | − | 1717 | protein_codin | [symbol] | — |
| ENSMUSG0 | [symbol] | [symbol] | [symbol] | [symbol] | [symbol] | [symbol] | 9 | [symbol] | [symbol] | + | 6378 | protein_codin | [symbol] | — |
| ENSMUSG0 | [symbol] | [symbol] | [symbol] | [symbol] | [symbol] | [symbol] | 6 | [symbol] | [symbol] | + | 5992 | protein_codin | [symbol] | — |
| ENSMUSG0 | [symbol] | [symbol] | [symbol] | [symbol] | [symbol] | [symbol] | 17 | [symbol] | [symbol] | + | 8333 | protein_codin | [symbol] | — |
| ENSMUSG0 | [symbol] | [symbol] | [symbol] | [symbol] | [symbol] | [symbol] | 6 | [symbol] | [symbol] | + | 12640 | protein_codin | [symbol] | — |
| ENSMUSG0 | [symbol] | [symbol] | [symbol] | [symbol] | [symbol] | [symbol] | 10 | [symbol] | [symbol] | − | 5011 | protein_codin | [symbol] | — |
| ENSMUSG0 | [symbol] | [symbol] | [symbol] | [symbol] | [symbol] | [symbol] | 3 | [symbol] | [symbol] | + | 8105 | protein_codin | [symbol] | — |
| ENSMUSG0 | [symbol] | [symbol] | [symbol] | [symbol] | [symbol] | [symbol] | 10 | [symbol] | [symbol] | + | 4435 | protein_codin | [symbol] | — |
| ENSMUSG0 | [symbol] | [symbol] | [symbol] | [symbol] | [symbol] | [symbol] | 3 | [symbol] | [symbol] | − | 4333 | protein_codin | [symbol] | — |
| ENSMUSG0 | [symbol] | [symbol] | [symbol] | [symbol] | [symbol] | [symbol] | 11 | [symbol] | [symbol] | + | 1872 | protein_codin | [symbol] | — |
| ENSMUSG0 | [symbol] | [symbol] | [symbol] | [symbol] | [symbol] | [symbol] | 14 | [symbol] | [symbol] | − | 7734 | protein_codin | [symbol] | — |
| ENSMUSG0 | [symbol] | [symbol] | [symbol] | [symbol] | [symbol] | [symbol] | 2 | [symbol] | [symbol] | − | 3463 | protein_codin | [symbol] | — |
| ENSMUSG0 | [symbol] | [symbol] | [symbol] | [symbol] | [symbol] | [symbol] | 9 | [symbol] | [symbol] | + | 7786 | protein_codin | [symbol] | — |
| ENSMUSG0 | [symbol] | [symbol] | [symbol] | [symbol] | [symbol] | [symbol] | 16 | [symbol] | [symbol] | − | 3130 | protein_codin | [symbol] | — |
| ENSMUSG0 | [symbol] | [symbol] | [symbol] | [symbol] | [symbol] | [symbol] | 4 | [symbol] | [symbol] | + | 2140 | protein_codin | [symbol] | — |
| ENSMUSG0 | [symbol] | [symbol] | [symbol] | [symbol] | [symbol] | [symbol] | 15 | [symbol] | [symbol] | − | 1675 | protein_codin | [symbol] | — |
| ENSMUSG0 | [symbol] | [symbol] | [symbol] | [symbol] | [symbol] | [symbol] | 14 | [symbol] | [symbol] | + | 3498 | protein_codin | [symbol] | — |
| ENSMUSG0 | [symbol] | [symbol] | [symbol] | [symbol] | [symbol] | [symbol] | 12 | [symbol] | [symbol] | + | 5732 | protein_codin | [symbol] | — |
| ENSMUSG0 | [symbol] | [symbol] | [symbol] | [symbol] | [symbol] | [symbol] | 1 | [symbol] | [symbol] | + | 3826 | protein_codin | [symbol] | — |
| ENSMUSG0 | [symbol] | [symbol] | [symbol] | [symbol] | [symbol] | [symbol] | 4 | [symbol] | [symbol] | + | 5324 | protein_codin | [symbol] | — |
| ENSMUSG0 | [symbol] | [symbol] | [symbol] | [symbol] | [symbol] | [symbol] | 1 | [symbol] | [symbol] | + | 7265 | protein_codin | [symbol] | — |
| ENSMUSG0 | [symbol] | [symbol] | [symbol] | [symbol] | [symbol] | [symbol] | 6 | [symbol] | [symbol] | + | 4619 | protein_codin | [symbol] | — |
| ENSMUSG0 | [symbol] | [symbol] | [symbol] | [symbol] | [symbol] | [symbol] | 13 | [symbol] | [symbol] | − | 5771 | protein_codin | [symbol] | — |
| ENSMUSG0 | [symbol] | [symbol] | [symbol] | [symbol] | [symbol] | [symbol] | 17 | [symbol] | [symbol] | − | 8173 | protein_codin | [symbol] | — |
| ENSMUSG0 | [symbol] | [symbol] | [symbol] | [symbol] | [symbol] | [symbol] | 12 | [symbol] | [symbol] | + | 3261 | protein_codin | [symbol] | — |
| ENSMUSG0 | [symbol] | [symbol] | [symbol] | [symbol] | [symbol] | [symbol] | 13 | [symbol] | [symbol] | − | 6393 | protein_codin | [symbol] | — |
| ENSMUSG0 | [symbol] | [symbol] | [symbol] | [symbol] | [symbol] | [symbol] | 8 | [symbol] | [symbol] | − | 14828 | protein_codin | [symbol] | — |
| ENSMUSG0 | [symbol] | [symbol] | [symbol] | [symbol] | [symbol] | [symbol] | 1 | [symbol] | [symbol] | + | 4817 | protein_codin | [symbol] | — |
| ENSMUSG0 | [symbol] | [symbol] | [symbol] | [symbol] | [symbol] | [symbol] | 17 | [symbol] | [symbol] | − | 2147 | protein_codin | [symbol] | — |
| ENSMUSG0 | [symbol] | [symbol] | [symbol] | [symbol] | [symbol] | [symbol] | 3 | [symbol] | [symbol] | − | 4172 | protein_codin | [symbol] | — |
| ENSMUSG0 | [symbol] | [symbol] | [symbol] | [symbol] | [symbol] | [symbol] | 3 | [symbol] | [symbol] | − | 7423 | protein_codin | [symbol] | — |
| ENSMUSG0 | [symbol] | [symbol] | [symbol] | [symbol] | [symbol] | [symbol] | 6 | [symbol] | [symbol] | + | 4663 | protein_codin | [symbol] | — |
| ENSMUSG0 | [symbol] | [symbol] | [symbol] | [symbol] | [symbol] | [symbol] | 8 | [symbol] | [symbol] | + | 7018 | protein_codin | [symbol] | — |
| ENSMUSG0 | [symbol] | [symbol] | [symbol] | [symbol] | [symbol] | [symbol] | 11 | [symbol] | [symbol] | − | 7017 | protein_codin | [symbol] | — |
| ENSMUSG0 | [symbol] | [symbol] | [symbol] | [symbol] | [symbol] | [symbol] | 4 | [symbol] | [symbol] | − | 6447 | protein_codin | [symbol] | — |
| ENSMUSG0 | [symbol] | [symbol] | [symbol] | [symbol] | [symbol] | [symbol] | 7 | [symbol] | [symbol] | − | 3280 | protein_codin | [symbol] | — |
| ENSMUSG0 | [symbol] | [symbol] | [symbol] | [symbol] | [symbol] | [symbol] | 6 | [symbol] | [symbol] | − | 3943 | protein_codin | [symbol] | — |
| ENSMUSG0 | [symbol] | [symbol] | [symbol] | [symbol] | [symbol] | [symbol] | 11 | [symbol] | [symbol] | + | 6868 | protein_codin | [symbol] | — |
| ENSMUSG0 | [symbol] | [symbol] | [symbol] | [symbol] | [symbol] | [symbol] | 14 | [symbol] | [symbol] | − | 10124 | protein_codin | [symbol] | — |
| ENSMUSG0 | [symbol] | [symbol] | [symbol] | [symbol] | [symbol] | [symbol] | 14 | [symbol] | [symbol] | + | 6502 | protein_codin | [symbol] | — |
| ENSMUSG0 | [symbol] | [symbol] | [symbol] | [symbol] | [symbol] | [symbol] | 15 | [symbol] | [symbol] | + | 8106 | protein_codin | [symbol] | — |
| ENSMUSG0 | [symbol] | [symbol] | [symbol] | [symbol] | [symbol] | [symbol] | 4 | [symbol] | [symbol] | − | 10710 | protein_codin | [symbol] | ZBTB |
| ENSMUSG0 | [symbol] | [symbol] | [symbol] | [symbol] | [symbol] | [symbol] | 11 | [symbol] | [symbol] | − | 4698 | protein_codin | [symbol] | — |
| ENSMUSG0 | [symbol] | [symbol] | [symbol] | [symbol] | [symbol] | [symbol] | 11 | [symbol] | [symbol] | − | 4595 | protein_codin | [symbol] | — |
| ENSMUSG0 | [symbol] | [symbol] | [symbol] | [symbol] | [symbol] | [symbol] | 15 | [symbol] | [symbol] | + | 2414 | protein_codin | [symbol] | — |
| ENSMUSG0 | [symbol] | [symbol] | [symbol] | [symbol] | [symbol] | [symbol] | 11 | [symbol] | [symbol] | − | 6463 | protein_codin | [symbol] | — |
| ENSMUSG0 | [symbol] | [symbol] | [symbol] | [symbol] | [symbol] | [symbol] | 10 | [symbol] | [symbol] | − | 8933 | protein_codin | [symbol] | MYB |
| ENSMUSG0 | [symbol] | [symbol] | [symbol] | [symbol] | [symbol] | [symbol] | 11 | [symbol] | [symbol] | + | 13896 | protein_codin | [symbol] | — |
| ENSMUSG0 | [symbol] | [symbol] | [symbol] | [symbol] | [symbol] | [symbol] | 1 | [symbol] | [symbol] | − | 4515 | protein_codin | [symbol] | — |
| ENSMUSG0 | [symbol] | [symbol] | [symbol] | [symbol] | [symbol] | [symbol] | 7 | [symbol] | [symbol] | + | 2800 | protein_codin | [symbol] | — |
| ENSMUSG0 | [symbol] | [symbol] | [symbol] | [symbol] | [symbol] | [symbol] | 5 | [symbol] | [symbol] | + | 5505 | protein_codin | [symbol] | — |
| ENSMUSG0 | [symbol] | [symbol] | [symbol] | [symbol] | [symbol] | [symbol] | 1 | [symbol] | [symbol] | − | 6460 | protein_codin | [symbol] | — |
| ENSMUSG0 | [symbol] | [symbol] | [symbol] | [symbol] | [symbol] | [symbol] | 4 | [symbol] | [symbol] | − | 1334 | protein_codin | [symbol] | THAP |
| ENSMUSG0 | [symbol] | [symbol] | [symbol] | [symbol] | [symbol] | [symbol] | 15 | [symbol] | [symbol] | − | 23395 | protein_codin | [symbol] | — |
| ENSMUSG0 | [symbol] | [symbol] | [symbol] | [symbol] | [symbol] | [symbol] | 2 | [symbol] | [symbol] | + | 8561 | protein_codin | [symbol] | — |
| ENSMUSG0 | [symbol] | [symbol] | [symbol] | [symbol] | [symbol] | [symbol] | 2 | [symbol] | [symbol] | + | 9807 | protein_codin | [symbol] | zf- |
| | | | | | | | | | | | | | | C2H2 |
| ENSMUSG0 | [symbol] | [symbol] | [symbol] | [symbol] | [symbol] | [symbol] | 13 | [symbol] | [symbol] | + | 16704 | protein_codin | [symbol] | — |
| ENSMUSG0 | [symbol] | [symbol] | [symbol] | [symbol] | [symbol] | [symbol] | 3 | [symbol] | [symbol] | + | 6971 | protein_codin | [symbol] | — |
| ENSMUSG0 | [symbol] | [symbol] | [symbol] | [symbol] | [symbol] | [symbol] | 12 | [symbol] | [symbol] | − | 10049 | protein_codin | [symbol] | — |
| ENSMUSG0 | [symbol] | [symbol] | [symbol] | [symbol] | [symbol] | [symbol] | 11 | [symbol] | [symbol] | + | 3056 | protein_codin | [symbol] | — |
| ENSMUSG0 | [symbol] | [symbol] | [symbol] | [symbol] | [symbol] | [symbol] | 11 | [symbol] | [symbol] | + | 5631 | protein_codin | [symbol] | — |
| ENSMUSG0 | [symbol] | [symbol] | [symbol] | [symbol] | [symbol] | [symbol] | 5 | [symbol] | [symbol] | − | [symbol] | protein_codin | [symbol] | zf- |
| | | | | | | | | | | | | | | C2H2 |
| ENSMUSG0 | [symbol] | [symbol] | [symbol] | [symbol] | [symbol] | [symbol] | 5 | [symbol] | [symbol] | + | 8541 | protein_codin | [symbol] | zf- |
| | | | | | | | | | | | | | | C2H2 |
| ENSMUSG0 | [symbol] | [symbol] | [symbol] | [symbol] | [symbol] | [symbol] | 9 | [symbol] | [symbol] | − | 2578 | protein_codin | [symbol] | — |
| ENSMUSG0 | [symbol] | [symbol] | [symbol] | [symbol] | [symbol] | [symbol] | 2 | [symbol] | [symbol] | + | 8451 | protein_codin | [symbol] | — |
| ENSMUSG0 | [symbol] | [symbol] | [symbol] | [symbol] | [symbol] | [symbol] | 9 | [symbol] | [symbol] | + | 2458 | protein_codin | [symbol] | — |
| ENSMUSG0 | [symbol] | [symbol] | [symbol] | [symbol] | [symbol] | [symbol] | 8 | [symbol] | [symbol] | + | 9698 | protein_codin | [symbol] | — |
| ENSMUSG0 | [symbol] | [symbol] | [symbol] | [symbol] | [symbol] | [symbol] | 10 | [symbol] | [symbol] | − | 5949 | protein_codin | [symbol] | — |
| ENSMUSG0 | [symbol] | [symbol] | [symbol] | [symbol] | [symbol] | [symbol] | 5 | [symbol] | [symbol] | + | 4319 | protein_codin | [symbol] | — |
| ENSMUSG0 | [symbol] | [symbol] | [symbol] | [symbol] | [symbol] | [symbol] | 8 | [symbol] | [symbol] | − | 9131 | protein_codin | [symbol] | — |
| ENSMUSG0 | [symbol] | [symbol] | [symbol] | [symbol] | [symbol] | [symbol] | 15 | [symbol] | [symbol] | + | 2067 | protein_codin | [symbol] | — |
| ENSMUSG0 | [symbol] | [symbol] | [symbol] | [symbol] | [symbol] | [symbol] | 12 | [symbol] | [symbol] | − | 2715 | protein_codin | [symbol] | — |
| ENSMUSG0 | [symbol] | [symbol] | [symbol] | [symbol] | [symbol] | [symbol] | 19 | [symbol] | [symbol] | − | 4640 | protein_codin | [symbol] | — |
| ENSMUSG0 | [symbol] | [symbol] | [symbol] | [symbol] | [symbol] | [symbol] | 6 | [symbol] | [symbol] | + | 6449 | protein_codin | [symbol] | — |
| ENSMUSG0 | [symbol] | [symbol] | [symbol] | [symbol] | [symbol] | [symbol] | 7 | [symbol] | [symbol] | + | 2592 | protein_codin | [symbol] | — |
| ENSMUSG0 | [symbol] | [symbol] | [symbol] | [symbol] | [symbol] | [symbol] | 17 | [symbol] | [symbol] | + | 3903 | protein_codin | [symbol] | — |
| ENSMUSG0 | [symbol] | [symbol] | [symbol] | [symbol] | [symbol] | [symbol] | 4 | [symbol] | [symbol] | − | 2875 | protein_codin | [symbol] | — |
| ENSMUSG0 | [symbol] | [symbol] | [symbol] | [symbol] | [symbol] | [symbol] | 13 | [symbol] | [symbol] | − | 12106 | protein_codin | [symbol] | — |
| ENSMUSG0 | [symbol] | [symbol] | [symbol] | [symbol] | [symbol] | [symbol] | 9 | [symbol] | [symbol] | − | 5619 | protein_codin | [symbol] | — |
| ENSMUSG0 | [symbol] | [symbol] | [symbol] | [symbol] | [symbol] | [symbol] | 13 | [symbol] | [symbol] | − | 4322 | protein_codin | [symbol] | — |
| ENSMUSG0 | [symbol] | [symbol] | [symbol] | [symbol] | [symbol] | [symbol] | 11 | [symbol] | [symbol] | + | 5626 | protein_codin | [symbol] | — |
| ENSMUSG0 | [symbol] | [symbol] | [symbol] | [symbol] | [symbol] | [symbol] | 2 | [symbol] | [symbol] | + | 7496 | protein_codin | [symbol] | — |
| ENSMUSG0 | [symbol] | [symbol] | [symbol] | [symbol] | [symbol] | [symbol] | 8 | [symbol] | [symbol] | − | 4220 | protein_codin | [symbol] | — |
| ENSMUSG0 | [symbol] | [symbol] | [symbol] | [symbol] | [symbol] | [symbol] | 8 | [symbol] | [symbol] | − | 3490 | protein_codin | [symbol] | — |
| ENSMUSG0 | [symbol] | [symbol] | [symbol] | [symbol] | [symbol] | [symbol] | 31 | [symbol] | [symbol] | − | 7038 | protein_codin | [symbol] | — |
| ENSMUSG0 | [symbol] | [symbol] | [symbol] | [symbol] | [symbol] | [symbol] | 33 | [symbol] | [symbol] | + | 5395 | protein_codin | [symbol] | — |
| ENSMUSG0 | [symbol] | [symbol] | [symbol] | [symbol] | [symbol] | [symbol] | 11 | [symbol] | [symbol] | − | 5968 | protein_codin | [symbol] | — |
| ENSMUSG0 | [symbol] | [symbol] | [symbol] | [symbol] | [symbol] | [symbol] | 11 | [symbol] | [symbol] | − | 3329 | protein_codin | [symbol] | — |
| ENSMUSG0 | [symbol] | [symbol] | [symbol] | [symbol] | [symbol] | [symbol] | 18 | [symbol] | [symbol] | − | 5101 | protein_codin | [symbol] | HMG |
| ENSMUSG0 | [symbol] | [symbol] | [symbol] | [symbol] | [symbol] | [symbol] | 9 | [symbol] | [symbol] | − | 4538 | protein_codin | [symbol] | — |
| ENSMUSG0 | [symbol] | [symbol] | [symbol] | [symbol] | [symbol] | [symbol] | 17 | [symbol] | [symbol] | − | 2075 | protein_codin | [symbol] | — |
| ENSMUSG0 | [symbol] | [symbol] | [symbol] | [symbol] | [symbol] | [symbol] | 8 | [symbol] | [symbol] | + | 5530 | protein_codin | [symbol] | zf- |
| | | | | | | | | | | | | | | C2H2 |
| ENSMUSG0 | [symbol] | [symbol] | [symbol] | [symbol] | [symbol] | [symbol] | 33 | [symbol] | [symbol] | + | 7308 | protein_codin | [symbol] | zf- |
| | | | | | | | | | | | | | | C2H2 |
| ENSMUSG0 | [symbol] | [symbol] | [symbol] | [symbol] | [symbol] | [symbol] | 19 | [symbol] | [symbol] | + | 1968 | protein_codin | [symbol] | — |
| ENSMUSG0 | [symbol] | [symbol] | [symbol] | [symbol] | [symbol] | [symbol] | 2 | [symbol] | [symbol] | − | 5594 | protein_codin | [symbol] | — |
| ENSMUSG0 | [symbol] | [symbol] | [symbol] | [symbol] | [symbol] | [symbol] | X | [symbol] | [symbol] | + | 10048 | protein_codin | [symbol] | ESR- |
| | | | | | | | | | | | | | | like |
| ENSMUSG0 | [symbol] | [symbol] | [symbol] | [symbol] | [symbol] | [symbol] | 13 | [symbol] | [symbol] | − | 8017 | protein_codin | [symbol] | zf- |
| | | | | | | | | | | | | | | C2H2 |
| ENSMUSG0 | [symbol] | [symbol] | [symbol] | [symbol] | [symbol] | [symbol] | 18 | [symbol] | [symbol] | − | 15167 | protein_codin | [symbol] | zf- |
| | | | | | | | | | | | | | | C2H2 |
| ENSMUSG0 | [symbol] | [symbol] | [symbol] | [symbol] | [symbol] | [symbol] | 7 | [symbol] | [symbol] | − | 5239 | protein_codin | [symbol] | — |
| ENSMUSG0 | [symbol] | [symbol] | [symbol] | [symbol] | [symbol] | [symbol] | 1 | [symbol] | [symbol] | + | 16555 | protein_codin | [symbol] | ARID |
| ENSMUSG0 | [symbol] | [symbol] | [symbol] | [symbol] | [symbol] | [symbol] | 9 | [symbol] | [symbol] | + | 5845 | protein_codin | [symbol] | — |
| ENSMUSG0 | [symbol] | [symbol] | [symbol] | [symbol] | [symbol] | [symbol] | [symbol] | [symbol] | [symbol] | − | 3967 | protein_codin | [symbol] | — |
| ENSMUSG0 | [symbol] | [symbol] | [symbol] | [symbol] | [symbol] | [symbol] | 8 | [symbol] | [symbol] | + | 4108 | protein_codin | [symbol] | — |
| ENSMUSG0 | [symbol] | [symbol] | [symbol] | [symbol] | [symbol] | [symbol] | X | [symbol] | [symbol] | + | 3485 | protein_codin | [symbol] | Fork |
| ENSMUSG0 | [symbol] | [symbol] | [symbol] | [symbol] | [symbol] | [symbol] | 5 | [symbol] | [symbol] | + | 4937 | protein_codin | [symbol] | — |
| ENSMUSG0 | [symbol] | [symbol] | [symbol] | [symbol] | [symbol] | [symbol] | 15 | [symbol] | [symbol] | + | 3932 | protein_codin | [symbol] | — |
| ENSMUSG0 | [symbol] | [symbol] | [symbol] | [symbol] | [symbol] | [symbol] | X | [symbol] | [symbol] | − | 3891 | protein_codin | [symbol] | — |
| ENSMUSG0 | [symbol] | [symbol] | [symbol] | [symbol] | [symbol] | [symbol] | 6 | [symbol] | [symbol] | + | 5691 | protein_codin | [symbol] | — |
| ENSMUSG0 | [symbol] | [symbol] | [symbol] | [symbol] | [symbol] | [symbol] | 8 | [symbol] | [symbol] | + | 1824 | protein_codin | [symbol] | — |
| ENSMUSG0 | [symbol] | [symbol] | [symbol] | [symbol] | [symbol] | [symbol] | 6 | [symbol] | [symbol] | − | 6022 | protein_codin | [symbol] | — |
| ENSMUSG0 | [symbol] | [symbol] | [symbol] | [symbol] | [symbol] | [symbol] | 18 | [symbol] | [symbol] | − | 5473 | protein_codin | [symbol] | — |
| ENSMUSG0 | [symbol] | [symbol] | [symbol] | [symbol] | [symbol] | [symbol] | 8 | [symbol] | [symbol] | − | 1498 | protein_codin | [symbol] | — |
| ENSMUSG0 | [symbol] | [symbol] | [symbol] | [symbol] | [symbol] | [symbol] | 3 | [symbol] | [symbol] | + | 5946 | protein_codin | [symbol] | — |
| ENSMUSG0 | [symbol] | [symbol] | [symbol] | [symbol] | [symbol] | [symbol] | 2 | [symbol] | [symbol] | − | 2066 | protein_codin | [symbol] | — |
| ENSMUSG0 | [symbol] | [symbol] | [symbol] | [symbol] | [symbol] | [symbol] | 6 | [symbol] | [symbol] | − | 4740 | protein_codin | [symbol] | — |
| ENSMUSG0 | [symbol] | [symbol] | [symbol] | [symbol] | [symbol] | [symbol] | X | [symbol] | [symbol] | + | 3330 | protein_codin | [symbol] | — |
| ENSMUSG0 | [symbol] | [symbol] | [symbol] | [symbol] | [symbol] | [symbol] | 7 | [symbol] | [symbol] | − | 2734 | protein_codin | [symbol] | zf- |
| | | | | | | | | | | | | | | C2H2 |
| ENSMUSG0 | [symbol] | [symbol] | [symbol] | [symbol] | [symbol] | [symbol] | 10 | [symbol] | [symbol] | + | 7878 | protein_codin | [symbol] | — |
| ENSMUSG0 | [symbol] | [symbol] | [symbol] | [symbol] | [symbol] | [symbol] | 2 | [symbol] | [symbol] | + | 2604 | protein_codin | [symbol] | — |
| ENSMUSG0 | [symbol] | [symbol] | [symbol] | [symbol] | [symbol] | [symbol] | 4 | [symbol] | [symbol] | − | 2882 | protein_codin | [symbol] | — |
| ENSMUSG0 | [symbol] | [symbol] | [symbol] | [symbol] | [symbol] | [symbol] | 6 | [symbol] | [symbol] | + | 5041 | protein_codin | [symbol] | — |
| ENSMUSG0 | [symbol] | [symbol] | [symbol] | [symbol] | [symbol] | [symbol] | 15 | [symbol] | [symbol] | + | 2055 | protein_codin | [symbol] | — |
| ENSMUSG0 | [symbol] | [symbol] | [symbol] | [symbol] | [symbol] | [symbol] | 16 | [symbol] | [symbol] | − | 7361 | protein_codin | [symbol] | — |
| ENSMUSG0 | [symbol] | [symbol] | [symbol] | [symbol] | [symbol] | [symbol] | [symbol] | [symbol] | [symbol] | − | 4144 | protein_codin | [symbol] | — |
| ENSMUSG0 | [symbol] | [symbol] | [symbol] | [symbol] | [symbol] | [symbol] | X | [symbol] | [symbol] | − | 2820 | protein_codin | [symbol] | — |
| ENSMUSG0 | [symbol] | [symbol] | [symbol] | [symbol] | [symbol] | [symbol] | 1 | [symbol] | [symbol] | − | 6458 | protein_codin | [symbol] | — |
| ENSMUSG0 | [symbol] | [symbol] | [symbol] | [symbol] | [symbol] | [symbol] | 7 | [symbol] | [symbol] | + | 7556 | protein_codin | [symbol] | — |
| ENSMUSG0 | [symbol] | [symbol] | [symbol] | [symbol] | [symbol] | [symbol] | 18 | [symbol] | [symbol] | − | 3990 | protein_codin | [symbol] | — |
| ENSMUSG0 | [symbol] | [symbol] | [symbol] | [symbol] | [symbol] | [symbol] | 4 | [symbol] | [symbol] | − | 2768 | protein_codin | [symbol] | — |
| ENSMUSG0 | [symbol] | [symbol] | [symbol] | [symbol] | [symbol] | [symbol] | 5 | [symbol] | [symbol] | − | 8305 | protein_codin | [symbol] | ZBTB |
| ENSMUSG0 | [symbol] | [symbol] | [symbol] | [symbol] | [symbol] | [symbol] | 4 | [symbol] | [symbol] | + | 5999 | protein_codin | [symbol] | — |
| ENSMUSG0 | [symbol] | [symbol] | [symbol] | [symbol] | [symbol] | [symbol] | 5 | [symbol] | [symbol] | + | 7025 | protein_codin | [symbol] | — |
| ENSMUSG0 | [symbol] | [symbol] | [symbol] | [symbol] | [symbol] | [symbol] | 16 | [symbol] | [symbol] | − | 12996 | protein_codin | [symbol] | — |
| ENSMUSG0 | [symbol] | [symbol] | [symbol] | [symbol] | [symbol] | [symbol] | 9 | [symbol] | [symbol] | + | 1543 | protein_codin | [symbol] | — |
| ENSMUSG0 | [symbol] | [symbol] | [symbol] | [symbol] | [symbol] | [symbol] | 8 | [symbol] | [symbol] | + | 6153 | protein_codin | [symbol] | — |
| ENSMUSG0 | [symbol] | [symbol] | [symbol] | [symbol] | [symbol] | [symbol] | 9 | [symbol] | [symbol] | − | 11152 | protein_codin | [symbol] | — |
| ENSMUSG0 | [symbol] | [symbol] | [symbol] | [symbol] | [symbol] | [symbol] | 18 | [symbol] | [symbol] | − | 3520 | protein_codin | [symbol] | — |
| ENSMUSG0 | [symbol] | [symbol] | [symbol] | [symbol] | [symbol] | [symbol] | 12 | [symbol] | [symbol] | − | 2583 | protein_codin | [symbol] | — |
| ENSMUSG0 | [symbol] | [symbol] | [symbol] | [symbol] | [symbol] | [symbol] | 4 | [symbol] | [symbol] | + | 6216 | protein_codin | [symbol] | — |
| ENSMUSG0 | [symbol] | [symbol] | [symbol] | [symbol] | [symbol] | [symbol] | 7 | [symbol] | [symbol] | + | 1304 | protein_codin | [symbol] | T-box |
| ENSMUSG0 | [symbol] | [symbol] | [symbol] | [symbol] | [symbol] | [symbol] | 4 | [symbol] | [symbol] | − | 3516 | protein_codin | [symbol] | — |
| ENSMUSG0 | [symbol] | [symbol] | [symbol] | [symbol] | [symbol] | [symbol] | 17 | [symbol] | [symbol] | − | 1407 | protein_codin | [symbol] | — |
| ENSMUSG0 | [symbol] | [symbol] | [symbol] | [symbol] | [symbol] | [symbol] | 16 | [symbol] | [symbol] | − | 6700 | protein_codin | [symbol] | ZBTB |
| ENSMUSG0 | [symbol] | [symbol] | [symbol] | [symbol] | [symbol] | [symbol] | 19 | [symbol] | [symbol] | + | 4209 | protein_codin | [symbol] | — |
| ENSMUSG0 | [symbol] | [symbol] | [symbol] | [symbol] | [symbol] | [symbol] | 15 | [symbol] | [symbol] | − | 4662 | protein_codin | [symbol] | — |
| ENSMUSG0 | [symbol] | [symbol] | [symbol] | [symbol] | [symbol] | [symbol] | 4 | [symbol] | [symbol] | − | 8487 | protein_codin | [symbol] | zf- |
| | | | | | | | | | | | | | | C2H2 |
| ENSMUSG0 | [symbol] | [symbol] | [symbol] | [symbol] | [symbol] | [symbol] | 10 | [symbol] | [symbol] | − | 2789 | protein_codin | [symbol] | — |
| ENSMUSG0 | [symbol] | [symbol] | [symbol] | [symbol] | [symbol] | [symbol] | X | [symbol] | [symbol] | − | 1195 | protein_codin | [symbol] | — |
| ENSMUSG0 | [symbol] | [symbol] | [symbol] | [symbol] | [symbol] | [symbol] | 11 | [symbol] | [symbol] | − | 4847 | protein_codin | [symbol] | — |
| ENSMUSG0 | [symbol] | [symbol] | [symbol] | [symbol] | [symbol] | [symbol] | 6 | [symbol] | [symbol] | + | 3419 | protein_codin | [symbol] | — |
| ENSMUSG0 | [symbol] | [symbol] | [symbol] | [symbol] | [symbol] | [symbol] | 7 | [symbol] | [symbol] | + | 2497 | protein_codin | [symbol] | — |
| ENSMUSG0 | [symbol] | [symbol] | [symbol] | [symbol] | [symbol] | [symbol] | 11 | [symbol] | [symbol] | − | 1732 | protein_codin | [symbol] | — |
| ENSMUSG0 | [symbol] | [symbol] | [symbol] | [symbol] | [symbol] | [symbol] | 3 | [symbol] | [symbol] | + | 8815 | protein_codin | [symbol] | — |
| ENSMUSG0 | [symbol] | [symbol] | [symbol] | [symbol] | [symbol] | [symbol] | 15 | [symbol] | [symbol] | − | 4740 | protein_codin | [symbol] | — |
| ENSMUSG0 | [symbol] | [symbol] | [symbol] | [symbol] | [symbol] | [symbol] | 9 | [symbol] | [symbol] | − | 1582 | protein_codin | [symbol] | — |
| ENSMUSG0 | [symbol] | [symbol] | [symbol] | [symbol] | [symbol] | [symbol] | 12 | [symbol] | [symbol] | − | 6895 | protein_codin | [symbol] | — |
| ENSMUSG0 | [symbol] | [symbol] | [symbol] | [symbol] | [symbol] | [symbol] | 13 | [symbol] | [symbol] | − | 3682 | protein_codin | [symbol] | — |
| ENSMUSG0 | [symbol] | [symbol] | [symbol] | [symbol] | [symbol] | [symbol] | 11 | [symbol] | [symbol] | + | 3626 | protein_codin | [symbol] | T-box |
| ENSMUSG0 | [symbol] | [symbol] | [symbol] | [symbol] | [symbol] | [symbol] | 17 | [symbol] | [symbol] | − | 5505 | protein_codin | [symbol] | — |
| ENSMUSG0 | [symbol] | [symbol] | [symbol] | [symbol] | [symbol] | [symbol] | 8 | [symbol] | [symbol] | + | 2674 | protein_codin | [symbol] | — |
| ENSMUSG0 | [symbol] | [symbol] | [symbol] | [symbol] | [symbol] | [symbol] | 7 | [symbol] | [symbol] | + | 6078 | protein_codin | [symbol] | zf- |
| | | | | | | | | | | | | | | C2H2 |
| ENSMUSG0 | [symbol] | [symbol] | [symbol] | [symbol] | [symbol] | [symbol] | 2 | [symbol] | [symbol] | − | 3723 | protein_codin | [symbol] | — |
| ENSMUSG0 | 0 | [symbol] | [symbol] | [symbol] | [symbol] | [symbol] | 4 | [symbol] | [symbol] | + | 3609 | protein_codin | [symbol] | — |
| ENSMUSG0 | 0 | [symbol] | [symbol] | [symbol] | [symbol] | [symbol] | 11 | [symbol] | [symbol] | − | 5149 | protein_codin | [symbol] | — |
| ENSMUSG0 | 0 | [symbol] | [symbol] | [symbol] | [symbol] | [symbol] | 2 | [symbol] | [symbol] | + | 7639 | protein_codin | [symbol] | — |
| ENSMUSG0 | 0 | [symbol] | [symbol] | [symbol] | [symbol] | [symbol] | 6 | [symbol] | [symbol] | − | 6014 | protein_codin | [symbol] | Homeo- |
| | | | | | | | | | | | | | | box |
| ENSMUSG0 | 0 | [symbol] | [symbol] | [symbol] | [symbol] | [symbol] | 2 | [symbol] | [symbol] | + | 2580 | protein_codin | [symbol] | — |
| ENSMUSG0 | [symbol] | [symbol] | [symbol] | [symbol] | [symbol] | [symbol] | 5 | [symbol] | [symbol] | + | 10604 | protein_codin | [symbol] | — |
| ENSMUSG0 | 0 | [symbol] | [symbol] | [symbol] | [symbol] | [symbol] | X | [symbol] | [symbol] | − | 468 | protein_codin | [symbol] | — |
| ENSMUSG0 | 0 | [symbol] | [symbol] | [symbol] | [symbol] | [symbol] | 17 | [symbol] | [symbol] | − | 5703 | protein_codin | [symbol] | — |
| ENSMUSG0 | 0 | [symbol] | [symbol] | [symbol] | [symbol] | [symbol] | X | [symbol] | [symbol] | − | 627 | protein_codin | [symbol] | — |

[symbol]

indicates data missing or illegible when filed

TABLE 8

| Cellular Therapy | | Serum | | |
|---|---|---|---|---|
| Biobank ID | Sample collection time point | volume(uL) | Response | Treatment |
| 18-0025-035, RF | | | CR | Tisagenlecleucel |
| | Immediately prior to start of LD chemo (i.e. Day −5) | 800 | | |
| | d + 2 | 800 | | |
| | d + 14 | 800 | | |
| | d + 21 | 800 | | |
| 18-0025-033, PB | | | CR | Axicabtagene Ciloleucel |
| | Immediately prior to start of LD chemo (i.e. Day −5) | 800 | | |
| | d + 2 | 800 | | |
| | d + 14 | 800 | | |
| | d + 28 | 800 | | |
| 18-0025-031, EG | | | CR | Axicabtagene Ciloleucel |
| | Immediately prior to start of LD chemo (i.e. Day −5) | 800 | | |
| | d + 4 | 800 | | |
| | d + 14 | 800 | | |
| | d + 30 | 800 | | |
| 18-0025-049, MC | | | CR | Axicabtagene Ciloleucel |
| | Immediately prior to start of LD chemo (i.e. Day −5) | 800 | | |
| | d + 2 | 800 | | |
| | d + 12 | 800 | | |
| | d + 28 | 800 | | |
| 18-0025-086, LT | | | CR | Tisagenlecleucel |
| | Immediately prior to start of LD chemo (i.e. Day −5) | 800 | | |
| | d + 6 | 800 | | |
| | d + 14 | 800 | | |
| | d + 28 | 800 | | |
| 18-0025-032, LB | | | PD | Axicabtagene Ciloleucel |
| | Immediately prior to start of LD chemo (i.e. Day −5) | 800 | | |
| | d + 4 | 800 | | |
| | d + 10 | 800 | | |
| | d + 21 | 800 | | |
| 18-0025-051, MH | | | PD | Tisagenlecleucel |
| | Immediately prior to start of LD chemo (i.e. Day −5) | 800 | | |
| | d + 4 | 800 | | |
| | d + 10 | 800 | | |
| | d + 28 | 800 | | |
| 18-0025-075, KY | | | PD | Tisagenlecleucel |
| | Immediately prior to start of LD chemo (i.e. Day −5) | 800 | | |
| | d + 2 | 800 | | |
| | d + 10 | 800 | | |
| | d + 28 | 800 | | |
| 18-0025-112, ND | | | PD | Axicabtagene Ciloleucel |
| | Immediately prior to start of LD chemo (i.e. Day −5) | 800 | | |
| | d + 4 | 800 | | |
| | d + 14 | 800 | | |
| | d + 28 | 800 | | |
| 18-0025-122, TM | | | Suspected | Lisocaptagene maraleucel |
| | Immediately prior to start of LD chemo (i.e. Day −5) | 800 | | |
| | d + 4 | 800 | | |
| | d + 14 | 800 | | |
| | d + 28 | 800 | | |

1. A method of treating, preventing, reducing the likelihood, or reducing the severity of cancer in a subject in need thereof comprising administering an effective amount of a trans-vaccenic acid (TVA) or an active TVA derivative to the subject.

2. The method of claim 1, wherein the active TVA derivative can enhance CD8+ T cell activity and/or can enhance antitumor immunity.

3. The method of claim 1 or 2, wherein the TVA or the active TVA derivative comprises a compound of formula (I): 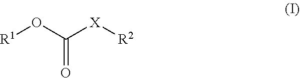
wherein:
R¹ is selected from hydrogen and C₁-C₄ alkyl;
X is selected from C₁-C₂₄ alkylene, C₂-C₂₄ alkenylene, and C₂-C₂₄ alkynylene;
R² is selected from hydrogen, optionally substituted aryl, optionally substituted heteroaryl, C₁-C₄ alkoxy, hydroxy, and halo.

4. The method of claim 3, wherein R¹ is hydrogen.

5. The method of claim 3, wherein X is selected from C₈-C₂₄ alkylene, C₈-C₂₄ alkenylene, C₈-C₂₄ alkynylene.

6. The method of claim 3, wherein R² is selected from hydrogen and optionally substituted aryl.

7. The method of claim 3, wherein the TVA or the active TVA derivative is selected from:

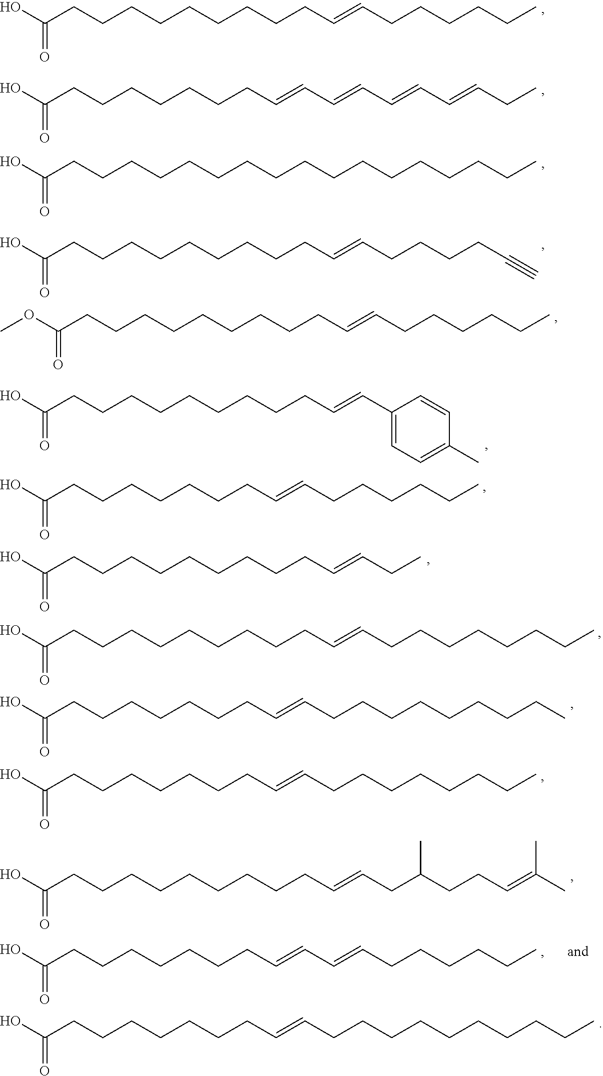

8. The method of claim 1, wherein the TVA or the active TVA derivative is:

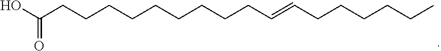 [SMILES: CCCCCC/C=C/CCCCCCCCCC(=O)O]

9. The methods of any of claims 1-8, wherein administration of the TVA or the active TVA derivative comprises oral, intravenous, transdermal, or subcutaneous administration.

10. The methods of any of claims 1-9, wherein administration of the TVA or the active TVA derivative occurs once or more weekly.

11. The methods of any of claims 1-9, wherein administration of the TVA or the active TVA derivative occurs once or more daily.

12. The method of claim 1, wherein the cancer is melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma.

13. The method of claim 1, wherein the subject is a human.

14. The methods of any of claims 1-13, wherein the TVA or the active TVA derivative is co-administered with one or more additional therapeutic or prophylactic agents.

15. The method of claim 14, wherein the one or more additional therapeutic agents are selected from chemotherapeutics and immunotherapeutics.

16. The method of any of claims 1-15, wherein the TVA or the active TVA derivative is co-administered with a T-cell-based immunotherapeutic.

17. The method of claim 16, wherein the T-cell-based immunotherapeutic is selected from a therapy comprising the administration of immune checkpoint inhibitor, CAR-T cell therapy, monoclonal antibody therapy, and bispecific T-cell engager therapy.

18. The method of any of claims 1-17, wherein the TVA or active TVA derivative is co-administered with:
an immune checkpoint inhibitor that binds to and inhibits the activity of an immune checkpoint protein; and wherein the immune checkpoint protein is selected from the group consisting of CTLA4, PD-1, PD-L1, PD-L2, A2AR, B7-H3, B7-H4, BTLA, KIR, LAG3, TIM-3, VISTA.

19. The method of any of claims 1-18, wherein the TVA or active TVA derivative is co-administered with an immune checkpoint inhibitor selected from the group consisting of nivolumab, pembrolizumab, pidilizumab, AMP-224, AMP-514, STI-A1110, TSR-042, RG-7446, BMS-936559, BMS-936558, MK-3475, MPDL3280A, MEDI-4736, MSB-0020718C, AUR-012, STI-A1010.

20. Use of an effective dose of trans-vaccenic acid (TVA) or active TVA derivative for treating a subject suffering from cancer.

21. The use of claim 20, wherein the TVA or active TVA derivative is co-administered with an immunotherapeutic selected from a therapy comprising the administration of immune checkpoint inhibitor, CAR-T cell therapy, monoclonal antibody therapy, and bispecific T-cell engager therapy.

22. Use of an effective dose of trans-vaccenic acid (TVA) or active TVA derivative in the manufacture of a medicament for use in a method of treating a subject suffering from cancer.

21. The use of claim 22, wherein the method further comprises an immune checkpoint inhibitor, CAR-T cell therapy, monoclonal antibody therapy, and bispecific T-cell engager therapy.

24. A composition or kit comprising a trans-vaccenic acid (TVA) or an active TVA derivative and an additional therapeutic or prophylactic agent for the treatment of cancer.

25. The composition or kit of claim 24, wherein the active TVA derivative can enhance CD8+ T cell activity and/or can enhance antitumor immunity.

26. The composition or kit of claim 24 or 25, wherein the TVA or an active TVA derivative is a compound of formula (I): 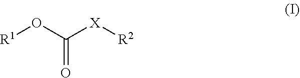
wherein:
R¹ is selected from hydrogen and C₁-C₄ alkyl;
X is selected from C₁-C₂₄ alkylene, C₂-C₂₄ alkenylene, and C₂-C₂₄ alkynylene; and
R² is selected from hydrogen, optionally substituted aryl, optionally substituted heteroaryl, C₁-C₄ alkoxy, hydroxy, and halo.

27. The composition or kit of claim 26, wherein R¹ is hydrogen.

28. The composition or kit of claim 26, wherein X is selected from C₈-C₂₄ alkylene, C₈-C₂₄ alkenylene, and C₈-C₂₄ alkynylene.

29. The composition or kit of claim 26, wherein R² is selected from hydrogen and optionally substituted aryl.

30. The composition or kit of claim 26, wherein the TVA or an active TVA derivative is selected from:

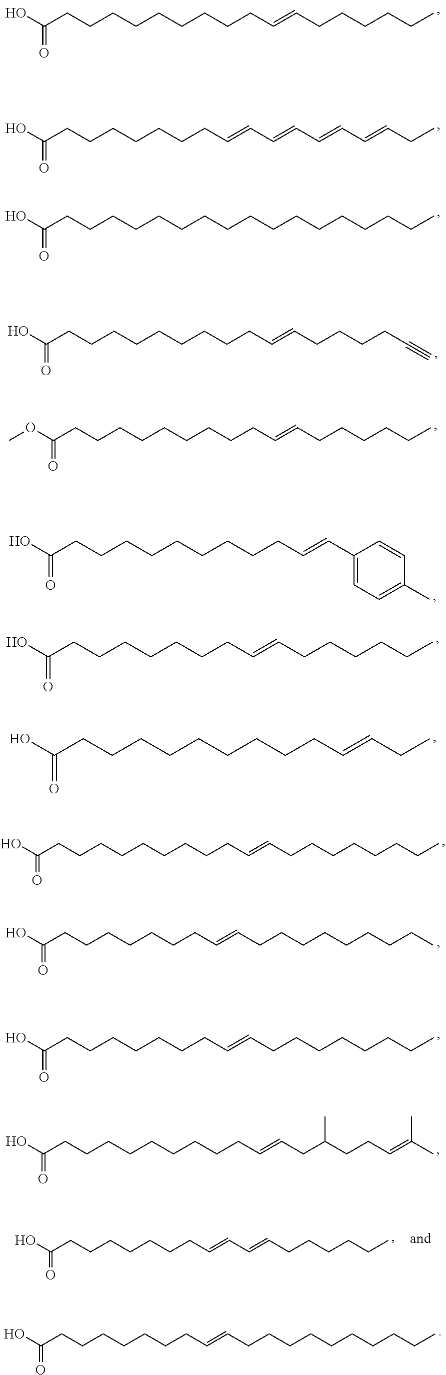

31. The composition or kit of claim 24, wherein the TVA or an active TVA derivative is:

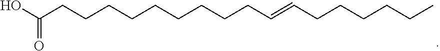 [SMILES: CCCCCC/C=C/CCCCCCCCCC(=O)O]

32. The composition or kit of claim 24, wherein the additional therapeutic agent is selected from a chemotherapeutic or immunotherapeutic.

33. The composition of claim 32, wherein the additional therapeutic agent is a T-cell-based immunotherapeutic.

34. The composition of claim 33, wherein the T-cell-based immunotherapeutic is selected from a therapy comprising the administration of immune checkpoint inhibitor, CAR-T cell therapy, monoclonal antibody therapy, and bispecific T-cell engager therapy.

35. A TVA-containing or active-TVA-derivative-containing composition for use in a method of treating cancer in a human subject.

36. The composition of claim 35, wherein the method comprises co-administering the TVA or active TVA derivative to the subject with an immunotherapeutic selected from a therapy comprising the administration of immune checkpoint inhibitor, CAR-T cell therapy, monoclonal antibody therapy, and bispecific T-cell engager therapy.

37. A method of preventing, reducing the likelihood, or reducing the severity of cancer in a subject comprising administering an effective amount of a trans-vaccenic acid (TVA) or an active TVA derivative to the subject.

38. A method of treating or preventing, reducing the likelihood, or reducing the severity of cancer in a subject comprising administering a composition that results in inhibition of GPR43 activity or expression.

39. The method of claim 38, wherein the composition comprises a trans-vaccenic acid (TVA) or an active TVA derivative.

40. The method of claim 38, wherein the composition comprises a nucleic acid inhibitor of GPR43 expression or activity.

41. The method of claim 40, wherein the composition comprises a siRNA, shRNA, antisense RNA, or CRIPSR system to inhibit expression of GPR43.

42. The method of claim 38, wherein the composition comprises a small molecule inhibitor of GPR43 activity.

43. The method of claim 42, wherein the small molecule inhibitor of GPR43 activity is a GPR43 antagonist.

44. The method of claim 43, wherein the GPR43 antagonist has the structure:

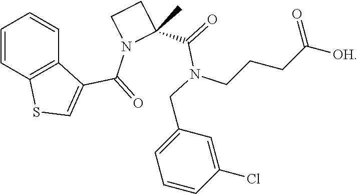 [SMILES: C[C@]1(C(=O)N(CCCC(=O)O)Cc2cccc(Cl)c2)CCN1C(=O)c1csc2ccccc12]

45. The method of claim 42, wherein the small molecule inhibitor of GPR43 activity is an 5 inverse agonist of GPR43.

46. The method of claim 43, wherein the inverse agonist of GPR43 has the structure: 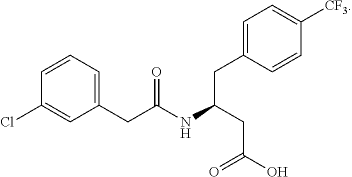 [SMILES: O=C(O)C[C@H](Cc1ccc(C(F)(F)F)cc1)NC(=O)Cc1cccc(Cl)c1]


SEQUENCE LISTING (44 sequences)

SEQ ID NO: 1 - DNA, 25 bp, Synthetic construct
  source 1..25: mol_type = other DNA
caccggcgcgcacacgatctttggt

SEQ ID NO: 2 - DNA, 25 bp, Synthetic construct
  source 1..25: mol_type = other DNA
aaacaccaaagatcgtgtgcgcgcc

SEQ ID NO: 3 - DNA, 25 bp, Synthetic construct
  source 1..25: mol_type = other DNA
caccggcagcgatcactccatacag

SEQ ID NO: 4 - DNA, 25 bp, Synthetic construct
  source 1..25: mol_type = other DNA
aaacctgtatggagtgatcgctgcc

SEQ ID NO: 5 - DNA, 25 bp, Synthetic construct
  source 1..25: mol_type = other DNA
caccgctgaactcaactgagcaggt

SEQ ID NO: 6 - DNA, 25 bp, Synthetic construct
  source 1..25: mol_type = other DNA
aaacacctgctcagttgagttcagc

SEQ ID NO: 7 - DNA, 22 bp, Synthetic construct
  source 1..22: mol_type = other DNA
ggatgggagggaggtgaactgg

SEQ ID NO: 8 - DNA, 22 bp, Synthetic construct
  source 1..22: mol_type = other DNA
ggtcttcttgtggcaggcgatg

SEQ ID NO: 9 - DNA, 22 bp, Synthetic construct
  source 1..22: mol_type = other DNA
aggtcgtgaggtcgtggagaac

SEQ ID NO: 10 - DNA, 24 bp, Synthetic construct
  source 1..24: mol_type = other DNA
ggataagaggctggtggtgttgac

SEQ ID NO: 11 - DNA, 24 bp, Synthetic construct
  source 1..24: mol_type = other DNA
gcctcttctcattcctgcttgtgg

SEQ ID NO: 12 - DNA, 24 bp, Synthetic construct
  source 1..24: mol_type = other DNA
gtggtttgtgagtgtgagggtctg

SEQ ID NO: 13 - DNA, 25 bp, Synthetic construct
  source 1..25: mol_type = other DNA
gatgggtctttgtcagcaggagaag

SEQ ID NO: 14 - DNA, 23 bp, Synthetic construct
  source 1..23: mol_type = other DNA
agcaggaggtggtggtgtatcag

SEQ ID NO: 15 - DNA, 22 bp, Synthetic construct
  source 1..22: mol_type = other DNA
agccagcacctgatgagacctc

SEQ ID NO: 16 - DNA, 25 bp, Synthetic construct
  source 1..25: mol_type = other DNA
tgtcaccaaagaaaggatggaagcc

SEQ ID NO: 17 - DNA, 24 bp, Synthetic construct
  source 1..24: mol_type = other DNA
atcactaagcaaggacggcgaatg

SEQ ID NO: 18 - DNA, 25 bp, Synthetic construct
  source 1..25: mol_type = other DNA
accaagaccacatccacaaacatcc

SEQ ID NO: 19 - DNA, 23 bp, Synthetic construct
  source 1..23: mol_type = other DNA
caaagtgccagtgaaccccagac

SEQ ID NO: 20 - DNA, 22 bp, Synthetic construct
  source 1..22: mol_type = other DNA
acagagagggtcacagccagtc

SEQ ID NO: 21 - DNA, 25 bp, Synthetic construct
  source 1..25: mol_type = other DNA
ccacactgctcatcttcttcgtctg

SEQ ID NO: 22 - DNA, 23 bp, Synthetic construct
  source 1..23: mol_type = other DNA
acggactctcacggctgacatag

SEQ ID NO: 23 - DNA, 24 bp, Synthetic construct
  source 1..24: mol_type = other DNA
ctgtatggagtgatcgctgctctg

SEQ ID NO: 24 - DNA, 25 bp, Synthetic construct
  source 1..25: mol_type = other DNA
ctgctcttgggtgaagttctcgtag

SEQ ID NO: 25 - DNA, 21 bp, Synthetic construct
  source 1..21: mol_type = other DNA
gcagctcgcatcctgtgtcac

SEQ ID NO: 26 - DNA, 22 bp, Synthetic construct
  source 1..22: mol_type = other DNA
ctgctgtgcttccgctgtagag

SEQ ID NO: 27 - DNA, 24 bp, Synthetic construct
  source 1..24: mol_type = other DNA
gctggctaacaatggtacggatgg

SEQ ID NO: 28 - DNA, 25 bp, Synthetic construct
  source 1..25: mol_type = other DNA
aacttggttgctgggcactagaatc

SEQ ID NO: 29 - DNA, 23 bp, Synthetic construct
  source 1..23: mol_type = other DNA
ggctctcggagtcctcatctacg

SEQ ID NO: 30 - DNA, 22 bp, Synthetic construct
  source 1..22: mol_type = other DNA
cgcagcaggtccttcaagtcag

SEQ ID NO: 31 - DNA, 24 bp, Synthetic construct
  source 1..24: mol_type = other DNA
gccatctcgttctcgttctcatcc

SEQ ID NO: 32 - DNA, 24 bp, Synthetic construct
  source 1..24: mol_type = other DNA
ttctccaccagtgaaagccaaagg

SEQ ID NO: 33 - DNA, 23 bp, Synthetic construct
  source 1..23: mol_type = other DNA
agagccgacagagacagacacag

SEQ ID NO: 34 - DNA, 25 bp, Synthetic construct
  source 1..25: mol_type = other DNA
tcggtgagctgtttgatctctttgc

SEQ ID NO: 35 - DNA, 22 bp, Synthetic construct
  source 1..22: mol_type = other DNA
tcaagtaccgactccgctaccg

SEQ ID NO: 36 - DNA, 25 bp, Synthetic construct
  source 1..25: mol_type = other DNA
ctgagctgacacctggatgcaatac

SEQ ID NO: 37 - DNA, 23 bp, Synthetic construct
  source 1..23: mol_type = other DNA
gaagctggctggacaacagtacc

SEQ ID NO: 38 - DNA, 25 bp, Synthetic construct
  source 1..25: mol_type = other DNA
aagggcagaaagaagagcagaatgg

SEQ ID NO: 39 - DNA, 24 bp, Synthetic construct
  source 1..24: mol_type = other DNA
aatcgcacccacttccctttcttc

SEQ ID NO: 40 - DNA, 22 bp, Synthetic construct
  source 1..22: mol_type = other DNA
gcccagcagtgagacgacaaag

SEQ ID NO: 41 - DNA, 24 bp, Synthetic construct
  source 1..24: mol_type = other DNA
cagcctttctccgtggacacatac

SEQ ID NO: 42 - DNA, 25 bp, Synthetic construct
  source 1..25: mol_type = other DNA
tagagcaatgagacagagggtgagg

SEQ ID NO: 43 - DNA, 25 bp, Synthetic construct
  source 1..25: mol_type = other DNA
tccatattccagcagaccacctacc

SEQ ID NO: 44 - DNA, 25 bp, Synthetic construct
  source 1..25: mol_type = other DNA
agaggcaatcttgagcatgtcacag